SEQUENCE LISTING (12 sequences)

SEQ ID NO: 1 - protein, 24 aa, Artificial Sequence
  T2A
LEGGGEGRGSLLTCGDVEENPGPR

SEQ ID NO: 2 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 3 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 4 - protein, 18 aa, Artificial Sequence
  T2A
EGRGSLLTCGDVEENPGP

SEQ ID NO: 5 - protein, 22 aa, Artificial Sequence
  P2A
GSGATNFSLLKQAGDVEENPGP

SEQ ID NO: 6 - protein, 19 aa, Artificial Sequence
  P2A
ATNFSLLKQAGDVEENPGP

SEQ ID NO: 7 - protein, 20 aa, Artificial Sequence
  E2A
QCTNYALLKLAGDVESNPGP

SEQ ID NO: 8 - protein, 22 aa, Artificial Sequence
  F2A
VKQTLNFDLLKLAGDVESNPGP

SEQ ID NO: 9 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 10 - protein, 22 aa, Artificial Sequence
  GMCSFR alpha chain signal sequence
MLLLVTSLLLCELPHPAFLLIP

SEQ ID NO: 11 - protein, 18 aa, Artificial Sequence
  CD8 alpha signal peptide
MALPVTALLLPLALLLHA

SEQ ID NO: 12 - protein, 16 aa, Artificial Sequence
  CD33 signal peptide
MPLLLLLPLLWAGALA

(19) United States
(12) Patent Application Publication
(10) Pub. No.: US 2022/0412954 A1
(43) Pub. Date: Dec. 29, 2022

(54) METHODS OF DETERMINING ATTRIBUTES OF THERAPEUTIC T CELL COMPOSITIONS

(71) Applicant: Juno Therapeutics, Inc., Seattle, WA (US)

(72) Inventors: Ronald James HAUSE, JR., Seattle, WA (US); Afshin MASHADI-HOSSEIN, Summit, NJ (US); Yue JIANG, Seattle, WA (US)

(73) Assignee: Juno Therapeutics, Inc., Seattle, WA (US)

(21) Appl. No.: 17/774,464

(22) PCT Filed: Nov. 4, 2020
(86) PCT No.: PCT/US2020/058985

§ 371 Date: May 4, 2022

Related U.S. Application Data
(60) Provisional application No. 62/945,091, filed on Dec. 6, 2019.
(60) Provisional application No. 62/931,194, filed on Nov. 5, 2019.

(51) Int. Cl. G01N 33/50; A61K 35/17; C07K 14/725; C12N 5/0783; G16H 20/17; G16H 50/70

(52) U.S. Cl. CPC G01N 33/505; A61K 35/17; C07K 14/7051; C12N 5/0636; G16H 20/17; G16H 50/70; C12N 2510/00

(57) ABSTRACT

Provided are methods for determining or predicting attributes of therapeutic cell compositions in connection with cell therapy. The cells of the therapeutic cell composition express recombinant receptors such as chimeric receptors, e.g. chimeric antigen receptors (CARs) or other transgenic receptors such as T cell receptors (TCRs). The methods provide for the identification of correlations between input composition (e.g., starting material derived from subjects for producing a cell therapy) attributes and therapeutic cell composition attributes.

### CROSS-REFERENCE TO RELATED APPLICATIONS

This application claims priority from U.S. provisional application 62/931,194, filed Nov. 5, 2019, entitled “METHODS OF DETERMINING ATTRIBUTES OF THERAPEUTIC T CELL COMPOSITIONS,” and U.S. provisional application No. 62/945,091, filed Dec. 6, 2019, entitled “METHODS OF DETERMINING ATTRIBUTES OF THERAPEUTIC T CELL COMPOSITIONS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042014040SeqList.txt, created Nov. 4, 2020, which is 8,933 bytes in size. The information in the electronic format of the Sequence Listing is incorporated by reference in its entirety.

### FIELD

The present disclosure relates to methods for determining or predicting attributes of therapeutic cell compositions in connection with cell therapy. The cells of the therapeutic cell composition express recombinant receptors such as chimeric receptors, e.g. chimeric antigen receptors (CARs) or other transgenic receptors such as T cell receptors (TCRs). The methods provide for the identification of correlations between input composition (e.g., starting material derived from subjects for producing a cell therapy) attributes and therapeutic cell composition attributes.

### BACKGROUND

Various immunotherapy and/or cell therapy methods are available for treating diseases and conditions. For example, adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be beneficial in the treatment of cancer or other diseases or disorders. Improved approaches are needed for characterizing effective therapeutic compositions, such as in connection with methods for ex vivo production of the compositions, and for treating a subject with a cell therapy. Provided herein are methods that address such needs.

### SUMMARY

Provided herein are methods of predicting attributes of a cell composition, the methods including: (a) determining a percentage, number, ratio, and/or proportion of cells in an input cell composition that have first attributes, wherein the first attributes include cell phenotypes, and wherein the input composition includes T cells selected from a biological sample from a subject; and (b) applying the first attributes as input to a process configured to predict, based on the first attributes, a percentage, number, ratio and/or proportion of cells in a therapeutic cell composition that have second attributes, wherein the second attributes include cell phenotypes and recombinant receptor-dependent activity, and wherein: the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells.

In some aspects, provided herein are methods of predicting attributes of a therapeutic cell composition, the method including: (a) determining a percentage, number, ratio, and/or proportion of T cells in an input composition that have first attributes, wherein the first attributes include T cell phenotypes, and wherein the input composition includes T cells selected from a biological sample from a subject; and (b) applying the first attributes as input to a process configured to predict, based on the first attributes, a percentage, number, ratio and/or proportion of T cells in a therapeutic cell composition that have second attributes, wherein: the therapeutic cell composition includes T cells expressing the recombinant receptor and is to be produced from cells of the input composition; the second attributes include T cell phenotypes and recombinant receptor-dependent activity; and the process includes a canonical correlation analysis statistical learning model trained on training data including (i) the percentage, number, ratio and/or proportion of T cells that have the first attributes from each of a plurality of input compositions including T cells and (ii) the percentage, number, ratio and/or proportion of T cells that have the second attributes from each of a plurality of therapeutic cell compositions, wherein each of the therapeutic cells compositions includes T cells expressing the recombinant receptor and has been produced from one of the input compositions.

In some embodiments, the methods further include (c) determining, based on the predicted second attributes, whether the therapeutic cell composition is predicted to have a desired attribute.

In some aspects, provided herein are methods of predicting attributes of a therapeutic cell composition, the method including: (a) determining a percentage, number, ratio, and/or proportion of T cells in an input composition that have first attributes, wherein the first attributes include T cell phenotypes, and wherein the input composition includes T cells selected from a biological sample from a subject; (b) applying the first attributes as input to a process configured to predict, based on the first attributes, a percentage, number, ratio and/or proportion of T cells in a therapeutic cell composition that have one second attribute, wherein: the therapeutic cell composition includes T cells expressing the recombinant receptor and is to be produced from cells of the input composition; the one second attribute includes a cell phenotype or recombinant receptor-dependent activity; and the process includes a lasso regression statistical learning model trained on training data including (i) the percentage, number, ratio and/or proportion of T cells that have the first attributes from each of a plurality of input compositions including T cells and (ii) the percentage, number, ratio and/or proportion of T cells that have the one second attribute from each of a plurality of therapeutic cell compositions, wherein each of the therapeutic cells compositions includes T cells expressing the recombinant receptor and has been produced from one of the input compositions. In some embodiments, the method further include (c) determining, based on the one predicted second attribute, whether the therapeutic cell composition is predicted to have a desired attribute.

In some embodiments, if the therapeutic cell composition is predicted to have the desired attribute, the therapeutic cell composition is manufactured from the input composition using a first manufacturing process; or if the therapeutic cell composition is predicted to not have the desired attribute, selecting a second manufacturing process to manufacture the therapeutic cell composition from the input composition. In some embodiments, the second manufacturing process is associated with producing a therapeutic cell composition that has the desired attribute. In some embodiments, the second manufacturing process has an increased likelihood of producing a therapeutic cell composition that has the desired attribute. In some embodiments, the second manufacturing process increases the likelihood of producing a therapeutic cell composition that has the desired attribute. In some embodiments, the second manufacturing process includes one or more steps that are altered compared to steps of the first manufacturing process.

In some embodiments, if the therapeutic cell composition is predicted to have a desired attribute, a predetermined treatment regimen including the therapeutic cell composition is administered to a subject; or if the therapeutic cell composition is predicted to not have a desired attribute, the predetermined treatment regimen including the therapeutic cell composition is altered and the altered treatment regimen including the therapeutic cell composition is administered to the subject. In some embodiments, if the therapeutic cell composition is predicted to have the desired attribute, a subject is selected to be administered a predetermined treatment regimen including the therapeutic cell composition. In some embodiments, if the therapeutic cell composition is predicted to not have the desired attribute, a subject is selected to be administered an altered treatment regimen including the therapeutic cell composition. In some aspects, provided is a method of treating a subject selected to be administered the predetermined therapeutic regiment, and the method includes administering the therapeutic cell composition in accord with the predetermined treatment regimen. In some aspects, provided is a method of treating a subject selected to be administered the altered treatment regiment, and the method includes administering the therapeutic cell composition in accord with the altered treatment regimen.

In some of any embodiments, the first attributes include T cell phenotypes that are phenotypes positive or negative for CCR7, CD27, CD28, CD45RA, or an apoptotic marker. In some of any embodiments, the T cell phenotype(s) of the second attributes are phenotypes positive or negative for CCR7, CD27, CD28, CD45RA, an apoptotic marker, positive recombinant receptor expression (recombinant receptor+), optionally CAR+, viability, viable cell concentration, vector copy number (VCN); and/or the recombinant receptor-dependent activity of the second attribute is recombinant receptor-dependent production of a cytokine or a cytotoxic activity. In some embodiments, the apoptotic marker is activated caspase 3 (3CAS) or annexin V.

Provided herein are methods of manufacturing a therapeutic cell composition, the methods including: (a) selecting T cells from a biological sample from a subject to produce an input composition including T cells; (b) determining a percentage, number, ratio, or proportion of T cells in the input composition having first attributes, wherein the first attributes include T cell phenotypes; (c) applying the first attributes as input to a process configured to predict, based on the first attributes, a percentage, number, ratio, or proportion of T cells in a therapeutic cell composition that have second attributes, wherein: the therapeutic cell composition includes T cells expressing the recombinant receptor and is to be produced from cells of the input composition; the second attributes include T cell phenotypes and recombinant receptor-dependent activity; and the process includes a canonical correlation analysis statistical learning model trained on training data including (i) the percentage, number, ratio and/or proportion of T cells that have the first attributes from each of a plurality of input compositions including T cells and (ii) the percentage, number, ratio and/or proportion of T cells that have the second attributes from each of a plurality of therapeutic cell compositions, wherein each of the therapeutic cells compositions includes T cells expressing the recombinant receptor and has been produced from one of the input compositions; (d) determining, based on the predicted second attributes, whether the T cells of the therapeutic cell composition will have a desired attribute; and (e) manufacturing the therapeutic cell composition, wherein: (i) if the therapeutic cell composition is predicted to have the desired attribute, the therapeutic cell composition is manufactured from the input composition using a first manufacturing process; or (ii) if the therapeutic cell composition is predicted to not have the desired attribute, selecting a second manufacturing process to manufacture the therapeutic cell composition from the input composition. In some embodiments, the second manufacturing process is associated with producing a therapeutic cell composition that has the desired attribute. In some embodiments, the second manufacturing process has an increased likelihood of producing a therapeutic cell composition that has the desired attribute. In some embodiments, the second manufacturing process increases the likelihood of producing a therapeutic cell composition that has the desired attribute. In some embodiments, the second manufacturing process includes one or more steps that are altered compared to steps of the first manufacturing process.

Provided herein are methods of manufacturing a therapeutic cell composition, the methods including: (a) selecting T cells from a biological sample from a subject to produce an input composition including T cells; (b) determining a percentage, number, ratio, or proportion of T cells in the input composition having first attributes, wherein the first attributes include T cell phenotypes; (c) applying the first attributes as input to a process configured to predict, based on the first attributes, a percentage, number, ratio, or proportion of T cells in a therapeutic cell composition that have one second attribute, wherein: the therapeutic cell composition includes T cells expressing the recombinant receptor and is to be produced from cells of the input composition; the one second attributes include T cell phenotypes and recombinant receptor-dependent activity; and the process includes a lasso regression statistical learning model trained on training data including (i) the percentage, number, ratio and/or proportion of T cells that have the first attributes from each of a plurality of input compositions including T cells and (ii) the percentage, number, ratio and/or proportion of T cells that have the one second attribute from each of a plurality of therapeutic cell compositions, wherein each of the therapeutic cells compositions includes T cells expressing the recombinant receptor and has been produced from one of the input compositions; (d) determining, based on the predicted one second attribute, whether the T cells of the therapeutic cell composition will have a desired attribute; and (e) manufacturing the therapeutic cell composition, wherein: (i) if the therapeutic cell composition is predicted to have the desired attribute, the therapeutic cell composition is manufactured from the input composition using a first manufacturing process; or (ii) if the therapeutic cell composition is predicted to not have the desired attribute, selecting a second manufacturing process to manufacture the therapeutic cell composition from the input composition. In some embodiments, the second manufacturing process has an increased likelihood of producing a therapeutic cell composition that has the desired attribute. In some embodiments, the second manufacturing process increases the likelihood of producing a therapeutic cell composition that has the desired attribute. In some embodiments, the second manufacturing process includes one or more steps that are altered compared to steps of the first manufacturing process.

In some embodiments, the second manufacturing process includes one or more steps that are altered compared to steps of the first manufacturing process. Provided herein are methods of predicting attributes of a cell composition, the method comprising: (a) determining a percentage, number, ratio, and/or proportion of cells in an input cell composition that have first attributes, wherein the first attributes comprise cell phenotypes, and wherein the input composition comprises T cells selected from a biological sample from a subject; and (b) applying the first attributes as input to a process configured to predict, based on the first attributes, a percentage, number, ratio and/or proportion of cells in a therapeutic cell composition that have second attributes, wherein the second attributes comprise cell phenotypes and recombinant receptor-dependent activity, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the second attributes are predicted for the therapeutic cell composition from the first attributes; or (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the second attributes are predicted for CD4+ and CD8+ T cells of each of the separate compositions of the therapeutic composition from the first attributes; or (iii) the input composition comprises separate compositions of CD4+ and CD8+ T cells and the therapeutic cell composition comprises a mixed composition of CD4+ and CD8+ T cells expressing the recombinant receptor, and is produced from the CD4+ and CD8+ T cell compositions of the input composition, wherein the first attributes comprise first attributes from the CD4+ and CD8+ T cell compositions of the input composition, and the second attributes are predicted for CD4+ and CD8+ cells of each of the separate compositions of therapeutic composition from the first attributes.

Provided are methods of predicting attributes of a cell composition, the methods including: (a) determining a percentage, number, ratio, and/or proportion of cells in an input cell composition that have first attributes, wherein the first attributes include cell phenotypes, and wherein the input composition includes T cells selected from a sample from a subject; (b) applying the first attributes as input to a process configured to predict, based on the first attributes, a percentage, number, ratio and/or proportion of cells in an therapeutic cell composition that have one second attribute, wherein the one second attribute includes a cell phenotype or recombinant receptor-dependent activity, and wherein: the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells. In some embodiments, the method further includes (c) determining, based on the one predicted second attribute, whether the therapeutic cell composition is predicted to have a desired attribute. In some embodiments, if the therapeutic cell composition is predicted to have a desired attribute, a predetermined treatment regimen including the therapeutic cell composition is administered to a subject; or if the therapeutic cell composition is predicted to not have a desired attribute, the predetermined treatment regimen including the therapeutic cell composition is altered and the altered treatment regimen including the therapeutic cell composition is administered to the subject.

Provided herein are methods of predicting attributes of a cell composition, the method comprising: (a) determining a percentage, number, ratio, and/or proportion of cells in an input cell composition that have first attributes, wherein the first attributes comprise cell phenotypes, and wherein the input composition comprises T cells selected from a sample from a subject; (b) applying the first attributes as input to a process configured to predict, based on the first attributes, a percentage, number, ratio and/or proportion of cells in an therapeutic cell composition that have one second attribute, wherein the one second attribute comprises a cell phenotype or recombinant receptor-dependent activity, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the one second attribute is predicted for the therapeutic cell composition from the first attributes; or (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the one second attribute is predicted for CD4+ or CD8+ T cells of the separate compositions of the therapeutic composition from the first attributes; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+ T cell compositions of the input composition, wherein the first attributes comprise first attributes from the CD4+ and CD8+ T cell compositions of the input composition, and the one second attribute is predicted for CD4+ or CD8+ T cells of the separate compositions of therapeutic composition from the first attributes.

Provided herein are methods of predicting attributes of a cell composition, the method comprising: (a) determining a percentage, number, ratio, and/or proportion of cells in an input cell composition having one or more first attributes comprising CD4+/CCR7+/CD27+, CD4+/CCR7+/CD45RA+, CD4+/CD28+/CD27-, CD8+/CCR7+CD45RA+, CD8+/CCR7+, CD4+/CCR7−/CD27−, CD8+/CCR7−/CD45RA+, and CD4+/CD28+, and wherein the input composition comprises T cells selected from a sample from a subject; (b) applying the first attributes as input to a process configured to predict, based on the first attributes, a percentage, number, ratio and/or proportion of cells in an therapeutic cell composition that have one second attribute, wherein the one second attribute comprises a cell phenotype or recombinant receptor-dependent activity, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the one second attribute is predicted for the therapeutic cell composition from the first attributes; or (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the one second attribute is predicted for CD4+ or CD8+ T cells of the separate compositions of the therapeutic composition from the first attributes; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+ T cell compositions of the input composition, wherein the first attributes comprise first attributes from the CD4+ and CD8+ T cell compositions of the input composition, and the one second attribute is predicted for CD4+ or CD8+ T cells of the separate compositions of therapeutic composition from the first attributes.

Provided are methods of treating a subject, the methods including: (a) selecting T cells from a sample from a subject to produce an input composition including T cells; (b) determining a percentage, number, ratio, and/or proportion of T cells in the input composition having first attributes, wherein the first attributes include cell phenotypes; (c) applying the first attributes as input to a process configured to predict, based on the first attributes, a percentage, number, ratio, and/or proportion of cells in a therapeutic cell composition that have second attributes, wherein the second attributes include a cell phenotype and recombinant receptor-dependent activity, and wherein the therapeutic cell composition includes the recombinant receptor, and wherein: the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells; (d) determining, based on the predicted second attributes, whether the therapeutic cell composition is predicted to have a desired attribute; and (e) administering a treatment to the subject wherein: (i) if the therapeutic cell composition is predicted to have the desired attribute, a predetermined treatment regimen including the therapeutic cell composition is administered; or (ii) if the therapeutic cell composition is predicted to not have the desired attribute, administering to the subject a treatment regimen including the therapeutic cell composition that is altered compared to the predetermined treatment regimen including the therapeutic cell composition.

Provided herein are method of treating a subject, the method comprising: (a) selecting T cells from a sample from a subject to produce an input composition comprising T cells; (b) determining a percentage, number, ratio, and/or proportion of T cells in the input composition having first attributes, wherein the first attributes comprise cell phenotypes; (c) applying the first attributes as input to a process configured to predict, based on the first attributes, a percentage, number, ratio, and/or proportion of cells in a therapeutic cell composition that have second attributes, wherein the second attributes comprises a cell phenotype and recombinant receptor-dependent activity, and wherein the therapeutic cell composition comprises the recombinant receptor, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the second attributes are predicted for the therapeutic cell composition from the first attributes; or (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the second attributes are predicted for CD4+ and CD8+ T cells of each of the separate compositions of the therapeutic composition from the first attributes; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+ T cell compositions of the input composition, wherein the first attributes comprise first attributes from the CD4+ and CD8+ T cell compositions of the input composition, and the second attributes are predicted for CD4+ and CD8+ cells of each of the separate compositions of therapeutic composition from the first attributes; (d) determining, based on the predicted second attributes, whether the therapeutic cell composition is predicted to have a desired attribute; and (e) administering a treatment to the subject wherein: (i) if the therapeutic cell composition is predicted to have the desired attribute, a predetermined treatment regimen comprising the therapeutic cell composition is administered; or (ii) if the therapeutic cell composition is predicted to not have the desired attribute, administering to the subject a treatment regimen comprising the therapeutic cell composition that is altered compared to the predetermined treatment regimen comprising the therapeutic cell composition.

Provided are methods of treating a subject, the methods including: (a) selecting T cells from a sample from a subject to produce an input composition including T cells; (b) determining a percentage, number, ratio, and/or proportion of T cells in the input composition having first attributes, wherein the first attributes include cell phenotypes; (c) applying the first attributes as input to a process configured to predict, based on the first attributes, a percentage, number, ratio and/or proportion of cells in an therapeutic cell composition that have one second attribute, wherein the one second attribute includes a cell phenotype or recombinant receptor-dependent activity, and wherein the therapeutic cell composition includes the recombinant receptor, and wherein the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells; (d) determining, based on the one predicted second attribute, whether the therapeutic cell composition is predicted to have a desired attribute; and (e) administering a treatment to the subject wherein: (i) if the therapeutic cell composition is predicted to have the desired attribute, a predetermined treatment regimen including the therapeutic cell composition is administered; or (ii) if the therapeutic cell composition is predicted to not have the desired attribute, administering to the subject a treatment regimen including the therapeutic cell composition that is altered compared to the predetermined treatment regimen including the therapeutic cell composition.

Provided herein are method of treating a subject, the method comprising: (a) selecting T cells from a sample from a subject to produce an input composition comprising T cells; (b) determining a percentage, number, ratio, and/or proportion of T cells in the input composition having first attributes, wherein the first attributes comprise cell phenotypes; (c) applying the first attributes as input to a process configured to predict, based on the first attributes, a percentage, number, ratio and/or proportion of cells in an therapeutic cell composition that have one second attribute, wherein the one second attribute comprises a cell phenotype or recombinant receptor-dependent activity, and wherein the therapeutic cell composition comprises the recombinant receptor, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the one second attribute is predicted for the therapeutic cell composition from the first attributes; or (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the one second attribute is predicted for CD4+ or CD8+ T cells of the separate compositions of the therapeutic composition from the first attributes; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+ T cell compositions of the input composition, wherein the first attributes comprise first attributes from the CD4+ and CD8+ T cell compositions of the input composition, and the one second attribute is predicted for CD4+ or CD8+ T cells of the separate compositions of therapeutic composition from the first attributes; (d) determining, based on the one predicted second attribute, whether the therapeutic cell composition is predicted to have a desired attribute; and (e) administering a treatment to the subject wherein: (i) if the therapeutic cell composition is predicted to have the desired attribute, a predetermined treatment regimen comprising the therapeutic cell composition is administered; or (ii) if the therapeutic cell composition is predicted to not have the desired attribute, administering to the subject a treatment regimen comprising the therapeutic cell composition that is altered compared to the predetermined treatment regimen comprising the therapeutic cell composition.

Provided are methods including (a) determining a percentage, number, ratio, and/or proportion of cells in an input cell composition that have first attributes, wherein the first attributes include cell phenotypes, and wherein the input composition includes T cells selected from a sample from a subject; (b) determining a percentage, number, ratio, and/or proportion of cells in a therapeutic cell composition that have second attributes, wherein the second attributes include cell phenotypes and recombinant receptor-dependent activity, wherein the therapeutic cell composition includes the recombinant receptor, and wherein the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells; (c) training a canonical correlation analysis statistical learning model on the first and second attributes. In some embodiments, the process includes a canonical correlation analysis statistical learning model trained according to methods provided herein; and applying the first attributes as input to the process includes applying the first attributes to the canonical correlation analysis statistical learning model.

Provided herein is a method comprising: (a) determining a percentage, number, ratio, and/or proportion of cells in an input cell composition that have first attributes, wherein the first attributes comprise cell phenotypes, and wherein the input composition comprises T cells selected from a sample from a subject; (b) determining a percentage, number, ratio, and/or proportion of cells in a therapeutic cell composition that have second attributes, wherein the second attributes comprise cell phenotypes and recombinant receptor-dependent activity, wherein the therapeutic cell composition comprises the recombinant receptor, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the second attributes comprise second attributes from the therapeutic cell composition; (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the second attributes comprise second attributes from CD4+ and CD8+ T cells of each of the separate compositions of the therapeutic composition; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+ T cell compositions of the input composition, wherein the first attributes comprise first attributes from the CD4+ and CD8+ T cell compositions of the input composition, and the second attributes comprise second attributes from CD4+ and CD8+ cells of each of the separate compositions of therapeutic composition; and (c) training a canonical correlation analysis statistical learning model on the first and second attributes.

Provided are methods including (a) determining a percentage, number, ratio, and/or proportion of cells in an input cell composition that have first attributes, wherein the first attributes include cell phenotypes, and wherein the input composition includes T cells selected from a sample from a subject; (b) determining a percentage, number, ratio, and/or proportion of cells in an therapeutic cell composition that have one second attribute, wherein the one second attribute includes cell phenotypes and recombinant receptor-dependent activity, wherein the therapeutic cell composition includes the recombinant receptor, and wherein: the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells; (c) training a lasso regression statistical learning model on the first attributes and the one second attribute. In some embodiments, the process includes a lasso regression statistical learning model trained according to the methods provided herein; and applying the first attributes as input to the process including applying the first attributes to the lasso regression statistical learning model.

Provided herein are methods comprising: (a) determining a percentage, number, ratio, and/or proportion of cells in an input cell composition that have first attributes, wherein the first attributes comprise cell phenotypes, and wherein the input composition comprises T cells selected from a sample from a subject; (b) determining a percentage, number, ratio, and/or proportion of cells in an therapeutic cell composition that have one second attribute, wherein the one second attribute comprises cell phenotypes and recombinant receptor-dependent activity, wherein the therapeutic cell composition comprises the recombinant receptor, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the one second attribute comprises one second attribute from the therapeutic cell composition; (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the one second attribute comprises one second attribute CD4+ or CD8+ T cells of the separate compositions of the therapeutic composition; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+ T cell compositions of the input composition, wherein the first attributes comprise first attributes from the CD4+ and CD8+ T cell compositions of the input composition, and the one second attribute comprises one second attribute from CD4+ or CD8+ T cells of the separate compositions of therapeutic composition; (c) training a lasso regression statistical learning model on the first attributes and the one second attribute.

Provided are methods of determining attributes of an input cell composition correlated with attributes of an therapeutic cell composition, the method including: (a) determining a percentage, number, ratio and/or proportion of cells in an input cell composition that have first attributes, wherein the first attributes include cell phenotypes, and wherein the input composition includes T cells selected from a sample from a subject; (b) determining a percentage, number, ratio, and/or proportion of cells in a therapeutic cell composition that have second attributes, wherein the second attributes include cell phenotypes and recombinant receptor-dependent activity, wherein the therapeutic cell composition includes the recombinant receptor, and wherein: the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells; (c) performing canonical correlation analysis (CCA) between the first attributes and the second attributes; and (d) identifying, based on the canonical correlation analysis, the first attributes correlated with the second attributes. In some embodiments, the CCA includes a penalty function capable of regularizing the first and second attributes. In some embodiments, the penalty function includes a constant, said constant determined by performing permutations on the first and second attributes, independently, and performing canonical correlation analysis. In some embodiments, the penalty function is lasso regularization. In some embodiments, the method further incudes constraining the square of the L2 norm of canonical vectors to be less than or equal to 1.

Provided herein is a method of determining attributes of an input cell composition correlated with attributes of a therapeutic cell composition, the method comprising: (a) determining a percentage, number, ratio and/or proportion of cells in an input cell composition that have first attributes, wherein the first attributes comprise cell phenotypes, and wherein the input composition comprises T cells selected from a sample from a subject; (b) determining a percentage, number, ratio, and/or proportion of cells in a therapeutic cell composition that have second attributes, wherein the second attributes comprise cell phenotypes and recombinant receptor-dependent activity, wherein the therapeutic cell composition comprises the recombinant receptor, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the second attributes comprise second attributes from the therapeutic cell composition; (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the second attributes comprise second attributes from CD4+ and CD8+ T cells of each of the separate compositions of the therapeutic composition; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+ T cell compositions of the input composition, wherein the first attributes comprise first attributes from the CD4+ and CD8+ T cell compositions of the input composition, and the second attributes comprise second attributes from CD4+ and CD8+ cells of each of the separate compositions of therapeutic composition; (c) performing canonical correlation analysis (CCA) between the first attributes and the second attributes; and (d) identifying, based on the canonical correlation analysis, the first attributes correlated with the second attributes.

Provided are methods of determining attributes of an input composition correlated with attributes of an therapeutic cell composition, the method including: (a) determining a percentage, number, ratio, and/or proportion of cells in an input cell composition that have first attributes, wherein the first attributes include cell phenotypes, and wherein the input composition includes T cells selected from a sample from a subject; (b) determining a percentage, number, ratio, and/or proportion of cells in a therapeutic cell composition that have one second attribute, wherein the one second attribute includes a cell phenotype or a recombinant receptor-dependent activity, wherein the therapeutic cell composition includes the recombinant receptor, and wherein the input composition includes CD4+, CD8+, or CD4+ and CD8+ T cells and the therapeutic cell composition includes the recombinant receptor and is produced from the input composition; or the input composition is a first input composition including CD4+ or CD8 T cells and the output cell composition includes the recombinant receptor and is produced from another input composition including the other of the CD4+ or CD8+ T cells; (c) performing lasso regression between the first attributes and the second attributes; and (d) identifying, based on the lasso regression, the first attributes correlated with the one second attribute.

Provided herein is a method of determining attributes of an input composition correlated with attributes of an therapeutic cell composition, the method comprising: (a) determining a percentage, number, ratio, and/or proportion of cells in an input cell composition that have first attributes, wherein the first attributes comprises cell phenotypes, and wherein the input composition comprises T cells selected from a sample from a subject; (b) determining a percentage, number, ratio, and/or proportion of cells in a therapeutic cell composition that have one second attribute, wherein the one second attribute comprises a cell phenotype or a recombinant receptor-dependent activity, wherein the therapeutic cell composition comprises the recombinant receptor, and wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the one second attribute comprises one second attribute from the therapeutic cell composition; (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the one second attribute comprises one second attribute CD4+ or CD8+ T cells of the separate compositions of the therapeutic composition; or (iii) the input composition comprises separate compositions of CD4+ and CD8+ T cells and the therapeutic cell composition comprises a mixed composition of CD4+ and CD8+ T cells expressing the recombinant receptor, and produced from the respective CD4+ and CD8+T cell compositions of the input composition, wherein the first attributes comprise first attributes from the CD4+ and CD8+ T cell compositions of the input composition, and the one second attribute comprises one second attribute from CD4+ or CD8+ T cells of the separate compositions of therapeutic composition; (c) performing lasso regression between the first attributes and the second attributes; and (d) identifying, based on the lasso regression, the first attributes correlated with the one second attribute.

In some embodiments, the method further includes prior to (a) selecting T cells from the sample from the subject to produce the input composition including CD4, CD8, or CD4 and CD8 T cells. In some embodiments, the sample includes a whole blood sample, a buffy coat sample, a peripheral blood mononuclear cell (PBMC) sample, an unfractionated T cell sample, a lymphocyte sample, a white blood cell sample, an apheresis product, or a leukapheresis product. In some embodiments, the sample is an apheresis product or leukapheresis product. In some embodiments, the apheresis product or leukapheresis product has been previously cryopreserved. In some embodiments, the T cells include primary cells obtained from the subject. In some embodiments, the recombinant receptor is a chimeric antigen receptor (CAR).

In some embodiments, the input composition includes CD4+, CD8+, or CD4+ and CD8+ T cells, and the therapeutic cell composition includes CD4+ and/or CD8+ T cells expressing the recombinant receptor and is to be produced from the input composition; and the first attributes include first attributes from the input composition, and the second attributes are predicted for the therapeutic cell composition from the first attributes. In some embodiments, the input composition includes separate compositions of CD4+ and CD8+ T cells, and the therapeutic cell composition includes separate compositions of CD4+ and CD8+ T cells expressing the recombinant receptor and is to be produced from the respective CD4+ or CD8+ T cell composition of the input composition; and the first attributes include first attributes from the CD4+ and CD8+ T cell compositions of the input composition, and the second attributes are predicted for CD4+ and CD8+ T cells of each of the separate CD4+ and CD8+ T cell compositions of the therapeutic cell composition from the first attributes. In some embodiments, the input composition includes separate compositions of CD4+ and CD8+ T cells, and the therapeutic cell composition includes a mixed composition of CD4+ and CD8+ T cells expressing the recombinant receptor and is to be produced from the CD4+ and CD8+ T cell compositions of the input composition; and the first attributes include first attributes from the CD4+ and CD8+ T cell compositions of the input composition, and the second attributes are predicted for CD4+ and CD8+ cells of each of the separate CD4+ and CD8+ T cell compositions of the therapeutic cell composition from the first attributes. In some embodiments, the input composition includes CD4+, CD8+, or CD4+ and CD8+ T cells, and the therapeutic cell composition includes CD4+ and/or CD8+ T cells expressing the recombinant receptor and is to be produced from the input composition; and the first attributes include first attributes from the input composition, and the one second attribute is predicted for the therapeutic cell composition from the first attributes. In some embodiments, the input composition includes separate compositions of CD4+ and CD8+ T cells, and the therapeutic cell composition includes separate compositions of CD4+ and CD8+ T cells expressing the recombinant receptor and is to be produced from the respective CD4+ or CD8+ T cell composition of the input composition; and the first attributes include first attributes from the CD4+ and CD8+ T cell compositions of the input composition, and the one second attribute is predicted for CD4+ or CD8+ T cells of the separate CD4+ and CD8+ T cell compositions of the therapeutic cell composition from the first attributes. In some embodiments, the input composition includes separate compositions of CD4+ and CD8+ T cells and the therapeutic cell composition includes a mixed composition of CD4+ and CD8+ T cells expressing the recombinant receptor, and is to be produced from the respective CD4+ and CD8+ T cell compositions of the input composition; and the first attributes include first attributes from the CD4+ and CD8+ T cell compositions of the input composition, and the one second attribute is predicted for CD4+ or CD8+ T cells of the separate CD4+ or CD8+ composition of the therapeutic cell composition from the first attributes.

In some embodiment, each of the plurality of input compositions included in the training data includes CD4+, CD8+, or CD4+ and CD8+ T cells and each of the plurality of therapeutic cell compositions included in the training data includes CD4+ and/or CD8+ T cells expressing the recombinant receptor and has been produced from one of the plurality of input compositions; and the first attributes include first attributes from each of the plurality of input compositions included in the training data, and the second attributes include second attributes of each of the plurality of therapeutic cell compositions included in the training data. In some embodiments, each of the plurality of input compositions included in the training data includes separate compositions of CD4+ and CD8+ T cells, and each of the plurality of therapeutic cell compositions included in the training data includes separate compositions of CD4+ and CD8+ T cells expressing the recombinant receptor, and has been produced from the respective CD4+ or CD8+ T cell composition of one of the plurality of input compositions; and the first attributes include first attributes from the CD4+ and CD8+ T cell compositions of each of the plurality of input compositions included in the training data, and the second attributes include second attributes from CD4+ and CD8+ T cells of each of the separate CD4+ and CD8+ T cell compositions of each of the plurality of therapeutic cell compositions included in the training data. In some embodiments, each of the plurality of input compositions included in the training data includes separate compositions of CD4+ and CD8+ T cells, and each of the plurality of therapeutic cell compositions included in the training data includes a mixed composition of CD4+ and CD8+ T cells expressing the recombinant receptor, and has been produced from the respective CD4+ and CD8+ T cell compositions of one of the plurality of input compositions; and the first attributes include first attributes from the CD4+ and CD8+ T cell compositions of each of the plurality of input compositions includes in the training data, and the second attributes include second attributes from CD4+ and CD8+ T cells of each of the separate CD4+ and CD8+ T cell compositions of each of the plurality of therapeutic cell compositions included in the training data. In some embodiments, each of the plurality of input compositions included in the training data includes CD4+, CD8+, or CD4+ and CD8+ T cells, and each of the plurality of therapeutic cell compositions included in the training data includes CD4+ and/or CD8+ T cells expressing the recombinant receptor and has produced from one of the plurality of input compositions; and the first attributes include first attributes from each of the plurality of input compositions included in the training data, and the one second attribute includes one second attribute of each of the plurality of therapeutic cell compositions included in the training data. In some embodiments, each of the plurality of input compositions of the training data includes separate compositions of CD4+ and CD8+ T cells, and each of the plurality of therapeutic cell compositions included in the training data includes separate compositions of CD4+ and CD8+ T cells expressing the recombinant receptor and has been produced from the respective CD4+ or CD8+ T cell composition of one of the plurality of input compositions; and the first attributes include first attributes from the CD4+ and CD8+ T cell compositions of each of the plurality of input compositions included in the training data, and the one second attribute includes one second attribute of the CD4+ or CD8+ T cells of the separate CD4+ and CD8+ T cell compositions of each of the plurality of therapeutic cell compositions included in the training data. In some embodiments, each of the plurality of input compositions included in the training data includes separate compositions of CD4+ and CD8+ T cells, and each of the plurality of therapeutic cell compositions included in the training data includes a mixed composition of CD4+ and CD8+ T cells expressing the recombinant receptor, and has been produced from the respective CD4+ and CD8+ T cell compositions of one of the plurality of input compositions; and the first attributes include first attributes from the CD4+ and CD8+ T cell compositions of each of the plurality of input compositions included in the training data, and the one second attribute includes one second attribute from CD4+ or CD8+ T cells of the separate CD4+ or CD8+ T cell compositions of each of the plurality of therapeutic cell compositions included in the training data.

In some embodiments, the first attributes include one or more cell phenotypes including 3CAS−/CCR7−/CD27−, 3CAS−/CCR7−/CD27+, 3CAS−/CCR7+, 3CAS−/CCR7+/CD27, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and CAS+/CD3+. In some embodiments, the first attributes include one or more cell phenotypes including 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and CAS+/CD3+ of an input composition that is CD8+ cells.

In some of any of the embodiments, the first attribute comprises one or more cell phenotypes comprising CD4+/CCR7+/CD27+, CD4+/CCR7+/CD45RA+, CD4+/CD28+/CD27−, CD8+/CCR7+/CD45RA−, CD8+/CCR7+/CD45RA+, CD8+/CCR7+, CD4+/CCR7−/CD27−, CD8+/CCR7−/CD45RA+, CD4+/CD28+/CD27−, CD4+/CD28+, and CD28+/CD27−. In some of any of the embodiments, the first attribute comprises one or more cell phenotypes comprising CD4+/CCR7+/CD27+, CD4+/CCR7+/CD45RA+, CD4+/CD28+/CD27−, CD8+/CCR7+CD45RA−, and CD8+/CCR7+CD45RA+. In some of any of the embodiments, the first attribute comprises one or more cell phenotypes comprising CD8+/CCR7+, CD4+/CCR7−/CD27−, CD8+/CCR7−/CD45RA+, and CD4+/CD28+.

In some of any of the embodiments, the first attribute comprises or is CD4+/CCR7+/CD45RA+.

In some embodiments, the second attributes include one or more cell phenotypes and/or recombinant receptor-dependent activity including 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+/CD19+, CD3+/CAR+, CD3+/CD56+, CAR+, IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+, IFNG+, IL10+, IL13+, IL2+, IL5+, MIP1A+, MIP1B+, sCD137+, and TNFa+.

In some embodiments, wherein the second attributes include one or more cell phenotypes and/or recombinant receptor-dependent activity including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL 2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL−17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+/CAR+, IL-2+ of CD8+/CAR+, IL-2+/TNFA+/CD8+/CAR+, cytolytic CD8+, TNFA+ of CD8+CAR+, GMCSF+, IFNG+, IL10+, IL13+, IL2+, IL5+, MIP1A+, MIP1B+, sCD137+, and TNFa+.

In some of any of the embodiments, the second attributes comprise one or more cell phenotypes and/or recombinant receptor-dependent activity comprising CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, CCR7+/CD45RA+/CD4+/CAR+, CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, and CCR7+/CD45RA+/CD8+/CAR+. In some of any of the embodiments, the second attributes comprise one or more cell phenotypes and/or recombinant receptor-dependent activity comprising CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, and CCR7+/CD45RA+/CD4+/CAR+. In some of any of the embodiments, the second attributes comprise one or more cell phenotypes and/or recombinant receptor-dependent activity comprising CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, and CCR7+/CD45RA+/CD8+/CAR+.

In some embodiments, the first attributes include or include about 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 cell phenotypes. In some embodiments, the first attributes include or include about or at least 2, 4, 6, 8, 10, 12, or more cell phenotypes. In some embodiments, the first attributes include greater than or greater than about 5, 10, 15, or 20 cell attributes. In some embodiments, the second attributes include or include about 101, 90, 80, 70, 60, 50, 40, 30, 20, 15, 10, 5, 4, 3, 2, or 1 cell phenotypes and recombinant receptor-dependent activity. In some embodiments, the second attributes include about or at least 1, 2, 4, 6, 8, 10, 12, or more cell phenotypes and recombinant receptor-dependent activity. In some embodiments, the second attributes include about or at least 15, 20, 30, 40, 50, 60, 70, 80, 90, or more T cell phenotypes and recombinant receptor-dependent activity. In some embodiments, the second attributes include 1 cell phenotype or recombinant receptor-dependent activity.

In some embodiments, the desired attribute is at least one attribute that is correlated to clinical response of the therapeutic cell composition. In some embodiments, wherein the desired attribute is an attribute that is correlated to clinical response of the therapeutic cell composition. In some embodiments, the clinical response is a durable response and/or progression free survival. In some embodiments, the desired attribute is at least one attribute that is correlated with a positive clinical response to treatment with the therapeutic cell composition. In some embodiments, the desired attribute is an attribute that is correlated with a positive clinical response to treatment with the therapeutic cell composition. In some embodiments, the positive clinical response is a durable response and/or progression free survival.

In some embodiments, the desired attribute is or includes a threshold percentage of naïve-like T cells or central memory T cells. In some embodiments, the threshold percentage is at least or at least about 40% of the cells in the therapeutic cell composition that are naïve-like T cells or central memory T cells. In some embodiments, the threshold percentage is at least or at least about 50% of the cells in the therapeutic cell composition that are naïve-like T cells or central memory T cells. In some embodiments, the threshold percentage is at least or at least about 60% of the cells in the therapeutic cell composition that are naïve-like T cells or central memory T cells. In some embodiments, the threshold percentage is at least or at least about 65% of the cells in the therapeutic cell composition that are naïve-like T cells or central memory T cells. In some embodiments, the threshold percentage is at least or at least about 70% of the cells in the therapeutic cell composition that are naïve-like T cells or central memory T cells. In some embodiments, the naïve-like T cells or central memory T cells have a phenotype including T cells surface positive for CD27+, CD28+, CD62L+, and/or CCR7+. In some embodiments, the naïve-like T cells or central memory T cells have the phenotype CD62L+/CCR7+, CD27+/CCR7+, CD62L+/CD45RA−, CCR7+/CD45RA−, CD62L+/CCR7+/CD45RA−, CD27+/CD28+/CD62L+/CD45RA−, CD27+/CD28+/CCR7+/CD45RA−, CD27+/CD28+/CD62L+/CCR7+, or CD27+/CD28+/CD62L+/CCR7+/CD45RA−.

In some embodiments, the desired attribute is a threshold percentage of CD27+/CCR7+ T cells in the therapeutic cell composition. In some embodiments, the threshold percentage is at least or at least about 60%, 70%, 80%, 85%, 90%, 91%, 92%, 93%, 94%, 95%, 96%, 97%, 98%, 99% or 100% of the total number of cells in the therapeutic cell composition are CD27+/CCR7+. In some embodiments, the threshold percentage is at least or at least about 60% of the cells in the therapeutic cell composition are CD27+/CCR7+. In some embodiments, the CD27+/CCR7+ cells are CD4+/CAR+ T cells and/or CD8+/CAR+ T cells. In some embodiments, the CD27+/CCR7+ cells are CD4+/CAR+ T cells and CD8+/CAR+ T cells. In some embodiments, the CD27+/CCR7+ cells are CD4+/CAR+ T cells. In some embodiments, the CD27+/CCR7+ cells are CD8+/CAR+ T cells.

In some embodiments, the desired attribute is a threshold percentage of IL-2+ of CD4+/CAR+ and IL-2+/TNFA+/CD4+/CAR+ T cells in the therapeutic cell composition. In some embodiments, the threshold percentage is at least at or about 70%, 80%, 85%, 90%, 91%, 92%, 93%, 94%, 95%, 96%, 97%, 98%, 99% or 100% of the total number of CD4+ T cells in the therapeutic cell composition. In some embodiments, the desired attribute is a threshold percentage of IL-2+ of CD8+/CAR+ and IL-2+/TNFA+/CD8+/CAR+ T cells in the therapeutic cell composition. In some embodiments, the threshold percentage is at least at or about 70%, 80%, 85%, 90%, 91%, 92%, 93%, 94%, 95%, 96%, 97%, 98%, 99% or 100% of the total number of CD8+ T cells in the therapeutic cell composition.

In some embodiments, the desired attribute is a threshold percentage of IFNG+/IL-2+/CD4+/CAR+, IFNG+/IL-2+/IL-17+/TNFA+/CD4+/CAR+, IFNG+/IL-2+/TNFA+/CD4+/CAR+, IFNG+/TNFA+/CD4+/CAR+, IL-17+ of CD4+CAR+, IL-2+ of CD4+CAR+, and/or IL-2+/TNFA+/CD4+/CAR+ T cells in the therapeutic cell composition. In some embodiments, wherein the threshold percentage is at least at or about 10%, 15%, 20%, 25%, 30%, 40%, 50%, 60% or more of the total number of CAR+/CD4+ T cells in the therapeutic cell composition. In some embodiments, the desired attribute is a threshold percentage of IFNG+/IL-2+/CD8+/CAR+, IFNG+/IL-2+/IL-17+/TNFA+/CD8+/CAR+, IFNG+/IL-2+/TNFA+/CD8+/CAR+, IFNG+/TNFA+/CD8+/CAR+, IL-17+ of CD8+CAR+, IL-2+ of CD8+CAR+, and/or IL-2+/TNFA+/CD8+/CAR+ T cells in the therapeutic cell composition. In some embodiments, wherein the threshold percentage is at least at or about 10%, 15%, 20%, 25%, 30%, 40%, 50%, 60% or more of the total number of CAR+/CD8+ T cells in the therapeutic cell composition.

In some embodiments, altering the predetermined treatment regimen includes increasing a dosing frequency or the volume of a unit dose. In some embodiments, increasing the dosing frequency or the volume of the unit dose improves clinical response. In some embodiments, altering the predetermined treatment regimen includes administering the therapeutic cell composition in combination with a second therapeutic agent. In some embodiments, the second therapeutic agent is a cytokine. In some embodiments, the cytokine is IL-2. In some embodiments, the second therapeutic agent is a chemotherapeutic agent.

Provided are methods of determining attributes of a therapeutic cell composition, the method including assessing an input composition including T cells for a phenotype, or a percentage, number, ratio and/or proportion of cells of the phenotype, thereby determining, from the phenotype, the likelihood or presence of an attribute in a therapeutic cell composition, or a percentage, number, ratio and/or proportion of cells having the attribute in the therapeutic cell composition, wherein: the therapeutic cell composition includes a recombinant receptor, and wherein the input composition includes CD4+, CD8+, or CD4+ and CD8+ T cells and the therapeutic cell composition includes a recombinant receptor and is produced from the input composition; or the input composition is a first input composition including CD4+ or CD8 T cells and the output cell composition includes a recombinant receptor and is produced from another input composition including the other of the CD4+ or CD8+ T cells. Also provided are methods of determining attributes of a therapeutic cell composition, the method comprising assessing an input composition comprising T cells for a first attribute, or a percentage, number, ratio and/or proportion of cells of the first attribute, thereby determining, from the first attribute, the likelihood or presence of a second attribute in a therapeutic cell composition, or a percentage, number, ratio and/or proportion of cells having the second attribute in the therapeutic cell composition, wherein: (i) the input composition comprises CD4+, CD8+, or CD4+ and CD8+ T cells and the therapeutic cell composition comprises CD4+ and/or CD8+ T cells expressing the recombinant receptor and is produced from the input composition, wherein the first attributes comprise first attributes from the input composition, and the second attributes are determined for the therapeutic cell composition from the first attributes; or (ii) the input composition comprises separate compositions of CD4+ and CD8+ T cells and the therapeutic cell composition comprises separate compositions of CD4+ and CD8+ T cells expressing the recombinant receptor, and produced from the respective CD4+ or CD8+ T cell composition of the input composition, wherein the first attributes comprise first attributes from the CD4+ and CD8+ T cell compositions of the input composition, and the second attributes are determined for CD4+ and CD8+ T cells of each of the separate compositions of the therapeutic composition from the first attributes; or (iii) the input composition comprises separate compositions of CD4+ and CD8+ T cells and the therapeutic cell composition comprises a mixed composition of CD4+ and CD8+ T cells expressing the recombinant receptor, and is produced from the CD4+ and CD8+ T cell compositions of the input composition, wherein the first attributes comprise first attributes from the CD4+ and CD8+ T cell compositions of the input composition, and the second attributes are determined for CD4+ and CD8+ cells of each of the separate compositions of therapeutic composition from the first attributes. In any of such embodiments,

In any of such embodiments for determining attributes of a therapeutic cell composition, the phenotype and attribute is selected from: (a) a phenotype that is CD27+/CCR7+, CD27+, CCR7+, or CCR7+/CD45RA+ of CD4+ T cells in the input composition and an attribute that is CD27+/CCR7+, CD27+, CCR7+, CCR7+/CD45RA+ of CD4+ T cells and CD8+ T cells in the therapeutic cell composition; (b) a phenotype that is CD27+/CCR7+, CD27+, CCR7+, CD28+/CD27+, or CD28+ of CD4+ T cells in the input composition and an attribute that is CD27+/CCR7+, CD27+, CCR7+, or CCR7+/CD45RA+ of CD8+ T cells in the therapeutic cell composition; (c) a phenotype that is CD28−/CD27−, CCR7−/CD27−, or CCR7+/CD45RA+ of CD4+ T cells in the input composition and an attribute that is CD28−/CD27−, CCR7−/CD27−, or CCR7+/CD45RA+ of CD8+ T cells in the therapeutic cell composition; (d) a phenotype that is CD27+/CCR7+, CD27+, CCR7+, CD28+/CD27+, or CD28+ of CD8+ cells in the input composition and an attribute that is CD28−/CD27−, CCR7−/CD27−, or CCR7+/CD45RA+ of CD8+ T cells in the therapeutic cell composition; (e) a phenotype that is CD28−/CD27−, CCR7−/CD27−, or CCR7+/CD45RA+ of CD8+ T cells in the input composition and an attribute that is CD28−/CD27−, CCR7−/CD27−, or CCR7+/CD45RA+ of CD8+ T cells of the therapeutic T cell composition; (f) a phenotype that is CCR7−/CD45RA−, CCR7−/CD27−, or CD28+/CD27- of CD4+ T cells in the input composition and an attribute that is IFNg+, IL-5+, or GMCSF+ of CD4+ T cells of the therapeutic cell composition; (g) a phenotype that is CCR7−/CD45RA−, CCR7−/CD27−, or CD28+/CD27- of CD4+ T cells in the input composition and an attribute that is IL-2+ or TNFa+ of CD8+ T cells of the output composition; (h) a phenotype that is CCR7+/CD27−, CD28+/CD27−, or CCR7+/CD45RA− of CD8 T cells in the input composition and an attribute that is IL-5+, IL-13+, TNF-a+, or IL-2+ of CD8+ T cells in the output composition (i) a phenotype that is CCR7+CD27+ or CCR7+CD45RA+ of CD8+ and CD4+ cells in the input composition and an attribute that is CCR7+/CD27+ or CCR7+CD45RA+ of CD8+ T cells in the therapeutic cell composition; (j) a phenotype that is CCR7−/CD27− of CD4+ and CD8+ T cells in the input composition and an attribute that is IFNg+, TNF-a+, IL-13+, IL-2+, or IL-5+ of CD8+ T cells in the therapeutic cell composition. In some embodiments, wherein the method further includes selecting T cells from the sample from the subject to produce the input composition including CD4, CD8, or CD4 and CD8 T cells.

In any of such embodiments for determining attributes of a therapeutic cell composition, the (a) the first attribute is a percentage, number, ratio and/or proportion of CD27+/CCR7+, CD27+, CCR7+, or CCR7+/CD45RA+ of CD4+ T cells in the input composition and the second attribute is a percentage, number, ratio and/or proportion of CD27+/CCR7+, CD27+, CCR7+, CCR7+/CD45RA+ of CD4+ T cells and CD8+ T cells in the therapeutic cell composition; (b) the first attribute is a percentage, number, ratio and/or proportion of CD27+/CCR7+, CD27+, CCR7+, CD28+/CD27+, or CD28+ of CD4+ T cells in the input composition and the second attribute is a percentage, number, ratio and/or proportion of CD27+/CCR7+, CD27+, CCR7+, or CCR7+/CD45RA+ of CD8+ T cells in the therapeutic cell composition; (c) the first attribute is a percentage, number, ratio and/or proportion of CD28−/CD27−, CCR7−/CD27−, or CCR7+/CD45RA+ of CD4+ T cells in the input composition and the second attribute is a percentage, number, ratio and/or proportion of CD28−/CD27−, CCR7−/CD27−, or CCR7+/CD45RA+ of CD8+ T cells in the therapeutic cell composition; (d) the first attribute is a percentage, number, ratio and/or proportion of CD27+/CCR7+, CD27+, CCR7+, CD28+/CD27+, or CD28+ of CD8+ cells in the input composition and the second attribute is a percentage, number, ratio and/or proportion of CD28−/CD27−, CCR7−/CD27−, or CCR7+/CD45RA+ of CD8+ T cells in the therapeutic cell composition; (e) the first attribute is a percentage, number, ratio and/or proportion of CD28−/CD27−, CCR7−/CD27−, or CCR7+/CD45RA+ of CD8+ T cells in the input composition and the second attribute is a percentage, number, ratio and/or proportion of CD28−/CD27−, CCR7−/CD27−, or CCR7+/CD45RA+ of CD8+ T cells of the therapeutic T cell composition; (f) the first attribute is a percentage, number, ratio and/or proportion of CCR7−/CD45RA−, CCR7−/CD27−, or CD28+/CD27− of CD4+ T cells in the input composition and the second attribute is a percentage, number, ratio and/or proportion of IFNg+, IL-5+, or GMCSF+ of CD4+ T cells of the therapeutic cell composition; (g) the first attribute is a percentage, number, ratio and/or proportion of CCR7−/CD45RA−, CCR7−/CD27−, or CD28+/CD27− of CD4+ T cells in the input composition and the second attribute is a percentage, number, ratio and/or proportion of IL-2+ or TNFa+ of CD8+ T cells of the output composition; (h) the first attribute is a percentage, number, ratio and/or proportion of CCR7+/CD27−, CD28+/CD27−, or CCR7+/CD45RA− of CD8 T cells in the input composition and the second attribute is a percentage, number, ratio and/or proportion of IL-5+, IL-13+, TNF-a+, or IL-2+ of CD8+ T cells in the output composition (i) the first attribute is a percentage, number, ratio and/or proportion of CCR7+/CD27+ or CCR7+/CD45RA+ of CD8+ and CD4+ cells in the input composition and the second attribute is a percentage, number, ratio and/or proportion of CCR7+/CD27+ or CCR7+/CD45RA+ of CD8+ T cells in the therapeutic cell composition; (j) the first attribute is a percentage, number, ratio and/or proportion of CCR7−/CD27− of CD4+ and CD8+ T cells in the input composition and second attribute is a percentage, number, ratio and/or proportion of IFNg+, TNF-a+, IL-13+, IL-2+, or IL-5+ of CD8+ T cells in the therapeutic cell composition; (k) the first attribute is a percentage, number, ratio and/or proportion of CCR7+/CD27−, CD28+/CD27−, CCR7+/CD45RA− of CD4+ and CD8+ T cells in the input composition second attribute is a percentage, number, ratio and/or proportion of CCR7+/CD27−, CD28+/CD27−, CCR7+/CD45RA− of CD4+ and CD8+ T cells in the therapeutic cell composition; (1) the first attribute is a percentage, number, ratio and/or proportion of CCR7+/CD45RA− CD8+ T cells in the input composition and second attribute is a percentage, number, ratio and/or proportion of TNF-a+ or IL-2+ of CD8+ T cells in the therapeutic cell composition; (m) the first attribute is a percentage, number, ratio and/or proportion of CCR7+, CCR7+/CD27+, CD27+CD4+ T cells in the input composition and second attribute is a percentage, number, ratio and/or proportion of CCR7+, CCR7+/CD27+, CD27+ of CD4+ and CD8+ T cells in the therapeutic cell composition; (n) the first attribute is a percentage, number, ratio and/or proportion of CD4+/CCR7+/CD27+, CD4+/CCR7+/CD45RA+, CD4+/CD28+/CD27−, CD8+/CCR7+/CD45RA−, CD8+/CCR7+/CD45RA+, CD8+/CCR7+, CD4+/CCR7−/CD27−, CD8+/CCR7−/CD45RA+, CD4+/CD28+/CD27−, CD4+/CD28+, and CD28+/CD27− T cells in the input composition and second attribute is a percentage, number, ratio and/or proportion of CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, CCR7+/CD45RA+/CD4+/CAR+, CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, CCR7+/CD45RA+/CD8+/CAR+ T cells in the therapeutic cell composition; (o) the first attribute is a percentage, number, ratio and/or proportion of CD4+/CCR7+/CD27+, CD4+/CCR7+/CD45RA+, and CD4+/CD28+/CD27 T cells in the input composition and second attribute is a percentage, number, ratio and/or proportion of comprising CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, and CCR7+/CD45RA+/CD4+/CAR+ T cells in the therapeutic cell composition; (p) the first attribute is a percentage, number, ratio and/or proportion of CD4+/CCR7+/CD27+, CD4+/CCR7+/CD45RA+, CD4+/CD28+/CD27−, CD8+/CCR7+CD45RA−, and CD8+/CCR7+CD45RA+ T cells in the input composition and second attribute is a percentage, number, ratio and/or proportion of comprising CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, and CCR7+/CD45RA+/CD8+/CAR+ T cells in the therapeutic cell composition; (q) the first attribute is a percentage, number, ratio and/or proportion of CD4+/CCR7+/CD45RA+ T cells in the input composition and second attribute is a percentage, number, ratio and/or proportion of comprising CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, CCR7+/CD45RA+/CD4+/CAR+, CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, CCR7+/CD45RA+/CD8+/CAR+ T cells in the therapeutic cell composition.

In some embodiments, the therapeutic cell composition is generated by manufacturing the input composition. In some embodiments, the manufacturing includes stimulating the input cell composition. In some embodiments, the manufacturing includes transducing the input composition with a vector including a recombinant receptor. In some embodiments, the recombinant receptor is a chimeric antigen receptor (CAR). In some embodiments, the phenotypes of the input composition are assessed or determined prior to stimulation. In some aspects, the manufacturing process is selected to be a first manufacturing process, for example based on the prediction of desired attributes in a therapeutic composition. In other aspects, the manufacturing process is selected to be a second manufacturing process, for example based on the prediction of desired attributed in a therapeutic composition.

In some embodiments, the first manufacturing process is a process that includes a step of introducing T cells of the input composition with a nucleic acid encoding a recombinant receptor to generate an engineered T cell composition, and cultivating the engineered T cell compositions under conditions for expansion of T cells. In some embodiments, the first manufacturing process is a process wherein the input composition is not enriched or selected for an increased percentage of naïve-like T cells or T cells having a central memory phenotype from the biological sample. In some embodiments, the first manufacturing process is a process wherein obtaining the input composition does not include enriching or selecting for naïve-like T cells or T cells having a central memory phenotype from the biological sample. In some of any of the provided, the first manufacturing process is a process wherein obtaining the input composition does not include depleting T cells having a phenotype of a terminally differentiated T cell or cell with reduced proliferative capacity, for example wherein the phenotype of a terminally differentiated T cell or cell with reduced proliferative capacity is CD57+. In some of any embodiments, the first manufacturing process is an expanded process resulting in more than 2-fold increase in cells in the therapeutic cell composition compared to the input composition. In some embodiments, the first manufacturing process is an expanded process resulting in more than 4-fold increase in cells in the therapeutic cell composition compared to the input composition. In some embodiments, the first manufacturing process is a process that exhibits any combination of the above features.

In some embodiments, the second manufacturing process is a process that includes a step of introducing T cells of the input composition with a nucleic acid encoding a recombinant receptor to generate an engineered T cell composition, and incubating the engineered T cell composition under condition that do not expand T cells in the composition or that minimally expand T cells in the composition. In some embodiments, the second manufacturing process includes obtaining the input composition by enriching or selecting for naïve-like T cells or T cells having a central memory phenotype from the biological sample. In some embodiments, the second manufacturing process is a process wherein the input composition includes a threshold number of naïve-like cells or central memory T cells. In some embodiments, the second manufacturing process is a process wherein obtaining the input composition includes depleting T cells having a phenotype of a terminally differentiated T cell or a cell with reduced proliferative capacity, for examples wherein the phenotype of a terminally differentiated T cell or cell with reduced proliferative capacity is CD57+. In some embodiments, the second manufacturing process is a non-expanded or minimally expanded process resulting in less than 2-fold more cells in the output composition compared to the input composition. In some embodiments, the first manufacturing process is a process that exhibits any combination of the above features.

In some aspects, the manufacturing process, such as independently the first manufacturing process or second manufacturing process, include: stimulating the input cell composition with a T cell stimulatory agent(s), optionally wherein the T cell stimulatory agent(s) is or includes an anti-CD3 antibody, an anti-CD28 antibody and one or more recombinant cytokines selected from IL-2, IL-15, IL-7 and IL-21 to produce a stimulated composition; and introducing into cells of the stimulated composition a polynucleotide encoding the recombinant receptor. In some embodiments, the introducing includes transducing cells with a viral vector encoding the recombinant receptor. In some embodiments, the first manufacturing process further includes cultivating cells introduced with the polynucleotide under conditions for expansion of T cells in the composition. In some embodiments, the second manufacturing process further includes cultivating cells introduced with the polynucleotide under conditions for expansion of T cells in the composition. In some embodiments, the second manufacturing process, does not include cultivating cells introduced with the polynucleotide under conditions for expansion of T cells in the composition.

In some of any of the embodiments, the manufacturing comprises stimulating the input cell composition with a T cell stimulatory agent(s), optionally wherein the T cell stimulatory agent(s) is or comprises an anti-CD3 antibody, an anti-CD28 antibody and one or more recombinant cytokines selected from IL-2, IL-15, IL-7 and IL-21 to produce a stimulated composition; and introducing into cells of the input composition a polynucleotide encoding the recombinant receptor, optionally wherein the transducing cells with a viral vector encoding the recombinant receptor.

In some of any of the embodiments, cells of the input composition are selected or enriched from a biological sample from a subject, optionally a human subject. In some embodiments, the biological sample is a blood, apheresis or leukapheresis sample. In some embodiments, the subject is a human subject.

In some embodiments, the biological sample includes a whole blood sample, a buffy coat sample, a peripheral blood mononuclear cell (PBMC) sample, an unfractionated T cell sample, a lymphocyte sample, a white blood cell sample, an apheresis product, or a leukapheresis product. In some embodiments, the biological sample is an apheresis product or leukapheresis product. In some embodiments, the apheresis product or leukapheresis product has been previously cryopreserved. In some embodiments, the T cells include primary cells obtained from the subject. In some embodiments, the recombinant receptor is a chimeric antigen receptor (CAR). In some embodiments, cells of the input composition are selected or enriched from a biological sample from a subject. In some embodiments, the subject is human. In some embodiments, the CD4+, CD8+, or CD4+ and CD8+ T cells in the input composition, or in each separate composition of the input composition, is enriched from a biological sample, optionally wherein the enriched composition comprises at or great than about 85%, 86%, 87%, 88%, 89%, 90%, 91%, 92%, 93%, 94%, 95%, 96%, 97%, 98%, or 99% or more of the respective CD4+, CD8+, or CD4+ and CD8+ T cells.

### BRIEF DESCRIPTION OF THE DRAWINGS

FIGS. 1A-1H show the first four pairs of input composition and therapeutic cell composition attributes identified by two penalized canonical correlation analysis (pCCA) runs. FIGS. 1A-1D correspond to the first four pairs of input composition and therapeutic cell composition attributes identified in a first pCCA run. FIGS. 1E-1H correspond to the first four pairs of input composition and therapeutic cell composition attributes identified in a second pCCA run.

FIG. 2 shows exemplary accuracy of the lasso regression model for predicting the therapeutic cell composition attribute CCR7−/CD27− of CD4+/CAR+ cells.

FIG. 3 shows a heatmap depicting the number of times an input composition attribute was identified as relevant for predicting a given therapeutic cell composition attribute. The bar plot along the top shows the average nested cross-validation R-squared value across 100 iterations. The bar plot on the right side of the heatmap shows the total number of times an input composition attribute was identified as predictive of a therapeutic composition attribute over 100 iterations. The legend for the x and y axis labels is shown in the Tables 1 and 2 below.

TABLE 1
X Axis Label

| | |
|---|---|
| 1 | CD4 CAR: CCR7− CD27− |
| 2 | CD4 CAR: CD28+ CD27− |
| 3 | CD4 CAR: CD27+ |
| 4 | CD4 CAR: CD28+ CD27+ |
| 5 | CD4 CAR: CCR7+ |
| 6 | CD4 CAR: CCR7+ CD27+ |
| 7 | CD4 CAR: CCR7− CD45RA+ |
| 8 | CD4 CAR: CCR7− CD45RA− |
| 9 | CD4 CAR: CCR7+ CD45RA+ |
| 10 | CD4 CAR: Polyfunctionality |
| 11 | CD4 CAR: IL17+ |
| 12 | CD8 CAR: IL13+ |
| 13 | CDR CAR: CD28+ CD27− |
| 14 | CD8 CAR: CD27+ |
| 15 | CD8 CAR: CD28+ CD27+ |
| 16 | CD8 CAR: CCR7+ |
| 17 | CD8 CAR: CCR7− CD27− |
| 18 | CD8 CAR: CCR7+ CD27+ |
| 19 | CD8 CAR: CCR7− CD45RA− |
| 20 | CD8 CAR: CCR7+ CD45RA+ |
| 21 | CD8 CAR: IL-13 Secretion |
| 22 | CD8 CAR: IL-5 Secretion |
| 23 | CD8 CAR: IFNγ+ |
| 24 | CD8 CAR: IFNγ+ TNFα+ |
| 25 | CD8 CAR: CD28− CD27− |
| 26 | CD8 CAR: CD28+ |
| 27 | CD8 CAR: Cell Health |
| 28 | CD8 CAR: IL-2 Secretion |
| 29 | CD4 CAR: IL2+ TNFα+ |
| 30 | CD4 CAR: MIP-1α Secretion |
| 31 | CD4 CAR: MIP-1β Secretion |
| 32 | CD8+ T Cell Transduction |
| | Frequency |
| 33 | CD3+ T Cell Transduction |
| | Frequency |
| 34 | CD4 CAR: CCR7+ CD27− |
| 35 | CD4 CAR: CCR7+ CD45RA− |
| 36 | CD8 CAR: Activated T Cells |
| 37 | CD8 CAR: IL2+ IFNγ+ TNFα+ |
| 38 | CD4 CAR: IFNγ+ TNFα+ |
| 39 | CD4 CAR: Activated T Cells |
| 40 | CD4 CAR: IFNγ+ |
| 41 | CD8 CAR: CD4+ CAR+ |
| 42 | CD4 CAR: Viable Cell Count |
| 43 | CD8 CAR: TNFα Secretion |
| 44 | CD4 CAR: CD3+ CD4+ |
| 45 | CD4 CAR: IL2+ IFNγ+ TNFα+ |
| 46 | CD8 CAR: Viable Percentage |
| 47 | CD8 CAR: CCR7+ CD45RA− |
| 48 | CD8 CAR: CD3+ CAR+ |
| 49 | CD4 CAR: CD28− CD27− |
| 50 | CD4 CAR: CD28+ |
| 51 | CD4 CAR: IL2+ |
| 52 | CD4 CAR: IL-2 Secretion |
| 53 | CD8 CAR: IL17+ |
| 54 | CD4 CAR: IFNγ+ Secretion |
| 55 | CD4 CAR: Cell Health |
| 56 | CD8 CAR: IL2+ TNFα+ |
| 57 | CD8 CAR: IL-10 Secretion |
| 58 | CD4 CAR: CCR7− CD27+ |
| 59 | CD8 CAR: CCR7− CD27+ |
| 60 | CD4+ T Cell Transduction |
| | Frequency |
| 61 | CD3+ T Cell Transduction |
| | Frequency |
| 62 | CD8 CAR: CCR7+ CD27− |
| 63 | CD8 CAR: IFNγ Secretion |
| 64 | CD8 CAR: Hypofunctionality |
| 65 | CD4 CAR: TNFα+ |
| 66 | CD8 CAR: MIP-1α Secretion |
| 67 | CD4 CAR: GM-CSF Secretion |
| 68 | CD8 CAR: GM-CSF Secretion |
| 69 | CD4 CAR: Hypofunctionality |
| 70 | CD4 CAR: TNFα Secretion |
| 71 | CD8 CAR: Soluble CD137 |
| | Secretion |
| 72 | CD4 CAR: Soluble CD137 |
| | Secretion |
| 73 | CD4 CAR: IL-5 Secretion |
| 74 | CD4 CAR: IL-13 Secretion |
| 75 | CD4 CAR: IL-10 Secretion |
| 76 | CD4 CAR: Viable Percentage |
| 77 | CD8 CAR: Vector Copy Number |
| 78 | CD8 CAR: TNFα |
| 79 | CD8 CAR: IL2+ |
| 80 | CD4 CAR: IL13+ |
| 81 | CD8 CAR: IFNγ+ IL2+ |
| 82 | CD8 CAR: Potency |
| 83 | CD4 CAR: Potency |
| 84 | CD8 CAR: CD3+ |
| 85 | CD4 CAR: CD3+ |
| 86 | CD8 CAR: CD19+ |
| 87 | CD4 CAR: CD19+ |
| 88 | CD8 CAR: CD28− CD27+ |
| 89 | CD8 CAR: CCR7− CD45RA+ |
| 90 | CD4 CAR: CD28− CD27+ |
| 91 | CD8 CAR: Polyfunctionality |
| 92 | CD8 CAR: Viable Copy Number |
| 93 | CD8 CAR: CD3+ CD4+ |
| 94 | CD8 CAR: Viable Cell Count |
| 95 | CD4 CAR: IFNγ+ IL2+ |
| 96 | CD8 CAR: MIP-1β Secretion |
| 97 | CD8 CAR: Cytolytic Activity |
| 98 | CD4 CAR: CD3+ CAR+ |
| 99 | CD4 CAR: CD4+ CAR+ |

TABLE 2
Y Axis Label

| | |
|---|---|
| A | CD4: CCR7+ CD27+ |
| B | CD4: CCR7+ CD45RA+ |
| C | CD4: CD28+ CD27− |
| D | CD8: CCR7+ CD45RA− |
| E | CD8: CCR7+ CD45RA+ |
| F | CD8: CCR7+ CD27− |
| G | CD4: Cell Health |
| H | CD3: Cell Health |
| I | CD4: CCR7+ CD45RA− |
| J | CD4: CD28− CD27+ |
| K | CD4: CCR7+ |
| L | CD8: CCR7− CD45RA− |
| M | CD4: CD27+ |
| N | CD4: CCR7+ CD27− |
| O | CD4: CCR7− CD27+ |
| P | CD8: CD28+ |
| Q | CD8: CD28− CD27+ |
| R | CD8: Cell Health |
| S | CD8: CD28− CD27− |
| T | CD8: CCR7− CD27− |
| U | CD8: CD27+ |
| V | CD8: CD28+ CD27+ |
| W | CD4: CD28+ CD27+ |
| X | CD8: CCR7− CD27+ |
| Y | CD4: CCR7− CD45RA+ |
| Z | CD8: CCR7+ CD27+ |
| AA | CD4: CCR7− CD45RA− |
| BB | CD8: CCR7+ |
| CC | CD4: CCR7− CD27− |
| DD | CD8: CCR7− CD45RA+ |
| EE | CD4: CD28− CD27− |
| FF | CD4: CD28+ |
| GG | CD8: CD28+ CD27− |

FIG. 4 shows the exemplary predictive accuracy of two statistical learning models (lasso regression and canonical correlation analysis (CCA)) for the therapeutic cell composition attribute 3CAS−/CCR7+/CD45RA+ of CD4+/CAR+ cells.

FIG. 5 shows canonical variates for an exemplary attribute pair for a given patient lot plotted against the maximum CAR+ T cell concentration in the blood for the same patient who had received treatment with the therapeutic cell composition. CI is 95%.

FIGS. 6A-6D show the first four attribute pairs, respectively, for CD4+ and CD8+ T cells in the input composition and therapeutic cell composition determined by pCCA using a subset of attributes as shown by asterisks in Table E2.

FIGS. 7A-7D show the first four attribute pairs, respectively, for CD4+ T cell-specific attributes in the input composition and therapeutic cell composition determined by pCCA.

FIGS. 8A-8D show the first four attribute pairs, respectively, for CD8+ T cell-specific attributes in the input composition and therapeutic cell composition determined by pCCA.

### DETAILED DESCRIPTION

Provided herein are methods and compositions for use in connection with producing a cell therapy, such as an engineered T cell therapy (e.g., therapeutic cell composition) for the treatment of diseases and conditions, including various cancers. The provided embodiments relate to therapeutic T cell compositions containing engineered T cells such as those engineered to express recombinant proteins such as expressing recombinant receptors designed to recognize and/or specifically bind to molecules associated with the disease or condition and result in a response, such as an immune response against such molecules upon binding to such molecules. The receptors may include chimeric receptors, e.g., chimeric antigen receptors (CARs), and other transgenic antigen receptors including transgenic T cell receptors (TCRs). The methods provided herein allow for the identification of input composition (e.g., starting material derived from a subject) attributes that correlate with the attributes of the resulting therapeutic cell composition. In some embodiments, one or more statistical methods are used to identify correlations.

The provided methods and embodiments also relate to predicting attributes of a T cell composition prior to its production to produce an engineered (recombinant receptor-expressed) T cell composition (hereinafter also called therapeutic T cell composition). For example, in some embodiments, attributes of input compositions (e.g., starting materials derived from a subject) for the production of a therapeutic cell composition are assessed, for example by statistical learning models (e.g., machine learning models) to predict attributes of the therapeutic cell composition before subjecting the input compositions to a manufacturing process for engineering the cells with a recombinant receptor, including one or more steps of transducing the T cells, activating or stimulating the T cells, or incubating the T cells or cultivating the T cells under conditions for expansion.

In some embodiments, the input composition contains cells selected from a sample (e.g., leukapheresis or apheresis) taken from a subject. In some embodiments, the input composition is enriched for CD3+ T cells. In some embodiments, the input composition is enriched for CD4+, CD8+, or CD4+ and CD8+ T cells. In some embodiments, the attributes of the input composition (e.g., CD4+, CD8+, CD4+/CD8+ T cells of the input composition) are cell phenotype attributes, including, but not limited to, cell health (e.g., viable cell concentration, number of dead cells), the presence and/or expression of a surface marker, and/or the absence or lack of expression of a surface marker.

In some embodiments, the therapeutic cell composition is a therapeutic T cell composition produced from an input composition. In some embodiments, the therapeutic cell composition contains enriched CD3+ T cells. In some embodiments, the therapeutic cell composition contains enriched CD4+, CD8+, or CD4+ and CD8+ T cells. In some embodiments, the attributes of the therapeutic cell composition are cell phenotype attributes, including, but not limited to, cell health (e.g., viable cell count, number of dead cells), the presence and/or expression of a surface marker, the absence or lack of expression of a surface marker, the presence and/or expression of a cytokine, the absence or lack of expression of a cytokine, recombinant receptor expression (e.g., CAR+), and/or recombinant receptor-dependent activity (e.g., cytolytic activity, cytokine production).

In some embodiments, identifying correlations between input composition attributes and therapeutic cell composition attributes is useful for predicting the success of manufacturing an effective therapeutic cell composition. In some embodiments, predicting the attributes of the therapeutic cell composition before it is manufactured can inform treatment of the subject. In some embodiments, determining therapeutic cell composition attributes in advance of manufacturing may be useful for developing a treatment regimen for a subject in need thereof. In some embodiments, predicting attributes of the therapeutic composition prior to manufacturing can inform whether a subject is administered a standard and/or predetermined treatment regimen or whether and how a predetermined treatment regimen should be altered to improve clinical response. For example, if input composition attributes predict reduced or suboptimal therapeutic cell composition attributes, e.g., reduced or suboptimal attributes compared to attributes known to positively correlate with clinical outcome (e.g., response (e.g., durable response, progression free survival)), a treatment regimen may be developed to bolster or improve the effects of the therapeutic composition. For example, in some embodiments, the therapeutic cell composition may be administered to the subject as part of a combination therapy. In some embodiments, the dose or dosing (e.g., unit size or frequency of administration) of the therapeutic composition may be altered to achieve positive clinical outcome (e.g., durable response, progression free survival).

In some embodiments, predicting the attributes of the therapeutic cell composition before it is manufactured can inform the manufacturing process. For example, in some embodiments, determining therapeutic cell composition attributes in advance of manufacturing may be useful for determining whether a specific manufacturing process should be used to generate the therapeutic cell composition. In some cases, selecting a manufacturing process based on the predicted attributes of the therapeutic cell composition is a form of diagnostic manufacturing. In some embodiments, diagnostic manufacturing takes into account the predicted attributes of the therapeutic cell composition prior to manufacturing, in order to determine, e.g., select, a manufacturing process that will promote the generation of a therapeutic cell composition having desired attributes, such as attributes related to a particular percentage or threshold percentage of naïve-like T cells, including central memory T cells, or attributes that correlate with positive clinical outcomes (e.g., response (e.g., durable response, progression free survival)).

In some embodiments, manufacturing, e.g., diagnostic manufacturing, informed by the statistical learning methods described herein, can decrease the risk of manufacturing failure and/or increase the probability of manufacturing an effective therapeutic cell composition. In some embodiments, manufacturing, e.g., diagnostic manufacturing, informed by the statistical learning methods described herein reduces the impact of starting material heterogeneity, e.g., heterogeneity in starting materials derived from a subject, e.g., input compositions, on the production of a therapeutic cell composition. In this way, use of the statistical learning models provided herein to predict therapeutic cell composition attributes from an input composition can increase the number of subjects, e.g., patients, that can be successfully treated by providing guidance on which manufacturing procedures should be used to produce an effective therapeutic cell composition. For instance, manufacturing failures or low manufacturing success rate, e.g. the ability to meet a threshold harvest criteria of engineered cells, is a problem with a variety of T cell therapy products (e.g. CAR-T cell therapy products), which can be due to high variability in incoming patient material and the complex nature of generating a T cell therapy and other factors (Roddie et al. Cytotherapy, 2019, 21:327-240). Failure rates have been estimated to range from 2-14%, including an estimated 9% failure in the first approved CAR-T cell product (Seimetz, Cell Med., 2019, 11: 1-16).

In some aspects, the provided embodiments are based on the observation that certain attributes in the therapeutic cell composition, such as cell phenotype, e.g., expression of one or more surface markers; cell health; recombinant receptor expression; and recombinant receptor-dependent activity, e.g., production of one or more cytokines and/or cytolytic activity, are associated with pharmacokinetic parameters, likelihood of response and/or likelihood of developing a toxicity. In some aspects, phenotypes associated with a less differentiated phenotype, such as a high percentage of naïve-like or central memory T cells, may be associated with improved persistence and response in subjects administered a therapeutic cell composition containing such cells. In some embodiments, the expression and/or absence of expression of cell surface markers such as C-C chemokine receptor type 7 (CCR7), CD27 and CD45RA or combinations thereof in the therapeutic T cell composition for administration are positively or negatively correlated with pharmacokinetic parameters and/or response or toxicity outcomes. In some aspects, it is observed herein that phenotype and functional attributes associated with a less differentiated therapeutic T cell product or of a product enriched in naïve, naïve-like or central memory T cell subsets correlate with or exhibit a relationship with improved pharmacokinetic properties or responses, such as durability of response and/or progression free survival, following administration to a subject.

In some embodiments, the provided methods are based on observations that it can be advantageous to take into account certain attributes such as cell phenotype, e.g., expression of surface markers and combinations thereof, when determining an appropriate dose of cell therapy and/or releasing or generating cell compositions for therapy. In certain available methods, doses are based on numbers of particular cell types, such as those engineered to exhibit a particular activity, such as those positive for an engineered receptor. For example, in certain available methods and doses, dose is based upon an observed or suspected relationship between therapeutic cell composition attributes, such as the number (or number per patient weight) of cells of a certain phenotype or function, or of a subset thereof, such as of viable, cytotoxic (e.g., CD8⁺) engineered T cells. In various contexts, such numbers can have a relationship with efficacy and/or safety outcomes, such as response and/or risk of toxicities, such as neurotoxicity, cerebral edema and CRS.

In some aspects, the provided embodiments permit the administration of a controlled and consistent dose of cells, thereby minimizing variation in efficacy and/or safety outcomes in the subjects. In some aspects, controlling the dose of cells based on a defined number, ratio, percentage and/or proportions of particular subset of cells, e.g., based on cell phenotypes and recombinant receptor-dependent activity, permit the understanding of the impact of a subset of cells having particular phenotypes on the health, potency and/or efficacy of the cells contained in the therapeutic compositions. Such approaches can be used to determine and/or calculate consistent and precise effective doses of the cells in the cell therapy and/or control the pharmacokinetic parameters of the cell therapy. Provided are methods of determining such doses, including unit doses for administration, based on therapeutic cell composition attributes, including, but not limited to, the number, ratio, percentage and/or proportions of cells having particular phenotypes and/or recombinant receptor-dependent activity. In some aspects, the provided embodiments allow the identification of attributes of cells in the input composition that produce therapeutic cell compositions with attributes that correlate with pharmacokinetics (PK) and clinical outcomes, such as response and toxicity.

Common methods used in the field for correlating and/or predicting attributes include univariate analyses, such as linear regression. A univariate approach, however, fails to take into account the complex and dynamic interactions between multiple variables (e.g., attributes), which, particularly in biological fields, are important considerations. The methods provided herein take advantage of statistical methods and statistical learning models that accommodate high dimensional data sets. For example, as described herein, canonical correlation analysis (CCA) can handle high dimensional data sets containing a plurality of variables (e.g., attributes) and provide correlations and/or predictions that are not limited by or to one to one relationships. CCA can provide correlations between data sets (e.g., input attributed and therapeutic cell composition attributes) that indicate the contribution (e.g., weight) and directionality of the relationships between attributes. As such, CCA is well suited to identifying relationships between groups of variables, and predicting outcomes (e.g., therapeutic cell composition attributes) from a plurality of input variables (e.g., input composition attributes). In some embodiments, the CCA may be a penalized CCA (pCCA). In some embodiment, pCCA is used to reduce model complexity (e.g., dimensionality). In some embodiments, pCCA is used to identify attributes of the input and therapeutic compositions that are correlated. In some embodiments, pCCA identifies sets of the input and therapeutic composition attributes that are correlated. In some embodiments, CCA is used to determine (e.g., predict) attributes of a therapeutic cell composition from input composition attributes. In some embodiments, CCA predicts a plurality of therapeutic cell composition attributes from a plurality of input composition attributes.

Lasso regression is able to accommodate a plurality of variables but uses regularization to identify only those input variables that correlate with a single output variable. As such, lasso regression is useful for predicting a single variable (e.g., therapeutic cell composition attribute) from a plurality of input variables (e.g., input composition attributes).

In some embodiments, the statistical learning methods and models described herein, when used in connection with a manufacturing process, e.g., as described herein, result in a cell therapy (e.g., therapeutic cell composition) that is more effective, efficacious, and/or less toxic than alternative manufacturing processes. In certain embodiments, the statistical learning methods and models provided herein, when used in connection with a manufacturing process, for example as described herein, result in a higher rate of success for generating or producing therapeutic compositions useful for a broader population of subjects than what may be possible with alternative processes. In certain embodiments, the statistical learning methods and models provided herein, when used in connection with a manufacturing process, for example as described herein, result in improved treatments for a broader population of subjects than what may be possible using alternative processes. In some embodiments, the improved treatments are combination treatments (e.g., a therapeutic cell composition and a second therapy to increase response (e.g., durable response, progression free survival)). In some embodiments, the improved treatments are treatment doses that increase the probability of response (e.g., durable response, progression free survival). In certain embodiments, the therapeutic cell compositions produced or generated in connection with the provided methods may have greater health, viability, activation, and may have greater expression of the recombinant receptor than cells produced by alternative methods. In certain embodiments, the therapeutic cell compositions produced or generated in connection with the provided methods for correlating and predicting may be more effective than therapeutic cell compositions produced by alternative methods. Thus, the methods provided herein, when used in connection with a manufacturing process, for example as described herein, allow for the identification of subjects at risk of producing therapeutic cell compositions with poor effectiveness, efficacy, and/or safety, thereby allowing steps to be taken to improve treatment outcome. In some embodiments, the methods provided herein, when used in connection with a diagnostic manufacturing process, allow for the identification of subjects at risk of producing therapeutic cell compositions with poor effectiveness, efficacy, and/or safety, thereby allowing steps to be taken during manufacturing to improve the quality of the therapeutic cell composition. In some embodiments, the methods provided herein, including embodiments thereof, allow for a broader population of subjects to be successfully treated. In some embodiments, the methods provided herein, including embodiments thereof, account for variability (e.g., donor-to-donor variability) in the input composition, resulting in a more consistent therapeutic composition and/or effective treatment.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods for Determining Therapeutic Cell Composition Attributes

The methods provided herein allow for the identification of input composition attributes that correlate with therapeutic cell composition attributes, and further allow for the prediction of therapeutic cell composition attributes prior to the production of the therapeutic cell composition. The provided methods can be used in connection with a manufacturing procedure, for example as described herein, to produce cell therapies useful (e.g., effective) in a broad population of subjects. For example, understanding the relationship between input composition attributes and therapeutic cell composition attributes, and predicting therapeutic cell attributes prior to manufacturing allows for determining the therapeutic cell composition quality and efficacy in advance of manufacturing the composition and treatment of the subject. Having this type of information at an early stage, e.g., prior to manufacturing and treatment, allows for the development of treatment strategies (e.g., combination treatment, dosing) prior to treating the subject, thereby increasing the probability of subject response (e.g., durable response, progression free survival). In some cases, the ability to predict therapeutic cell composition attributes prior to manufacturing can inform the manufacturing process itself, such that one or more steps of the manufacturing process can be altered to increase the likelihood of producing an effective therapeutic cell composition.

In some embodiments, statistical methods are used to identify input composition and therapeutic cell attributes that are correlated (e.g., positively or negatively). In some embodiments, one or more statistical methods, for example statistical methods as described below, are used to identify correlations between input composition attributes and therapeutic cell composition attributes. In some embodiments, therapeutic cell composition attributes are predicted using a process that incorporates a statistical learning model (e.g., a machine learning model). In some embodiments, a process may incorporate one or more types of statistical learning models, for example statistical learning models as described below. In some embodiments, the statistical learning models are trained, for example on training data, to relate the attributes of input compositions to attributes of therapeutic cell compositions. In some embodiments, the statistical learning models trained as described herein can provide, e.g., predict, a quantitative profile, e.g., percentage, number, ratio, and/or proportion, of T cells in a therapeutic cell composition having particular attributes, e.g., desired attributes (see, e.g., Section I-A-2-a).

In some aspects, the provided embodiments are based on the observation that certain attributes of the therapeutic cell composition, such as cell phenotype, e.g., expression of one or more surface markers; cell health; recombinant receptor expression; and recombinant receptor-dependent activity, e.g., production of one or more cytokines and/or cytolytic activity, are associated with pharmacokinetic parameters, likelihood of response, and/or likelihood of developing a toxicity. In some aspects, large-scale or genome-wide methods, can be used to identify molecular signatures that are associated with outcomes of therapy, e.g., efficacy and safety, or pharmacokinetic parameters. As such, in some embodiments, the attributes of the input compositions and therapeutic cell compositions assessed are those shown to be correlated with positive clinical outcomes. In some embodiments, quantified attributes of the input and therapeutic compositions, for example as described below, are used as input to statistical methods (e.g., for correlation analyses) and/or statistical learning models (e.g., for predictions).

The methods provided herein include generating therapeutic cell compositions that include engineered CD3+, CD4+, CD8+, or CD4+ and CD8+ cells, and the therapeutic cell compositions are produced from input compositions that include CD3+, CD4+, CD8+, or CD4+ and CD8+ T cells. In some embodiments, the methods provided herein for generating therapeutic cell compositions include generating both CD4+ and CD8+ engineered cells for therapeutic cell compositions. For example, subjects to be treated with the therapeutic cell composition will be administered engineered CD4+ therapeutic cell compositions and engineered CD8+ therapeutic cell compositions. In some embodiments, the engineered CD4+ and CD8+ T cells are present in a single therapeutic cell composition. In some embodiments, the single therapeutic cell composition contains CD3+ T cells that are also CD4+ or CD8+. In some embodiments, the engineered CD4+ and CD8+ T cells are present in separate therapeutic cell compositions. In some embodiments, when there are separate therapeutic cell compositions, one therapeutic composition is a first therapeutic cell composition and the second therapeutic cell composition is a second therapeutic cell composition. In some embodiments, when there are first and second therapeutic cell compositions there are corresponding first and second input compositions from which are produced the first and second therapeutic cell compositions. In some embodiments, the first input and therapeutic cell compositions contain one of CD4+ or CD8+ cells and the second input and therapeutic cell compositions contain the remaining cell population (e.g., CD4+ or CD8+ T cells). In some embodiments, the therapeutic cell compositions including CD4+ and CD8+ cells are derived from mixing therapeutic cell compositions independently including CD4+ or CD8+ cells. In some embodiments, the therapeutic cell compositions including CD4+ and CD8+ cells are CD3+ T cell compositions, where the CD3+ T cells are also CD4+ or CD8+. It should be appreciated that attributes of input compositions and therapeutic cell compositions may be cell type specific (e.g., CD4+ or CD8+ specific).

A. Composition Attributes

The methods provided herein are directed to assessing the relationship between attributes of the input composition and therapeutic composition. It is contemplated that the attributes of the therapeutic cell composition (e.g., engineered T cell composition) can, in some cases, depend upon many factors, including, but not limited to, the attributes of the starting cellular material (e.g., apheresis product or leukapheresis product or cells selected therefrom (e.g., input composition)) used to generate the therapeutic cell composition. Thus, in some embodiments, attributes are also assessed in cells of the starting material (e.g., input composition) used to generate the final therapeutic cell composition. In some embodiments, the attributes assessed herein include cell phenotypes and, for example in therapeutic cell compositions, recombinant receptor-dependent activity. In some embodiments the attributes assessed are known or are suspected of correlating with clinical response.

In some embodiments, the attributes include cell phenotypes. In some embodiments, cell phenotype is determined by assessing the presence or absence of one or more specific molecules, including surface molecules and/or molecules that may accumulate or be produced by the cells or a subpopulation of cells within an input composition or therapeutic cell composition, e.g., therapeutic T cell composition. In some embodiments, cell phenotype may include cell activity, such as production of a factor (e.g., cytokine) in response to a stimulus. In some embodiments, the production of a factor (e.g., cytokine) is in response to recombinant receptor-dependent activation. In some embodiments, recombinant receptor-dependent activity of cells of a therapeutic cell composition is determined by assessing one or more specific molecules (e.g., cytokines) that may accumulate or be produced by the cells or a subpopulation of cells within a therapeutic cell composition, e.g., therapeutic T cell composition. In some embodiments, recombinant receptor-dependent activity is assessed by determining the cytolytic activity of the cells of the therapeutic composition.

In some embodiments, assessment of the attributes in the composition (e.g., input composition, therapeutic cell composition) is performed to identify, detect, or quantify a phenotype of the cell composition (e.g., surface molecule, cytokine, recombinant receptor). In particular embodiments, an assessment of the attributes of the composition (e.g., input composition, therapeutic cell composition) is performed to identify, detect, or quantify the presence, absence, degree of expression or level of a specific molecule (e.g., surface molecule, cytokine, recombinant receptor). In some embodiments, the percentage, number, ratio, and/or proportion of cells having an attribute is determined. In some embodiments, the percentage, number, ratio, and/or proportion of cells having an attribute is used as input to the statistical methods or the statistical learning models included in a process. In some embodiments, the statistical methods or the statistical learning models are those described herein.

In some embodiments, the phenotype is indicative of viability of a cell. In some embodiments, the phenotype is indicative of absence of apoptosis, absence of early stages of apoptosis or absence of late stages of apoptosis. In some embodiments, the phenotype is the absence of a factor indicative of absence of apoptosis, early apoptosis or late stages of apoptosis. In some embodiments, the phenotype is a phenotype of a sub-population or subset of T cells, such as recombinant receptor-expressing T cells (e.g. CAR⁺ T cells), CD8⁺ T cells, or CD4⁺ T cells in the therapeutic cell composition. In some embodiments, the phenotype is a phenotype of cells that are not activated and/or that lack or are reduced for or low for expression of one or more activation marker. In some embodiments, the phenotype is a phenotype of cells that are not exhausted and/or that lack or are reduced for or low for expression of one or more exhaustion markers.

In certain embodiments, the phenotype is the production of one or more cytokines. In some embodiments, for example when the cytokine is produced and/or secreted by an engineered cell of a therapeutic cell composition in response to engagement of a recombinant receptor expressed by the cell with its antigen, this activity is referred to as recombinant receptor-dependent activity. In some embodiments, the attribute is recombinant receptor-dependent activity.

In particular embodiments, the production of one or more cytokines is measured, detected, and/or quantified by intracellular cytokine staining. In particular embodiments, the phenotype is the lack of the production of the cytokine. In particular embodiments, the phenotype is positive for or is a high level of production of a cytokine. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. ICS can also be used in combination with other flow cytometry protocols for immunephenotyping using cell surface markers or with MHC multimers to access cytokine production in a particular subgroup of cells, making it an extremely flexible and versatile method. Other single-cell techniques for measuring or detecting cytokine production include, but are not limited to ELISPOT, limiting dilution, and T cell cloning.

In particular embodiments, for example in the therapeutic cell composition, the attribute includes recombinant receptor-dependent activity. In some embodiments, the activity is a recombinant receptor, e.g., a CAR, dependent activity that is or includes the production and/or secretion of a soluble factor. In certain embodiments, the soluble factor is a cytokine or a chemokine.

Suitable techniques for the measurement of the production or secretion of a soluble factor are known in the art. Production and/or secretion of a soluble factor can be measured by determining the concentration or amount of the extracellular amount of the factor, or determining the amount of transcriptional activity of the gene that encodes the factor. Suitable techniques include, but are not limited to assays such as an immunoassay, an aptamer-based assay, a histological or cytological assay, an mRNA expression level assay, an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay, protein microarrays, high-performance liquid chromatography (HPLC), Meso Scale Discovery (MSD) electrochemiluminescence and bead based multiplex immunoassays (MIA). In some embodiments, the suitable technique may employ a detectable binding reagent that specifically binds the soluble factor.

In some embodiments, the phenotype is indicated by the presence, absence, or level of expression in a cell of one or more specific molecules, such as certain surface markers indicative of the phenotype, e.g., surface proteins; intracellular markers indicative of the phenotype; or nucleic acids indicative of the phenotype or other molecules or factors indicative of the phenotype. In some embodiments, the phenotype is or comprises a positive or negative expression of the one or more of specific molecules. In some embodiments, the specific molecules include, but are not limited to, a surface marker, e.g., a membrane glycoprotein or a receptor; a marker associated with apoptosis or viability; or a specific molecule that indicates the status of an immune cells, e.g., a marker associated with activation, exhaustion, or a mature or naïve phenotype. In some embodiments, any known method for assessing or measuring, counting, and/or quantifying cells based on specific molecules can be used to determine the number of cells of the phenotype in the composition (e.g., input composition, therapeutic cell composition).

In some embodiments, a phenotype is or includes a positive or negative expression of one or more specific molecules in a cell. In some embodiments, the positive expression is indicated by a detectable amount of the specific molecule in the cell. In certain embodiments, the detectable amount is any detected amount of the specific molecule in the cell. In particular embodiments, the detectable amount is an amount greater than a background, e.g., background staining, signal, etc., in the cell. In certain embodiments, the positive expression is an amount of the specific molecule that is greater than a threshold, e.g., a predetermined threshold. Likewise, in particular embodiments, a cell with negative expression of a specific molecule may be any cell not determined to have positive expression, or is a cell that lacks a detectable amount of the specific molecule or a detectable amount of the specific molecule above background. In some embodiments, the cell has negative expression of a specific molecule if the amount of the specific molecule is below a threshold. One of skill in the art will understand how to define a threshold to define positive and/or negative expression for a specific molecule as a matter of routine skill, and that the thresholds may be defined according to specific parameters of, for example, but not limited to, the assay or method of detection, the identity of the specific molecule, reagents used for detection, and instrumentation.

Examples of methods that can be used to detect a specific molecule and/or analyze a phenotype of the cells include, but are not limited to, biochemical analysis; immunochemical analysis; image analysis; cytomorphological analysis; molecule analysis such as PCR, sequencing, high-throughput sequencing, determination of DNA methylation; proteomics analysis such as determination of protein glycosylation and/or phosphorylation pattern; genomics analysis; epigenomics analysis (e.g., ChIP-seq or ATAC-seq); transcriptomics analysis (e.g., RNA-seq); and any combination thereof. In some embodiments, the methods can include assessment of immune receptor repertoire, e.g., repertoire of T cell receptors (TCRs). In some aspects, determination of any of the phenotypes can be assessed in high-throughput, automated and/or by single-cell-based methods. In some aspects, large-scale or genome-wide methods, can be used to identify one or more molecular signatures. In some aspects, one or more molecular signatures, e.g., expression of specific RNA or proteins in the cell, can be determined. In some embodiments, molecular features of the phenotype analyzed by image analysis, PCR (including the standard and all variants of PCR), microarray (including, but not limited to DNA microarray, MMchips for microRNA, protein microarray, cellular microarray, antibody microarray, and carbohydrate array), sequencing, biomarker detection, or methods for determining DNA methylation or protein glycosylation pattern. In particular embodiments, the specific molecule is a polypeptide, i.e. a protein. In some embodiments, the specific molecule is a polynucleotide.

In some embodiments, positive or negative expression of a specific molecule is determined by incubating cells with one or more antibodies or other binding agent that specifically bind to one or more surface markers expressed or expressed (marker⁺) at a relatively higher level (marker^{high}) on the positively or negatively selected cells, respectively. In particular embodiments, the positive or negative expression is determined by flow cytometry, immunohistochemistry, or any other suitable method for detecting specific markers.

In particular embodiments, expression of a specific molecule is assessed with flow cytometry. Flow cytometry is a laser- or impedance-based, biophysical technology employed in cell counting, cell sorting, biomarker detection and protein engineering, by suspending cells in a stream of fluid and passing them by an electronic detection apparatus. It allows simultaneous multiparametric analysis of the physical and chemical characteristics of up to thousands of particles per second.

The data generated by flow-cytometers can be plotted in a single dimension, to produce a histogram, or in two-dimensional dot plots or even in three dimensions. The regions on these plots can be sequentially separated, based on fluorescence intensity, by creating a series of subset extractions, termed “gates.” Specific gating protocols exist for diagnostic and clinical purposes especially in relation to immunology. Plots are often made on logarithmic scales. Because different fluorescent dyes' emission spectra overlap, signals at the detectors have to be compensated electronically as well as computationally. Data accumulated using the flow cytometer can be analyzed using software, e.g., JMP (statistical software), WinMDI, Flowing Software, and web-based Cytobank), Cellcion, FCS Express, FlowJo, FACSDiva, CytoPaint (aka Paint-A-Gate), VenturiOne, CellQuest Pro, Infinicyt or Cytospec.

Flow Cytometry is a standard technique in the art and one of skill would readily understand how to design or tailor protocols to detect one or more specific molecules and analyze the data to determine the expression of one or more specific molecules in a population of cells. Standard protocols and techniques for flow cytometry are found in Loyd “Flow Cytometry in Microbiology; Practical Flow Cytometry by Howard M. Shapiro; Flow Cytometry for Biotechnology by Larry A. Sklar, Handbook of Flow Cytometry Methods by J. Paul Robinson, et al., Current Protocols in Cytometry, Wiley-Liss Pub, Flow Cytometry in Clinical Diagnosis, v4, (Carey, McCoy, and Keren, eds), ASCP Press, 2007, Ormerod, M. G. (ed.) (2000) Flow Cytometry—A practical approach. 3rd edition. Oxford University Press, Oxford, UK, Ormerod, M. G. (1999) Flow Cytometry. 2nd edition. BIOS Scientific Publishers, Oxford., and Flow Cytometry—A basic introduction. Michael G. Ormerod, 2008.

In some embodiments, cells are sorted by phenotype for further analysis. In some embodiments, cells of different phenotypes within the same cell composition, e.g., input composition or therapeutic cell composition, are sorted by Fluorescence-activated cell sorting (FACS). FACS is a specialized type of flow cytometry that allows for sorting a heterogeneous mixture of cells into two or more containers, one cell at a time, based upon the specific light scattering and fluorescent characteristics of each cell. It is a useful scientific instrument as it provides fast, objective and quantitative recording of fluorescent signals from individual cells as well as physical separation of cells of particular interest.

1. Input Composition Attributes

In some embodiments, the input composition contains cells isolated from samples (e.g., biological samples), such as those obtained from or derived from a subject, such as one having a particular disease or condition or in need of a cell therapy or to which cell therapy will be administered. Methods for isolating cells from samples (e.g., biological samples) are described, for example, in Section II-A. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. In some embodiments, the input composition contains CD4+ and CD8+ T cells. In some embodiments, the input composition contains CD4+ or CD8+ T cells.

In some embodiments, attributes of the input composition include cell phenotypes. In some embodiments, the phenotype is the number of total T cells. In some embodiments, the phenotype is the number of total CD3⁺ T cells. In some embodiments, the phenotype is or includes the identity of a T cell subtype. Different populations or subtypes of T cells include, but are not limited to effector T cells, helper T cells, memory T cell, Regulatory T cells, naïve T cells, CD4⁺ cells, and CD8⁺ T cells. In certain embodiments, a T cell subtype may be identified by detecting the presence or absence of a specific molecule. In certain embodiments, the specific molecule is a surface marker that can be used to identify a T cell subtype.

In some embodiments, the phenotype is positive or high level expression of one or more specific molecule that are surface markers, e.g., CD3, CD4, CD8, CD28, CD62L, CCR7, CD27, CD127, CD4, CD8, CD45RA, and/or CD45RO. In certain embodiments, the phenotype is a surface marker of T cells or of a subpopulation or subset of T cells, such as based on positive surface marker expression of one or more surface markers, e.g., CD3⁺, CD4⁺, CD8⁺, CD28⁺, CD62L⁺, CCR7⁺, CD27⁺, CD127⁺, CD4⁺, CD8⁺, CD45RA⁺, and/or CD45RO⁺. In some embodiments, the phenotype is positive or high level expression of one or more specific molecule that are surface markers, e.g., C-C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin. In some embodiments, the phenotype is negative or the absence of expression of one or more specific molecules that are surface markers, e.g., CD3, CD4, CD8, CD28, CD62L, CCR7, CD27, CD127, CD4, CD8, CD45RA, and/or CD45RO. In certain embodiments, the phenotype is a surface marker of T cells or of a subpopulation or subset of T cells, such as based on the absence of surface marker expression of one or more surface markers, e.g., CD3⁻, CD4⁻, CD8⁻, CD28⁻, CD62L⁻, CCR7⁻, CD27⁻, CD127⁻, CD4⁻, CD8⁻, CD45RA⁻, and/or CD45RO⁻. In some embodiments, the phenotype is negative or the absence of expression of one or more specific molecule that are surface markers, e.g., C—C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin.

In certain embodiments, the phenotype is or includes positive or negative expression of CD27, CCR7 and/or CD45RA. In some embodiments, the phenotype is CCR7⁺. In some embodiments, the phenotype is CD27⁺. In some embodiments, the phenotype is CCR7⁻. In some embodiments, the phenotype is CD27⁻. In some embodiments, the phenotype is CCR7⁺/CD27⁺. In some embodiments, the phenotype is CCR7⁻/CD27⁺. In some embodiments, the phenotype is CCR7⁺/CD27⁻. In some embodiments, the phenotype is CCR7⁻/CD27⁻. In some embodiments, the phenotype is CD45RA⁻. In some embodiments, the phenotype is CD45RA⁺. In some embodiments, the phenotype is CCR7⁺/CD45RA⁻. In some embodiments, the phenotype is CD27⁺/CD45RA⁻. In some embodiments, the phenotype is CD27⁺/CD45RA⁺. In some embodiments, the phenotype is CD27⁻/CD45RA⁺. In some embodiments, the phenotype is CD27⁻/CD45RA⁻. In some embodiments, the phenotype is CCR7⁺/CD27⁺/CD45RA⁻. In some embodiments, the phenotype is CCR7⁺/CD27⁺/CD45RA+.

In some embodiments, the phenotype is viability. In certain embodiments, the phenotype is the positive expression of a marker that indicates that the cell undergoes normal functional cellular processes and/or has not undergone or is not under the process of undergoing necrosis or programmed cell death. In some embodiments, viability can be assessed by the redox potential of the cell, the integrity of the cell membrane, or the activity or function of mitochondria. In some embodiments, viability is the absence of a specific molecule associated with cell death, or the absence of the indication of cell death in an assay.

In some embodiments, the phenotype is or comprises cell viability. In certain embodiments, the viability of cells can be detected, measured, and/or assessed by a number of means that are routine in the art. Non-limiting examples of such viability assays include, but are not limited to, dye uptake assays (e.g., calcein AM assays), XTT cell viability assays, and dye exclusion assays (e.g., trypan blue, Eosin, or propidium dye exclusion assays). Viability assays are useful for determining the number or percentage (e.g., frequency) of viable cells in a cell dose, a cell composition, and/or a cell sample. In particular embodiments, the phenotype comprises cell viability along with other features, e.g., surface makers, molecules.

In certain embodiments, the phenotype is or includes cell viability, viable CD3⁺, viable CD4⁺, viable CD8⁺, viable CD4⁺/CCR7⁺, viable CD8⁺/CD27⁺, viable CD4⁺/CD27⁺, viable CD8⁺/CCR7⁺/CD27⁺, viable CD4⁺/CCR7⁺/CD27⁺, viable CD8⁺/CCR7⁺/CD45RA⁻ or viable CD4⁺/CCR7⁺/CD45RA⁻ cells or a combination thereof.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death. These changes include blebbing, cell shrinkage, nuclear fragmentation, chromatin condensation, chromosomal DNA fragmentation, and global mRNA decay. Apoptosis is a well characterized process, and specific molecules associated with various stages are well known in the art.

In some embodiments, the phenotype is the absence of an early stage of apoptosis, and/or an absence of an indicator and/or a specific molecule associated with an early stage of apoptosis. In the early stages of apoptosis, changes in the cellular and mitochondrial membrane become apparent. Biochemical changes are also apparent in the cytoplasm and nucleus of the cell. For example, the early stages of apoptosis can be indicated by activation of certain caspases, e.g., 2, 8, 9, and 10. In particular embodiments, the phenotype is the absence of a late stage of apoptosis, and/or an absence of an indicator and/or a specific molecule associated with a late stage of apoptosis. The middle to late stages of apoptosis are characterized by further loss of membrane integrity, chromatin condensation and DNA fragmentation, and include biochemical events such as activation of caspases 3, 6, and 7.

In certain embodiments, the phenotype is the negative expression of one or more factors associated with apoptosis, including pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In some embodiments, the phenotype is a negative or low amount of a marker of apoptosis. In certain embodiments, the phenotype is the negative expression of a marker of apoptosis. In certain embodiments, the phenotype is the absence of an indicator, e.g., staining with an Annexin V molecule, which will preferentially bind to cells undergoing apoptosis when incubated with or contacted to a cell composition. In some embodiments, the phenotype is or includes the expression of one or more markers that are indicative of an apoptotic state in the cell.

In some embodiments, the phenotype is the negative (or low) expression of a specific molecule that is a marker for apoptosis. Various apoptosis markers are known to those of ordinary skill in the art and include, but are not limited to, an increase in activity of one or more caspases i.e. an activated caspase (e.g., an active caspase, CAS), an increase in PARP cleavage, activation and/or translocation of Bcl-2 family proteins, members of the cell death pathway, e.g., Fas and FADD, presence of nuclear shrinkage (e.g., monitored by microscope) and presence of chromosome DNA fragmentation (e.g., presence of chromosome DNA ladder) or with apoptosis assays that include TUNEL staining, and Annexin V staining.

Caspases are enzymes that cleave proteins after an aspartic acid residue, the term is derived from “cysteine-aspartic acid proteases.” Caspases are involved in apoptosis, thus activation of caspases, such as caspase-3 is indicative of an increase or revival of apoptosis. In some embodiments, activated caspase-3 is referred to herein as 3CAS. In certain embodiments, caspase activation can be detected by methods known to the person of ordinary skill. In some embodiments, an antibody that binds specifically to an activated caspase (i.e., binds specifically to the cleaved polypeptide) can be used to detect caspase activation. In another example, a fluorochrome inhibitor of caspase activity (FLICA) assay can be utilized to detect caspase-3 activation by detecting hydrolysis of acetyl Asp-Glu-Val-Asp 7-amido-4-methylcoumarin (Ac-DEVD-AMC) by caspase-3 (i.e., detecting release of the fluorescent 7-amino-4-methylcoumarin (AMC)). FLICA assays can be used to determine caspase activation by a detecting the product of a substrate processed by multiple caspases (e.g., FAM-VAD-FMK FLICA). Other techniques include The CASPASE-GLO® caspase assays (PROMEGA) that use luminogenic caspase-8 tetrapeptide substrate (Z-LETD-aminoluciferin), the caspase-9 tetrapeptide substrate (Z-LEHD-aminoluciferin), the caspase-3/7 substrate (Z-DEVD-aminoluciferin), the caspase-6 substrate (Z-VEID-aminoluciferin), or the caspase-2 substrate (Z-VDVAD-aminoluciferin).

In certain embodiments, the phenotype is or includes negative expression of activated caspase-1, activated caspase-2, activated caspase-3, activated caspase-7, activated caspase-8, activated caspase-9, activated caspase-10 and/or activated caspase-13 in a cell. In particular embodiments, the phenotype is or includes activated caspase 3-. In some embodiments, the proform (zymogen cleaved) form of a caspase, such as any above, also is a marker indicating the presence of apoptosis. In some embodiments, the phenotype is or includes the absence of or negative expression of a proform of a caspase, such as the proform of caspase-3.

In some embodiments, the marker of apoptosis is cleaved the Poly ADP-ribose polymerase 1 (PARP). PARP is cleaved by caspase during early stages of apoptosis. Thus, detection of a cleaved PARP peptide is a marker for apoptosis. In particular embodiments, the phenotype is or includes positive or negative expression of cleaved PARP.

In some embodiments, the marker of apoptosis is a reagent that detects a feature in a cell that is associated with apoptosis. In certain embodiments, the reagent is an annexin V molecule. During the early stages of apoptosis the lipid phosphatidylserine (PS) translocates from the inner to the outer leaflet of the plasma membrane. PS is normally restricted to the internal membrane in healthy and/or non-apoptotic cells. Annexin V is a protein that preferentially binds phosphatidylserine (PS) with high affinity. When conjugated to a fluorescent tag or other reporter, Annexin V can be used to rapidly detect this early cell surface indicator of apoptosis. In some embodiments, the presence of PS on the outer membrane will persist into the late stages of apoptosis. Thus in some embodiments, annexin V staining is an indication of both early and late stages of apoptosis. In certain embodiments, an Annexin, e.g. Annexin V, is tagged with a detectable label and incubated with, exposed to, and/or contacted with cells of a cell composition to detect cells that are undergoing apoptosis, for example by flow cytometry. In some embodiments, fluorescence tagged annexins, e.g., annexin V, are used to stain cells for flow cytometry analysis, for example with the annexin⁻V/7⁻ AAD assay. Alternative protocols suitable for apoptosis detection with annexin include techniques and assays that utilize radiolabeled annexin V. In certain embodiments, the phenotype is or includes negative staining by annexin, e.g. annexin V⁻. In particular embodiments, the phenotype is or includes the absence of PS on the outer plasma membrane. In certain embodiments, the phenotype is or includes cells that are not bound by annexin e.g. annexin V. In certain embodiments, the cell that lacks detectable PS on the outer membrane is annexin V⁻. In particular embodiments, the cell that is not bound by annexin V⁻ in an assay, e.g., flow cytometry after incubation with labeled annexin V, is annexin V⁻.

In particular embodiments, the phenotype is annexin V⁻, annexin V⁻CD3⁺, annexin V⁻CD4⁺, annexin V⁻CD8⁺, annexin V⁻CD3⁺, annexin V⁻CD4+, annexin V⁻CD8+, activated caspase 3⁻, activated caspase 3⁻/CD3⁺, activated caspase 3⁻/CD4⁺, activated caspase 3⁻/CD8⁺, activated caspase 3⁻/CD3⁺, activated caspase 3⁻/CD4⁺, activated caspase 3⁻/CD8⁺, annexin V⁻/CD4⁺/CCR7⁺, annexin V⁻/CD8⁺/CD27⁺, annexin V⁻/CD4⁺/CD27⁺, annexin V⁻/CD8⁺/CCR7⁺/CD27⁺, annexin V⁻/CD4⁺/CCR7⁺/CD27⁺, annexin V⁻/CD8⁺/CCR7⁺/CD45RA⁻ or annexin V−/CD4⁺/CCR7⁺/CD45RA⁻; activated caspase 3⁻/CD4⁺/CCR7⁺, activated caspase 3−/CD8⁺/CD27⁺, activated caspase 3⁻/CD4⁺/CD27⁺, activated caspase 3-/CD8+/CCR7+/CD27+, activated caspase 3⁻/CD4⁺/CCR7⁺/CD27⁺, activated caspase 3⁻/CD8⁺/CCR7⁺/CD45RA⁻ or activated caspase 3⁻/CD4⁺/CCR7⁺/CD45RA⁻ or a combination thereof. In some embodiments, the phenotype is 3CAS−/CCR7−/CD27−. In some embodiments, the phenotype is 3CAS−/CCR7−/CD27+. In some embodiments, the phenotype is 3CAS−/CCR7+. In some embodiments, the phenotype is 3CAS−/CCR7+/CD27−. In some embodiments, the phenotype is 3CAS−/CCR7+/CD27+. In some embodiments, the phenotype is 3CAS−/CD27+. In some embodiments, the phenotype is 3CAS−/CD28−/CD27−. In some embodiments, the phenotype is 3CAS−/CD28−/CD27+. In some embodiments, the phenotype is 3CAS−/CD28+. In some embodiments, the phenotype is 3CAS−/CD28+/CD27−, In some embodiments, the phenotype is 3CAS−/CD28+/CD27+. In some embodiments, the phenotype is 3CAS−/CCR7−/CD45RA−. In some embodiments, the phenotype is 3CAS−/CCR7−/CD45RA+. In some embodiments, the phenotype is 3CAS−/CCR7+/CD45RA−. In some embodiments, the phenotype is 3CAS−/CCR7+/CD45RA+. In some embodiments, the phenotype further is CD4+. In some embodiments, the phenotype further is CD8+.

Particular embodiments contemplate that cells positive for expression of a marker for apoptosis are undergoing programmed cell death, show reduced or no immune function, and have diminished capabilities if any to undergo activation, expansion, and/or bind to an antigen to initiate, perform, or contribute to an immune response or activity. In particular embodiments, the phenotype is defined by negative expression for an activated caspase and/or negative staining with annexin V.

In certain embodiments, the phenotype is or includes activated caspase 3 (caspase 3, 3CAS) and/or annexin V.

Among the phenotypes are the expression or surface expression of one or more markers generally associated with one or more sub-types or subpopulations of T cells, or phenotypes thereof. T cell subtypes and subpopulations may include CD4⁺ and/or of CD8⁺ T cells and subtypes thereof that may include naïve T (T_{N}) cells, naïve-like cells, effector T cells (T_{EFF}), memory T cells and sub-types thereof, such as stem cell memory T cells (T_{SCM}), central memory T cells (T_{CM}), effector memory T (T_{EM}), T_{EMRA} cells or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some aspects, among the phenotypes include expression or markers or functions, e.g. antigen-specific functions such as cytokine secretion, that are associated with a less differentiated cell subset or a more differentiated subset. In some embodiments, the phenotypes are those associated with a less differentiated subset, such as one or more of CCR7⁺, CD27⁺ and interleukin-2 (IL-2) production. In some aspects, less differentiated subsets can also be related to therapeutic efficacy, self-renewal, survival functions or graft-versus-host disease. In some aspects, less differentiated cells, e.g., central memory cells, are longer lived and exhaust less rapidly, thereby increasing persistence and durability. In some embodiments, the phenotypes are those associated with a more differentiated subset, such as one or more of interferon-gamma (IFN-γ) or IL-13 production. In some aspects, more differentiated subsets can also be related to senescence and effector function.

In some embodiments, the phenotype is or includes a phenotype of a memory T cell or memory T cell subset exposed to their cognate antigen. In some embodiments the phenotype is or includes a phenotype of a memory T cell (or one or more markers associated therewith), such as a T_{CM} cell, a T_{EM} cell, or a T_{EMRA} cell, a T_{SCM} cell, or a combination thereof. In particular embodiments, the phenotype is or includes the expression of one or more specific molecules that is a marker for memory and/or memory T cells or subtypes thereof. In some aspects, exemplary phenotypes associated with T_{CM} cells can include one or more of CD45RA⁻, CD62L⁺, CCR7⁺, CD27+, CD28+ and CD95⁺. In some aspects, exemplary phenotypes associated with T_{EM} cells can include one or more of CD45RA⁻, CD62L⁻, CCR7⁻, CD27−, CD28−, and CD95⁺.

In particular embodiments, the phenotype is or includes the expression of one or more specific molecules that is a marker for naïve T cells.

In some embodiments, the phenotype is or includes a memory T cell or a naïve T cell. In certain embodiments, the phenotype is the positive or negative expression of one or more specific molecules that are markers for memory. In some embodiments, the memory marker is a specific molecule that may be used to define a memory T cell population.

In some embodiments, the phenotype is or includes a phenotype of or one or more marker associated with a non-memory T cell or sub-type thereof; in some aspects, it is or includes a phenotype or marker(s) associated with a naïve cell. In some aspects, exemplary phenotypes associated with naïve T cells can include one or more of CCR7+, CD45RA+, CD27+, and CD28+. In some embodiments, the phenotype is CCR7⁺/CD27⁺/CD28⁺/CD45RA⁺. In certain embodiments, the phenotype is or includes CCR7⁺/CD45RA⁺. In certain embodiments, the phenotype is or includes CCR7⁺/CD27⁺. In certain embodiments, the phenotype is or includes CD27⁺/CD28+. In some embodiments, the phenotype is or includes a phenotype of a central memory T cell. In particular embodiments, the phenotype is or includes CCR7⁺/CD27⁺/CD28⁺/CD45RA⁻. In some embodiments, the phenotype is or includes CCR7⁻/CD27⁺/CD28⁺/CD45RA⁻. In some embodiments, the phenotype is or includes CCR7⁺/CD27⁺. In some embodiments, the phenotype is or includes CD27⁺/CD28⁺. In certain embodiments, the phenotype is or includes that of a T_{EMRA} cell or a T_{SCM} cell. In certain embodiments, the phenotype is or includes CD45RA⁺. In particular embodiments, the phenotype is or includes CCR7⁻/CD27⁻/CD28⁻/CD45RA⁺. In some embodiments, the phenotype is or includes one of CD27⁺/CD28⁺, CD27⁻/CD28⁺, CD27⁺/CD28⁻, or CD27⁻/CD28⁻. In some embodiments, the phenotype is CCR7⁺/CD27⁺/CD45RA⁺. In certain embodiments, the phenotype is or includes CCR7⁺/CD45RA⁺. In certain embodiments, the phenotype is or includes CD27−/CD28−. In particular embodiments, the phenotype is or includes CCR7⁺/CD27⁺/CD45RA⁻. In some embodiments, the phenotype is or includes CCR7⁻/CD27⁺/CD45RA⁻. In certain embodiments, the phenotype is or includes CD45RA⁺. In particular embodiments, the phenotype is or includes CCR7⁻/CD27⁻/CD45RA⁺. In particular embodiments, the phenotype is or includes CCR7⁺/CD27⁺/CD28⁺/CD45RA⁻; CCR7⁻/CD27⁺/CD28⁺/CD45RA⁻; CCR7⁻/CD27⁻/CD28⁻/CD45RA⁺; CD27⁺/CD28⁺; CD27⁻/CD28⁺; CD27⁺/CD28⁻; or CD27⁻/CD28⁻. In particular embodiments, the phenotype is or includes CCR7⁺/CD27⁺/CD45RA⁻; CCR7⁻/CD27⁺/CD45RA⁻; CCR7⁻/CD27⁻/CD28⁻/CD45RA⁺; CD27⁺; CD27⁻; CD27⁺/CD28⁻; or CD27⁻/CD28⁻.

In some embodiments, the phenotype is or includes a phenotype of one or more markers associated with a naïve-like T cell. In some embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+/CCR7+, where the cells are CD45RA+ or CD45RA−. In certain embodiments, naïve-like T cells or the T cells that are surface positive for a marker expressed on naïve-like T cells are CD62L−CCR7+.

In some embodiments, the phenotype is or includes a phenotype of one or more markers associated with an intermediate type T cell. In some embodiments, intermediate T cells may be characterized by positive or high expression (e.g., surface expression or intracellular expression) of certain cell markers and/or negative or low expression (e.g., surface expression or intracellular expression) of other cell markers. In some cases, the markers are markers that classify a T cell as an intermediate T cell, which are T cells that share features of naïve/memory T cells as well as terminally differentiated effector T cells, in that the cells are able to produce IFN-gamma and exhibit cytolytic activity, and also retain the ability to produce IL-2 and proliferate. In some aspects, intermediate T cells are characterized by positive or high expression of CCR7 and/or CD28. In some embodiments, intermediate T cells are CCR7+/CD45RA−, CD28+, or CD28+/CD27− cells.

In certain embodiments, the phenotype is or includes a phenotype of a T cell that is negative for a marker of apoptosis. In certain embodiments, the phenotype is or includes a naïve cell that is negative for a marker of apoptosis. In some embodiments, the marker of apoptosis is activated caspase 3 (3CAS). In some embodiments, the marker of apoptosis is positive staining by annexin V. In particular embodiments, the phenotype is or includes CD27⁺/CD28⁺, CD27⁻/CD28⁺, CD27⁺/CD28⁻, CD27⁻/CD28⁻, or a combination thereof.

In certain embodiments, the phenotype is or includes activated caspase 3⁻/CD27⁺/CD28⁺, activated caspase 3⁻/CD27⁻/CD28⁺, activated caspase 3⁻/CD27⁺/CD28⁻, activated caspase 3⁻/CD27⁻/CD28⁻, or a combination thereof. In particular embodiments, the phenotype is or includes annexin V⁻/CD27⁺/CD28⁺, annexin V⁻/CD27⁻/CD28⁺, annexin V⁻/CD27⁺/CD28⁻, annexin V⁻/CD27⁻/CD28⁻, or a combination thereof. In particular embodiments, the phenotype is or includes CD27⁺, CD27⁻, CD27⁺, CD27⁻, or a combination thereof. In some embodiments, the phenotype is or includes CD27⁺, CD27⁻, CD27⁺, CD27⁻, or a combination thereof. In certain embodiments, the phenotype is or includes activated caspase 3⁻/CD27⁺, activated caspase 3⁻/CD27⁻, activated caspase 3⁻/CD27⁺, activated caspase 3⁻/CD27⁻, or a combination thereof. In particular embodiments, the phenotype is or includes annexin V⁻/CD27⁺, annexin V⁻/CD27⁻, annexin V⁻/CD27⁺, annexin V⁻/CD27−, or a combination thereof.

In particular embodiments, the phenotype is or includes CCR7+/CD28+, CCR7⁻/CD28⁺, CCR7⁺/CD28⁻, CCR7⁻/CD28⁻, or a combination thereof. In some embodiments, the phenotype is or includes CCR7⁺/CD28⁺, CCR7⁻/CD28⁺, CCR7⁺/CD28⁻, CCR7⁻/CD28⁻, or a combination thereof. In certain embodiments, the phenotype is or includes activated caspase 3⁻/CCR7⁺/CD28⁺, activated caspase 3⁻/CCR7⁻/CD28⁺, activated caspase 3⁻/CCR7⁺/CD28⁻, activated caspase 3⁻/CCR7⁻/CD28⁻, or a combination thereof. In particular embodiments, the phenotype is or includes annexin V⁻/CCR7⁺/CD28⁺, annexin V⁻/CCR7⁻/CD28⁺, annexin V⁻/CCR7⁺/CD28⁻, annexin V⁻/CCR7⁻/CD28⁻, or a combination thereof. In particular embodiments, the phenotype is or includes CCR7⁺, CCR7⁻, CCR7⁺, CCR7⁻, or a combination thereof. In some embodiments, the phenotype is or includes CCR7⁺, CCR7⁻, CCR7⁺, CCR7⁻, or a combination thereof. In certain embodiments, the phenotype is or includes activated caspase 3⁻/CCR7⁺, activated caspase 3⁻/CCR7⁻, activated caspase 3⁻/CCR7⁺, activated caspase 3⁻/CCR7⁻, or a combination thereof. In particular embodiments, the phenotype is or includes annexin V⁻/CCR7⁺, annexin V⁻/CCR7⁻, annexin V⁻/CCR7⁺, annexin V⁻/CCR7₋, or a combination thereof.

In some embodiments, the input composition phenotypes include 3CAS−/CCR7−/CD27−, 3CAS−/CCR7−/CD27+, 3CAS−/CCR7+, 3CAS−/CCR7+/CD27−, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and/or CAS+/CD3+. In some embodiments, for example when the input composition is CD4+ T cells, the input composition phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS/−CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, CAS+/CD4+, CAS+/CD3+, and/or 3CAS−/CCR7+/CD45RA+/CD4+.

In some embodiments, for example when the input composition is CD8+ T cells, the input composition phenotypes include 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD8+, and/or CAS+/CD3+.

In some embodiments, for example when the input composition is CD4+ and CD8+ T cells or the input composition separately contains CD4+ T cells or CD8+ T cells, the input composition phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, and/or CAS+/CD3+.

The term phenotype may be referred to as a cell phenotype or T cell phenotype herein.

In some embodiments, the input composition attributes are those shown in Table E2 below. In some embodiments, the input composition attributes are any one or more of those shown in Table E2 below.

In some of any of the above embodiments, the percentage, number, and/or proportion of cells having an attribute that is a phenotype as described above, is determined, measured, obtained, detected, observed, and/or identified. In certain embodiments, the number of cells of the phenotype is the total amount of cells of the phenotype of the input composition. In some embodiments, the number of the cells of the phenotype may be expressed as a frequency, ratio, and/or a percentage of cells of the phenotype present in the input composition.

In some embodiments, the number, multiple, or fraction of the cells of a phenotype is transformed, for example to compress the range of relevant values of the number, multiple, or fraction. In some embodiments, the transformation is any application of a deterministic mathematical function to each point in a data set, such as, each data point x is replaced with the transformed value y=f(x), where f is a function. In general, transforms may be applied so that the data appear to more closely meet the assumptions of a statistical inference procedure that is to be applied, or to improve the interpretability or appearance of graphs. In most cases the function that is used to transform the data is invertible, and generally is continuous. The transformation is usually applied to a collection of comparable measurements. Examples of suitable transformations include, but are not limited to, logarithm and square root transformation, reciprocal transformations, and power transformations. In certain embodiments, the number, multiple, or fraction of the cells of a phenotype is transformed by a logarithmic transformation. In certain embodiments, the logarithmic transformation is a common log (log 10(x)), a natural log (ln(x)) or a binary log (log 2(x)).

2. Therapeutic Cell Composition Attributes

In some embodiments, a therapeutic cell composition is generated (e.g., as described herein) from an input composition, for example as described above. In some embodiments, the therapeutic cell composition contains CD4+ T cells. In some embodiments, the therapeutic cell composition contains CD8+ T cells. In some embodiments, the therapeutic cell composition contains CD4+ and CD8+ T cells. In some embodiments, attributes of the therapeutic cell composition, such as cell phenotypes and/or recombinant receptor-dependent activity, e.g., production of one or more cytokines and/or cytolytic activity, or the level or percentage of such phenotypes and/or recombinant receptor-dependent activity in the therapeutic composition of cells, correlate to or are associated with activity and/or function of the cells and/or the likelihood of developing a toxicity, such as cytokine release syndrome (CRS) or neurotoxicity (NT) and/or an outcome of the cell therapy, e.g., response to the cell therapy, in a subject administered the T cell composition, such as durability of response and progression free survival. In some embodiments, compositions including cells with specific phenotypes and/or recombinant receptor-dependent activity, and, more particularly, percentages of cells with such specific phenotypes and/or recombinant receptor-dependent activity, correlate with clinical outcomes, such as durable response and/or progression free survival. Thus, in some embodiments, attributes associated with positive clinical outcome are assessed in both input composition and therapeutic cell compositions. In some embodiments, therapeutic cell composition attributes associated with positive clinical outcome, e.g., response, are referred to as desired attributes.

In some embodiments, attributes of the therapeutic cell composition include cell phenotypes. In some embodiments, the phenotype is the number of total T cells. In some embodiments, the phenotype is the number of total CD3+ T cells. In particular embodiments, phenotype includes cells that express a recombinant receptor or a CAR. In some embodiments, the phenotype includes one or more different subtypes of T cells. In some embodiments, the one or more different subtypes further express a recombinant receptor or a CAR. In some embodiments, the phenotype is or includes the identity of a T cell subtype. Different populations or subtypes of T cells include, but are not limited to effector T cells, helper T cells, memory T cell, effector memory T cells, Regulatory T cells, naïve T cells, naïve-like T cells, CD4+ cells, and CD8+ T cells. In certain embodiments, a T cell sub-type may be identified by detecting the presence or absence of a specific molecule. In certain embodiments, the specific molecule is a surface marker that can be used to identify a T cell subtype.

In some embodiments, the phenotype is positive or high level expression of one or more specific molecule that are surface markers, e.g., CD3, CD4, CD8, CD28, CD62L, CCR7, CD27, CD127, CD4, CD8, CD45RA, and/or CD45RO. In certain embodiments, the phenotype is a surface marker of T cells or of a subpopulation or subset of T cells, such as based on positive surface marker expression of one or more surface markers, e.g., CD3⁺, CD4⁺, CD8⁺, CD28⁺, CD62L⁺, CCR7⁺, CD27⁺, CD127⁺, CD4⁺, CD8⁺, CD45RA⁺, and/or CD45RO⁺. In some embodiments, the phenotype is positive or high level expression of one or more specific molecules that are surface markers, e.g., C-C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin. In some embodiments, the phenotype is negative or the absence of expression of one or more specific molecule that are surface markers, e.g., CD3, CD4, CD8, CD28, CD62L, CCR7, CD27, CD127, CD45RA, and/or CD45RO. In certain embodiments, the phenotype is a surface marker of T cells or of a subpopulation or subset of T cells, such as based on the absence of surface marker expression of one or more surface markers, e.g., CD3⁻, CD4⁻, CD8⁻, CD28⁻, CD62L⁻, CCR7⁻, CD27⁻, CD127⁻, CD4⁻, CD8⁻, CD45RA⁻, and/or CD45RO⁻. In some embodiments, the phenotype is negative or the absence of expression of one or more specific molecule that are surface markers, e.g., C-C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin.

In certain embodiments, the phenotype is or includes positive or negative expression of CD27, CCR7 and/or CD45RA. In some embodiments, the phenotype is CCR7⁺. In some embodiments, the phenotype is CD27⁺. In some embodiments, the phenotype is CCR7⁻. In some embodiments, the phenotype is CD27⁻. In some embodiments, the phenotype is CCR7⁺/CD27⁺. In some embodiments, the phenotype is CCR7⁻/CD27⁺. In some embodiments, the phenotype is CCR7⁺/CD27⁻. In some embodiments, the phenotype is CCR7⁻/CD27⁻. In some embodiments, the phenotype is CD45RA⁻. In some embodiments, the phenotype is CD45RA⁺. In some embodiments, the phenotype is CCR7⁺/CD45RA⁻. In some embodiments, the phenotype is CD27⁺/CD45RA⁺. In some embodiments, the phenotype is CD27−/CD45RA+. In some embodiments, the phenotype is CD27⁻/CD45RA⁻. In some embodiments, the phenotype is CD27⁺/CD45RA⁻. In some embodiments, the phenotype is CCR7⁺/CD27⁺/CD45RA⁻. In some embodiments, the phenotype is CCR7⁺/CD27⁺/CD45RA+.

In certain embodiments, the surface marker indicates expression of a recombinant receptor, e.g., a CAR. In particular embodiments, the surface marker is expression of the recombinant receptor, e.g. CAR, which, in some aspects, can be determined using an antibody, such as an anti-idiotype antibody. In some embodiments, the surface marker that indicates expression of the recombinant receptor is a surrogate marker. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A (e.g., SEQ ID NOS: 1 and 4), a P2A (e.g., SEQ ID NOS: 5 and 6), a E2A (e.g., SEQ ID NO: 7) or a F2A (e.g., SEQ ID NO: 8). Extrinsic marker genes may in some cases be utilized in connection with engineered cells to permit detection or selection of cells and, in some cases, also to promote cell suicide.

Exemplary surrogate markers can include truncated cell surface polypeptides, such as a truncated human epidermal growth factor receptor 2 (tHER2), a truncated epidermal growth factor receptor (EGFRt, exemplary EGFRt sequence set forth in SEQ ID NO:2 or 3) or a prostate-specific membrane antigen (PSMA) or modified form thereof. EGFRt may contain an epitope recognized by the antibody cetuximab (Erbitux®) or other therapeutic anti-EGFR antibody or binding molecule, which can be used to identify or select cells that have been engineered with the EGFRt construct and a recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR, EGFRt) that is, optionally, linked to a linker sequence, such as a T2A cleavable linker sequence. An exemplary polypeptide for a truncated EGFR (e.g. tEGFR, EGFRt) comprises the sequence of amino acids set forth in SEQ ID NO: 2 or 3 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 2 or 3. In some embodiments, the phenotype is EGFRt+.

In some embodiments, the marker is or comprises a fluorescent protein, such as green fluorescent protein (GFP), enhanced green fluorescent protein (EGFP), such as super-fold GFP, red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In certain embodiments, the phenotype comprises expression, e.g. surface expression, of one or more of the surface markers CD3, CD4, CD8, and/or a recombinant receptor (e.g. CAR) or its surrogate marker indicating or correlating to expression of a recombinant receptor (e.g. CAR). In some embodiments, the surrogate marker is EGFRt.

In particular embodiments, the phenotype is identified by the expression of one or more specific molecules that are surface markers. In certain embodiments, the phenotype is or includes positive or negative expression of CD3, CD4, CD8, and/or a recombinant receptor, e.g. a CAR. In certain embodiments, the recombinant receptor is a CAR. In particular embodiments the phenotype comprises CD3⁺/CAR⁺, CD4⁺/CAR⁺, and/or CD8⁺/CAR⁺.

In certain embodiments, the phenotype is or includes positive or negative expression of CD27, CCR7 and/or CD45RA, and/or a recombinant receptor, e.g. a CAR. In some embodiments, the phenotype is CCR7⁺/CAR⁺. In some embodiments, the phenotype is CD27⁺/CAR⁺. In some embodiments, the phenotype is CCR7⁺/CD27⁺/CAR⁺. In some embodiments, the phenotype is CD45RA⁻/CAR⁺. In some embodiments, the phenotype is CCR7⁺/CD45RA⁻/CAR⁺. In some embodiments, the phenotype is CD27⁺/CD45RA⁻/CAR+. In some embodiments, the phenotype is CCR7⁺/CD27⁺/CD45RA⁻/CAR⁺. In some embodiments, the phenotype is CCR7−/CD27−/CAR+. In some embodiments, the phenotype is CCR7−/CD27+/CAR+. In some embodiments, the phenotype is CCR7+/CD27−/CAR+. In some embodiments, the phenotype is CD28−/CD27−/CAR+. In some embodiments, the phenotype is CD28−/CD27+/CAR+. In some embodiments, the phenotype is CD28+/CAR+. In some embodiments, the phenotype is CD28+/CD27−/CAR+. In some embodiments, the phenotype is CD28+/CD27+/CAR+. In some embodiments, the phenotype is CCR7−/CD45RA−/CAR+. In some embodiments, the phenotype is CCR7−/CD45RA+/CAR+. In some embodiments, the phenotype is CCR7+/CD45RA−/CAR+. In some embodiments, the phenotype is CCR7+/CD45RA+/CAR+. In some embodiments, the phenotype further is CD4+. In some embodiments, the phenotype further is CD8+.

In some embodiments, the phenotype is or includes positive expression of CD19. In some embodiments, CD19 expression indicates a tumor cell. In some embodiments, the phenotype is or includes positive expression of CD56. In some embodiments, CD56 expression is indicative of a natural killer cell.

In some embodiments, the phenotype is viability. In certain embodiments, the phenotype is the positive expression of a marker that indicates that the cell undergoes normal functional cellular processes and/or has not undergone or is not under the process of undergoing necrosis or programmed cell death. In some embodiments, viability can be assessed by the redox potential of the cell, the integrity of the cell membrane, or the activity or function of mitochondria. In some embodiments, viability is the absence of a specific molecule associated with cell death, or the absence of the indication of cell death in an assay. In some embodiments, the phenotype is viable cell concentration.

In some embodiments, the phenotype is or comprises cell viability. In certain embodiments, the viability of cells can be detected, measured, and/or assessed by a number of means that are routine in the art. Non-limiting examples of such viability assays include, but are not limited to, dye uptake assays (e.g., calcein AM assays), XTT cell viability assays, and dye exclusion assays (e.g., trypan blue, Eosin, or propidium dye exclusion assays). Viability assays are useful for determining the number or percentage (e.g., frequency) of viable cells in a cell dose, a cell composition, and/or a cell sample. In particular embodiments, the phenotype comprises cell viability along with other features, e.g., recombinant receptor expression. In some embodiments, the phenotype is or includes soluble CD137 (sCD137, 4-IBB). In some embodiments, sCD137 indicates activation induced cell death. In some embodiments, sCD137 is detected in supernatant.

In certain embodiments, the phenotype is or includes cell viability, viable CD3⁺, viable CD4⁺, viable CD8⁺, viable CD3⁺/CAR⁺, viable CD4⁺/CAR⁺, viable CD8+/CAR+, viable CD4⁺/CCR7⁺/CAR⁺, viable CD8⁺/CD27⁺/CAR⁺, viable CD4⁺/CD27⁺/CAR⁺, viable CD8⁺/CCR7⁺/CD27⁺/CAR⁺, viable CD4⁺/CCR7⁺/CD27⁺/CAR⁺, viable CD8⁺/CCR7⁺/CD45RA⁻/CAR⁺ or viable CD4⁺/CCR7⁺/CD45RA⁻ or a combination thereof.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death. These changes include blebbing, cell shrinkage, nuclear fragmentation, chromatin condensation, chromosomal DNA fragmentation, and global mRNA decay. Apoptosis is a well characterized process, and specific molecules associated with various stages are well known in the art.

In some embodiments, the phenotype is the absence of an early stage of apoptosis, and/or an absence of an indicator and/or a specific molecule associated with an early stage of apoptosis. In the early stages of apoptosis, changes in the cellular and mitochondrial membrane become apparent. Biochemical changes are also apparent in the cytoplasm and nucleus of the cell. For example, the early stages of apoptosis can be indicated by activation of certain caspases, e.g., 2, 8, 9, and 10. In particular embodiments, the phenotype is the absence of a late stage of apoptosis, and/or an absence of an indicator and/or a specific molecule associated with a late stage of apoptosis. The middle to late stages of apoptosis are characterized by further loss of membrane integrity, chromatin condensation and DNA fragmentation, and include biochemical events such as activation of caspases 3, 6, and 7.

In certain embodiments, the phenotype is the negative expression of one or more factors associated with apoptosis, including pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In some embodiments, the phenotype is a negative or low amount of a marker of apoptosis. In certain embodiments, the phenotype is the negative expression of a marker of apoptosis. In certain embodiments, the phenotype is the absence of an indicator, e.g., an Annexin V molecule, which will preferentially bind to cells undergoing apoptosis when incubated with or contacted to a cell composition. In some embodiments, the phenotype is or includes the expression of one or more markers that are indicative of an apoptotic state in the cell.

In some embodiments, the phenotype is the negative (or low) expression of a specific molecule that is a marker for apoptosis. Various apoptosis markers are known to those of ordinary skill in the art and include, but are not limited to, an increase in activity of one or more caspases i.e. an activated caspase (e.g., an active caspase), an increase in PARP cleavage, activation and/or translocation of Bcl-2 family proteins, members of the cell death pathway, e.g., Fas and FADD, presence of nuclear shrinkage (e.g., monitored by microscope) and presence of chromosome DNA fragmentation (e.g., presence of chromosome DNA ladder) or with apoptosis assays that include TUNEL staining, and Annexin V staining.

Caspases are enzymes that cleave proteins after an aspartic acid residue, the term is derived from “cysteine-aspartic acid proteases.” Caspases are involved in apoptosis, thus activation of caspases, such as caspase-3 is indicative of an increase or revival of apoptosis. In certain embodiments, caspase activation can be detected by methods known to the person of ordinary skill. In some embodiments, an antibody that binds specifically to an activated caspase (i.e., binds specifically to the cleaved polypeptide) can be used to detect caspase activation. In another example, a fluorochrome inhibitor of caspase activity (FLICA) assay can be utilized to detect caspase-3 activation by detecting hydrolysis of acetyl Asp-Glu-Val-Asp 7-amido-4-methylcoumarin (Ac-DEVD-AMC) by caspase-3 (i.e., detecting release of the fluorescent 7-amino-4-methylcoumarin (AMC)). FLICA assays can be used to determine caspase activation by a detecting the product of a substrate processed by multiple caspases (e.g., FAM-VAD-FMK FLICA). Other techniques include The CASPASE-GLO® caspase assays (PROMEGA) that use luminogenic caspase-8 tetrapeptide substrate (Z-LETD-aminoluciferin), the caspase-9 tetrapeptide substrate (Z-LEHD-aminoluciferin), the caspase-3/7 substrate (Z-DEVD-aminoluciferin), the caspase-6 substrate (Z-VEID-aminoluciferin), or the caspase-2 substrate (Z-VDVAD-aminoluciferin).

In certain embodiments, the phenotype is or includes negative expression of activated caspase-1, activated caspase-2, activated caspase-3, activated caspase-7, activated caspase-8, activated caspase-9, activated caspase-10 and/or activated caspase-13 in a cell. In particular embodiments, the phenotype is or includes activated caspase 3⁻. In some embodiments, the proform (zymogen cleaved) form of a caspase, such as any above, also is a marker indicating the presence of apoptosis. In some embodiments, the phenotype is or includes the absence of or negative expression of a proform of a caspase, such as the proform of caspase-3.

In some embodiments, the marker of apoptosis is cleaved the Poly ADP-ribose polymerase 1 (PARP). PARP is cleaved by caspase during early stages of apoptosis. Thus, detection of a cleaved PARP peptide is a marker for apoptosis. In particular embodiments, the phenotype is or includes positive or negative expression of cleaved PARP.

In some embodiments, the marker of apoptosis is a reagent that detects a feature in a cell that is associated with apoptosis. In certain embodiments, the reagent is an annexin V molecule. During the early stages of apoptosis the lipid phosphatidylserine (PS) translocates from the inner to the outer leaflet of the plasma membrane. PS is normally restricted to the internal membrane in healthy and/or non-apoptotic cells. Annexin V is a protein that preferentially binds phosphatidylserine (PS) with high affinity. When conjugated to a fluorescent tag or other reporter, Annexin V can be used to rapidly detect this early cell surface indicator of apoptosis. In some embodiments, the presence of PS on the outer membrane will persist into the late stages of apoptosis. Thus in some embodiments, annexin V staining is an indication of both early and late stages of apoptosis. In certain embodiments, an Annexin, e.g. Annexin V, is tagged with a detectable label and incubated with, exposed to, and/or contacted with cells of a cell composition to detect cells that are undergoing apoptosis, for example by flow cytometry. In some embodiments, fluorescence tagged annexins, e.g., annexin V, are used to stain cells for flow cytometry analysis, for example with the annexin⁻V/7⁻ AAD assay. Alternative protocols suitable for apoptosis detection with annexin include techniques and assays that utilize radiolabeled annexin V. In certain embodiments, the phenotype is or includes negative staining by annexin, e.g. annexin V⁻. In particular embodiments, the phenotype is or includes the absence of PS on the outer plasma membrane. In certain embodiments, the phenotype is or includes cells that are not bound by annexin e.g. annexin V. In certain embodiments, the cell that lacks detectable PS on the outer membrane is annexin V⁻. In particular embodiments, the cell that is not bound by annexin V⁻ in an assay, e.g., flow cytometry after incubation with labeled annexin V, is annexin V⁻.

In particular embodiments, the phenotype is annexin V⁻, annexin V⁻CD3⁺, annexin V⁻CD4⁺, annexin V⁻CD8⁺, annexin V⁻CD3⁺/CAR⁺, annexin V⁻CD4⁺/CAR⁺, annexin V⁻ CD8⁺/CAR⁺, activated caspase 3⁻, activated caspase 3⁻/CD3⁺, activated caspase 3⁻/CD4⁺, activated caspase 3⁻/CD8⁺, activated caspase 3⁻/CD3⁺/CAR⁺, activated caspase 3⁻/CD4⁺/CAR⁺, activated caspase 3⁻/CD8⁺/CAR⁺, annexin V⁻/CD4⁺/CCR7⁺/CAR⁺, annexin V⁻/CD8⁺/CD27⁺/CAR⁺, annexin V−/CD4⁺/CD27⁺/CAR⁺, annexin V⁻/CD8⁺/CCR7⁺/CD27⁺/CAR⁺, annexin V−/CD4⁺/CCR7⁺/CD27⁺/CAR⁺, annexin V⁻/CD8⁺/CCR7⁺/CD45RA⁻/CAR⁺ or annexin V−/CD4⁺/CCR7⁺/CD45RA⁻; activated caspase 3⁻/CD4⁺/CCR7⁺/CAR⁺, activated caspase 3⁻/CD8⁺/CD27⁺/CAR⁺, activated caspase 3⁻/CD4⁺/CD27⁺/CAR⁺, activated caspase 3⁻/CD8⁺/CCR7⁺/CD27⁺/CAR⁺, activated caspase 3⁻/CD4⁺/CCR7⁺/CD27⁺/CAR⁺, activated caspase 3⁻/CD8⁺/CCR7⁺/CD45RA⁻/CAR⁺ or activated caspase 3⁻/CD4⁺/CCR7⁺/CD45RA⁻ or a combination thereof. In some embodiments, the phenotype is 3CAS−/CCR7−/CD27−/CAR+. In some embodiments, the phenotype is 3CAS−/CCR7−/CD27+/CAR+. In some embodiments, the phenotype is 3CAS−/CCR7+/CAR+. In some embodiments, the phenotype is 3CAS−/CCR7+/CD27−/CAR+. In some embodiments, the phenotype is 3CAS−/CCR7+/CD27+/CAR+. In some embodiments, the phenotype is 3CAS−/CD27+/CAR+. In some embodiments, the phenotype is 3CAS−/CD28−/CD27−/CAR+. In some embodiments, the phenotype is 3CAS−/CD28−/CD27+/CAR+. In some embodiments, the phenotype is 3CAS−/CD28+/CAR+. In some embodiments, the phenotype is 3CAS−/CD28+/CD27−/CAR+, In some embodiments, the phenotype is 3CAS−/CD28+/CD27+/CAR+. In some embodiments, the phenotype is 3CAS−/CCR7−/CD45RA−/CAR+. In some embodiments, the phenotype is 3CAS−/CCR7−/CD45RA+/CAR+. In some embodiments, the phenotype is 3CAS−/CCR7+/CD45RA−/CAR+. In some embodiments, the phenotype is 3CAS−/CCR7+/CD45RA+/CAR+. In some embodiments, the phenotype further is CD4+. In some embodiments, the phenotype further is CD8+.

Particular embodiments contemplate that cells positive for expression of a marker for apoptosis are undergoing programmed cell death, show reduced or no immune function, and have diminished capabilities if any to undergo activation, expansion, and/or bind to an antigen to initiate, perform, or contribute to an immune response or activity. In particular embodiments, the phenotype is defined by negative expression for an activated caspase and/or negative staining with annexin V.

In certain embodiments, the phenotype is or includes activated caspase 3⁻ (3CAS−, caspase 3⁻) and/or annexin V⁻.

Among the phenotypes are the expression or surface expression of one or more markers generally associated with one or more sub-types or subpopulations of T cells, or phenotypes thereof. T cell subtypes and subpopulations may include CD4⁺ and/or of CD8⁺ T cells and subtypes thereof that may include naïve T (T_{N}) cells, naïve-like T cells, effector T cells (T_{EFF}), memory T cells and sub-types thereof, such as stem cell memory T (T_{SCM}), central memory T (T_{CM}), effector memory T (T_{EM}), T_{EMRA} cells or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some aspects, among the phenotypes include expression or markers or functions, e.g. antigen-specific functions such as cytokine secretion, that are associated with a less differentiated cell subset or a more differentiated subset. In some embodiments, the phenotypes are those associated with a less differentiated subset, such as one or more of CCR7⁺, CD27⁺ and interleukin-2 (IL-2) production. In some aspects, less differentiated subsets can also be related to therapeutic efficacy, self-renewal, survival functions or graft-versus-host disease. In some embodiments, the phenotypes are those associated with a more differentiated subset, such as one or more of interferon-gamma (IFN-γ) or IL-13 production. In some aspects, more differentiated subsets can also be related to senescence and effector function.

In some embodiments, the phenotype is or includes a phenotype of a memory T cell or memory T cell subset exposed to their cognate antigen. In some embodiments the phenotype is or includes a phenotype of a memory T cell (or one or more markers associated therewith), such as a T_{CM} cell, a T_{EM} cell, or a T_{EMRA} cell, a T_{SCM} cell, or a combination thereof. In particular embodiments, the phenotype is or includes the expression of one or more specific molecules that is a marker for memory and/or memory T cells or subtypes thereof. In some aspects, exemplary phenotypes associated with T_{CM} cells can include one or more of CD45RA⁻, CD62L⁺, CCR7⁺, CD27+, CD28+, and CD95⁺. In some aspects, exemplary phenotypes associated with T_{EM} cells can include one or more of CD45RA⁻, CD62L⁻, CCR7⁻, CD27−, CD28−, and CD95⁺.

In particular embodiments, the phenotype is or includes the expression of one or more specific molecules that is a marker for naïve T cells.

In some embodiments, the phenotype is or includes a memory T cell or a naive T cell. In certain embodiments, the phenotype is the positive or negative expression of one or more specific molecules that are markers for memory. In some embodiments, the memory marker is a specific molecule that may be used to define a memory T cell population.

In some embodiments, the phenotype is or includes a phenotype of or one or more marker associated with a naïve-like T cell. In certain embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+CCR7+, where the cells are CD45RA+ or CD45RA−. In certain embodiments, naïve-like T cells or the T cells that are surface positive for a marker expressed on naïve-like T cells are CD62L−/CCR7+.

In some embodiments, the phenotype is or includes a phenotype of one or more markers associated with an intermediate type T cell. In some embodiments, intermediate T cells may be characterized by positive or high expression (e.g., surface expression or intracellular expression) of certain cell markers and/or negative or low expression (e.g., surface expression or intracellular expression) of other cell markers. In some aspects, intermediate T cells are characterized by positive or high expression of CCR7 and/or CD28. In some embodiments, intermediate T cells are CCR7+/CD45RA−, CD28+, or CD28+/CD27− cells.

In some embodiments, the phenotype is or includes a phenotype of or one or more marker associated with a non-memory T cell or sub-type thereof; in some aspects, it is or includes a phenotype or marker(s) associated with a naïve cell. In some aspects, exemplary phenotypes associated with naïve T cells can include one or more of CCR7+, CD45RA+, CD27+, and CD28+. In some embodiments, the phenotype is CCR7⁺/CD27⁺/CD28⁺/CD45RA⁺. In certain embodiments, the phenotype is or includes CCR7⁺/CD45RA⁺. In certain embodiments, the phenotype is or includes CCR7⁺/CD27+. In certain embodiments, the phenotype is or includes CD27+/CD28+. In some embodiments, the phenotype is or includes a phenotype of a central memory T cell. In particular embodiments, the phenotype is or includes CCR7⁺/CD27⁺/CD28⁺/CD45RA⁻. In some embodiments, the phenotype is or includes CCR7⁻/CD27⁺/CD28⁺/CD45RA⁻. In some embodiments, the phenotype is or includes CCR7⁺/CD27⁺. In some embodiments, the phenotype is or includes CD27⁺/CD28⁺. In certain embodiments, the phenotype is or includes that of a T_{EMRA} cell or a T_{SCM} cell. In certain embodiments, the phenotype is or includes CD45RA⁺. In particular embodiments, the phenotype is or includes CCR7⁻/CD27⁻/CD28⁻/CD45RA⁺. In some embodiments, the phenotype is or includes one of CD27⁺/CD28⁺, CD27⁻/CD28⁺, CD27⁺/CD28⁻, or CD27⁻/CD28⁻. In some embodiments, the phenotype is CCR7⁺/CD27⁺/CD45RA⁺. In certain embodiments, the phenotype is or includes CCR7⁺/CD45RA⁺. In certain embodiments, the phenotype is or includes CD27⁻/CD28⁻. In particular embodiments, the phenotype is or includes CCR7⁺/CD27⁺/CD45RA⁻. In some embodiments, the phenotype is or includes CCR7⁻/CD27⁺/CD45RA⁻. In certain embodiments, the phenotype is or includes CD45RA⁺. In particular embodiments, the phenotype is or includes CCR7⁻/CD27⁻/CD45RA⁺.

In some embodiments the phenotype is or includes any of the foregoing phenotypic properties and further includes the expression of a recombinant receptor, such as phenotype associated with a memory T cell or memory subtype and that expresses a CAR, or a phenotype associated with a naïve cell that expresses a CAR. In certain embodiments, the phenotype is or includes that of a central memory T cell or stem central memory T cell that expresses a CAR. In particular embodiments, the phenotype is or includes that of an effector memory cell that expresses a CAR. In some embodiments, the phenotype is or includes that of a T_{EMRA} cell that expresses a CAR. In particular embodiments, the phenotype is or includes CAR⁺/CCR7⁺/CD27⁺/CD28⁺/CD45RA⁻; CAR⁺/CCR7⁻/CD27⁺/CD28⁺/CD45RA⁻; CAR⁺/CCR7⁻/CD27⁻/CD28⁻/CD45RA⁺; CAR⁺/CD27⁺/CD28⁺; CAR⁺/CD27⁻/CD28⁺; CAR⁺/CD27⁺/CD28⁻; or CAR⁺/CD27⁻/CD28⁻. In particular embodiments, the phenotype is or includes CAR⁺/CCR7⁺/CD27⁺/CD45RA⁻; CAR⁺/CCR7⁻/CD27⁺/CD45RA⁻; CAR⁺/CCR7⁻/CD27⁻/CD28⁻/CD45RA⁺; CAR⁺/CD27⁺; CAR⁺/CD27⁻; CAR⁺/CD27⁺/CD28⁻; or CAR⁺/CD27⁻/CD28⁻.

In certain embodiments, the phenotype is or includes a phenotype of a T cell that is negative for a marker of apoptosis. In certain embodiments, the phenotype is or includes a naïve cell that is negative for a marker of apoptosis. In some embodiments, the marker of apoptosis is activated caspase 3 (3CAS). In some embodiments, the marker of apoptosis is positive staining by annexin V.

In particular embodiments, the phenotype is or includes that of a memory T cell or subtype thereof that is negative for a marker of apoptosis that expresses a CAR. In particular embodiments, the phenotype is or includes that of a memory T cell or particular subtype that is negative for a marker of apoptosis that expresses a CAR. In certain embodiments, the phenotype is or includes a naïve cell that is negative for a marker of apoptosis that expresses a CAR. In certain embodiments, the phenotype is or includes that of a central memory T cell or T_{SCM} cell or naïve cell that is negative for a marker of apoptosis that expresses a CAR. In particular embodiments, the phenotype is or includes that of an effector memory cell that is negative for a marker of apoptosis that expresses a CAR. In certain embodiments, the phenotype is or includes annexin V⁻/CAR⁺/CCR7⁺/CD27⁺/CD28⁺/CD45RA⁻; annexin V⁻/CAR⁺/CCR7⁻/CD27⁺/CD28⁺/CD45RA⁻; annexin V⁻/CAR⁺/CCR7⁻/CD27⁻/CD28⁻/CD45RA⁺; annexin V⁻/CAR+/CD27+/CD28+; annexin V⁻/CAR⁺/CD27⁻/CD28⁺; annexin V⁻/CAR⁺/CD27⁺/CD28⁻; or annexin V⁻/CAR⁺/CD27⁻/CD28⁻. In certain embodiments, the phenotype is or includes activated caspase 3⁻/CAR⁺/CCR7⁺/CD27⁺/CD28⁺/CD45RA⁻; activated caspase 3⁻/CAR⁺/CCR7⁻/CD27⁺/CD28⁺/CD45RA⁻; activated caspase 3⁻/CAR⁺/CCR7⁻/CD27⁻/CD28⁻/CD45RA⁺; activated caspase 3⁻/CAR⁺/CD27⁺/CD28⁺; activated caspase 3⁻/CAR⁺/CD27⁻/CD28⁺; activated caspase 3⁻/CAR⁺/CD27⁺/CD28⁻; or activated caspase 3⁻/CAR⁺/CD27⁻/CD28⁻. In certain embodiments, the phenotype is or includes annexin V⁻/CAR⁺/CCR7⁺/CD27⁺/CD45RA⁻; annexin V⁻/CAR⁺/CCR7⁻/CD27⁺/CD45RA⁻; annexin V⁻/CAR⁺/CCR7⁻/CD27⁻/CD45RA⁺; annexin V⁻/CAR⁺/CD27⁺/CD28⁺; annexin V⁻/CAR⁺/CD27⁻/CD28⁺; annexin V⁻/CAR⁺/CD27⁺; or annexin V⁻/CAR⁺/CD27⁻. In certain embodiments, the phenotype is or includes activated caspase 3⁻/CAR⁺/CCR7⁺/CD27⁺/CD45RA⁻; activated caspase 3⁻/CAR⁺/CCR7⁻/CD27⁺/CD45RA⁻; activated caspase 3⁻/CAR⁺/CCR7⁻/CD27⁻/CD45RA⁺; activated caspase 3⁻/CAR⁺/CD27⁺/CD28⁺; activated caspase 3⁻/CAR⁺/CD27⁻/CD28⁺; activated caspase 3⁻/CAR⁺/CD27⁺; or activated caspase 3⁻/CAR⁺/CD27⁻.

In particular embodiments, the phenotype is or includes CD27⁺/CD28⁺, CD27⁻/CD28⁺, CD27⁺/CD28⁻, CD27⁻/CD28⁻, or a combination thereof. In some embodiments, the phenotype is or includes CAR⁺/CD27⁺/CD28⁺, CAR⁺/CD27⁻/CD28⁺, CAR+/CD27+/CD28−, CAR⁺/CD27⁻/CD28⁻, or a combination thereof. In certain embodiments, the phenotype is or includes activated caspase 3⁻/CAR⁺/CD27⁺/CD28⁺, activated caspase 3⁻/CAR⁺/CD27⁻/CD28⁺, activated caspase 3⁻/CAR⁺/CD27⁺/CD28⁻, activated caspase 3⁻/CAR⁺/CD27⁻/CD28⁻, or a combination thereof. In particular embodiments, the phenotype is or includes annexin V⁻/CAR⁺/CD27⁺/CD28⁺, annexin V⁻/CAR⁺/CD27⁻/CD28⁺, annexin V⁻/CAR⁺/CD27⁺/CD28⁻, annexin V⁻/CAR⁺/CD27⁻/CD28⁻, or a combination thereof. In particular embodiments, the phenotype is or includes CD27⁺, CD27⁻, CD27⁺, CD27⁻, or a combination thereof. In some embodiments, the phenotype is or includes CAR⁺/CD27⁺, CAR⁺/CD27⁻, CAR⁺/CD27⁺, CAR⁺/CD27⁻, or a combination thereof. In certain embodiments, the phenotype is or includes activated caspase 3⁻/CAR⁺/CD27⁺, activated caspase 3⁻/CAR⁺/CD27⁻, activated caspase 3⁻/CAR⁺/CD27⁺, activated caspase 3⁻/CAR⁺/CD27⁻, or a combination thereof. In particular embodiments, the phenotype is or includes annexin V⁻/CAR⁺/CD27⁺, annexin V⁻/CAR⁺/CD27⁻, annexin V⁻/CAR⁺/CD27⁺, annexin V⁻/CAR⁺/CD27⁻, or a combination thereof.

In particular embodiments, the phenotype is or includes CCR7+/CD28+, CCR7⁻/CD28⁺, CCR7⁺/CD28⁻, CCR7⁻/CD28⁻, or a combination thereof. In some embodiments, the phenotype is or includes CAR⁺/CCR7⁺/CD28⁺, CAR⁺/CCR7⁻/CD28⁺, CAR⁺/CCR7⁺/CD28⁻, CAR⁺/CCR7⁻/CD28⁻, or a combination thereof. In certain embodiments, the phenotype is or includes activated caspase 3⁻/CAR⁺/CCR7⁺/CD28⁺, activated caspase 3⁻/CAR⁺/CCR7⁻/CD28⁺, activated caspase 3⁻/CAR⁺/CCR7⁺/CD28⁻, activated caspase 3⁻/CAR⁺/CCR7⁻/CD28⁻, or a combination thereof. In particular embodiments, the phenotype is or includes annexin V⁻/CAR⁺/CCR7⁺/CD28⁺, annexin V⁻/CAR⁺/CCR7⁻/CD28⁺, annexin V⁻/CAR⁺/CCR7⁺/CD28⁻, annexin V⁻/CAR⁺/CCR7⁻/CD28⁻, or a combination thereof. In particular embodiments, the phenotype is or includes CCR7⁺, CCR7⁻, CCR7⁺, CCR7⁻, or a combination thereof. In some embodiments, the phenotype is or includes CAR⁺/CCR7⁺, CAR⁺/CCR7⁻, CAR⁺/CCR7⁺, CAR⁺/CCR7⁻, or a combination thereof. In certain embodiments, the phenotype is or includes activated caspase 3⁻/CAR⁺/CCR7⁺, activated caspase 3⁻/CAR⁺/CCR7⁻, activated caspase 3⁻/CAR⁺/CCR7⁺, activated caspase 3⁻/CAR⁺/CCR7⁻, or a combination thereof. In particular embodiments, the phenotype is or includes annexin V⁻/CAR⁺/CCR7⁺, annexin V⁻/CAR⁺/CCR7⁻, annexin V⁻/CAR⁺/CCR7⁺, annexin V⁻/CAR⁺/CCR7⁻, or a combination thereof.

In some embodiments, the phenotype is assessed by a response to a stimulus, for example a stimulus that triggers, induces, stimulates, or prolongs an immune cell function. In certain embodiments, the cells are incubated in the presence of stimulating conditions or a stimulatory agent, the phenotype is or includes the response to the stimulation. In particular embodiments, the phenotype is or includes the production or secretion of a soluble factor in response to one or more stimulations. In some embodiments, the phenotype is or includes a lack or production or secretion of a soluble factor in response to one or more stimulations. In certain embodiments, the soluble factor is a cytokine. In some embodiments, the cytokine is IL-2. In some embodiments, the cytokine is TNFa. In some embodiments, the cytokine is IL-17. In some embodiments, the cytokine is IFNG. In some embodiments, the cytokine is IL-13. In some embodiments, the cytokine is IL-5. In some embodiments, the cytokine is IL-10. In some embodiments, the cytokine is GMCSF. In some embodiments, the cell does not produce cytokines (cyto-). In some embodiments, the cell phenotype is cytokine negative (Cyto-).

The conditions used for stimulating cells can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the cells are stimulated and the phenotype is determined by whether or not a soluble factor, e.g., a cytokine or a chemokine, is produced or secreted. In some embodiments, the stimulation is nonspecific, i.e., is not an antigen-specific stimulation. In some embodiments, the stimulation comprises PMA and ionomycin. In some embodiments, cells are incubated in the presence of stimulating conditions or a stimulatory agent for about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 48 hours, or for a duration of time between 1 hour and 4 hours, between 1 hour and 12 hours, between 12 hours and 24 hours, or for more than 24 hours.

In some embodiments, the attributes include recombinant receptor-dependent activity. For example, in some embodiments, the cells of the therapeutic cell composition are stimulated with an agent that is an antigen or an epitope thereof that is specific to the recombinant receptor, or is an antibody or fragment thereof that binds to and/or recognizes the recombinant receptor, or a combination thereof. Particular embodiments contemplate that a recombinant receptor-dependent activity, e.g., a CAR dependent activity, is an activity that occurs in a cell that expresses a recombinant receptor which does not and/or cannot occur in a cell that does not express the recombinant receptor. In some embodiments, the recombinant receptor-dependent activity is an activity that depends on an activity or presence of the recombinant receptor. The recombinant receptor-dependent activity may be any cellular process that is directly or indirectly influenced by the expression and/or presence of the recombinant receptor or by a change in activity, such as receptor stimulation, of the recombinant receptor. In some embodiments, the recombinant receptor-dependent activity may include, but is not limited to cellular processes such as cell division, DNA replication, transcription, protein synthesis, membrane transport, protein translocation, and/or secretion, or it may be an immune cell function, e.g., a cytolytic activity. In certain embodiments, recombinant receptor-dependent activity may be measured by a change in the confirmation of the CAR receptor, the phosphorylation of an intracellular signaling molecule, degradation of a protein, transcription, translation, translocation of a protein, and/or production and secretion of a factor, such as a protein, or growth factor, cytokine.

In some embodiments, the recombinant receptor is a CAR, and the agent is an antigen or an epitope thereof that is specific to the CAR, or is an antibody or fragment thereof that binds to and/or recognizes the CAR, or a combination thereof. In particular embodiments, the cells are stimulated by incubating the cells in the presence of target cells with surface expression of the antigen that is recognized by the CAR. In certain embodiments, the recombinant receptor is a CAR, and the agent is an antibody or an active fragment, variant, or portion thereof that binds to the CAR. In certain embodiments, the antibody or the active fragment, variant, or portion thereof that binds to the CAR is an anti-idiotypic (anti-ID) antibody. In certain embodiments, the recombinant receptor specific agent is a cell, e.g., target cell, that expresses the antigen on its surface. In some embodiments, the recombinant receptor dependent activity is stimulated by an antigen or an epitope thereof that is bound by and/or recognized by (e.g., engages) the recombinant receptor.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to a solid support such as a bead, and/or one or more cytokines. In some embodiments, the one or more agents are PMA and ionomycin.

In certain embodiments, the recombinant receptor-dependent activity, e.g., a CAR dependent activity is a measurement of a factor, e.g., an amount or concentration, or a change in the amount or concentration following stimulation of the cell composition. In certain embodiments, the factor may be a protein, a phosphorylated protein, a cleaved protein, a translocated protein, a protein in an active confirmation, a polynucleotide, an RNA polynucleotide, an mRNA, and/or an shRNA. In certain embodiments, the measurement may include, but is not limited to, an increase or decrease of kinase activity, protease activity, phosphatase activity, cAMP production, ATP metabolism, translocation, e.g., a nuclear localization of a protein, an increase in transcriptional activity, an increase in translational activity, production and/or secretion of a soluble factor, cellular uptake, ubiquitination, and/or protein degradation. In particular embodiments, the factor is a soluble factor that is secreted, such as a hormone, a growth factor, a chemokine, and/or a cytokine.

In some embodiments, the recombinant receptor-dependent activity, e.g., a CAR dependent activity, is a response to stimulation. In certain embodiments, the cells are incubated in the presence of stimulating conditions or a stimulatory agent, and the activity is or includes at least one aspect of a response to the stimulation. A response may include, but is not limited to, an intracellular signaling event, such as an increased activity of a receptor molecule, an increased kinase activity of one or more kinases, an increase in the transcription of one or more genes, increased protein synthesis of one or more proteins, and/or an intracellular signaling molecule e.g., an increased kinase activity of a protein. In some embodiments, the response or activity is associated with an immune activity, and may include, but is not limited to, production and/or section of a soluble factor, e.g., a cytokine, an increase in antibody production, and/or an increase in cytolytic activity.

In particular embodiments, the response to a stimulation of a cell composition is assessed by measuring, detecting, or quantifying a response to a stimulus, i.e. at least one activity that is initiated, triggered, supported, prolonged, and/or caused by the stimulus. In certain embodiments, the cells are stimulated and the response to the stimulation is an activity that is specific to cells that express a recombinant receptor. In certain embodiments, the activity is a recombinant receptor specific activity and the activity occurs in cells that express the recombinant receptor, but does not occur, or only minimally occurs, in cells that do not express the receptor. In particular embodiments, the recombinant receptor is a CAR. In some embodiments, the activity is a CAR dependent activity.

The conditions used for stimulating cells, e.g., immune cells or T cells, can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the cells are stimulated and the activity is determined by whether or not a soluble factor, e.g., a cytokine or a chemokine, is produced or secreted.

In some embodiments, the activity is specific to cells that express a recombinant receptor. In some embodiments, an activity that is specific to cells that express a recombinant receptor does not occur in cells that lack expression of the recombinant receptor. In certain embodiments, the recombinant receptor is a CAR, and the activity is a CAR dependent activity. In particular embodiments, the activity is not present in cells that lack expression of the recombinant receptor under the same conditions where the activity is present in cells that express the recombinant receptor. In certain embodiments, the CAR dependent activity is about 10%, about 20%, about 30%, about 40%, about 50%, about 60% about 70%, about 75%, about 80%, about 85%, about 90%, about 95%, about 97%, about 98%, about 99%, or about 99% less than the CAR dependent activity in CAR− cells under the same conditions.

In some embodiments, the activity is specific to cells that express a recombinant receptor, e.g., a CAR, and the activity is produced by stimulation with an agent or under stimulatory conditions that are specific to cells that express the recombinant receptor. In some embodiments, the recombinant receptor is a CAR, and a CAR specific stimulation stimulates, triggers, initiates, and/or prolongs an activity in CAR+ cells, but does not stimulate, trigger, initiate, and/or prolong the activity in CAR− cells. In some embodiments, the CAR dependent activity is about 10%, about 20%, about 30%, about 40%, about 50%, about 60% about 70%, about 75%, about 80%, about 85%, about 90%, about 95%, about 97%, about 98%, about 99%, or about 99% less in CAR− cells than in the CAR+ cells following stimulation by the CAR specific stimulus.

In some embodiments, the activity is measured in the cell composition containing cells expressing a recombinant receptor, e.g., a CAR, and the measurement is compared to one or more controls. In certain embodiments, the control is a similar or identical composition of cells that was not stimulated. For example, in some embodiments, the activity is measured in a cell composition following or during incubation with an agent, and the resulting measurement is compared to a control measurement of the activity from the similar or identical cell composition that is not incubated with the agent. In some embodiments, the activity is a recombinant receptor-dependent activity, and both the cell composition and the control cell composition contain cells that express the recombinant receptor. In some embodiments, the activity is a recombinant receptor-dependent activity, and the control is taken from a similar cell composition that does not contain cells that express the recombinant receptor, e.g., CAR+ cells. Thus in some embodiments, a cell composition that contains recombinant receptor expressing cells and a control cell composition that does not contain recombinant receptor expressing cells are contacted with a recombinant receptor expressing specific agent. In certain embodiments, the control is a measurement from the same cell composition that expresses a recombinant receptor that is taken prior to any stimulation. In certain embodiments, a control measurement is obtained to determine a background signal, and control measurement is subtracted from the measurement of the activity. In some embodiments, the measurement of the activity in the cell composition is divided by the control measurement, to obtain a value that is a ratio of the activity over a control level.

In particular embodiments, the activity is or includes the production and/or secretion of a soluble factor. In some embodiments, the activity is a recombinant receptor, e.g., a CAR, dependent activity that is or includes the production and/or secretion of a soluble factor. In certain embodiments, the soluble factor is a cytokine or a chemokine.

In particular embodiments, the measurement of the soluble factor is measured by ELISA (enzyme-linked immunosorbent assay). ELISA is a plate-based assay technique designed for detecting and quantifying substances such as peptides, cytokines, antibodies and hormones. In an ELISA, the soluble factor must be immobilized to a solid surface and then complexed with an antibody that is linked to an enzyme. Detection is accomplished by assessing the conjugated enzyme activity via incubation with a substrate to produce a detectable signal. In some embodiments, the CAR dependent activity is measured with an ELISA assay.

In some embodiments, the recombinant receptor-dependent activity is a secretion or production of the soluble factor. In certain embodiments, production or secretion is stimulated in a cell composition that contains recombinant receptor expressing cells, e.g., CAR expressing cells, by a recombinant receptor specific agent, e.g., a CAR+ specific agent. In some embodiments, the recombinant receptor specific agent that is an antigen or an epitope thereof that is specific to the recombinant receptor; a cell, e.g., a target cell, that expresses the antigen; or an antibody or a portion or variant thereof that binds to and/or recognizes the recombinant receptor; or a combination thereof. In certain embodiments, the recombinant receptor specific agent is a recombinant protein that comprises the antigen or epitope thereof that is bound by or recognized by the recombinant receptor.

In certain embodiments, the recombinant receptor dependent soluble factor production and/or secretion is measured by incubating the cell composition that contains cells expressing the recombinant receptor, e.g., a CAR, with a recombinant receptor specific agent, e.g., CAR+ specific agent. In certain embodiments, the soluble factor is a cytokine or a chemokine. In some embodiments, cells of the cell composition that contain recombinant receptor expressing cells are incubated in the presence of recombinant receptor specific agent for an amount of time, and the production and/or secretion of the soluble factor is measured at one or more time points during the incubation. In some embodiments, the cells are incubated with the CAR specific agent for up to or about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 19 hours, about 20 hours, about 21 hours, about 22 hours, about 23 hours, about 24 hours, about 48 hours, or for a duration of time between 1 hour and 4 hours, between 1 hour and 12 hours, between 12 hours and 24 hours, each inclusive, or for more than 24 hours and the amount of a soluble factor, e.g., a cytokine is detected.

In some embodiments, the recombinant receptor specific agent is a target cell that expresses an antigen recognized by the recombinant receptor. In some embodiments, the recombinant receptor is a CAR, and the cells of the cell composition are incubated with the target cells at ratio of total cells, CAR+ cells, CAR+/CD8+ cells, or Annexin-/CAR+/CD8+ cells of the cell composition to target cells of about 10:1, about 5:1, about 4:1, about 3:1, about 2:1, about 1:1, about 1:2, about 1:3, about 1:4, about 1:5, about 1:6, about 1:7, about 1:8, about 1:9, or about 1:10, or a range between any of the foregoing, such as at a ratio between 10:1 and 1:1, 3:1 and 1:3, or 1:1 and 1:10, each inclusive. In some embodiments, for example when the cells of the therapeutic composition contain an anti-CD19 CAR, the cells of the therapeutic composition are incubated with target cells expressing CD19 (e.g., CD19+). In some embodiments, cells of the therapeutic composition having the ability to engage and be stimulated by the CD19+ target cells, e.g., to produce a cytokine, execute cytolytic activity, are referred to as CD19+. For example, if a cell of the therapeutic composition engages CD19 on a target cell via a recombinant receptor (e.g., CAR) and produces or secretes a cytokine, the cell may be referred to as cytokine/CD19+. As a non-limiting example, in some embodiments, if upon engagement of CD19+ target cells the therapeutic cell expresses IFNg, the therapeutic cell composition recombinant receptor-dependent activity attribute may be referred to as IFNg+/CD19+.

In some embodiments, the cells of the therapeutic cell composition are incubated with the recombinant receptor specific agent, e.g., a CAR+ specific agent, in a volume of cell media. In certain embodiments, the cells are incubated with the recombinant receptor specific agent in a volume of at least or about 1 μL, at least or about 10 μL, at least or about 25 μL, at least or about 50 μL, at least or about 100 μL, at least or about 500 μL, at least or about 1 mL, at least or about 1.5 mL, at least or about 2 mL, at least or about 2.5 mL, at least or about 5 mL, at least or about 10 mL, at least or about 20 mL, at least or about 25 mL, at least or about 50 mL, at least or about 100 mL, or greater than 100 mL. In certain embodiments, the cells are incubated with the CAR+ specific agent in a volume that falls between about 1 μL and about 100 μL, between about 100 μL and about 500 μL, between about 500 μL and about 1 mL, between about 500 μL and about 1 mL, between about 1 mL and about 10 mL, between about 10 mL and about 50 mL, or between about 10 mL and about 100 mL, each inclusive. In certain embodiments, the cells are incubated with the recombinant receptor specific agent in a volume of between about 100 μL and about 1 mL, inclusive. In particular embodiments, the cells are incubated with the recombinant receptor specific agent in a volume of about 500 μL.

In some embodiments, the cells of the therapeutic cell composition are incubated with the CAR+ specific agent at an amount of between about 1 fmol and about 1 pmol, between about 1 pmol and about 1 nmol, between about 1 nmol and about 1 pmol, between about 1 pmol and about 1 mmol, or between about 1 mmol and 1 mol, each inclusive. In particular embodiments, the cells of the cell composition are incubated with the CAR+ specific agent at a concentration of between about 1 fM and about 1 pM, between about 1 pM and about 1 nM, between about 1 nM and about 1 μM, between about 1 μM and about 1 mM, or between about 1 mM and 1 mol, each inclusive. Exemplary units include, but are not limited to pg/mL, pg/(mL/hr), pg(mL×cell), pg/(mL×hr×cell), and pg/(mL×hr×10⁶ cells).

In certain embodiments, the measurement of the recombinant receptor-dependent activity, e.g., the CAR+ specific activity, is the amount or concentration, or a relative amount or concentration, of the soluble factor in the T cell composition at a time point during or at the end of the incubation. In particular embodiments, the measurement is subtracted by or normalized to a control measurement. In some embodiments, the control measurement is a measurement from the same cell composition taken prior to the incubation. In particular embodiments the control measurement is a measurement taken from an identical control cell composition that was not incubated with the recombinant receptor specific stimulation agent. In certain embodiments, the control is a measurement taken at an identical time point during incubation with the recombinant receptor specific agent from a cell composition that does not contain recombinant receptor positive cells.

In some embodiments, the measurement is a normalized ratio of the amount or concentration as compared to the control. In particular embodiments, the measurement is the amount or concentration of the soluble factor per an amount of time, e.g., per minute or per hour. In some embodiments, the measurement is an amount or concentration of the soluble factor per cell or per a set or reference number of cells, e.g., per 100 cells, per 10³ cells, per 10⁴ cells, per 10⁵ cells, per 10⁶ cells, etc. In certain the measurement is the amount or concentration of the soluble factor per an amount of time, per cell or per reference number of cells. In some embodiments, the measurement is the amount or concentration of the soluble factor per cell that expresses the recombinant receptor, CAR+ cell, CAR+/CD8+ cell, Annexin−/CAR+/CD8+ cell, 3CAS−/CAR+/CD8+ cell, CAR+/CD4+ cell, Annexin−/CAR+/CD4+ cell, or 3CAS−/CAR+/CD4+ cell of the cell composition. In certain embodiments, the measurement is the amount or concentration of the soluble factor per amount of time (e.g., per minute or per hour) per cell that expresses the recombinant receptor, CAR+ cell, CAR+/CD8+ cell, Annexin-/CAR+/CD8+ cell, 3CAS−/CAR+/CD8+ cell, CAR+/CD4+ cell, Annexin−/CAR+/CD4+ cell, or 3CAS−/CAR+/CD4+ cell of the cell composition. In some embodiments, the measurement is the amount or concentration of the soluble factor per an amount of time per amount or concentration of the recombinant receptor or CAR+ specific agent. In some embodiments, the measurement is an amount or concentration of the soluble factor per cell or per a set or reference number of cells per amount or concentration of the CAR+ specific agent. In certain the measurement is the amount or concentration of the soluble factor per an amount of time, per amount or concentration of the recombinant receptor or CAR+ specific agent, per cell or per reference number of cells. In some embodiments, the measurement is the amount or concentration of the soluble factor per amount or concentration of the recombinant receptor or CAR+ specific agent, per cell that expresses the recombinant receptor, CAR+ cell, CAR+/CD8+ cell, Annexin−/CAR+/CD8+ cell, 3CAS−/CAR+/CD8+ cell, CAR+/CD4+ cell, Annexin−/CAR+/CD4+ cell, or 3CAS−/CAR+/CD4+ cell of the cell composition. In certain embodiments, the measurement is the amount or concentration of the soluble factor per amount of time, per amount or concentration of the recombinant receptor or CAR+ specific agent, per amount of CAR+ cell, CAR+/CD8+ cell, Annexin−/CAR+/CD8+ cell, 3CAS−/CAR+/CD8+ cell, CAR+/CD4+ cell, Annexin−/CAR+/CD4+ cell, or 3CAS−/CAR+/CD4+ cells of the cell composition.

In particular embodiments, the recombinant receptor or CAR dependent activity is the production or secretion of two or more soluble factors. In certain embodiments, the recombinant receptor or CAR dependent activity is the production or secretion of two, three, four, five, six, seven, eight, nine, ten, or more than ten soluble factors. In some embodiments, the measurements of the two, three, four, five, six, seven, eight, nine, ten, or more than ten soluble factors are combined into an arithmetic mean or a geometric mean. In certain measurements, measurement of the recombinant receptor activity is the secretion of are composites of two, three, four, five, six, seven, eight, nine, ten, or more than ten soluble factors.

In particular embodiments, the measurement of the recombinant receptor-dependent activity is transformed, e.g., by a logarithmic transformation. In certain embodiments, the measurement of the recombinant receptor activity is transformed by a common log (log₁₀(x)), a natural log (ln(x)) or a binary log (log₂(x)). In some embodiments, the measurement of the recombinant receptor-dependent activity is a composite of measurement of the production or secretion of two more soluble factors. In some embodiments, two or more measurements of production or secretion of soluble factors are transformed prior to being combined into a composite measurement. In particular embodiments, the measurement of the recombinant receptor dependent activity is transformed prior to normalization to a reference measurement. In certain embodiments, the measurement of the recombinant receptor dependent activity is transformed prior to normalization to a reference measurement.

In certain embodiments, the soluble factor is a cytokine. In particular embodiments, the recombinant receptor-dependent activity is or includes the production or secretion of a cytokine in response to one or more stimulations. Cytokines are a large group of small signaling molecules that function extensively in cellular communication. Cytokines are most often associated with various immune modulating molecules that include interleukins, chemokines, and interferons. Alternatively cytokines may be characterized by their structure, which are categorized in four families, the four alpha helix family that includes the IL-2 subfamily, the IFN subfamily, and the IL-10 subfamily; the IL-1 family, the IL-17 family, and cysteine-knot cytokines that include members of the transforming growth factor beta family. The production and/or the secretion of cytokines contributes to immune responses, and is involved in different processes including the induction of anti-viral proteins and the induction of T cell proliferation. Cytokines are not pre-formed factors but are rapidly produced and secreted in response to cellular activation. The production or secretion of cytokines may be measured, detected, and/or quantified by any suitable technique known in the art. In some embodiments, the recombinant receptor-dependent activity is the production or secretion of one or more soluble factors that include interleukins, interferons, and chemokines. In particular embodiments, the recombinant receptor-dependent activity is the production or secretion of one or more of an IL-2 family member, an IFN subfamily member, an IL-10 subfamily member; an IL-1 family member, an IL-17 family member, a cysteine-knot cytokine, and/or a member of the transforming growth factor beta family.

In certain embodiments, the phenotype is the production of one or more cytokines. In some embodiments, the production of two or more cytokines from the same cell can be indicative of polyfunctional features of such cells. In particular embodiments, the production of one or more cytokines is measured, detected, and/or quantified by intracellular cytokine staining. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. In some embodiments, as described above, the stimulation can be performed using nonspecific stimulation, e.g., is not an antigen-specific stimulation. For example, PMA/ionomycin can be used for nonspecific cell stimulation. In some embodiments, the stimulation can be performed by an agent that is an antigen or an epitope thereof that is specific to the recombinant receptor (e.g., CAR), or is an antibody or fragment thereof that binds to and/or recognizes the recombinant receptor, or a combination thereof. ICS can also be used in combination with other flow cytometry protocols for immunephenotyping using cell surface markers or with MHC multimers to access cytokine production in a particular subgroup of cells, making it an extremely flexible and versatile method. Other single-cell techniques for measuring or detecting cytokine production include, but are not limited to ELISPOT, limiting dilution, and T cell cloning.

In some embodiments, the phenotype is the production of a cytokine, such as following stimulation of the recombinant receptor with an antigen specific to and/or recognized by the recombinant receptor. In particular embodiments, the phenotype is the lack of the production of the cytokine, such as following stimulation of the recombinant receptor with an antigen specific to and/or recognized by the recombinant receptor. In particular embodiments, the phenotype is positive for or is a high level of production of a cytokine. In certain embodiments, the phenotype is negative for or is a low level of production of a cytokine. Cytokines may include, but are not limited to, interleukin-1 (IL-1), IL-1β, IL-2, sIL-2Ra, IL-3, IL-5, IL-6, IL-7, IL-8, IL-10, IL-12, IL-13, IL-17, IL 27, IL-33, IL-35, TNF, tumor necrosis factor alpha (TNFA), CXCL2, CCL2, CCL3, CCL5, CCL17, CCL24, PGD2, LTB4, interferon gamma (IFNG), granulocyte macrophage colony stimulating factor (GMCSF), macrophage inflammatory protein MIP1α, MIP1β, Flt-3L, fractalkine, and/or IL-5. In some embodiments, the phenotype includes production of cytokines, e.g., cytokines associated with particular cell types, such as cytokines associated with Th1, Th2, Th17 and/or Treg subtypes. In some embodiments, exemplary Th1-related cytokines include IL-2, IFN-γ, and transforming growth factor beta (TGF-β), and in some cases are involved in cellular immune responses. In some embodiments, exemplary Th2-related cytokines include IL-4, IL-5, IL-6, IL-10 and IL-13, and in some cases are associated with humoral immunity and anti-inflammatory properties. In some embodiments, exemplary Th17-related cytokines include IL-17A and IL-17F, and in some cases are involved in recruiting neutrophils and macrophages, e.g., during an inflammatory reaction.

In particular embodiments, the recombinant receptor-dependent activity is the production and/or secretion of one or more of IL-1, IL-1β, IL-2, sIL-2Ra, IL-3, IL-5, IL-6, IL-7, IL-8, IL-10, IL-12, IL-13, IL 27, IL-33, IL-35, TNF, TNF alpha, CXCL2, CCL2, CCL3, CCL5, CCL17, CCL24, PGD2, LTB4, interferon gamma (IFN-γ), granulocyte macrophage colony stimulating factor (GM-CSF), macrophage inflammatory protein (MIP)-1a, MIP-1b, Flt-3L, fractalkine, and/or IL-5. In certain embodiments, the recombinant receptor-dependent activity production or secretion of a Th17 cytokine. In some embodiments, the Th17 cytokine is GMCSF. In some embodiments, the recombinant receptor-dependent activity comprises production or secretion of a Th2 cytokine, wherein the Th2 cytokine is IL-4, IL-5, IL-10, or IL-13.

In certain embodiments, the recombinant receptor-dependent activity is the production or secretion of a proinflammatory cytokine. Proinflammatory cytokines play a role in initiating the inflammatory response and to regulate the host defense against pathogens mediating the innate immune response. Proinflammatory cytokines include, but are not limited to, interleukins (IL), interleukin-1-beta (IL-1), interleukin-3 (IL-3), interleukin-5 (IL-5), interleukin-6 (IL-6), interleukin-13 (IL-13), tumor necrosis factor (TNF), CXC-chemokine ligand 2 (CXCL2), CC-chemokine ligand 2 (CCL2), CC-chemokine ligand 3 (CCL3), CC-chemokine ligand 5 (CCL5), CC-chemokine ligand 17 (CCL17), CC-chemokine ligand 24 (CCL24), prostaglandin D2 (PGD2) and leukotriene B4 (LTB4) as well as IL-33). In some embodiments, the CAR dependent activity is production and or secretion of an interleukin and/or a TNF family member. In particular embodiments, the CAR dependent activity is production and or secretion of IL-1, IL-6, IL-8, and IL-18, TNF-alpha or a combination thereof.

In particular embodiments the recombinant receptor-dependent activity is secretion of IL-2, IFN-gamma, TNF-alpha or a combination thereof.

In some embodiments, the phenotype (e.g., recombinant receptor-dependent activity) is or includes the production of a cytokine. In certain embodiments, the phenotype is or includes the production of more than one cytokine (e.g., polyfunctional). In certain embodiments, the recombinant receptor-dependent activity is or includes a lack of a production of one or more cytokines. In certain embodiments, the phenotype is or includes the production, or lack thereof, of one or more of IL-2, IL-5, IL-10, IL-13, IL-17, IFNG, or TNFA. In certain embodiments, the recombinant receptor-dependent activity is or includes the production, or lack thereof, of one or more of IL-2, IL-13, IFNG, or TNFA. In some embodiments, the recombinant receptor-dependent activity is the presence of a production, and/or the presence of a high level of production of the cytokine. In some embodiments, the phenotype is a low, reduced, or absent production of a cytokine.

In some embodiments, the phenotype is or includes the internal (intracellular) production of a cytokine, for example, as assessed in the presence of a stimulatory agent or under stimulatory conditions when secretion is prevented or inhibited. In some embodiments, the stimulatory agent is nonspecific stimulatory agent, e.g., a stimulatory agent that does not bind to an antigen binding domain, for example on a recombinant receptor (e.g., CAR). In some embodiments, the stimulatory agent is PMA/ionomycin, which can act as a nonspecific stimulatory agent. In some embodiments, the stimulatory agent is a specific stimulatory agent, e.g., is a stimulatory agent that is an antigen or an epitope thereof that is specific to the recombinant receptor (e.g., CAR), or is an antibody or fragment thereof that binds to and/or recognizes the recombinant receptor, or a combination thereof. In particular embodiments, the phenotype is or includes the lack or absence of an internal production of a cytokine. In certain embodiments, the phenotype is or includes the internal amount of one or more cytokines when the production of more than one cytokines as assessed with an ICS assay. In certain embodiments, the phenotype is or includes the internal amount of one or more of IL-2, IL-5, IL-13, IFNG, or TNFA as assessed with an ICS assay. In some embodiments, the phenotype is or includes a low internal amount or a lack of a detectable amount of one or more cytokines as assessed with an ICS assay. In certain embodiments, phenotype is or includes a low internal amount or a lack of a detectable amount of IL-2, IL-5, IL-13, IFNG, or TNFA as assessed with an ICS assay. In some embodiments, the phenotype includes assessment of multiple cytokines, e.g., by multiplexed assays or assays to assess polyfunctionality (see, e.g., Xue et al., (2017) Journal for ImmunoTherapy of Cancer 5:85). In some embodiments, the lack of cytokine expression is inversely correlated with or associated with activity and/or function of the cells and/or durability of response and progression free survival. In some embodiments, cells with reduced, minimal or no cytokine production, assessed according to any known method or method described herein, are reduced in the cell composition (e.g., output composition, therapeutic cell composition).

Particular embodiments contemplate that the phenotype may include the production of a cytokine or a lack of or a low amount of production for a cytokine. This may depend on several factors that include, but are not limited to, the identity of the cytokine, the assay performed to detect the cytokine, and the stimulatory agent or condition used with the assay. For example, in some embodiments it is contemplated that the phenotype is or includes a lack of, or a low level of IL-13 production as indicated by ICS while in some embodiments, the phenotype is or includes production of IFN-gamma as indicated by ICS.

In some embodiments, the phenotype is or includes production of one or more cytokines and either CD3⁺, CD4⁺, CD8⁺, CD3⁺/CAR⁺, CD4⁺/CAR⁺, CD8⁺/CAR⁺, annexin V⁻, annexin V⁻CD3⁺, annexin V⁻CD4⁺, annexin V⁻CD8⁺, annexin V⁻CD3⁺/CAR⁺, annexin V⁻ CD4⁺/CAR⁺, annexin V⁻CD8⁺/CAR⁺, activated caspase 3⁻, activated caspase 3⁻/CD3⁺, activated caspase 3⁻/CD4⁺, activated caspase 3⁻/CD8⁺, activated caspase 3⁻/CD3⁺/CAR⁺, activated caspase 3⁻/CD4⁺/CAR⁺, or activated caspase 3⁻/CD8⁺/CAR⁺, or a combination thereof. In particular embodiments, the phenotype is or includes production of one or more cytokines in CD4⁺/CAR⁺ and/or CD8⁺/CAR⁺. In some embodiments, the one or more cytokines are IL-2, IFN-gamma, and/or TNF-alpha. In some embodiments, the phenotype is or includes production of IL-2 in CD4⁺/CAR⁺ cells. In some embodiments, the phenotype is or includes production of TNF-alpha in CD4⁺/CAR⁺ cells. In some embodiments, the phenotype is or includes production of IL-2 and TNF-alpha in CD4⁺/CAR⁺ cells. In some embodiments, the phenotype is or includes production of IL-2 and IFN-gamma in CD4⁺/CAR⁺ cells. In some embodiments, the phenotype is or includes production of TNF-alpha in CD8⁺/CAR⁺ cells. In some embodiments, the phenotype is or includes production of IFN-gamma and TNF-alpha in CD8⁺/CAR⁺ cells. In some embodiments, the phenotype is or includes production of IL-2 in activated caspase 3⁻/CD4⁺/CAR⁺ cells. In some embodiments, the phenotype is or includes production of TNF-alpha in activated caspase 3⁻/CD4⁺/CAR⁺ cells. In some embodiments, the phenotype is or includes production of IL-2 and TNF-alpha in activated caspase 3⁻/CD4⁺/CAR⁺ cells. In some embodiments, the phenotype is or includes production of IL-2 and IFN-gamma in activated caspase 3⁻/CD4⁺/CAR⁺ cells. In some embodiments, the phenotype is or includes production of TNF-alpha in activated caspase 3⁻/CD8⁺/CAR⁺ cells. In some embodiments, the phenotype is or includes production of IFN-gamma and TNF-alpha in activated caspase 3⁻/CD8⁺/CAR⁺ cells. In some embodiments, the phenotype is or includes production of TNF-alpha in annexin V⁻/CD4⁺/CAR⁺ cells. In some embodiments, the phenotype is or includes production of IL-2 and TNF-alpha in annexin V⁻/CD4⁺/CAR⁺ cells. In some embodiments, the phenotype is or includes production of IL-2 and IFN-gamma in annexin V⁻/CD4⁺/CAR⁺ cells. In some embodiments, the phenotype is or includes production of TNF-alpha in annexin V⁻/CD8⁺/CAR⁺ cells. In some embodiments, the phenotype is or includes production of IFN-gamma and TNF-alpha in annexin V⁻/CD8⁺/CAR⁺ cells. In some embodiments, the phenotypes described in this paragraph are positively correlated with durable response and progression free survival. Thus, in some embodiments, cells including these phenotypes are maximized or increased in the cell composition (e.g., output composition, therapeutic cell composition).

In some embodiments, the phenotype is or includes a lack of production of one or more cytokines. In certain embodiments, the phenotype is or includes a lack of a production of one or more cytokines and either CD3⁺, CD4⁺, CD8⁺, CD3⁺/CAR⁺, CD4⁺/CAR⁺, CD8⁺/CAR⁺, annexin V⁻, annexin V⁻CD3⁺, annexin V⁻CD4⁺, annexin V⁻CD8⁺, annexin V⁻CD3⁺/CAR⁺, annexin V⁻CD4⁺/CAR⁺, annexin V⁻CD8⁺/CAR⁺, activated caspase 3⁻, activated caspase 3⁻/CD3⁺, activated caspase 3⁻/CD4⁺, activated caspase 3⁻/CD8⁺, activated caspase 3⁻/CD3⁺/CAR⁺, activated caspase 3⁻/CD4⁺/CAR⁺, or activated caspase 3⁻/CD8⁺/CAR⁺, or a combination thereof. In some embodiments, the one or more cytokines are IL-2, IFN-gamma, and/or TNF-alpha. In some embodiments, the phenotype is or includes the lack of production of IL-2 in activated caspase 3⁻/CD4⁺/CAR⁺ cells. In some embodiments, the phenotype is or includes the lack of production of TNF-alpha in activated caspase 3⁻/CD4⁺/CAR⁺ cells. In some embodiments, the phenotype is or includes the lack of production of IL-2 and TNF-alpha in activated caspase 3⁻/CD4⁺/CAR⁺ cells. In some embodiments, the phenotype is or includes the lack of production of IL-2 and IFN-gamma in activated caspase 3⁻/CD4⁺/CAR⁺ cells. In some embodiments, the phenotype is or includes the lack of production of TNF-alpha in activated caspase 3⁻/CD8⁺/CAR⁺ cells. In some embodiments, the phenotype is or includes the lack of production of INF-gamma and TNF-alpha in activated caspase 3⁻/CD8⁺/CAR⁺ cells. In some embodiments, the phenotypes described in this paragraph are negatively correlated with durable response and progression free survival.

In particular embodiments, the phenotype is or includes the presence or absence of an internal amount of one or more of IL-2, IL-13, IFN-gamma, or TNF-alpha as assessed with an ICS assay and one or more specific markers for a subset of cells or cells of a particular cell type. In some embodiments, the phenotype is or includes production, or lack thereof, of one or more of IL-2, IL-13, IFN-gamma, or TNF-alpha and CD4⁺/CAR⁺ and/or CD8⁺/CAR⁺. In certain embodiments, the phenotype is or includes production of IL-2 and CD4⁺/CAR⁺ and/or CD8⁺/CAR⁺. In some embodiments, the phenotype is or includes a lack of or low production of IL-2 and CD4⁺/CAR⁺ and/or CD8⁺/CAR⁺. In some embodiments, the phenotype is or includes production of IL-13 and CD4⁺/CAR⁺ and/or CD8⁺/CAR⁺. In some embodiments, the phenotype is or includes production of IL-13 and CD4⁺/CAR⁺ and/or CD8⁺/CAR⁺. In certain embodiments, the phenotype is or includes the lack of or low production of IL-13 and CD4⁺/CAR⁺ and/or CD8⁺/CAR⁺. In some embodiments, the phenotype is or includes production of IFN-gamma and CD4⁺/CAR⁺ and/or CD8⁺/CAR⁺. In certain embodiments, the phenotype is or includes production of TNF-alpha and CD4⁺/CAR⁺ and/or CD8⁺/CAR⁺. In certain embodiments, the phenotype is or includes a lack of or low production of TNF-alpha and CD4⁺/CAR⁺ and/or CD8⁺/CAR⁺.

Any one or more of the phenotypes, alone or in combination, can be assessed or determined in accord with the provided methods. In some embodiments, the phenotype is CD3⁺, CD3⁺/CAR⁺, CD4⁺/CAR⁺, CD8⁺/CAR⁺, or a combination thereof.

In certain embodiments, the phenotype is or includes CD3⁺. In certain embodiments, the phenotype is or includes CD3⁺/CAR⁺. In some embodiments, the phenotype is or includes CD8⁺/CAR⁺. In certain embodiments, the phenotype is or includes CD4⁺/CAR⁺.

In particular embodiments, the phenotype is or includes Annexin⁻/CD3⁺/CAR⁺. In some embodiments, the phenotype is or includes Annexin⁻/CD4⁺/CAR⁺ In particular embodiments, the phenotype is Annexin⁻/CD8⁺/CAR.

In particular embodiments, the phenotype is or includes a lack of or a low amount of intracellular IL-2 and CD4⁺/CAR⁺. In particular embodiments, the phenotype is a lack of or a low amount of intracellular IL-13 and CD4⁺/CAR⁺. In some embodiments, the phenotype is a lack of or a low amount of intracellular expression of IL-13 and CD8⁺/CAR⁺ cells. In particular embodiments, the phenotype is a lack of or a low amount of intracellular TNF-alpha CD4⁺/CAR⁺.

In certain embodiments, the phenotype is or includes CD8⁺/CAR⁺. In certain embodiments, the phenotype is or includes annexin⁻/CD8⁺/CAR⁺.

In some embodiments, the phenotype comprises an indicator of production of one or a combination of cytokines, optionally non-specific to the antigen or the recombinant receptor and/or that is polyclonally produced, wherein the one or more cytokines is IL-2, IL-13, IL-17, IFN-gamma or TNF-alpha. In some embodiments, the indicator of production is measured in an assay, optionally an intracellular cytokine staining assay, comprising incubating a sample of the T cell composition with a polyclonal agent, an antigen-specific agent or an agent that binds the recombinant receptor, optionally CAR. In some embodiments, the agent is or comprises PMA and ionomycin or is or comprises a T cell receptor or T cell receptor complex agonist. In some embodiments, the phenotype comprises a naïve phenotype or a memory phenotype, optionally wherein the memory phenotype comprises a T effector memory phenotype, a T central memory phenotype, or a T effector memory phenotype expressing CD45RA (Temra).

In some embodiments, the recombinant receptor-dependent (e.g., CAR) activity is a measure of the production or accumulation of a proinflammatory cytokine, optionally, one of or a combination of TNF-alpha, IFN-gamma, and IL-2. In some embodiments, the recombinant receptor-dependent (e.g., CAR) activity is a measure of the production or accumulation of a combination of TNF-alpha, IFN-gamma, and IL-2, and IL-17. In some embodiments, the recombinant receptor-dependent (e.g., CAR) activity is a measure of the production or accumulation of IFN-gamma, and IL-2. In some embodiments, the recombinant receptor-dependent (e.g., CAR) activity is a measure of the production or accumulation of IFN-gamma, TNFA, and IL-2. In some embodiments, the recombinant receptor-dependent (e.g., CAR) activity is a measure of the production or accumulation of IFN-gamma and TNFA.

In some embodiments, the recombinant receptor activity is recombinant receptor-specific killing (e.g., cytolytic behavior). In some embodiments, the cytolytic activity of engineered CD8+ cells is assessed (e.g., quantified). In some embodiments, recombinant receptor-dependent cytolytic activity is assessed by exposing, incubating, and/or contacting cells expressing the recombinant receptor, or a cell composition containing cells that express the recombinant receptor, with a target cell that expresses the antigen and/or an epitope that is bound by and/or recognized by the recombinant receptor. The cytolytic activity can be measured by directly or indirectly measuring the target cell number over time. For example, the target cells may be incubated with a detectable marker prior to being incubated with recombinant receptor expressing cells, such a marker that is detectable then the target cell is lysed, or a detectable marker that is detectable in viable target cells. These readouts provide direct or indirect of target cell number and/or target cell death, and can be measured at different time points during the assay. A reduction of target cell number and/or an increase of target cell death indicate the cytolytic activity of the cells. Suitable methods for performing cytolytic assays are known in the art, and include, but are not limited to chromium-51 release assays, non-radioactive chromium assays, flow cytometric assays that use fluorescent dyes such as carboxyfluorescein succinimidyl ester (CFSE), PKH-2, and PKH-26.

In certain embodiments, the recombinant receptor, e.g., CAR, dependent cytolytic activity is measured by incubating the cell composition that contains cells expressing the recombinant receptor with target cells that express an antigen or an epitope thereof the is bound by or recognized by the recombinant receptor. In certain embodiments, the recombinant receptor is a CAR. In some embodiments, the cells of the cell composition are incubated with the target cells at a ratio of about 10:1, about 5:1, about 4:1, about 3:1, about 2:1, about 1:1, about 1:2, about 1:3, about 1:4, about 1:5, about 1:6, about 1:7, about 1:8, about 1:9, or about 1:10, or at a ratio between 10:1 and 1:1, 3:1 and 1:3, or 1:1 and 1:10, each inclusive. In some embodiments, the cells of the cell composition are incubated with the target cells at ratio of CAR+ cells, CAR+/CD8+ cells, or Annexin−/CAR+/CD8+ cells of the cell composition to target cells of about 10:1, about 5:1, about 4:1, about 3:1, about 2:1, about 1:1, about 1:2, about 1:3, about 1:4, about 1:5, about 1:6, about 1:7, about 1:8, about 1:9, or about 1:10, or at a ratio between 10:1 and 1:1, 3:1 and 1:3, or 1:1 and 1:10, each inclusive.

In certain embodiments, cells of the cell composition are incubated with the target cells for up to or about 1 hour, about 2 hours, about 3 hours, about 4 hours, about 5 hours, about 6 hours, about 8 hours, about 12 hours, about 18 hours, about 24 hours, about 48 hours, or greater than 48 hours. In some embodiments, the cell compositions are incubated for about 18 hours, 19 hours, 20 hours, 21 hours, 22 hours, 23 hours, or 24 hours. In some embodiments, between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, or between about 1×10⁸ and about 1×10¹⁰ cells of the cell composition, each inclusive, are incubated with the target cells. In certain embodiments, the between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, or between about 1×10⁸ and about 1×10¹⁰ CAR+ cells, CAR+/CD8+ cells, or Annexin−/CAR+/CD8+ cells of the cell composition, each inclusive, are incubated with the target cells.

In some embodiments, the measurement of the activity is compared to a control. In certain embodiments, the control is a culture of target cells that are not incubated with the cell composition. In some embodiments, the control is a measurement from a control cell composition that does not contain CAR+ cells that are incubated with the target cells at the same ratio.

In certain embodiments, the measurement of the cytolytic activity assay is the number of target cells that are viable at a time point during or at the end of the incubation. In certain embodiments, the measurement is an amount of a marker of target cell death, e.g., chromium-51, that is released during the incubation. In some embodiments, the measurement is an amount of target cell death that is determined by subtracting the amount of target cells in the co-incubation at a given time point from the amount of target cells of the control that was incubated alone. In some embodiments, the measurement is the percentage of target cells that remain at a time point compared to the starting amount of target cells. In particular embodiments, the measurement is the amount of cells killed over an amount of time. In certain embodiments, the measurement is the amount of cells killed per each cell of the cell composition. In some embodiments, the measurement is the amount of cells killed per cell, or the amount of cells killed per a set number or reference of cells, for example but not limited to, the amount of target cells killed per 100 cells, per 10³ cells, per 10⁴ cells, per 10⁵ cells, per 10⁶ cells, per 10⁷ cells, per 10⁸ cells, per 10⁹ cells, or per 10¹⁰ cells of the composition. In particular embodiments, the measurement is the amount of cells killed per each CAR+ cell, CAR+/CD8+ cell, or Annexin−/CAR+/CD8+ cell, or a reference or set number thereof, of the cell composition. In certain embodiments, the measurement is the amount of cells killed over an amount of time per cell of the cell composition. In particular embodiments, the measurement is the amount of cells killed over an amount of time per CAR+ cells, CAR+/CD8+ cells, or Annexin−/CAR+/CD8+ cells of the cell composition.

In some embodiments, the cell phenotype includes assessing the genomic integration of transgene sequences, such as transgene sequences encoding a recombinant receptor, e.g. a CAR. In some embodiments, the cell phenotype is an integrated copy number, e.g., vector copy number, which is the copy number of the transgene sequence integrated into the chromosomal DNA or genomic DNA of cells. In some embodiments, the vector copy number can be expressed as an average or mean copy number. In some aspects, the vector copy number of a particular integrated transgene includes the number of integrants (containing transgene sequences) per cell. In some embodiments, the vector copy number of a particular integrated transgene includes the number of integrants (containing transgene sequences) per diploid genome. In some aspects, the vector copy number of transgene sequence is expressed as the number of integrated transgene sequences per cell. In some aspects, the vector copy number of transgene sequence is expressed as the number of integrated transgene sequences per diploid genome. In some embodiments, the copy number is an average or mean copy number per diploid genome or per cell among the population of cells.

In some embodiments, the attributes of the therapeutic cell composition include cell phenotypes 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+, CD3+/CAR+, CD3+/CD56+, and/or CAR+.

In some embodiments, for example when the cells of the therapeutic cell composition contain an anti-CD19 CAR, the attributes of the therapeutic cell composition include cell phenotypes 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+/CD19+, CD3+/CAR+, CD3+/CD56+, and/or CAR+.

In some embodiments, for example when the therapeutic cell composition is engineered CD4+ cells, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, and/or CD4+/CAR+.

In some embodiments, for example when the therapeutic cell composition is engineered CD4+ cells containing an anti-CD19 CAR, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, and/or CD4+/CAR+.

In some embodiments, for example when the therapeutic cell composition is engineered CD8+ cells, the attributes of the therapeutic cell composition include cell phenotypes including 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, and/or CD8+/CAR+.

In some embodiments, for example when the therapeutic cell composition is engineered CD8+ cells containing an anti-CD19 CAR, the attributes of the therapeutic cell composition include cell phenotypes including 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells containing an anti-CD19 CAR, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD19+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, the attributes of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+, IFNG+, IL10+, IL13+, IL2+, IL5+, MIP1A+, MIP1B+, sCD137+, and/or TNFa+.

In some embodiments, the attributes of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+/CD19+, IFNG+/CD19+, IL10+/CD19+, IL13+/CD19+, IL2+/CD19+, IL5+/CD19+, MIP1A+/CD19+, MIP1B+/CD19+, sCD137+/CD19+, and/or TNFa+/CD19+, when the cells of the therapeutic cell composition contain an anti-CD19 CAR.

In some embodiments, for example when the therapeutic cell composition is engineered CD4+ T cells, the attributes of the therapeutic cell composition include recombinant receptor-dependent activity including IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+, IL-10+, IL-13+, IL-2+/CD19+, IL-5+, MIP1A+, MIP1B+, sCD137+, and/or TNFa+.

In some embodiments, for example when the therapeutic cell composition is engineered CD4+ T cells containing an anti-CD19 CAR, the attributes of the therapeutic cell composition include recombinant receptor-dependent activity including IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, for example when the therapeutic cell composition is engineered CD8+ T cells, the attributes of the therapeutic cell composition include recombinant receptor-dependent activity including IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+.

In some embodiments, for example when the therapeutic cell composition is engineered CD8+ T cells containing an anti-CD19 CAR, the attributes of the therapeutic cell composition include recombinant receptor-dependent activity including IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, for example when the therapeutic cell composition is engineered CD4+ and CD8+ T cells or there are separate therapeutic compositions of CD4+ and CD8+ engineered cells, the attributes of the therapeutic cell composition include recombinant receptor-dependent activity including IFNG+/IL2+/CD4+/CAR+, IFNG+/IL2+/IL17+/TNFA+/CD4+/CAR+, IFNG+/IL2+/TNFA+/CD4+/CAR+, IFNG+ of CD4+/CAR+, IFNG+/TNFA+/CD4+/CAR+, IL13+ of CD4+/CAR+, IL17+ of CD4+/CAR+, IL2+ of CD4+/CAR+, IL2+/TNFA+/CD4+/CAR+, TNFA+ of CD4+/CAR+, IFNG+/IL2+/CD8+/CAR+, IFNG+/IL2+/IL17+/TNFA+/CD8+/CAR+, IFNG+/IL2+/TNFA+/CD8+/CAR+, IFNG+ of CD8+/CAR+, IFNG+/TNFA+/CD8+/CAR+, IL13+ of CD8+/CAR+, IL17+ of CD8+/CAR+, IL2+ of CD8+/CAR+, IL2+/TNFA+/CD8+/CAR+, TNFA+ of CD8+/CAR+, cytolytic CD8+, GMCSF+, IFNG+, IL10+, IL13+, IL2+, IL5+, MIP1A+, MIP1B+, sCD137+, and/or TNFa+.

In some embodiments, the therapeutic cell composition attributes are those shown in Table E2 below. In some embodiments, the therapeutic cell composition attributes are one or more of those shown in Table E2 below.

In some of any of the above embodiments, the percentage, number, and/or proportion of cells having an attribute that is a phenotype as described above, is determined, measured, obtained, detected, observed, and/or identified. In certain embodiments, the number of cells of the phenotype is the total amount of cells of the phenotype of the cell composition. In some embodiments, the number of the cells of the phenotype may be expressed as a frequency, ratio, and/or a percentage of cells of the phenotype present in the therapeutic cell composition.

In some embodiments, the number, multiple, or fraction of the cells of a phenotype is transformed, for example to compress the range of relevant values of the number, multiple, or fraction. In some embodiments, the transformation is any application of a deterministic mathematical function to each point in a data set, such as, each data point x is replaced with the transformed value y=f(x), where f is a function. In general, transforms may be applied so that the data appear to more closely meet the assumptions of a statistical inference procedure that is to be applied, or to improve the interpretability or appearance of graphs. In most cases the function that is used to transform the data is invertible, and generally is continuous. The transformation is usually applied to a collection of comparable measurements. Examples of suitable transformations include, but are not limited to, logarithm and square root transformation, reciprocal transformations, and power transformations. In certain embodiments, the number, multiple, or fraction of the cells of a phenotype is transformed by a logarithmic transformation. In certain embodiments, the logarithmic transformation is a common log (log₁₀(x)), a natural log (ln(x)) or a binary log (log₂(x)).

a. Desired Attributes

In some cases, the attributes of the therapeutic cell composition may be considered desired attributes. In some embodiments, a desired attribute is a cell phenotype or recombinant-receptor dependent activity that is known or suspected of being positively correlating with positive clinical outcomes (also referred to herein as positive clinical response). In some embodiments, the positive clinical response is one or more of a complete response (CR); a partial response (PR); a durable response, e.g., of greater than 3 months; progression free survival (PFS), e.g., for more than 3 months; a pharmacokinetic response that is or is greater than a target pharmacokinetic response; and no or a mild toxicity response (optionally wherein the toxicity is grade 2 or less CRS or grade 2 or less neurotoxicity).

In some embodiments, cell phenotypes and functional attributes, e.g., recombinant receptor-dependent activity, associated with less differentiated T cells or naïve, naïve-like or central memory T cells, or T cell subsets thereof, correlate with or exhibit a relationship with improved pharmacokinetic properties or responses, such as durability of response and/or progression free survival, following administration to a subject. Thus, in some cases, a desired attribute is a cell phenotype or functional attribute, e.g., recombinant receptor-dependent activity, associated with less differentiated T cells or naïve, naïve-like or central memory T cells.

In some embodiments, a desired attribute is a marker of cell persistence, e.g., T cell persistence. In some embodiments, a desired attribute is cytolytic activity, for example effective cell killing at or below expected successful effector to target ratios.

In some embodiments, a desired attribute is the production or one or more cytokines. In some embodiments, a desired attribute is polyfunctionality, wherein a cell, e.g., T cell, produces two or more cytokines. In some embodiments, cytokine production by the cell, e.g., T cell, is induced by stimulation of the recombinant receptor (e.g., recombinant receptor-dependent activity).

In some embodiments, a desired attribute is a threshold level of cells, e.g., T cells, having an attribute, for example an attribute described in this Section, or an attribute described in Section I-A-2 above that is known or suspected of positively correlating with one or more positive clinical responses. In some embodiments, the threshold level is a percentage, number, ratio, and/or proportion of cells, e.g., T cells, in the therapeutic cell composition having a desired attribute. It should be appreciate that a threshold can be expressed by any known unit of measure, e.g., as described herein, by using proper conversion methods according to mathematical principles.

In some embodiments, a desired attribute is at least one attribute that is correlated with a positive clinical response to treatment with the therapeutic cell composition. In some embodiments, the positive clinical response is a durable response and/or progression free survival.

In some embodiments, a desired attribute is or includes a threshold percentage of naïve-like T cells or central memory T cells. In some embodiments, wherein the threshold percentage is at least or at least about 40% of the cells in the therapeutic cell composition that are naïve-like T cells or central memory T cells. In some embodiments, the threshold percentage is at least or at least about 50% of the cells in the therapeutic cell composition that are naïve-like T cells or central memory T cells. In some embodiments, the threshold percentage is at least or at least about 60% of the cells in the therapeutic cell composition that are naïve-like T cells or central memory T cells. In some embodiment, the threshold percentage is at least or at least about 65% of the cells in the therapeutic cell composition that are naïve-like T cells or central memory T cells. the threshold percentage is at least or at least about 70% of the cells in the therapeutic cell composition that are naïve-like T cells or central memory T cells. In some embodiments, the naïve-like T cells or central memory T cells have a phenotype comprising T cells surface positive for CD27+, CD28+, CD62L+, and/or CCR7+. In some embodiments, the naïve-like T cells or central memory T cells have the phenotype CD62L+/CCR7+, CD27+/CCR7+, CD62L+/CD45RA−, CCR7+/CD45RA−, CD62L+/CCR7+/CD45RA−, CD27+/CD28+/CD62L+/CD45RA−, CD27+/CD28+/CCR7+/CD45RA−, CD27+/CD28+/CD62L+/CCR7+, or CD27+/CD28+/CD62L+/CCR7+/CD45RA−.

In some embodiments, a desired attribute is or includes a threshold percentage of CD27+/CCR7+ T cells in the therapeutic cell composition. In some embodiments, the threshold percentage is at least or at least about 60% of the cells in the therapeutic cell composition are CD27+/CCR7+. In some embodiments, the threshold percentage is or is about 60%, 70,%, 75%, 80%, 85%, 90%, 91%, 92%, 93%, 94%, 95%, 96%, 97%, 98%, 99%, 100%, or any intervening value of the foregoing, of the cells in the therapeutic cell composition are CD27+/CCR7+. In some embodiments, the threshold percentage is or is about 60% of the cells in the therapeutic cell composition are CD27+/CCR7+. In some embodiments, the threshold percentage is or is about 70% of the cells in the therapeutic cell composition are CD27+/CCR7+. In some embodiments, the threshold percentage is or is about 75% of the cells in the therapeutic cell composition are CD27+/CCR7+. In some embodiments, the threshold percentage is or is about 80% of the cells in the therapeutic cell composition are CD27+/CCR7+. In some embodiments, the threshold percentage is or is about 85% of the cells in the therapeutic cell composition are CD27+/CCR7+. In some embodiments, the threshold percentage is or is about 90% of the cells in the therapeutic cell composition are CD27+/CCR7+. In some embodiments, the threshold percentage is or is about 95% of the cells in the therapeutic cell composition are CD27+/CCR7+. In some embodiments, the threshold percentage is or is about 96% of the cells in the therapeutic cell composition are CD27+/CCR7+. In some embodiments, the threshold percentage is or is about 97% of the cells in the therapeutic cell composition are CD27+/CCR7+. In some embodiments, the threshold percentage is or is about 98% of the cells in the therapeutic cell composition are CD27+/CCR7+. In some embodiments, the threshold percentage is or is about 99% of the cells in the therapeutic cell composition are CD27+/CCR7+. In some embodiments, the CD27+/CCR7+ cells are CD4+/CAR+ T cells and CD8+/CAR+ T cells. In some embodiments, the CD27+/CCR7+ cells are CD4+/CAR+ T cells. In some embodiments, the CD27+/CCR7+ cells are CD8+/CAR+ T cells.

In some embodiments, the threshold percentage is or is about 60% of the cells in the therapeutic cell composition are CD27+/CCR7+/CD4+/CAR+/CD4+/CAR+. In some embodiments, the threshold percentage is or is about 70% of the cells in the therapeutic cell composition are CD27+/CCR7+/CD4+/CAR+. In some embodiments, the threshold percentage is or is about 75% of the cells in the therapeutic cell composition are CD27+/CCR7+/CD4+/CAR+. In some embodiments, the threshold percentage is or is about 80% of the cells in the therapeutic cell composition are CD27+/CCR7+/CD4+/CAR+. In some embodiments, the threshold percentage is or is about 85% of the cells in the therapeutic cell composition are CD27+/CCR7+/CD4+/CAR+. In some embodiments, the threshold percentage is or is about 90% of the cells in the therapeutic cell composition are CD27+/CCR7+/CD4+/CAR+. In some embodiments, the threshold percentage is or is about 95% of the cells in the therapeutic cell composition are CD27+/CCR7+/CD4+/CAR+. In some embodiments, the threshold percentage is or is about 96% of the cells in the therapeutic cell composition are CD27+/CCR7+/CD4+/CAR+. In some embodiments, the threshold percentage is or is about 97% of the cells in the therapeutic cell composition are CD27+/CCR7+/CD4+/CAR+. In some embodiments, the threshold percentage is or is about 98% of the cells in the therapeutic cell composition are CD27+/CCR7+/CD4+/CAR+. In some embodiments, the threshold percentage is or is about 99% of the cells in the therapeutic cell composition are CD27+/CCR7+/CD4+/CAR+.

In some embodiments, the threshold percentage is or is about 60% of the cells in the therapeutic cell composition are CD27+/CCR7+/CD8+/CAR+/CD4+/CAR+. In some embodiments, the threshold percentage is or is about 70% of the cells in the therapeutic cell composition are CD27+/CCR7+/CD8+/CAR+. In some embodiments, the threshold percentage is or is about 75% of the cells in the therapeutic cell composition are CD27+/CCR7+/CD8+/CAR+. In some embodiments, the threshold percentage is or is about 80% of the cells in the therapeutic cell composition are CD27+/CCR7+/CD8+/CAR+. In some embodiments, the threshold percentage is or is about 85% of the cells in the therapeutic cell composition are CD27+/CCR7+/CD8+/CAR+. In some embodiments, the threshold percentage is or is about 90% of the cells in the therapeutic cell composition are CD27+/CCR7+/CD8+/CAR+. In some embodiments, the threshold percentage is or is about 95% of the cells in the therapeutic cell composition are CD27+/CCR7+/CD8+/CAR+. In some embodiments, the threshold percentage is or is about 96% of the cells in the therapeutic cell composition are CD27+/CCR7+/CD8+/CAR+. In some embodiments, the threshold percentage is or is about 97% of the cells in the therapeutic cell composition are CD27+/CCR7+/CD8+/CAR+. In some embodiments, the threshold percentage is or is about 98% of the cells in the therapeutic cell composition are CD27+/CCR7+/CD8+/CAR+. In some embodiments, the threshold percentage is or is about 99% of the cells in the therapeutic cell composition are CD27+/CCR7+/CD8+/CAR+.

In some embodiments, a desired attribute is or includes a threshold percentage of IL-2+ of CD4+/CAR+ and IL-2+/TNFA+/CD4+/CAR+ T cells in the therapeutic cell composition. In some embodiments, a desired attribute is or includes a threshold percentage of IL-2+ of CD4+/CAR+ T cells in the therapeutic cell composition. In some embodiments, a desired attribute is or includes a threshold percentage of IL-2+/TNFA+/CD4+/CAR+ T cells in the therapeutic cell composition. In some embodiments, the threshold percentage is at least at or about 70%, 80%, 85%, 90%, 91%, 92%, 93%, 94%, 95%, 96%, 97%, 98%, 99% or 100% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 70% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 80% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 85% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 90% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 91% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 93% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 85% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 94% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 95% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 96% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 97% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 98% of the total number of CD4+ T cells in the therapeutic cell composition. In some embodiments, the threshold percentage of IL-2+ of CD4+/CAR+ and IL-2+/TNFA+/CD4+/CAR+ T cells is at least at or about 99% of the total number of CD4+ T cells in the therapeutic cell composition.

In some embodiments, a desired attribute is or includes a threshold percentage of IL-2+ of CD8+/CAR+ and IL-2+/TNFA+/CD8+/CAR+ T cells in the therapeutic cell composition. In some embodiments, a desired attribute is or includes a threshold percentage of IL-2+ of CD8+/CAR+ T cells in the therapeutic cell composition. In some embodiments, a desired attribute is or includes a threshold percentage of IL-2+/TNFA+/CD8+/CAR+ T cells in the therapeutic cell composition. In some embodiments, the threshold percentage is at least at or about 70%, 80%, 85%, 90%, 91%, 92%, 93%, 94%, 95%, 96%, 97%, 98%, 99% or 100% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 70% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 80% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 85% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 90% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 91% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 93% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 85% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 94% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 95% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 96% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 97% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 98% of the total number of CD8+ T cells in the therapeutic cell composition. In some embodiments, the threshold percentage of IL-2+ of CD8+/CAR+ and IL-2+/TNFA+/CD8+/CAR+ T cells is at least at or about 99% of the total number of CD8+ T cells in the therapeutic cell composition.

In some embodiments, a desired attribute is or comprises a threshold percentage of IFNG+/IL-2+/CD4+/CAR+, IFNG+/IL-2+/IL-17+/TNFA+/CD4+/CAR+, IFNG+/IL-2+/TNFA+/CD4+/CAR+, IFNG+/TNFA+/CD4+/CAR+, IL-17+ of CD4+CAR+, IL-2+ of CD4+CAR+, and/or IL-2+/TNFA+/CD4+/CAR+ T cells in the therapeutic cell composition. In some embodiments, a desired attribute is or comprises a threshold percentage of IFNG+/IL-2+/CD4+/CAR+ T cells in the therapeutic cell composition. In some embodiments, a desired attribute is or comprises a threshold percentage of IFNG+/IL-2+/IL-17+/TNFA+/CD4+/CAR+ T cells in the therapeutic cell composition. In some embodiments, a desired attribute is or comprises a threshold percentage of IFNG+/IL-2+/TNFA+/CD4+/CAR+ T cells in the therapeutic cell composition. In some embodiments, a desired attribute is or comprises a threshold percentage of IFNG+/TNFA+/CD4+/CAR+ T cells in the therapeutic cell composition. In some embodiments, a desired attribute is or comprises a threshold percentage of IL-17+ of CD4+CAR+ T cells in the therapeutic cell composition. In some embodiments, a desired attribute is or comprises a threshold percentage of IL-2+/TNFA+/CD4+/CAR+ T cells in the therapeutic cell composition. In some embodiments, the threshold percentage is at least at or about 10%, 15%, 20%, 25%, 30%, 40%, 50%, 60% or more of the total number of CAR+/CD4+ T cells in the therapeutic cell composition. In some embodiments, the threshold percentage is at least at or about 10% of the total number of CAR+/CD4+ T cells in the therapeutic cell composition. In some embodiments, the threshold percentage is at least at or about 15% or more of the total number of CAR+/CD4+ T cells in the therapeutic cell composition. In some embodiments, the threshold percentage is at least at or about 20% of the total number of CAR+/CD4+ T cells in the therapeutic cell composition. In some embodiments, the threshold percentage is at least at or about 30% of the total number of CAR+/CD4+ T cells in the therapeutic cell composition. In some embodiments, the threshold percentage is at least at or about 40% of the total number of CAR+/CD4+ T cells in the therapeutic cell composition. In some embodiments, the threshold percentage is at least at or about 50% of the total number of CAR+/CD4+ T cells in the therapeutic cell composition. In some embodiments, the threshold percentage is at least at or about 60% of the total number of CAR+/CD4+ T cells in the therapeutic cell composition. In some embodiments, the threshold percentage is at least at or about 70% of the total number of CAR+/CD4+ T cells in the therapeutic cell composition. In some embodiments, the threshold percentage is at least at or about 80% of the total number of CAR+/CD4+ T cells in the therapeutic cell composition. In some embodiments, the threshold percentage is at least at or about 90% of the total number of CAR+/CD4+ T cells in the therapeutic cell composition. In some embodiments, the threshold percentage is at least at or about 95% of the total number of CAR+/CD4+ T cells in the therapeutic cell composition. In some embodiments, the threshold percentage is at least at or about 96% of the total number of CAR+/CD4+ T cells in the therapeutic cell composition. In some embodiments, the threshold percentage is at least at or about 97% of the total number of CAR+/CD4+ T cells in the therapeutic cell composition. In some embodiments, the threshold percentage is at least at or about 97% of the total number of CAR+/CD4+ T cells in the therapeutic cell composition. In some embodiments, the threshold percentage is at least at or about 98% of the total number of CAR+/CD4+ T cells in the therapeutic cell composition. In some embodiments, the threshold percentage is at least at or about 99% of the total number of CAR+/CD4+ T cells in the therapeutic cell composition.

In some embodiments, a desired attribute is or comprises a threshold percentage of IFNG+/IL-2+/CD8+/CAR+, IFNG+/IL-2+/IL-17+/TNFA+/CD8+/CAR+, IFNG+/IL-2+/TNFA+/CD8+/CAR+, IFNG+/TNFA+/CD8+/CAR+, IL-17+ of CD8+CAR+, IL-2+ of CD8+CAR+, and/or IL-2+/TNFA+/CD8+/CAR+ T cells in the therapeutic cell composition. In some embodiments, a desired attribute is or comprises a threshold percentage of IFNG+/IL-2+/CD8+/CAR+ T cells in the therapeutic cell composition. In some embodiments, a desired attribute is or comprises a threshold percentage of IFNG+/IL-2+/IL-17+/TNFA+/CD8+/CAR+ T cells in the therapeutic cell composition. In some embodiments, a desired attribute is or comprises a threshold percentage of IFNG+/IL-2+/TNFA+/CD8+/CAR+ T cells in the therapeutic cell composition. In some embodiments, a desired attribute is or comprises a threshold percentage of IFNG+/TNFA+/CD8+/CAR+ T cells in the therapeutic cell composition. In some embodiments, a desired attribute is or comprises a threshold percentage of IL-17+ of CD8+CAR+ T cells in the therapeutic cell composition. In some embodiments, a desired attribute is or comprises a threshold percentage of IL-2+/TNFA+/CD8+/CAR+ T cells in the therapeutic cell composition. In some embodiments, the threshold percentage is at least at or about 10%, 15%, 20%, 25%, 30%, 40%, 50%, 60% or more of the total number of CAR+/CD8+ T cells in the therapeutic cell composition. In some embodiments, the threshold percentage is at least at or about 10% of the total number of CAR+/CD8+ T cells in the therapeutic cell composition. In some embodiments, the threshold percentage is at least at or about 15% or more of the total number of CAR+/CD8+ T cells in the therapeutic cell composition. In some embodiments, the threshold percentage is at least at or about 20% of the total number of CAR+/CD8+ T cells in the therapeutic cell composition. In some embodiments, the threshold percentage is at least at or about 30% of the total number of CAR+/CD8+ T cells in the therapeutic cell composition. In some embodiments, the threshold percentage is at least at or about 40% of the total number of CAR+/CD8+ T cells in the therapeutic cell composition. In some embodiments, the threshold percentage is at least at or about 50% of the total number of CAR+/CD8+ T cells in the therapeutic cell composition. In some embodiments, the threshold percentage is at least at or about 60% of the total number of CAR+/CD8+ T cells in the therapeutic cell composition. In some embodiments, the threshold percentage is at least at or about 70% of the total number of CAR+/CD8+ T cells in the therapeutic cell composition. In some embodiments, the threshold percentage is at least at or about 80% of the total number of CAR+/CD8+ T cells in the therapeutic cell composition. In some embodiments, the threshold percentage is at least at or about 90% of the total number of CAR+/CD8+ T cells in the therapeutic cell composition. In some embodiments, the threshold percentage is at least at or about 95% of the total number of CAR+/CD8+ T cells in the therapeutic cell composition. In some embodiments, the threshold percentage is at least at or about 96% of the total number of CAR+/CD8+ T cells in the therapeutic cell composition. In some embodiments, the threshold percentage is at least at or about 97% of the total number of CAR+/CD8+ T cells in the therapeutic cell composition. In some embodiments, the threshold percentage is at least at or about 97% of the total number of CAR+/CD8+ T cells in the therapeutic cell composition. In some embodiments, the threshold percentage is at least at or about 98% of the total number of CAR+/CD8+ T cells in the therapeutic cell composition. In some embodiments, the threshold percentage is at least at or about 99% of the total number of CAR+/CD8+ T cells in the therapeutic cell composition.

In some embodiments, a desired attribute is any one or more of a desired attribute described in this Section, including threshold values thereof, or any one of the attributes, e.g., cell phenotypes and recombinant receptor-dependent activity, described in Section I-A-2, including threshold values thereof.

In some embodiments, the statistical methods described herein may predict the presence and/or quantity of a desired attribute in a therapeutic cell composition from attributes of an input composition prior to the input composition being manufactured into a therapeutic cell composition. In some embodiments, the predicted presence and/or quantity of a desired attribute in a therapeutic cell composition may inform methods of manufacturing the therapeutic cell composition, e.g., inform selection of a manufacturing process with a likelihood of producing a therapeutic cell composition with desired attributes. See, for example, Section I-C-4 below. In some embodiments, the predicted presence and/or quantity of a desired attribute in a therapeutic cell composition may inform methods of treating a subject in need thereof, e.g., to maximize therapeutic efficacy and/or effectiveness. See, for example, Section I-C-3 below.

B. Methods of Identifying Correlated Attributes

It is contemplated that the attributes of the therapeutic cell composition (e.g., engineered T cell composition) can, in some cases, depend upon many factors, including, but not limited to, the attributes of the starting cellular material (e.g., apheresis product or leukapheresis product or cells selected therefrom (e.g., input composition)) used to generate the therapeutic cell composition. Thus, in some embodiments, input composition attributes and attributes of the therapeutic cell composition produced from the input composition are assessed (e.g., quantified) and used as input for statistical methods capable of determining correlations between sets of data including multiple variables (e.g., input and therapeutic cell composition attributes). In some embodiments, input composition attributes and attributes of the therapeutic cell composition produced from the input composition are assessed (e.g., quantified) and used as input for statistical methods capable of correlating a single variable (e.g., therapeutic cell composition attribute) from a plurality of input variables (e.g., input composition attributes). In some embodiments, the attributes are cell phenotypes. In some embodiments, the attributes, for example in the therapeutic cell composition are recombinant receptor-dependent activity. In some embodiments, the attributes, e.g., cell phenotypes, recombinant receptor-dependent activity, are quantified to provide a number, percentage, proportion, and/or ratio of cells having an attribute in the composition (e.g., input composition, therapeutic cell composition).

As described above, input and therapeutic cell compositions may contain CD3+, CD4+, CD8+ or CD4+ and CD8+ cells. Thus, in some embodiments, the attributes of the input and therapeutic cell compositions may be cell type specific. In some embodiments, for example when input compositions separately contain CD4+ or CD8+ cells from which the therapeutic T cell composition (e.g., CD4+ or CD8+) will be independently produced, attributes can be assess for each input and therapeutic cell composition and compared using statistical methods described herein. For example, when CD4+ and CD8+ cells are contained in separate input compositions and independently processed to generate separate CD4+ and CD8+ therapeutic cell compositions, statistical analyses of each of their attributes are not limited to assessing only cell type specific attribute relationships. Even when manufacturing is carried out on separate cell populations, the attributes of the populations can be combined in statistical analyses. In some embodiments, attributes from a separately processed cell type specific input and therapeutic cell composition are correlated with attributes from a different separately processed cell specific input and therapeutic cell composition. For example, attributes determined from an input composition containing CD4+ T cells, which is separately processed to produce a CD4+ therapeutic cell composition, can be used (e.g., as input) to determine correlations between attributes of the resultant CD4+ therapeutic composition and a CD8+ therapeutic cell composition produced from an input composition containing CD8+ T cells, and vice versa.

1. Penalized Canonical Correlation Analysis

In some embodiments, the statistical method for determining correlations between input and therapeutic composition attributes is canonical correlation analysis (CCA), and more particularly penalized canonical correlation analysis (pCCA). CCA can handle high dimensional data sets containing a plurality of variables (e.g., attributes) and identify correlations that are not limited by or to one to one relationships. As such, CCA is well suited to identifying relationships between groups of variables (e.g., therapeutic cell composition attributes) from a plurality of input variables (e.g., input composition attributed).

In some embodiments, CCA finds linear combinations of input composition attributes and linear combinations of therapeutic composition attributes that maximize the correlation between the input composition attributes and therapeutic composition attributes. In some embodiments, the linear combinations indicate the contribution (e.g., weight (e.g., canonical vector)) and directionality (positive, negative) of attributes (e.g., input composition attributes, therapeutic cell composition attributes) that maximize the correlation. In some embodiments, CCA identifies multiple linear combinations, e.g., multiple pairs of canonical variables. In some embodiments, the number of linear combinations (e.g., pairs of canonical variables) is equal to the length of the data set with the fewest number of variables. In some embodiments, the order in which the multiple linear combinations are found (e.g., first, second, third, etc., canonical pairs) indicates the strength of the canonical correlation and how much of the variance is captured by the canonical correlation, with the first pair having the highest canonical correlation and capturing the highest explained variance. In some embodiments, the explained variance is shared variance. In some embodiments, the explained variance is covariance.

In some embodiments, the CCA is pCCA. In some embodiments, pCCA, like CCA, is able to identify correlations between high dimensional data sets, but includes a convex penalty function that down-weights or sets to zero (e.g., removes) variables with small, independent effects. In some embodiment, pCCA is used to reduce model complexity (e.g., dimensionality).

In some embodiments, pCCA is captured by Equation 2:

argmax_{u,v}u^{T}X^{T}Yv subject to ∥u∥₂²≤1,∥v∥₂²≤1,P₁(u)≤C₁,P₂(v)≤C₂ (Eq. 2)

where X and Y represent sets of high dimensional variables (e.g., input attributes and therapeutic composition attributes) and u and v are canonical vectors (e.g., list of weights for each variable); P₁ and P₂ are convex penalty functions; and C₁ and C₂ are constants determined using a permutation scheme. In some embodiments, the convex penalty functions are lasso regularization, e.g., L1 regularization. In some embodiments, the canonical vectors are constrained by a requirement that the square of the L2 norm of the canonical vectors to be less than or equal to 1. In some embodiments, the pCCA is computed in R v3.5 or 3.6 using PMA package. In some embodiments, C₁ and C₂ are found using cca.permute in R v3.5 or 3.6. Examples of pCCA can be found in Witten et al., 2009.

In some embodiments, pCCA is performed using a first set of attributes (e.g., first attributes) determined from an input composition and a second set of attributes (e.g., second attributes) determined from a therapeutic cell composition produced from the input composition. In some embodiments, the input composition contains CD4+, CD8+, or CD4+ and CD8+ cells selected from a from a subject, and the therapeutic cell composition contains the engineered CD4+, CD8+, or CD4+ and CD8+ cells, respectively. In some embodiments, the first attributes are cell phenotypes. In some embodiments, the first attributes of the input composition include cell phenotypes, for example as described in Section I-A-1. In some embodiments, the input composition attributes are first attributes. In some embodiments, the first attributes include cell phenotype attributes. In some embodiments, the cell phenotypes include 3CAS−/CCR7−/CD27−, 3CAS−/CCR7−/CD27+, 3CAS−/CCR7+, 3CAS−/CCR7+/CD27−, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and/or CAS+/CD3+. In some embodiments, for example when the input composition is CD4+ T cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS/−CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, CAS+/CD4+, CAS+/CD3+, and/or 3CAS−/CCR7+/CD45RA+/CD4+. In some embodiments, for example when the input composition is CD8+ T cells, the cell phenotypes include 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD8+, and/or CAS+/CD3+. In some embodiments, for example when the input composition is CD4+ and CD8+ T cells or there are separate therapeutic compositions of CD4+ and CD8+ engineered cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, and/or CAS+/CD3+. In some embodiments, the input composition attributes (e.g., first attributes) are 34 cell phenotypes. In some embodiments, the 34 cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and/or CAS+/CD3+ of an input composition that is CD8+ cells. In some embodiments, the input composition attributes (e.g., first attributes) include a subset of any of the above cell phenotypes. In some embodiments, the input composition attributes (e.g., first attributes) include or include about 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 cell phenotypes. In some embodiments, the input composition attributes (e.g., first attributes) include or comprise about or at least 2, 4, 6, 8, 10, 12, or more cell phenotypes. In some embodiments, the input composition attributes (e.g., first attributes) include great than or great than about 5, 10, 15, or 20 cell attributes.

In some embodiments, the first attributes include input composition attributes shown in Table E2, or a subset thereof. In some embodiments, the first attributes include one or more input composition attributes shown in Table E2.

In some embodiments, the attributes of the therapeutic cell composition include cell phenotypes, for example as described in Section I-A-2. In some embodiments, the therapeutic cell composition attributes are second attributes. In some embodiments, the second attributes include cell phenotype attributes. In some embodiments, the cell phenotypes include 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CYTO−/CAR+, EGFRt+, IFNG+, viable cell concentration (VCC), vector copy number (VCN), viability, GMCSF+, CD3+/CAR+, CD3+/CD56+, and/or CAR+. In some embodiments, the cell phenotypes include 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+/CD19+, CD3+/CAR+, CD3+/CD56+, and/or CAR+.

In some embodiments, for example when the therapeutic cell composition is engineered CD4+ cells, the cell phenotypes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, and/or CD4+/CAR+.

In some embodiments, for example when the therapeutic cell composition is engineered CD4+ cells containing an anti-CD19 CAR, the cell phenotypes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, and/or CD4+/CAR+.

In some embodiments, for example when the therapeutic cell composition is engineered CD8+ cells, the cell phenotypes include 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, and/or CD8+/CAR+.

In some embodiments, for example when the therapeutic cell composition is engineered CD8+ cells containing an anti-CD19 CAR, the cell phenotypes include 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD19+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells containing an anti-CD19 CAR, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD19+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, the attributes (e.g., second attributes) of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+, IFNG+, IL10+, IL13+, IL2+, IL5+, MIP1A+, MIP1B+, sCD137+, and/or TNFa+.

In some embodiments, the attributes (e.g., second attributes) of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+/CD19+, IFNG+/CD19+, IL10+/CD19+, IL13+/CD19+, IL2+/CD19+, IL5+/CD19+, MIP1A+/CD19+, MIP1B+/CD19+, sCD137+/CD19+, and/or TNFa+/CD19+, when the cells contain an anti-CD19 CAR.

In some embodiments, for example when the therapeutic cell composition is engineered CD4+ T cells, the recombinant receptor-dependent activity includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+, IL-10+, IL-13+, IL-2+, IL-5+, MIP1A+, MIP1B+, sCD137+, and/or TNFa+.

In some embodiments, for example when the therapeutic cell composition is engineered CD4+ T cells, the recombinant receptor-dependent activity includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+, when the cells contain an anti-CD19 CAR.

In some embodiments, for example when the therapeutic cell composition is engineered CD8+ T cells, the recombinant receptor-dependent activity IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+.

In some embodiments, for example when the therapeutic cell composition is engineered CD8+ T cells, the recombinant receptor-dependent activity IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+, when the cells contain an anti-CD19 CAR.

In some embodiments, for example when the therapeutic cell composition is engineered CD4+ and CD8+ T cells or there are separate therapeutic compositions of CD4+ and CD8+ engineered cells, the recombinant receptor-dependent activity including IFNG+IL2+CD4+CAR+, IFNG+IL2+IL17+TNFA+CD4+CAR+, IFNG+IL2+TNFA+CD4+CAR+, IFNG+ of CD4+CAR, IFNG+TNFA+CD4+CAR+, IL13+ of CD4+CAR+, IL17+ of CD4+CAR+, IL2+ of CD4+CAR+, IL2+TNFA+CD4+CAR+, TNFA+ of CD4+CAR+, IFNG+IL2+CD8+CAR+, IFNG+IL2+IL17+TNFA+CD8+CAR+, IFNG+IL2+TNFA+CD8+CAR+, IFNG+ of CD8+CAR, IFNG+TNFA+CD8+CAR+, IL13+ of CD8+CAR+, IL17+ of CD8+CAR+, IL2+ of CD8+CAR+, IL2+TNFA+CD8+CAR+, TNFA+ of CD8+CAR+, cytolytic CD8+, GMCSF+, IFNG+, IL10+, IL13+, IL2+, IL5+, MIP1A+, MIP1B+, sCD137+, and/or TNFa+.

In some embodiments, the second attributes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+.

In some embodiments, the second attributes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+, when the cell contains an anti-CD19 CAR.

In some embodiments, the second attributes include therapeutic composition attributes shown in Table E2, or a subset thereof. In some embodiments, the second attributes include one or more therapeutic composition attributes shown in Table E2.

In some embodiments, the therapeutic cell composition attributes (e.g., second attributes) include or include about 101, 90, 80, 70, 60, 50, 40, 30, 20, 15, 10, 5, 4, 3, 2, or 1 cell phenotypes and recombinant receptor-dependent activity. In some embodiments, the therapeutic cell composition attributes (e.g., second attributes) include or include about or at least 1, 2, 4, 6, 8, 10, 12, or more cell phenotypes and recombinant receptor-dependent activity. In some embodiments, the therapeutic cell composition attributes (e.g., second attributes) includes 1 cell phenotype or recombinant receptor activity.

In some aspects, the methods provided herein include determining attributes of an input cell composition correlated with attributes of a therapeutic cell composition, the method including, determining a percentage, number, or proportion of cells in an input cell composition that have first attributes, wherein the first attributes comprise cell phenotypes, and wherein the input composition comprises T cells selected from a sample from a subject; determining a percentage, number, or proportion of cells in a therapeutic cell composition that have second attributes, wherein the second attributes comprise cell phenotypes and recombinant receptor-dependent activity, wherein the therapeutic cell composition comprises the recombinant receptor and is produced from the input composition; performing pCCA between the first attributes and the second attributes; and identifying, based on the penalized canonical correlation analysis, the first attributes correlated with the second attributes. In some embodiments, missing attributes may be imputed.

In some embodiments, proportions of naïve (e.g., CD27+/CCR7+, CD27+, CCR7+, CCR7+/CD45RA+, CD28+/CD27+) CD4 T cells in the input composition are positively correlated with proportions of naïve CD4 (e.g., CCR7+/CD27+, CCR7+, CD28+/CD27+, CD27+, CCR7+/CD45RA+) CAR T cells and naïve CD8 (e.g., CD28+/CD27+, CD27+, CCR7+, CCR7+/CD27+, CCR7+/CD45RA+, CD28+) CAR+ T cells in the therapeutic composition. In some embodiments, proportions of naïve (e.g., CD27+/CCR7+, CD27+, CCR7+, CCR7+/CD45RA+, CD28+/CD27+) CD4 T cells in the input composition are negatively (e.g., inversely) correlated with CD4+ effector memory (e.g., CD28+/CD27−, CCR7−/CD27−, CCR7−/CD45RA−) CAR+ T cells and CD8+ effector memory (e.g., CD28−/CD27−, CCR7−/CD27−, CCR7−/CD45RA−) CAR+ T cells in the therapeutic cell composition. In some embodiments, proportions of CD4+ effector memory cells (e.g., CCR7−/CD27−, CD28+/CD27−, CCR7−/CD45RA−) are negatively (e.g., inversely) correlated with proportions of naïve CD4 (e.g., CCR7+/CD27+, CCR7+, CD28+/CD27+, CD27+, CCR7+/CD45RA+) CAR T cells and naïve CD8 (e.g., CD28+/CD27+, CD27+, CCR7+, CCR7+/CD27+, CCR7+/CD45RA+, CD28+) CAR+ T cells in the therapeutic composition. In some embodiments, proportions of CD4+ effector memory (e.g., CCR7−/CD27−, CD28+/CD27−, CCR7−/CD45RA−) are positively correlated with CD4+ effector memory (e.g., CD28+/CD27−, CCR7−/CD27−, CCR7−/CD45RA−) CAR+ T cells and CD8+ effector memory (e.g., CD28−/CD27−, CCR7−/CD27−, CCR7−/CD45RA−) CAR+ T cells in the therapeutic cell composition. In some embodiments, proportions of naïve (e.g., CD27+/CCR7+, CD27+, CCR7+, CD28+/CD27+, CD28+) CD4+ T cells in the input composition are negatively correlated with stem cell memory (e.g., CD28−/CD27−, CCR7−/CD27−, CCR7+/CD45RA+) CD8+ proportions in the therapeutic composition. In some embodiments, CD4+ stem cell memory cell (e.g., CD28−/CD27−, CCR7−/CD27−, CCR7+/CD45RA+) proportions in the input composition are positively correlated with stem cell memory (e.g., CD28−/CD27−, CCR7−/CD27−, CCR7+/CD45RA+) CD8+ proportions in the therapeutic composition. In some embodiments, naïve (e.g., CD27+/CCR7+, CD27+, CCR7+, CD28+/CD27+, CD28+) CD8+ T cell proportions in the input composition are negatively correlated with stem cell memory (e.g., CD28−/CD27−, CCR7−/CD27−, CCR7+/CD45RA+) CD8+ proportions in the therapeutic composition. In some embodiments, CD4+ effector memory (e.g., CD28+/CD27−, CCR7−/CD27−, CCR7−/CD45RA−) T cell and CD8+ effector memory (e.g., CD28−/CD27−, CCR7−/CD27−, CCR7−/CD45RA−) T cell proportions in the input composition are positively correlated with CD4+ and CD8+CAR+T effector memory cell (e.g., CCR7−/CD27−, CD28+/CD27−, CCR7−/CD45RA−) proportions and proportions of recombinant receptor-dependent IFNg-expressing CD4+ and CD8+ T cells in the therapeutic composition. In some embodiments, CD4+ effector memory (e.g., CD28+/CD27−, CCR7−/CD27−, CCR7−/CD45RA−) T cell and CD8+ effector memory (e.g., CD28−/CD27−, CCR7−/CD27−, CCR7−/CD45RA−) T cell proportions in the input composition are negatively (e.g., inversely) correlated with the proportion of CD4+ and CD8+CAR+ recombinant receptor-dependent IL-2-expressing cells in the therapeutic composition. In some embodiments, proportions of CD8+ central memory cells (e.g., CCR7+/CD27+, CD27+/CD28+) in the input composition are positively correlated with the proportion of CD8+CAR+ recombinant receptor-dependent IL-2- and TNFa-expressing cells in the therapeutic composition. In some embodiments, proportions of CD8+ central memory cells in the input composition are negatively (e.g., inversely) correlated with the proportion of CD8+CAR+ recombinant receptor-dependent IFNg-expressing cells in the therapeutic composition. In some embodiments, proportions of CD8+ Temra cells (e.g., CD27−/CD28−, CCR7−/CD45RA+) in the input composition are negatively (e.g., inversely) correlated with the proportion of CD8+CAR+ recombinant receptor-dependent IL-2- and TNFa-expressing cells in the therapeutic composition. In some embodiments, proportions of CD8+ Temra cells (e.g., CD27−/CD28−, CCR7−/CD45RA+) in the input composition are positively correlated with the proportion of CD8+CAR+ recombinant receptor-dependent IFNg-expressing cells in the therapeutic composition. In some embodiments, CD8+ stem cell memory cell (e.g., CD28−/CD27−, CCR7−/CD27−, CCR7+/CD45RA+) proportions in the input composition are positively correlated with stem cell memory (e.g., CD28−/CD27−, CCR7−/CD27−, CCR7+/CD45RA+) CD8+ proportions in the therapeutic composition. In some embodiments, effector CD4 T cell (e.g., CCR7−/CD45RA−, CCR7−/CD27−, CD28+/CD27−) proportions in the input composition are positively correlated with proportions of CD4+ cells with recombinant receptor-dependent activity, including IFNg, IL-5, and GMCSF expression. In some embodiments, effector CD4 T cell (e.g., CCR7−/CD45RA−, CCR7−/CD27−, CD28+/CD27−) proportions in the input composition are negatively correlated with proportions of CD8+ cells with recombinant receptor-dependent activity, including IL-2 and TNFa expression. In some embodiments, effector CD8 T cell (CCR7+/CD27−, CD28+/CD27−, CCR7+/CD45RA−) proportions in the input composition are positively correlated with the proportion of CD8+ cells having recombinant receptor-dependent activity, including IL-5, IL-13, TNF-α, and IL-2, in the therapeutic composition. In some embodiments, CD4+ central memory T cell proportions in the input composition are positively correlated with CD4+ and CD8+ central memory CAR+ T cells (e.g., CCR7+/CD27+, CD27+/CD28+) and the proportion of CD4+ and CD8+CAR+ recombinant receptor-dependent IL-2-expressing cells in the therapeutic composition. In some embodiments, CD4+ central memory T cell (e.g., CCR7+/CD27+, CD27+/CD28+) proportions in the input composition are negatively (e.g., inversely) correlated with the proportion of CD4+CAR+ recombinant receptor-dependent IFNg-expressing cells in the therapeutic composition. In some embodiments, CD4+ effector memory T cell (e.g., CCR7−/CD45RA−, CCR7−/CD27−, CD28+/CD27−) proportions in the input composition are negatively (e.g., inversely) correlated with CD4+ and CD8+ central memory CAR+ T cells and the proportion of CD4+ and CD8+CAR+ recombinant receptor-dependent IL-2-expressing cells in the therapeutic composition. In some embodiments, CD4+ effector memory T cell (e.g., CCR7+/CD27+, CD27+/CD28+) proportions in the input composition are positively correlated with the proportion of CD4+CAR+ recombinant receptor-dependent IFNg-expressing cells in the therapeutic composition. In some embodiments, proportions of CCR7−/CD45RA−/CD4+, CCR7−/CD27−/CD4+, CD28+/CD27−/CD4+ in the input composition are positively correlated with the proportion of MIP1a+ or MIP1b CD4+ T cells in the therapeutic composition. In some embodiments, the proportions of CD28+/CD27+/CD4+, CD27+/CD4+, or CD28+/CD4+ T cells in the input composition are positively correlated with the proportion of IL-2+CD8+ T cells, CD8+/CAR+, CD28+/CD27+/CD4+, CD27+/CD4+, or CD28+/CD8+, or CD28+/CD27+/CD8+, CD27+/CD8+, or CD28+/CD8+ T cells in the therapeutic composition. In some embodiments, the proportions of CD28+/CD27+/CD4+, CD27+/CD4+, CD28+/CD8+, CD28+/CD27+/CD8+, CD27+/CD8+, or CD28+/CD8+ T cells in the input composition are positively correlated with the proportion of CD28+/CD27+/CD4+, CD27+/CD4+, or CD28+/CD8+, or CD28+/CD27+/CD8+, CD27+/CD8+, or CD28+/CD8+ T cells in the therapeutic composition. In some embodiments, the proportions of CCR7−/CD45RA−/CD4+ and CCR7−/CD27−/CD4+ T cells in the input composition are positively correlated with the proportion of CCR7−/CD45RA−/CD4+, CCR7−/CD27−/CD4+, MIP1a+ and MIP1b+ CD4+ T cells in the therapeutic composition. In some embodiments, the proportions of CD28−/CD27−/CD4+ T cells in the input composition are positively correlated with the proportion of CD28−/CD27−/CD4+, CD28+/CD27−/CD4+, CCR7−/CD45RA+/CD4+, MIP1a+, MIP1b, or IFNg CD4+ T cells in the therapeutic composition. In some embodiments, the proportions of CCR7+/CD45RA+/CD8+, CCR7+/CD8+, CD27+/CD8+, CD28+/CD27+/CD8+ T cells in the input composition are positively correlated with the proportion of CCR7+/CD45RA+/CD8+, CCR7+/CD8+, CD27+/CD8+, CD28+/CD27+/CD8+ T cells in the therapeutic composition. In some embodiments, the proportions of CCR7−/CD45RA−/CD8+ or CD28−/CD27−/CD8+ T cells in the input composition are positively correlated with the proportion of CCR7−/CD45RA−/CD8+ or CD28−/CD27−/CD8+, MIP1a+, MIP1b+ or CAS3+/CAR+CD8+ T cells in the therapeutic composition. In some embodiments, the proportions of CCR7−/CD45RA−/CD8+ or CD28−/CD27−/CD8+ T cells in the input composition are positively correlated with the proportion of CCR7−/CD45RA−/CD8+ or CD28−/CD27−/CD8+, MIP1a+, MIP1b+ or CAS3+/CAR+CD8+ T cells in the therapeutic composition. In some embodiments, the proportions of CD28+/CD27+, CD27+, CD28+CD4+ T cells in the input composition are positively correlated with the proportion of CD28+/CD27+, CD27+ and CD28+CD8+ or CD4+ T cells in the therapeutic composition. In some embodiments, the proportions of CD28+/CD27+, CD27+, or CD28+CD4+ T cells in the input composition are positively correlated with the proportion of IL-2+CD8+ T cells in the therapeutic composition. In some embodiments, the proportions of CD28+/CD27+/CD4+, CD27+/CD4+, and CD28+/CD4+ T cells in the input composition are positively correlated with the proportion of CD8+/CAR+ T cells in the therapeutic composition.

In some embodiments, the proportions of CD28+/CD27+/CD4+ and CD27+/CD4+ T cells in the input composition are positively correlated with the proportion of CD27+/CCR7+/CD4+, CCR7+/CD4+, CD28+/CD27+/CD4+, CD27+/CD4+, CD28+/CD4+, CCR7+/CD45RA+/CD4+ T cells in the therapeutic composition. In some embodiments, the proportions of CD28+/CD27+/CD4+ and CD27+/CD4+ T cells in the input composition are positively correlated with the proportion of IL-2+/CD4+ T cells in the therapeutic composition. In some embodiments, the proportions of CCR7+/CD45RA−/CD8+ and CD28+/CD8+ T cells in the input composition are positively correlated with the proportion of CD27+/CCR7+/CD8+, CCR7+/CD8+, CD28+/CD27+/CD8+, CD27+/CD8+, CD28+/CD8+, CCR7+/CD45RA+/CD8+ T cells in the therapeutic composition. In some embodiments, the proportions of CD28+/CD27+, CD27+, or CD28+CD4+ T cells in the input composition are positively correlated with the proportion of IL-2+ or TNFa+CD8+ T cells in the therapeutic composition. In some embodiments, the input compositions independently include CD4+ and CD8+ input compositions and are independently processed to generate engineered CD4+ and CD8+ therapeutic cell compositions.

2. Lasso Regression

Another statistical method contemplated for use in identifying correlated attributes is lasso regression. Lasso regression is able to accommodate a plurality of variables but uses regularization to identify only those input variables that correlate with a single output variable. As such, lasso regression is useful for determining how a single variable (e.g., a single therapeutic cell composition attribute) relates to a plurality of input variables (e.g., input composition attributes). In some embodiments, lasso regression is implemented in R v3.5 or 3.6 using the glmnet package.

In some embodiments, lasso is performed using a first set of attributes (e.g., first attributes) determined from an input composition and one attribute from a second set of attributes (e.g., second attributes) determined from a therapeutic cell composition produced from the input composition. In some embodiments, the input composition contains CD4+, CD8+, or CD4+ and CD8+ cells selected from a subject, and the therapeutic cell composition contains engineered CD4+, CD8+, or CD4+ and CD8+ cells, respectively. In some embodiments, the first attributes include cell phenotype attributes. In some embodiments, the cell phenotypes 3CAS−/CCR7−/CD27−, 3CAS−/CCR7−/CD27+, 3CAS−/CCR7+, 3CAS−/CCR7+/CD27−, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and/or CAS+/CD3+. In some embodiments, for example when the input composition is CD4+ T cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS/−CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, CAS+/CD4+, CAS+/CD3+, and/or 3CAS−/CCR7+/CD45RA+/CD4+. In some embodiments, for example when the input composition is CD8+ T cells, the cell phenotypes include 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD8+, and/or CAS+/CD3+. In some embodiments, for example when the input composition is CD4+ and CD8+ T cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, and/or CAS+/CD3+. In some embodiments, the input composition attributes (e.g., first attributes) are 34 cell phenotypes. In some embodiments, the 34 cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and/or CAS+/CD3+ of an input composition that is CD8+ cells. In some embodiments, the input composition attributes (e.g., first attributes) include a subset of any of the above cell phenotypes. In some embodiments, the input composition attributes (e.g., first attributes) include or include about 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 cell phenotypes. In some embodiments, the input composition attributes (e.g., first attributes) include or comprise about or at least 2, 4, 6, 8, 10, 12, or more cell phenotypes. In some embodiments, the input composition attributes (e.g., first attributes) include great than or great than about 5, 10, 15, or 20 cell attributes.

In some embodiments, the first attributes include input composition attributes shown in Table E2, or a subset thereof. In some embodiments, the first attributes include one or more input composition attributes shown in Table E2.

In some embodiments, the attributes of the therapeutic cell composition include cell phenotypes, for example as described in Section I-A-2. In some embodiments, the therapeutic cell composition attributes are second attributes. In some embodiments, the second attributes include cell phenotype attributes. In some embodiments, the cell phenotypes include 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CYTO−/CAR+, EGFRt+, IFNG+, viable cell concentration (VCC), vector copy number (VCN), viability, GMCSF+, CD3+/CAR+, CD3+/CD56+, and/or CAR+. In some embodiments, the cell phenotypes include 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+/CD19+, CD3+/CAR+, CD3+/CD56+, and/or CAR+.

In some embodiments, for example when the therapeutic cell composition is engineered CD4+ cells, the cell phenotypes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, and/or CD4+/CAR+.

In some embodiments, for example when the therapeutic cell composition is engineered CD4+ cells containing an anti-CD19 CAR, the cell phenotypes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, and/or CD4+/CAR+.

In some embodiments, for example when the therapeutic cell composition is engineered CD8+ cells, the cell phenotypes include 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, and/or CD8+/CAR+.

In some embodiments, for example when the therapeutic cell composition is engineered CD8+ cells containing an anti-CD19 CAR, the cell phenotypes include 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells containing an anti-CD19 CAR, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD19+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, the attributes (e.g., second attributes) of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+, IFNG+, IL10+, IL13+, IL2+, IL5+, MIP1A+, MIP1B+, sCD137+, and/or TNFa+.

In some embodiments, the attributes (e.g., second attributes) of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+/CD19+, IFNG+/CD19+, IL10+/CD19+, IL13+/CD19+, IL2+/CD19+, IL5+/CD19+, MIP1A+/CD19+, MIP1B+/CD19+, sCD137+/CD19+, and/or TNFa+/CD19+, when the cells contain an anti-CD19 CAR.

In some embodiments, for example when the therapeutic cell composition is engineered CD4+ T cells, the recombinant receptor-dependent activity includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+, IL-10+, IL-13+, IL-2+, IL-5+, MIP1A+, MIP1B+, sCD137+, and/or TNFa+.

In some embodiments, for example when the therapeutic cell composition is engineered CD4+ T cells containing an anti-CD19 CAR, the recombinant receptor-dependent activity includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, for example when the therapeutic cell composition is engineered CD8+ T cells, the recombinant receptor-dependent activity IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+.

In some embodiments, for example when the therapeutic cell composition is engineered CD8+ T cells, the recombinant receptor-dependent activity IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, for example when the therapeutic cell composition is engineered CD4+ and CD8+ T cells or there are separate therapeutic compositions of CD4+ and CD8+ engineered cells, the recombinant receptor-dependent activity including IFNG+IL2+CD4+CAR+, IFNG+IL2+IL17+TNFA+CD4+CAR+, IFNG+IL2+TNFA+CD4+CAR+, IFNG+ of CD4+CAR, IFNG+TNFA+CD4+CAR+, IL13+ of CD4+CAR+, IL17+ of CD4+CAR+, IL2+ of CD4+CAR+, IL2+TNFA+CD4+CAR+, TNFA+ of CD4+CAR+, IFNG+IL2+CD8+CAR+, IFNG+IL2+IL17+TNFA+CD8+CAR+, IFNG+IL2+TNFA+CD8+CAR+, IFNG+ of CD8+CAR, IFNG+TNFA+CD8+CAR+, IL13+ of CD8+CAR+, IL17+ of CD8+CAR+, IL2+ of CD8+CAR+, IL2+TNFA+CD8+CAR+, TNFA+ of CD8+CAR+, cytolytic CD8+, GMCSF+, IFNG+, IL10+, IL13+, IL2+, IL5+, MIP1A+, MIP1B+, sCD137+, and/or TNFa+.

In some embodiments, for example when the therapeutic cell composition is engineered CD4+ and CD8+ T cells or there are separate therapeutic compositions of CD4+ and CD8+ engineered cells, the recombinant receptor-dependent activity including IFNG+IL2+CD4+CAR+, IFNG+IL2+IL17+TNFA+CD4+CAR+, IFNG+IL2+TNFA+CD4+CAR+, IFNG+ of CD4+CAR, IFNG+TNFA+CD4+CAR+, IL13+ of CD4+CAR+, IL17+ of CD4+CAR+, IL2+ of CD4+CAR+, IL2+TNFA+CD4+CAR+, TNFA+ of CD4+CAR+, IFNG+IL2+CD8+CAR+, IFNG+IL2+IL17+TNFA+CD8+CAR+, IFNG+IL2+TNFA+CD8+CAR+, IFNG+ of CD8+CAR, IFNG+TNFA+CD8+CAR+, IL13+ of CD8+CAR+, IL17+ of CD8+CAR+, IL2+ of CD8+CAR+, IL2+TNFA+CD8+CAR+, TNFA+ of CD8+CAR+, cytolytic CD8+, GMCSF+CD19+, IFNG+, IL10+, IL13+, IL2+, IL5+, MIP1A+, MIP1B+, sCD137+, and/or TNFa+.

In some embodiments, the second attributes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+.

In some embodiments, the second attributes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, the second attributes include therapeutic composition attributes shown in Table E2, or a subset thereof. In some embodiments, the second attributes include one or more therapeutic composition attributes shown in Table E2.

In some embodiments, the method is run multiple times such that each second attribute is correlated with the first attributes of the input composition.

In some embodiments, naïve CD4 T cell (e.g., CCR7+/CD27+, CCR7+/CD45RA+) proportions in the input composition are positively correlated with naïve CD4+ cell proportions in the therapeutic composition. In some embodiments, naïve (e.g., CCR7+/CD27+, CCR7+/CD45RA+) CD4 and CD8 T cell proportions in input compositions are positively correlated with naïve CD8+ cell proportions in the therapeutic composition. In some embodiments, effector (e.g., CCR7−/CD27−) proportions of CD4 and CD8 cells in the input compositions are positively correlated with proportions of CD8+ cells having recombinant receptor activity including producing IFNg, TNF-α, IL-13, IL-2, and IL-5.

C. Predicting Therapeutic Cell Composition Attributes

It is contemplated that the attributes of the therapeutic cell composition (e.g., engineered T cell composition) can, in some cases, depend upon many factors, including, but not limited to, the attributes of the starting cellular material (e.g., apheresis product or leukapheresis product or cells selected therefrom (e.g., input composition)) used to generate the therapeutic cell composition. Thus, in some embodiments, input composition attributes and attributes of the therapeutic cell composition produced from the input composition are assessed (e.g., quantified) and used as training data to train processes including statistical learning models to predict therapeutic cell composition attributes from input composition attributes. In some embodiments, input composition attributes and attributes of the therapeutic cell composition produced from the input composition are assessed (e.g., quantified) and used as training data to train processes including statistical learning models capable of predicting a single variable (e.g., a therapeutic cell composition attribute) from a plurality of input variables (e.g., input composition attributes). In some embodiments, the attributes are cell phenotypes. In some embodiments, the attributes, for example in the therapeutic cell composition are recombinant receptor-dependent activity. In some embodiments, the attributes, e.g., cell phenotypes, recombinant receptor-dependent activity, are quantified to provide a number, percentage, proportion, and/or ratio of cells having an attribute in the composition (e.g., input composition, therapeutic cell composition). In some embodiments, the statistical learning models predict a number, percentage, proportion, and/or ratio of cells having an attribute in the therapeutic composition based on a number, percentage, proportion, and/or ratio of cells having an attribute in the input composition.

As described above, input and therapeutic cell compositions may contain CD3+, CD4+, CD8+ or CD4+ and CD8+ cells. Thus, in some embodiments, the attributes of the input and therapeutic cell compositions may be cell type specific. In some embodiments, for example when input compositions separately contain CD4+ or CD8+ cells from which the therapeutic T cell composition (e.g., CD4+ or CD8+ therapeutic cell compositions) will be independently produced, attributes can be assess for each input and therapeutic cell composition and used as training data for processes including statistical learning models described herein. For example, attributes determined from an input composition containing CD4+ T cells, which is separately processed to produce a CD4+ therapeutic cell composition, can be used (e.g., as input) to predict attributes of the resultant CD4+ therapeutic composition and a CD8+ therapeutic cell composition produced from an input composition containing CD8+ T cells, and vice versa.

1. Canonical Correlation Analysis

In some embodiments, the statistical learning model for predicting therapeutic composition attributes from input composition attributes is canonical correlation analysis (CCA). As described in Section I-B-1, CCA can handle high dimensional data sets containing a plurality of variables (e.g., attributes) and identify correlations that are not limited by or to one to one relationships. As such, CCA is well suited to identifying relationships between groups of variables (e.g., therapeutic cell composition attributes) from a plurality of input variables (e.g., input composition attributed) and further, when used as a learning model, capable of predicting variables (e.g., therapeutic cell composition attributes) from a plurality of input variables (e.g., input composition attributes).

When used as a statistical learning model, in some embodiments, CCA is captured by Equation 3:

argmax_{u,v}u^{T}X^{T}Yv (Eq. 3)

where X and Y represent sets of high dimensional variables (e.g., input attributes and therapeutic composition attributes) and u and v are canonical vectors (e.g., weights). In some embodiments, convex penalty functions are used. In some embodiments, the canonical vectors are constrained by a requirement that the square of the L2 norm of the canonical vectors to be less than or equal to 1.

In some embodiments, the CCA statistical learning model is a pCCA statistical learning model as described in Section I-B-1.

In some embodiments, the CCA statistical learning model is trained on labeled data. For example, the model may be trained on pairs of attributes from input compositions and the therapeutic composition produced from the input composition to relate input composition attributes to therapeutic cell composition attributes. In some embodiments, the CCA statistical learning model is trained to relate numbers, percentages, proportions, and/or ratios of input compositions attributes to numbers, percentages, proportions, and/or ratios of therapeutic cell composition attributes. In some embodiments, the trained CCA model predicts therapeutic cell composition attributes from input composition attributes. In some embodiments, predictions are computed in the telefit package in R v3.5.

In some embodiments, the CCA statistical learning model predicts therapeutic cell composition attributes from a first set of attributes (e.g., first attributes) determined from an input composition. In some embodiments, the input composition contains CD4+, CD8+, or CD4+ and CD8+ cells selected from a subject, and the therapeutic cell composition will contain engineered CD4+, CD8+, or CD4+ and CD8+ cells, respectively. In some embodiments, the first attributes are cell phenotypes. In some embodiments, the first attributes of the input composition include cell phenotypes, for example as described in Section I-A-1. In some embodiments, the input composition attributes are first attributes. In some embodiments, the first attributes include cell phenotype attributes. In some embodiments, the cell phenotypes 3CAS−/CCR7−/CD27−, 3CAS−/CCR7−/CD27+, 3CAS−/CCR7+, 3CAS−/CCR7+/CD27−, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and/or CAS+/CD3+. In some embodiments, for example when the input composition is CD4+ T cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS/−CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, CAS+/CD4+, CAS+/CD3+, and/or 3CAS−/CCR7+/CD45RA+/CD4+. In some embodiments, for example when the input composition is CD8+ T cells, the cell phenotypes include 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD8+, and/or CAS+/CD3+. In some embodiments, for example when the input composition is CD4+ and CD8+ T cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, and/or CAS+/CD3+. In some embodiments, the input composition attributes (e.g., first attributes) are 34 cell phenotypes. In some embodiments, the 34 cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and/or CAS+/CD3+ of an input composition that is CD8+ cells. In some embodiments, the input composition attributes (e.g., first attributes) include a subset of any of the above cell phenotypes. In some embodiments, the input composition attributes (e.g., first attributes) include or include about 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 cell phenotypes. In some embodiments, the input composition attributes (e.g., first attributes) include or comprise about or at least 2, 4, 6, 8, 10, 12, or more cell phenotypes. In some embodiments, the input composition attributes (e.g., first attributes) include great than or great than about 5, 10, 15, or 20 cell attributes. In some embodiments, the input composition attributes include or are CD4+/CCR7+/CD27+, CD4+/CCR7+/CD45RA+, CD4+/CD28+/CD27−, and CD8+/CCR7+CD45RA+. In some embodiments, the input composition attribute is CD4+/CCR7+/CD45RA+. In some embodiments, the input composition attributes include or are CD8+/CCR7+, CD4+/CCR7−/CD27−, CD8+/CCR7−/CD45RA+, and CD4+/CD28+.

In some embodiments, the first attributes include input composition attributes shown in Table E2, or a subset thereof. In some embodiments, the first attributes include one or more input composition attributes shown in Table E2.

In some embodiments, the attributes of the therapeutic cell composition, e.g., the attributes to be predicted, include cell phenotypes, for example as described in Section I-A-2. In some embodiments, the therapeutic cell composition attributes to be predicted are second attributes. In some embodiments, the second attributes include cell phenotype attributes. In some embodiments, the cell phenotypes include 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CYTO−/CAR+, EGFRt+, IFNG+, viable cell concentration (VCC), vector copy number (VCN), viability, GMCSF+, CD3+/CAR+, CD3+/CD56+, and/or CAR+. In some embodiments, the cell phenotypes include 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+/CD19+, CD3+/CAR+, CD3+/CD56+, and/or CAR+.

In some embodiments, for example when the therapeutic cell composition is engineered CD4+ cells, the cell phenotypes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, and/or CD4+/CAR+.

In some embodiments, for example when the therapeutic cell composition is engineered CD4+ cells containing an anti-CD19 CAR, the cell phenotypes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, and/or CD4+/CAR+.

In some embodiments, for example when the therapeutic cell composition is engineered CD8+ cells, the cell phenotypes include 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, and/or CD8+/CAR+.

In some embodiments, for example when the therapeutic cell composition is engineered CD8+ cells containing an anti-CD19 CAR, the cell phenotypes include 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, and/or CD8+/CAR+.

In some embodiments, for example when the therapeutic cell composition is engineered CD4+ and CD8+ cells or there are separate therapeutic compositions of CD4+ and CD8+ engineered cells containing an anti-CD19 CAR, the attributes of the therapeutic cell composition include cell phenotypes including 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD19+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, and/or CD8+/CAR+.

In some embodiments, the attributes (e.g., second attributes to be predicted) of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+, IFNG+, IL10+, IL13+, IL2+, IL5+, MIP1A+, MIP1B+, sCD137+, and/or TNFa+.

In some embodiments, the attributes (e.g., second attributes to be predicted) of the therapeutic cell composition include recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+/CD19+, IFNG+/CD19+, IL10+/CD19+, IL13+/CD19+, IL2+/CD19+, IL5+/CD19+, MIP1A+/CD19+, MIP1B+/CD19+, sCD137+/CD19+, and/or TNFa+/CD19+, when the cells contain an anti-CD19 CAR.

In some embodiments, for example when the therapeutic cell composition is engineered CD4+ T cells, the recombinant receptor-dependent activity includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+, IL-10+, IL-13+, IL-2+, IL-5+, MIP1A+, MIP1B+, sCD137+, and/or TNFa+.

In some embodiments, for example when the therapeutic cell composition is engineered CD4+ T cells containing an anti-CD19 CAR, the recombinant receptor-dependent activity includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, for example when the therapeutic cell composition is engineered CD8+ T cells, the recombinant receptor-dependent activity includes IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+.

In some embodiments, for example when the therapeutic cell composition is engineered CD8+ T cells containing an anti-CD19 CAR, the recombinant receptor-dependent activity includes IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, for example when the therapeutic cell composition is engineered CD4+ and CD8+ T cells, the recombinant receptor-dependent activity includes or there are separate therapeutic compositions of CD4+ and CD8+ engineered cells, the recombinant receptor-dependent activity including IFNG+IL2+CD4+CAR+, IFNG+IL2+IL17+TNFA+CD4+CAR+, IFNG+IL2+TNFA+CD4+CAR+, IFNG+ of CD4+CAR, IFNG+TNFA+CD4+CAR+, IL13+ of CD4+CAR+, IL17+ of CD4+CAR+, IL2+ of CD4+CAR+, IL2+TNFA+CD4+CAR+, TNFA+ of CD4+CAR+, IFNG+IL2+CD8+CAR+, IFNG+IL2+IL17+TNFA+CD8+CAR+, IFNG+IL2+TNFA+CD8+CAR+, IFNG+ of CD8+CAR, IFNG+TNFA+CD8+CAR+, IL13+ of CD8+CAR+, IL17+ of CD8+CAR+, IL2+ of CD8+CAR+, IL2+TNFA+CD8+CAR+, TNFA+ of CD8+CAR+, cytolytic CD8+, GMCSF+CD19+, IFNG+, IL10+, IL13+, IL2+, IL5+, MIP1A+, MIP1B+, sCD137+, and/or TNFa+.

In some embodiments, the second attributes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+.

In some embodiments, the second attributes include 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

In some embodiments, the attributes (e.g., the second attributes to be predicted) include therapeutic composition attributes shown in Table E2, or a subset thereof. In some embodiments, the attributes (e.g., the second attributes to be predicted) include one or more therapeutic composition attributes shown in Table E2.

In some embodiments, the therapeutic cell composition attributes (e.g., second attributes) to be predicted include or include about 101, 90, 80, 70, 60, 50, 40, 30, 20, 15, 10, 5, 4, 3, 2, or 1 cell phenotypes and recombinant receptor-dependent activity. In some embodiments, the therapeutic cell composition attributes (e.g., second attributes) include or include about or at least 1, 2, 4, 6, 8, 10, 12, or more cell phenotypes and recombinant receptor-dependent activity. In some embodiments, the therapeutic cell composition attributes (e.g., second attributes) includes 1 cell phenotype or recombinant receptor activity.

In some embodiments, the CCA statistical learning model predicts the number, percentage, proportion, and/or ratio of 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CD3+/EGFRt+, CYTO−/CAR+, EGFRt+, IFNG+, VCC, VCN, viability, GMCSF+, CD3+/CAR+, CD3+/CD56+/CAR+, 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CD3+/EGFRt+, CYTO−/CAR+, EGFRt+, IFNG+, VCC, VCN, viability, GMCSF+, CD3+/CAR+, CD3+/CD56+/CAR+, IFNG+/IL-2+/CAR+, IFNG+/IL-2+/IL-17+/TNFA+/CAR+, IFNG+/IL-2+/TNFA+/CAR+, IFNG+OF/CAR+, IFNG+/TNFA+/CAR+, IL-13+ of/CAR+, IL-17+ of CAR+, IL-2+ of CAR+, IL-2+/TNFA+/CAR+, TNFA+ of/CAR+, IFNG+/IL-2+/CAR+, IFNG+/IL-2+/IL-17+/TNFA+/CAR+, IFNG+/IL-2+/TNFA+/CAR+, IFNG+ of/CAR+, IFNG+/TNFA+/CAR+, IL-13+ of/CAR+, IL-17+ of CAR+, IL-2+ of CAR+, IL-2+/TNFA+/CAR+, cytolytic, TNFA+ of CAR+, IFNG+, IL-10+, IL-13+, IL-2+, IL-5+, MIP1A+, MIP1B+, sCD137+, and/or TNFa+ cells in the therapeutic cell composition from the input composition attributes.

In some embodiments, the CCA statistical learning model predicts the number, percentage, proportion, and/or ratio of 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CD3+/EGFRt+, CYTO−/CAR+, EGFRt+, IFNG+, VCC, VCN, viability, GMCSF+, CD3+/CAR+, CD3+/CD56+, /CAR+, 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CD3+/EGFRt+, CYTO−/CAR+, EGFRt+, IFNG+, VCC, VCN, viability, GMCSF+, CD3+/CAR+, CD3+/CD56+/CAR+, IFNG+/IL-2+/CAR+, IFNG+/IL-2+/IL-17+/TNFA+/CAR+, IFNG+/IL-2+/TNFA+/CAR+, IFNG+ of CAR+, IFNG+/TNFA+/CAR+, IL-13+ of/CAR+, IL-17+ of CAR+, IL-2+ of CAR+, IL-2+/TNFA+/CAR+, TNFA+ of CAR+, IFNG+/IL-2+/CAR+, IFNG+/IL-2+/IL-17+/TNFA+/CAR+, IFNG+/IL-2+/TNFA+/CAR+, IFNG+ of/CAR+, IFNG+/TNFA+/CAR+, IL-13+ of/CAR+, IL-17+ of CAR+, IL-2+ of CAR+, IL-2+/TNFA+/CAR+, cytolytic, TNFA+ of CAR+, IFNG+, IL-10+, IL-13+, IL-2+, IL-5+, MIP1A+, MIP1B+, sCD137+, and/or TNFa+ cells in the therapeutic cell composition from the input composition attributes.

In some embodiments, the CCA statistical learning model predicts the number, percentage, proportion, and/or ratio of 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CD3+/, /EGFRt+, CYTO−/CAR+, EGFRt+, IFNG+, VCC, VCN, viability, GMCSF+/CD19+, CD3+/CAR+, CD3+/CD56+, /CAR+, 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CD3+/, /EGFRt+, CYTO−/CAR+, EGFRt+, IFNG+, VCC, VCN, viability, GMCSF+/CD19+, CD3+/CAR+, CD3+/CD56+, /CAR+, IFNG+/IL-2+/CAR+, IFNG+/IL-2+/IL-17+/TNFA+/CAR+, IFNG+/IL-2+/TNFA+/CAR+, IFNG+OF/CAR+, IFNG+/TNFA+/CAR+, IL-13+ of/CAR+, IL-17+ of CAR+, IL-2+ of CAR+, IL-2+/TNFA+/CAR+, TNFA+ of/CAR+, IFNG+/IL-2+/CAR+, IFNG+/IL-2+/IL-17+/TNFA+/CAR+, IFNG+/IL-2+/TNFA+/CAR+, IFNG+ of/CAR+, IFNG+/TNFA+/CAR+, IL-13+ of/CAR+, IL-17+ of CAR+, IL-2+ of CAR+, IL-2+/TNFA+/CAR+, cytolytic, TNFA+ of CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+ cells in the therapeutic cell composition from the input composition attributes.

In some embodiments, the CCA statistical learning model predicts the number, percentage, proportion, and/or ratio of 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+/CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, and/or TNFa+ cells in the therapeutic cell composition from the input composition attributes.

In some embodiments, the CCA statistical learning model predicts the number, percentage, proportion, and/or ratio of 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+ cells in the therapeutic cell composition from the input composition attributes.

In some embodiments, the CCA statistical model predicts the number, percentage, proportion, and/or ratio of 101 second attributes, for example as shown in Table E2, of the therapeutic cell composition. In some embodiments, the CCA statistical model predicts the number, percentage, proportion, and/or ratio of, of about, or at least 90, 80, 70, 60, 50, 40, 30, 20, 25, 20, 15, 10, 9, 8, 7, 6, 5, 4, 3, 2, or 1 second attributes of the therapeutic cell composition. In some embodiments, the CCA statistical model predicts the number, percentage, proportion, and/or ratio of, of about, or at least 60, 50, 40, 30, 20, 25, 20, 15, 10, 9, 8, 7, 6, 5, 4, 3, 2, or 1 second attributes of the therapeutic cell composition. In some embodiments, the CCA statistical model predicts the number, percentage, proportion, and/or ratio of, of about, or at least 50, 40, 30, 20, 25, 20, 15, 10, 9, 8, 7, 6, 5, 4, 3, 2, or 1 second attributes of the therapeutic cell composition. In some embodiments, the CCA statistical model predicts the number, percentage, proportion, and/or ratio of, of about between 5 to 60, 5 to 50, 5 to 40, 5 to 30, 5 to 20, 5 to 15, or 5 to 10 attributes of the therapeutic cell composition.

In some embodiments, the CCA statistical learning model predicts proportions of CD4+CAR+naïve (CCR7+/CD45RA+) T cells in therapeutic compositions from input composition attributes. In some embodiments, the CCA statistical analysis model predicts T_{EMRA} T cells (e.g., CD27−/CD28−, CCR7−/CD45RA+) proportions in the therapeutic composition from input composition attributes. In some embodiments, the CCA statistical analysis model predicts proportions of MIP1A in CD4+ cells in the therapeutic cell composition from the input composition attributes. In some embodiments, the CCA statistical analysis model predicts proportions of MIP1B in CD4+ cells in the therapeutic cell composition from the input composition attributes. In some embodiments, the CCA statistical analysis model predicts proportions of IL-2+TNFa+ in CD4 cells in the therapeutic cell composition from the input composition attributes. In some embodiments, the CCA statistical analysis model predicts proportions of IL-2 in CD8+ cells in the therapeutic cell composition from the input composition attributes. In some embodiments, the CCA statistical analysis model predicts proportions of IFNg in CD8+ cells in the therapeutic cell composition from the input composition attributes. In some embodiments, the input composition attributes include 34 attributes. In some embodiments, the 34 composition attributes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and/or CAS+/CD3+ of an input composition that is CD8+ cells.

In some embodiments, the CCA statistical learning model predicts the number, percentage, proportion, and/or ratio of desired attributes of a therapeutic cell composition.

2. Lasso Regression

Another statistical learning model contemplated for use in predicting therapeutic cell compositions attributes from input composition attributes is lasso regression. Lasso regression is able to accommodate a plurality of variables but uses regularization to identify only those input variables that correlate with a single output variable. As such, lasso regression is useful for determining how a single variable (e.g., a single therapeutic cell composition attribute) relates to a plurality of input variables (e.g., input composition attributes). When used as a statistical learning model, the model can be trained to predict therapeutic cell composition attributes. In some embodiments, the lasso regression statistical learning model is trained on labeled data (e.g., supervised training). For example, the model may be trained on pairs of attributes from input compositions and the therapeutic compositions produced the input composition to relate input composition attributes with one therapeutic cell composition attribute. In some embodiments, the lasso regression statistical learning model is trained to relate numbers, percentages, proportions, and/or ratios of input compositions attributes to numbers, percentages, proportions, and/or ratios of one of the therapeutic cell composition attributes. In some embodiments, the trained lasso model predicts one therapeutic cell composition attribute from input composition attributes used as input to the trained model. In some embodiments, the trained lasso model identifies one or a subset of input composition attributes that predict a single therapeutic cell composition attribute. In some embodiments, lasso regression statistical learning model is implemented in R v3.5 using the glmnet package.

In some embodiments, the lasso model predicts one therapeutic cell composition attribute using a first set of attributes (e.g., first attributes) determined from an input composition. In some embodiments, the input composition contains CD4+, CD8+, or CD4+ and CD8+ cells selected from a subject, and the therapeutic cell composition contains engineered CD4+, CD8+, or CD4+ and CD8+ cells, respectively. In some embodiments, the first attributes are cell phenotypes. In some embodiments, the first attributes of the input composition include cell phenotypes, for example as described in Section I-A-1. In some embodiments, the input composition attributes are first attributes. In some embodiments, the input composition attributes are first attributes. In some embodiments, the first attributes include cell phenotype attributes. In some embodiments, the cell phenotypes 3CAS−/CCR7−/CD27−, 3CAS−/CCR7−/CD27+, 3CAS−/CCR7+, 3CAS−/CCR7+/CD27−, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and/or CAS+/CD3+. In some embodiments, for example when the input composition is CD4+ T cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS/−CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, CAS+/CD4+, CAS+/CD3+, and/or 3CAS−/CCR7+/CD45RA+/CD4+. In some embodiments, for example when the input composition is CD8+ T cells, the cell phenotypes include 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD8+, and/or CAS+/CD3+. In some embodiments, for example when the input composition is CD4+ and CD8+ T cells, the cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, and/or CAS+/CD3+. In some embodiments, the input composition attributes (e.g., first attributes) are 34 cell phenotypes. In some embodiments, the 34 cell phenotypes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and/or CAS+/CD3+ of an input composition that is CD8+ cells. In some embodiments, the input composition attributes (e.g., first attributes) include a subset of any of the above cell phenotypes. In some embodiments, the input composition attributes (e.g., first attributes) include or include about 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 cell phenotypes. In some embodiments, the input composition attributes (e.g., first attributes) include or comprise about or at least 2, 4, 6, 8, 10, 12, or more cell phenotypes. In some embodiments, the input composition attributes (e.g., first attributes) include great than or great than about 5, 10, 15, or 20 cell attributes. In some embodiments, the input composition attributes include or are CD4+/CCR7+/CD27+, CD4+/CCR7+/CD45RA+, CD4+/CD28+/CD27−, and CD8+/CCR7+CD45RA+. In some embodiments, the input composition attribute is CD4+/CCR7+/CD45RA+. In some embodiments, the input composition attributes include or are CD8+/CCR7+, CD4+/CCR7−/CD27−, CD8+/CCR7−/CD45RA+, and CD4+/CD28+.

In some embodiments, the first attributes include input composition attributes shown in Table E2, or a subset thereof.

In some embodiments, the one attribute of the therapeutic cell composition to be predicted includes cell phenotypes, for example as described in Section I-A-2. In some embodiments, the therapeutic cell composition attributes are second attributes. In some embodiments, the one second attribute to be predicted includes a cell phenotype. In some embodiments, the cell phenotype includes 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CYTO−/CAR+, EGFRt+, IFNG+, viable cell concentration (VCC), vector copy number (VCN), viability, GMCSF+, CD3+/CAR+, CD3+/CD56+, or CAR+. In some embodiments, the cell phenotype includes 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+/CD19+, CD3+/CAR+, CD3+/CD56+, or CAR+.

In some embodiments, for example when the therapeutic cell composition is engineered CD4+ cells, the one cell phenotype to be predicted includes 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, or CD4+/CAR+.

In some embodiments, for example when the therapeutic cell composition is engineered CD4+ cells containing an anti-CD19 CAR, the one cell phenotype to be predicted includes 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, or CD4+/CAR+.

In some embodiments, for example when the therapeutic cell composition is engineered CD8+ cells, the one cell phenotype to be predicted includes 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, or CD8+/CAR+.

In some embodiments, for example when the therapeutic cell composition is engineered CD8+ cells containing an anti-CD19 CAR, the one cell phenotype to be predicted includes 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, or CD8+/CAR+.

In some embodiments, for example when the therapeutic cell composition is engineered CD4+ and CD8+ cells or there are separate therapeutic compositions of CD4+ and CD8+ engineered cells, the one cell phenotype to be predicted includes 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, or CD8+/CAR+.

In some embodiments, for example when the therapeutic cell composition is engineered CD4+ and CD8+ cells or there are separate therapeutic compositions of CD4+ and CD8+ engineered cells containing an anti-CD19 CAR, the one cell phenotype to be predicted includes 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CD19+, CD3+, CD3+/CD4+, CD4+/EGFRt+, CYTO−/CD4+/CAR+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, or CD8+/CAR+.

In some embodiments, the one second attribute of the therapeutic cell composition to be predicted includes a recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+, IFNG+, IL10+, IL13+, IL2+, IL5+, MIP1A+, MIP1B+, sCD137+, or TNFa+.

In some embodiments, the one second attribute of the therapeutic cell composition to be predicted includes a recombinant receptor-dependent activity including IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+/CD19+, IFNG+/CD19+, IL10+/CD19+, IL13+/CD19+, IL2+/CD19+, IL5+/CD19+, MIP1A+/CD19+, MIP1B+/CD19+, sCD137+/CD19+, or TNFa+/CD19+.

In some embodiments, for example when the therapeutic cell composition is engineered CD4+ T cells, the one recombinant receptor-dependent activity to be predicted includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+, IL-10+, IL-13+, IL-2+, IL-5+, MIP1A+, MIP1B+, sCD137+, or TNFa+.

In some embodiments, for example when the therapeutic cell composition is engineered CD4+ T cells containing an anti-CD19 CAR, the one recombinant receptor-dependent activity to be predicted includes IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, or TNFa+/CD19+.

In some embodiments, for example when the therapeutic cell composition is engineered CD8+ T cells, the one recombinant receptor-dependent activity to be predicted includes IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, or TNFa+.

In some embodiments, for example when the therapeutic cell composition is engineered CD8+ T cells containing an anti-CD19 CAR, the one recombinant receptor-dependent activity to be predicted includes IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, or TNFa+/CD19+.

In some embodiments, for example when the therapeutic cell composition is engineered CD4+ and CD8+ T cells or there are separate therapeutic compositions of CD4+ and CD8+ engineered cells, the one recombinant receptor-dependent activity to be predicted includes IFNG+IL2+CD4+CAR+, IFNG+IL2+IL17+TNFA+CD4+CAR+, IFNG+IL2+TNFA+CD4+CAR+, IFNG+ of CD4+CAR, IFNG+TNFA+CD4+CAR+, IL13+ of CD4+CAR+, IL17+ of CD4+CAR+, IL2+ of CD4+CAR+, IL2+TNFA+CD4+CAR+, TNFA+ of CD4+CAR+, IFNG+IL2+CD8+CAR+, IFNG+IL2+IL17+TNFA+CD8+CAR+, IFNG+IL2+TNFA+CD8+CAR+, IFNG+ of CD8+CAR, IFNG+TNFA+CD8+CAR+, IL13+ of CD8+CAR+, IL17+ of CD8+CAR+, IL2+ of CD8+CAR+, IL2+TNFA+CD8+CAR+, TNFA+ of CD8+CAR+, cytolytic CD8+, GMCSF+, IFNG+, IL10+, IL13+, IL2+, IL5+, MIP1A+, MIP1B+, sCD137+, or TNFa+.

In some embodiments, for example when the therapeutic cell composition is engineered CD4+ and CD8+ T cells or there are separate therapeutic compositions of CD4+ and CD8+ engineered cells containing an anti-CD19 CAR, the one recombinant receptor-dependent activity to be predicted includes IFNG+IL2+CD4+CAR+, IFNG+IL2+IL17+TNFA+CD4+CAR+, IFNG+IL2+TNFA+CD4+CAR+, IFNG+ of CD4+CAR, IFNG+TNFA+CD4+CAR+, IL13+ of CD4+CAR+, IL17+ of CD4+CAR+, IL2+ of CD4+CAR+, IL2+TNFA+CD4+CAR+, TNFA+ of CD4+CAR+, IFNG+IL2+CD8+CAR+, IFNG+IL2+IL17+TNFA+CD8+CAR+, IFNG+IL2+TNFA+CD8+CAR+, IFNG+ of CD8+CAR, IFNG+TNFA+CD8+CAR+, IL13+ of CD8+CAR+, IL17+ of CD8+CAR+, IL2+ of CD8+CAR+, IL2+TNFA+CD8+CAR+, TNFA+ of CD8+CAR+, cytolytic CD8+, GMCSF+CD19+, IFNG+, IL10+, IL13+, IL2+, IL5+, MIP1A+, MIP1B+, sCD137+, or TNFa+.

In some embodiments, the one attribute to be predicted includes 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, GMCSF+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+, IL-10+, IL-13+, IL-2+, IL-5+, MIP1A+, MIP1B+, sCD137+, or TNFa+.

In some embodiments, the one attribute to be predicted includes 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+CAR+, IL-2+ of CD8+CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, or TNFa+/CD19+.

In some embodiments, the one attribute (e.g., the second attribute to be predicted) includes a therapeutic composition attribute shown in Table E2.

In some embodiments, the method is run multiple times such that each second attribute is predicted from the first attributes of the input composition.

In some embodiments, the lasso statistical learning model predicts proportions of CD4+CAR+ naïve (CCR7+CD45RA+) T cells in therapeutic compositions from input composition attributes. In some embodiments, the lasso statistical analysis model predicts T_{EMRA} T cells (e.g., CD27−CD28−, CCR7−CD45RA+) proportions in the therapeutic composition from input composition attributes. In some embodiments, the lasso statistical analysis model predicts proportions of MIP1A in CD4+ cells in the therapeutic cell composition from the input composition attributes. In some embodiments, the lasso statistical analysis model predicts proportions of MIP1B in CD4+ cells in the therapeutic cell composition from the input composition attributes. In some embodiments, the lasso statistical analysis model predicts proportions of IL-2+TNFa+ in CD4 cells in the therapeutic cell composition from the input composition attributes. In some embodiments, the lasso statistical analysis model predicts proportions of IL-2 in CD8+ cells in the therapeutic cell composition from the input composition attributes. In some embodiments, the lasso statistical analysis model predicts proportions of IFNg in CD8+ cells in the therapeutic cell composition from the input composition attributes. In some embodiments, the input composition attributes include 34 attributes. In some embodiments, the 34 composition attributes include 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and/or CAS+/CD3+ of an input composition that is CD8+ cells. In some embodiments, the input composition attributes include or are CD4+/CCR7+/CD27+, CD4+/CCR7+/CD45RA+, CD4+/CD28+/CD27−, and CD8+/CCR7+CD45RA+. In some embodiments, the input composition attribute is CD4+/CCR7+/CD45RA+. In some embodiments, the input composition attributes include or are CD8+/CCR7+, CD4+/CCR7−/CD27−, CD8+/CCR7−/CD45RA+, and CD4+/CD28+. In some embodiments, the one second attribute predicted from the first attributes is CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, CCR7+/CD45RA+/CD4+/CAR+, CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, or CCR7+/CD45RA+/CD8+/CAR+. In some embodiments, the one second attribute predicted from the first attributes is CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, or CCR7+/CD45RA+/CD4+/CAR+. In some embodiments, the one second attribute predicted from the first attributes is CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, or CCR7+/CD45RA+/CD8+/CAR+.

In some embodiments, the first attribute includes a percentage, number, ratio and/or proportion of CCR7+, CCR7+/CD27+, and/or CD27+CD4+ T cells in the input composition, and the first attribute is predictive of a percentage, number, ratio and/or proportion of a second attribute comprising CCR7+, CCR7+/CD27+, CD27+ of CD4+ or CD8+ T cells in the therapeutic cell composition.

In some embodiments, the first attribute includes a percentage, number, ratio and/or proportion of CD4+/CCR7+/CD27+, CD4+/CCR7+/CD45RA+, CD4+/CD28+/CD27−, CD8+/CCR7+/CD45RA−, CD8+/CCR7+/CD45RA+, CD8+/CCR7+, CD4+/CCR7−/CD27−, CD8+/CCR7−/CD45RA+, CD4+/CD28+/CD27−, CD4+/CD28+, and/or CD28+/CD27− T cells in the input composition, and the first attribute is predictive of a percentage, number, ratio and/or proportion of a second attribute comprising CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, CCR7+/CD45RA+/CD4+/CAR+, CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, or CCR7+/CD45RA+/CD8+/CAR+ T cells in the therapeutic cell composition.

In some embodiments, the first attribute includes a percentage, number, ratio and/or proportion of CD4+/CCR7+/CD27+, CD4+/CCR7+/CD45RA+, and/or CD4+/CD28+/CD27 T cells in the input composition, and the first attribute is predictive of a percentage, number, ratio and/or proportion of a second attribute comprising CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, or CCR7+/CD45RA+/CD4+/CAR+ T cells in the therapeutic cell composition.

In some embodiments, the first attribute includes a percentage, number, ratio and/or proportion of CD4+/CCR7+/CD27+, CD4+/CCR7+/CD45RA+, CD4+/CD28+/CD27−, CD8+/CCR7+CD45RA−, and/or CD8+/CCR7+CD45RA+ T cells in the input composition, and the first attribute is predictive of a percentage, number, ratio and/or proportion of comprising CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, or CCR7+/CD45RA+/CD8+/CAR+ T cells in the therapeutic cell composition.

In some embodiments, the first attribute includes a percentage, number, ratio and/or proportion of CD4+/CCR7+/CD45RA+ T cells in the input composition, and the first attribute is predictive of a percentage, number, ratio and/or proportion of a second attribute comprising CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, CCR7+/CD45RA+/CD4+/CAR+, CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, or CCR7+/CD45RA+/CD8+/CAR+ T cells in the therapeutic cell composition.

In some embodiments, the lasso regression statistical learning model predicts the number, percentage, proportion, and/or ratio of desired attributes of a therapeutic cell composition.

3. Methods of Treatment

In some embodiments, understanding the relationship (e.g., correlation) between input composition attributes and therapeutic cell composition attributes, as well as an ability to predict therapeutic cell composition attributes, can indicate the success of manufacturing an effective therapeutic cell composition from an input composition prior to producing the therapeutic composition. In some embodiments, predicting the attributes of the therapeutic cell composition before it is manufactured can inform treatment of the subject. In some embodiments, determining therapeutic cell composition attributes in advance of manufacturing may be useful for developing a treatment regimen or strategy for a subject in need thereof. For example, if input composition attributes predict reduced or suboptimal therapeutic cell composition attributes, e.g., reduced or suboptimal attributes known to correlate with positive clinical outcome, a treatment regimen may be developed to bolster or improve the effects of the therapeutic composition. For example, in some embodiments, the therapeutic cell composition may be administered to the subject as part of a combination therapy. In some embodiments, the dose of the therapeutic composition may be altered to achieve positive clinical outcome (e.g., response).

a. Combination Treatment

In some embodiments, if the therapeutic cell composition is predicted to have reduced or insufficient attributes, e.g., attributes correlated with positive clinical outcome (e.g., durable response, progression free survival), a treatment strategy that includes an additional treatment may be considered. In some embodiments, the therapeutic cell compositions (e.g., CD4+, CD8+ therapeutic T cell compositions) are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the therapeutic cell compositions (e.g., CD4+, CD8+ therapeutic T cell compositions) are co-administered with another therapy sufficiently close in time such that the therapeutic cell composition populations enhance the effect of one or more additional therapeutic agents, or vice versa. In some embodiments, the therapeutic cell compositions (e.g., CD4+, CD8+ therapeutic T cell compositions) are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration.

In some embodiments, the combination therapy includes administration of a kinase inhibitor, such as a BTK inhibitor (e.g. ibrutinib or acalabrutinib); an inhibitor or a tryptophan metabolism and/or kynurenine pathway, such as an inhibitor of indoleamine 2,3-dioxygenase-1 (IDO1) (e.g. epacadostat); an immunomodulatory agent, such as an immunomodulatory imide drug (IMiD), including a thalidomide or thalidomide derivative (e.g. lenalidomide or pomalidomide); or a check point inhibitor, such as an anti-PD-L1 antibody (e.g. durvalumab).

Exemplary combination therapies and methods are described in published international applications WO 2018/085731, WO 2018/102785, WO 2019/213184, WO 2018/071873, WO 2018/102786, WO 2018/204427, WO 2019/152743, which are incorporated by reference in their entirety.

b. Determining Dosing and Administration

In some embodiments, if the therapeutic cell composition is predicted to have reduced or insufficient attributes, e.g., attributes correlated with positive clinical outcome (e.g., durable response, progression free survival), a treatment strategy that optimizes dose may be considered. The therapeutic composition or a dose thereof, in some embodiments contains the cells in amounts effective to treat or prevent the disease or condition, such as a therapeutically effective or prophylactically effective amount. In some embodiments, the composition includes the cells in an amount effective to reduce burden of the disease or condition. In some embodiments, the composition includes cells in an amount that provides more consistent outcome, e.g., response and/or safety outcomes, among a group of subjects administered the composition, and/or more consistent pharmacokinetic parameters. In some embodiments, the composition includes the cells in an amount effective to promote durable response and/or progression free survival. In some aspects, the provided methods involve assessing a therapeutic composition containing T cells for cell phenotypes, and determining doses based on such outcomes.

In some embodiments, the dose is determined to encompass a relatively consistent number, proportion, ratio and/or percentage of engineered cells having a particular phenotype in one or more particular compositions. In some aspects, the consistency is associated with or related to a relatively consistent activity, function, pharmacokinetic parameters, toxicity outcome and/or response outcome. In some aspects, in a plurality of subjects, compositions and/or doses the numbers, proportion, ratio and/or percentage, are relatively consistent, e.g., the number or ratio of cells that have a particular phenotype, e.g., express CCR7 (CCR7⁺) or, that produce a cytokine, for example, produce IL-2, TNF-alpha, or IFN-gamma, in the composition or unit dose, varies by no more than 40%, by no more than 30%, by no more than 20%, by no more than 10% or by no more than 5%. In some aspects the number or ratio of cells that have a particular phenotype, e.g., express CCR7 (CCR7⁺), in the composition or unit dose, varies by no more than 20% or no more than 10% or no more than 5% from an average of said number or ratio in a plurality of T cell compositions produced by the process and/or varies from such average by no more than one standard deviation or varies by no more than 20% or no more than 10% or no more than 5% among a plurality of T cell compositions or doses determined. In some embodiments, the plurality of subjects includes at least 10 subjects, such as at least 15, at least 20, at least 25, at least 30, at least 40, at least 50, at least 60, at least 70, at least 80, at least 90, at least 100 or more subjects.

In some aspects, the dose, e.g., one or more unit dose(s) is determined based on the number, percentage, ratio, frequency and/or proportion of a particular subset of engineered T cells, e.g., cells having a particular phenotype, such as particular surface marker phenotype. In some aspects, the cell phenotype is determined based on expression and/or absence of expression of particular cell markers, e.g., surface markers. In some aspects, the cell marker includes markers indicative of viability and/or apoptotic state of the cells. In some aspects, exemplary markers include CD3, CD4, CD8, CCR7, CD27, CD45RA, annexin V, or activated caspase 3. In some aspects, an exemplary marker is CCR7. In some aspects, an exemplary marker is CD27. In some aspects, exemplary markers include CCR7 and/or CD27. In some aspects, exemplary markers include CCR7, CD27 and/or CD45RA.

In some embodiments, provided are methods involving administering to a subject one or more unit doses of a therapeutic T cell composition, such as any described herein and/or any unit dose determined by the methods provided herein.

In some embodiments, provided are methods involving administering to a subject having a disease or condition a unit dose of a T cell composition comprising cells comprising a recombinant receptor, such as a chimeric antigen receptor (CAR), that specifically binds to an antigen associated with the disease or condition, wherein either a defined number of total recombinant receptor-expressing cells (receptor⁺) of the therapeutic composition, total CD8⁺ recombinant receptor-expressing cells (receptor⁺/CD8⁺) are administered and/or a unit dose of such cells is administered in which the unit dose contains a defined number, percentage, ratio, frequency and/or proportion of cells with a certain phenotype, e.g., CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, CCR7⁻/CD27⁻/CD8⁺.

In some embodiments, the unit dose of cells comprises a defined number of recombinant receptor-expressing CD8⁺ T cells that express C-C chemokine receptor type 7 (CCR7) (receptor⁺/CD8⁺/CCR7⁺ cells) and/or a defined number of recombinant receptor-expressing CD4⁺ T cells that express CCR7 (receptor⁺/CD4⁺/CCR7⁺ cells) and/or a defined ratio of receptor⁺/CD8⁺/CCR7⁺ cells to receptor⁺/CD4⁺/CCR7⁺ cells and/or a defined ratio of receptor⁺/CD8⁺/CCR7⁺ cells and/or receptor⁺/CD4⁺/CCR7⁺ cells to another subset of cells in the composition. In some embodiments, the unit dose of cells comprises a defined number of CD8⁺/CCR7⁺ cells. In some embodiments, the unit dose of cells comprises a defined number of CD4⁺/CCR7⁺ cells. In some embodiments, the defined number or ratio is further based on expression or absence of expression of CD27 and/or CD45RA on the cells.

In some embodiments, the unit dose of cells comprises a defined number of recombinant receptor-expressing CD8⁺ T cells that express cluster of differentiation 27 (CD27) (receptor⁺/CD8⁺/CD27⁺ cells) and/or a defined number of recombinant receptor-expressing CD4⁺ T cells that express CD27 (receptor⁺/CD4⁺/CD27⁺ cells) and/or a defined ratio of receptor⁺/CD8⁺/CD27⁺ cells to receptor⁺/CD4⁺/CD27⁺ cells and/or a defined ratio of receptor⁺/CD8⁺/CD27⁺ cells and/or receptor⁺/CD4⁺/CD27⁺ cells to another subset of cells in the composition. In some embodiments, the unit dose of cells comprises a defined number of CD8⁺/CD27⁺ cells. In some embodiments, the unit dose of cells comprises a defined number of CD4⁺/CD27⁺ cells. In some embodiments, the defined number or ratio is further based on expression or absence of expression of CCR7 and/or CD45RA on the cells.

In some embodiments, the unit dose of cells comprises a defined number of recombinant receptor-expressing CD8⁺ T cells that express CCR7 and CD27 (receptor⁺/CD8⁺/CCR7⁺/CD27⁺ cells) and/or a defined number of recombinant receptor-expressing CD4⁺ T cells that express CCR7 and CD27 (receptor⁺/CD4⁺/CCR7⁺/CD27⁺ cells) and/or a defined ratio of receptor⁺/CD8⁺/CCR7⁺/CD27⁺ cells to receptor⁺/CD4⁺/CCR7⁺/CD27⁺ cells and/or a defined ratio of receptor⁺/CD8⁺/CCR7⁺/CD27⁺ cells and/or receptor⁺/CD4⁺/CCR7⁺/CD27⁺ cells to another subset of cells in the composition. In some embodiments, the unit dose of cells comprises a defined number of CD8⁺/CCR7⁺/CD27⁺ cells. In some embodiments, the unit dose of cells comprises a defined number of CD4⁺/CCR7⁺/CD27⁺ cells. In some embodiments, the defined number or ratio is further based on expression or absence of expression of CD45RA on the cells.

In some embodiments, the number of cells in the unit dose is the number of cells or number of recombinant receptor-expressing or CAR-expressing cells, or number, percentage, ratio, frequency and/or proportion of such cells of a certain phenotype, e.g. cells that express or do not express one or more markers selected from CD3 CD4, CD8, CCR7, CD27, CD45RA, annexin V, or activated caspase 3, that it is desired to administer to a particular subject in a dose, such as a subject from which the cells have been derived. In some embodiments, the number of cells in the unit dose is the number of cells or number of recombinant receptor-expressing or CAR-expressing cells, or number, percentage, ratio, frequency and/or proportion of such cells of a certain phenotype, e.g., CCR7⁺, CD27⁺, CD45RA⁺, CD45RA⁻, CD4⁺, CD8⁺, CD3⁺, apoptosis marker negative (e.g. Annexin V⁻ or Caspase 3⁻) cells, or cells that are positive or negative for one or more of any of the foregoing.

In some embodiments, the number of cells in the unit dose is the number of cells or number of recombinant receptor-expressing or CAR-expressing cells, or number, percentage, ratio and/or proportion of such cells of a certain phenotype, e.g., CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, CCR7⁻/CD27⁻/CD8⁺; and apoptosis marker negative (e.g. Annexin V⁻ or Caspase 3⁻) cells, that it is desired to administer to a particular subject in a dose, such as a subject from which the cells have been derived. In some embodiments, the unit dose contains a defined number of cells or number of recombinant receptor-expressing or CAR-expressing cells, or number, percentage, ratio and/or proportion of such cells of a certain phenotype e.g., CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, CCR7⁻/CD27⁻/CD8⁺; and apoptosis marker negative (e.g. Annexin V⁻ or Caspase 3⁻) cells, and/or any subset thereof.

In some embodiments, the unit dose is determined based on the number of cells or cell type(s) and/or a frequency, ratio, and/or percentage of cells or cell types, e.g., individual populations, phenotypes, or subtypes, in the cell composition, such as those with the phenotypes of annexin V⁻/CCR7⁺/CAR⁺; annexin V⁻/CCR7⁺/CAR⁺/CD4⁺; annexin V⁻/CCR7⁺/CAR⁺/CD8⁺; annexin V⁻/CD27⁺/CAR⁺; annexin V⁻/CD27⁺/CAR⁺/CD4⁺; annexin V⁻/CD27⁺/CAR⁺/CD8⁺; annexin V⁻/CCR7⁺/CD27⁺/CAR⁺; annexin V⁻/CCR7⁺/CD27⁺/CAR⁺/CD4⁺; annexin V⁻/CCR7⁺/CD27⁺/CAR⁺/CD8⁺; annexin V⁻/CCR7⁺/CD45RA⁻/CAR⁺; annexin V⁻/CCR7⁺/CD45RA⁻/CAR⁺/CD4⁺; annexin V⁻/CCR7⁺/CD45RA⁻/CAR⁺/CD8+; annexin V⁻/CCR7⁻/CD45RA⁻/CAR⁺; annexin V⁻/CCR7⁻/CD45RA⁻/CAR⁺/CD4⁺; annexin V⁻/CCR7⁻/CD45RA⁻/CAR⁺/CD8⁺; annexin V⁻/CCR7⁻/CD27⁻/CAR⁺, annexin V⁻/CCR7⁻/CD27⁻/CAR⁺/CD4⁺; annexin V⁻/CCR7⁻/CD27⁻/CAR⁺/CD8⁺; activated caspase 3⁻/CCR7⁺/CAR⁺; activated caspase 3⁻/CCR7⁺/CAR⁺/CD4⁺; activated caspase 3⁻/CCR7⁺/CAR⁺/CD8⁺; activated caspase 3⁻/CD27⁺/CAR⁺; activated caspase 3⁻/CD27⁺/CAR⁺/CD4⁺; activated caspase 3⁻/CD27⁺/CAR⁺/CD8⁺; activated caspase 3⁻/CCR7⁺/CD27⁺/CAR⁺; activated caspase 3⁻/CCR7⁺/CD27⁺/CAR⁺/CD4⁺; activated caspase 3⁻/CCR7⁺/CD27⁺/CAR⁺/CD8⁺; activated caspase 3⁻/CCR7⁺/CD45RA⁻/CAR⁺; activated caspase 3⁻/CCR7⁺/CD45RA⁻/CAR⁺/CD4⁺; activated caspase 3⁻/CCR7⁺/CD45RA⁻/CAR⁺/CD8⁺; activated caspase 3⁻/CCR7⁻/CD45RA⁻/CAR⁺; activated caspase 3⁻/CCR7⁻/CD45RA⁻/CAR⁺/CD4⁺; activated caspase 3⁻/CCR7⁻/CD45RA⁻/CAR⁺/CD8⁺; activated caspase 3⁻/CCR7⁻/CD27⁻/CAR⁺; activated caspase 3⁻/CCR7⁻/CD27⁻/CAR⁺/CD4⁺; and/or activated caspase 3⁻/CCR7⁻/CD27⁻/CAR⁺/CD8⁺; or a combination thereof.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells, each inclusive. In some embodiments, the unit dose comprises no more than about 1×10⁸, no more than about 5×10⁷, no more than about 1×10⁷, no more than about 5×10⁶, no more than about 1×10⁶, or no more than about 5×10⁵ total receptor⁺/CD8⁺ cells or total receptor⁺/CD4⁺ cells, total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells.

In some embodiments, the unit dose comprises between at or about 5×10⁵ and at or about 5×10⁷, between at or about 1×10⁶ and at or about 1×10⁷, or between at or about 5×10⁶ and at or about 1×10⁷ total receptor⁺/CD8⁺/CCR7⁺ cells or receptor⁺/CD4⁺/CCR7⁺ cells, each inclusive. In some embodiments, the unit dose comprises at least or at least about 5×10⁷, 1×10⁷, 5×10⁶, 1×10⁶, or at least about 5×10⁵ total receptor⁺/CD8⁺/CCR7⁺ cells or receptor⁺/CD4⁺/CCR7⁺ cells.

In some embodiments, the unit dose comprises between at or about 5×10⁵ and at or about 5×10⁷, between at or about 1×10⁶ and at or about 1×10⁷, or between at or about 5×10⁶ and at or about 1×10⁷ total receptor⁺/CD8⁺/CD27⁺ cells or receptor⁺/CD4⁺/CD27⁺ cells, each inclusive. In some embodiments, the unit dose comprises at least or at least about 5×10⁷, 1×10⁷, 5×10⁶, 1×10⁶, or at least about 5×10⁵ total receptor⁺/CD8⁺/CD27⁺ cells or receptor⁺/CD4⁺/CD27⁺ cells.

In some embodiments, the unit dose comprises at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD8⁺/CCR7⁺ cells and/or at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD4⁺/CCR7⁺ cells, each inclusive. In some embodiments, the unit dose comprises between at or about 3×10⁶ and at or about 2.5×10⁷, between at or about 4×10⁶ and at or about 2×10⁷, or between at or about 5×10⁶ and at or about 1×10⁷ total receptor⁺/CD8⁺/CCR7⁺ cells and/or between at or about 3×10⁶ and at or about 2.5×10⁷, between at or about 4×10⁶ and at or about 2×10⁷, or between at or about 5×10⁶ and at or about 1×10⁷ total receptor⁺/CD4⁺/CCR7⁺ cells, each inclusive.

In some embodiments, the unit dose comprises at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD8⁺/CD27⁺ cells and/or at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD4⁺/CD27⁺ cells, each inclusive. In some embodiments, the unit dose comprises unit dose comprises between at or about 3×10⁶ and at or about 2.5×10⁷, between at or about 4×10⁶ and at or about 2×10⁷, or between at or about 5×10⁶ and at or about 1×10⁷ total receptor⁺/CD8⁺/CD27⁺ cells and/or between at or about 3×10⁶ and at or about 2.5×10⁷, between at or about 4×10⁶ and at or about 2×10⁷, or between at or about 5×10⁶ and at or about 1×10⁷ total receptor⁺/CD4⁺/CD27⁺ cells, each inclusive.

In some embodiments, the unit dose comprises between at or about 5×10⁵ and at or about 5×10⁷, between at or about 1×10⁶ and at or about 1×10⁷, or between at or about 5×10⁶ and at or about 1×10⁷ total receptor⁺/CD8⁺/CCR7⁺/CD27⁺ cells or receptor⁺/CD4⁺/CCR7⁺/CD27⁺ cells, each inclusive. In some embodiments, the unit dose comprises at least or at least at or about 5×10⁷, 1×10⁷, 5×10⁶, 1×10⁶, or at least at or about 5×10⁵ total receptor⁺/CD8⁺/CCR7⁺/CD27⁺ cells or receptor⁺/CD4⁺/CCR7⁺/CD27⁺ cells.

In some embodiments, the unit dose comprises at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD8⁺/CCR7⁺/CD27⁺ cells and/or at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD4⁺/CCR7⁺/CD27⁺ cells, each inclusive. In some embodiments, the unit dose comprises between at or about 3×10⁶ and at or about 2.5×10⁷, between at or about 4×10⁶ and at or about 2×10⁷, or between at or about 5×10⁶ and at or about 1×10⁷ total receptor⁺/CD8⁺/CCR7⁺/CD27⁺ cells and/or between at or about 3×10⁶ and at or about 2.5×10⁷, between at or about 4×10⁶ and at or about 2×10⁷, or between at or about 5×10⁶ and at or about 1×10⁷ total receptor⁺/CD4⁺/CCR7⁺/CD27⁺ cells, each inclusive.

In some embodiments, the unit dose of cells comprises a defined ratio of receptor⁺/CD8⁺/CCR7⁺ cells to receptor⁺/CD4⁺/CCR7⁺ cells, which ratio optionally is or is approximately 1:1 or is between approximately 1:3 and approximately 3:1.

In some embodiments, the unit dose of cells comprises a defined ratio of receptor⁺/CD8⁺/CD27⁺ cells to receptor⁺/CD4⁺/CD27⁺ cells, which ratio optionally is or is approximately 1:1 or is between approximately 1:3 and approximately 3:1.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺/CD27⁺ cells, or total receptor⁺/CD4⁺/CCR7⁺/CD27⁺ cells, each inclusive. In some embodiments, the unit dose comprises no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD8⁺ cells or total receptor⁺/CD4⁺ cells, total receptor⁺/CD8⁺/CCR7⁺/CD27⁺ cells, or total receptor⁺/CD4⁺/CCR7⁺/CD27⁺ cells.

In some embodiments, the unit dose of cells comprises a defined ratio of receptor⁺/CD8⁺/CCR7⁺/CD27⁺ cells to receptor⁺/CD4⁺/CCR7⁺/CD27⁺ cells, which ratio optionally is or is approximately 1:1 or is between approximately 1:3 and approximately 3:1.

In some embodiments, the unit dose comprises between at or about 1×10⁵ and at or about 5×10⁸, between at or about 1×10⁵ and at or about 1×10⁸, between at or about 5×10⁵ and at or about 1×10⁷, or between at or about 1×10⁶ and at or about 1×10⁷ total CD3⁺ cells that express the recombinant receptor (receptor⁺/CD3⁺ cells) or total CD3⁺ cells, each inclusive. In some embodiments, the unit dose comprises no more than at or about 5×10⁸, no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD3⁺ cells or total CD3⁺ cells.

In some embodiments, the total number of CD3⁺ cells, total number of receptor⁺/CD3⁺ cells, total number of receptor⁺/CD8⁺ cells, total number of receptor⁺/CD4⁺ cells, total number of receptor⁺/CD8⁺/CCR7+ cells, total number of receptor⁺/CD4⁺/CCR7⁺ cells, total number of receptor⁺/CD8⁺/CD27⁺ cells, total number of receptor⁺/CD4⁺/CD27⁺ cells, total number of receptor⁺/CD8⁺/CCR7⁺/CD27⁺ cells, total number of receptor⁺/CD4⁺/CCR7⁺/CD27⁺ cells, total number of receptor⁺/CD8⁺/CCR7⁺/CD45RA⁻ cells and/or receptor⁺/CD4⁺/CCR7⁺/CD45RA⁻ cells is the total number of such cells that are live or viable. In some embodiments, the total number of CD3⁺ cells, total number of receptor⁺/CD3⁺ cells, total number of receptor⁺/CD8⁺ cells, total number of receptor⁺/CD4⁺ cells, total number of receptor⁺/CD8⁺/CCR7⁺ cells, total number of receptor⁺/CD4⁺/CCR7⁺ cells, total number of receptor⁺/CD8⁺/CD27⁺ cells, total number of receptor⁺/CD4⁺/CD27⁺ cells, total number of receptor⁺/CD8⁺/CCR7⁺/CD27⁺ cells, total number of receptor⁺/CD4⁺/CCR7⁺/CD27⁺ cells, total number of receptor⁺/CD8⁺/CCR7⁺/CD45RA⁻ cells and/or receptor⁺/CD4⁺/CCR7⁺/CD45RA⁻ cells is the total number of such cells that do not express an apoptotic marker and/or is the total number of such cells that are apoptotic marker negative (⁻), wherein the apoptotic marker is Annexin V or activated Caspase 3.

In some embodiments, in any of the composition comprising T cells expressing a recombinant receptor provided herein, at least at or about, or at or about, 15%, 20%, 30%, 40%, 50%, 60%, 70%, 80%, 85%, 90%, 91%, 92%, 93%, 94%, 95%, 96%, 97%, 98%, 99% or 100% of the total number of T cells in the composition (or of the total number of T cells in the composition expressing the recombinant receptor), are surface positive for CCR7 and/or CD27.

In some embodiments, in any of the composition comprising T cells expressing a recombinant receptor provided herein, at least at or about, or at or about, 70%, 80%, 85%, 90%, 91%, 92%, 93%, 94%, 95%, 96%, 97%, 98%, 99% or 100% of the total number of T cells in the composition (or of the total number of T cells in the composition expressing the recombinant receptor), are able to produce a cytokine selected from interleukin 2 (IL-2) and/or TNF-alpha. In some embodiments, the T cell able to produce IL-2 and/or TNF-alpha is a CD4+ T cell.

In some embodiments, in any of the composition comprising T cells expressing a recombinant receptor provided herein, at least at or about, or at or about, 15%, 20%, 30%, 40%, 50%, 60%, 70%, 80% or 90% of the total receptor⁺ cells in the unit dose, or between at or about 15% and at or about 90%, between at or about 20% and at or about 80%, between at or about 30% and at or about 70%, or between at or about 40% and at or about 60%, each inclusive, of the total receptor⁺ cells in the unit dose are receptor⁺/CD8⁺/CCR7⁺ or receptor⁺/CD8⁺/CD27⁺. In some embodiments, in any of the composition comprising T cells expressing a recombinant receptor provided herein, at least at or about 15%, 20%, 30%, 40%, 50%, 60%, 70%, 80% or 90% of the total receptor⁺ cells in the unit dose, or between at or about 15% and at or about 90%, between at or about 20% and at or about 80%, between at or about 30% and at or about 70%, or between at or about 40% and at or about 60%, each inclusive, of the total receptor⁺ cells in the unit dose are receptor⁺/CD4⁺/CCR7⁺ or receptor⁺/CD4⁺/CD27⁺. In some embodiments, at least at or about 15%, 20%, 30%, 40%, 50%, 60%, 70%, 80% or 90% of the total receptor⁺ cells in the unit dose, or between at or about 15% and at or about 90%, between at or about 20% and at or about 80%, between at or about 30% and at or about 70%, or between at or about 40% and at or about 60%, each inclusive, of the total receptor⁺ cells in the unit dose are receptor⁺/CD8⁺/CCR7⁺/CD27⁺, receptor⁺/CD8⁺/CCR7⁺/CD45RA⁻, receptor⁺/CD4⁺/CCR7⁺/CD27⁺ or receptor⁺/CD4⁺/CCR7⁺/CD45RA⁻.

In some embodiments, in any of the composition comprising T cells expressing a recombinant receptor provided herein, at least at or about 50%, 60%, 70%, 80% or 90% of the total receptor⁺/CD8+ cells in the composition or unit dose are or the unit dose, or between at or about 50% and at or about 90%, between at or about 60% and at or about 90%, between at or about 70% and at or about 80%, each inclusive, of the total receptor⁺/CD8+ cells in the composition or the unit dose are receptor⁺/CD8⁺/CCR7⁺ or receptor⁺/CD8⁺/CD27⁺ or receptor⁺/CD8⁺/CCR7⁺/CD27⁺. In some embodiments, in any of the composition comprising T cells expressing a recombinant receptor provided herein, at least at or about 50%, 60%, 70%, 80% or 90% of the total receptor⁺/CD4⁺ cells in the composition or unit dose are or the unit dose, or between at or about 50% and at or about 90%, between at or about 60% and at or about 90%, between at or about 70% and at or about 80%, each inclusive, of the total receptor⁺/CD4⁺ cells in the composition or the unit dose are receptor⁺/CD4⁺/CCR7⁺ or receptor⁺/CD4⁺/CD27⁺ or receptor⁺/CD4⁺/CCR7⁺/CD27⁺. receptor⁺/CD8⁺/CCR7⁺/CD27⁺, receptor⁺/CD8⁺/CCR7⁺/CD45RA⁻, receptor⁺/CD4⁺/CCR7⁺/CD27⁺ or receptor⁺/CD4⁺/CCR7⁺/CD45RA⁻. In some embodiments, at least at or about 50%, 60%, 70%, 80% or 90% of the total receptor⁺/CD8⁺ cells in the composition are receptor⁺/CD8⁺/CCR7⁺/CD27⁺; or at least at or about 15%, 20%, 30%, 40%, 50%, 60%, 70%, 80% or 90% of the total receptor⁺/CD4⁺ cells in the composition are receptor⁺/CD4⁺/CCR7⁺/CD27⁺.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells, each inclusive. In some embodiments, the unit dose comprises no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD8⁺ cells or total receptor⁺/CD4⁺ cells, total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells.

In some embodiments, the unit dose of cells comprises a defined ratio of receptor⁺/CD8⁺/CCR7⁺ cells to receptor⁺/CD4⁺/CCR7⁺ cells, which ratio optionally is or is approximately 1:1 or is between approximately 1:3 and approximately 3:1.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺/CD27⁺ cells, or total receptor⁺/CD4⁺/CCR7⁺/CD27⁺ cells, each inclusive. In some embodiments, the unit dose comprises no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD8⁺ cells or total receptor⁺/CD4⁺ cells, total receptor⁺/CD8⁺/CCR7⁺/CD27⁺ cells, or total receptor⁺/CD4⁺/CCR7⁺/CD27⁺ cells.

In some embodiments, the unit dose of cells comprises a defined ratio of receptor⁺/CD8⁺/CCR7⁺/CD27⁺ cells to receptor⁺/CD4⁺/CCR7⁺/CD27⁺ cells, which ratio optionally is or is approximately 1:1 or is between approximately 1:3 and approximately 3:1.

In some embodiments, the provided methods involve administering a dose containing a defined number of cells. In some embodiments, the dose, such as the defined number of cells, such as a defined number of CAR+ cells that are CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, or CCR7⁻/CD27⁻/CD8⁺, is between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷. In some embodiments, such dose, such as such defined number of cells refers to the total recombinant-receptor expressing cells in the administered composition. In some aspects, the defined number of recombinant receptor-expressing cells that are administered are cells that are apoptotic marker negative(−) and optionally wherein the apoptotic marker is Annexin V or activated Caspase 3.

In some embodiments, the dose of cells of the unit dose contains a number of cells, such as a defined number of cells, between at least or at least about 5×10⁶, 6×10⁶, 7×10⁶, 8×10⁶, 9×10⁶, 10×10⁶ and about 15×10⁶ recombinant-receptor expressing cells, such as recombinant-receptor expressing cells that are CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, or CCR7⁻/CD27⁻/CD8⁺, and/or that are apoptotic marker negative(−) and CD8⁺, optionally wherein the apoptotic marker is Annexin V or activated Caspase 3.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, the dose of cells is a flat dose of cells or fixed dose of cells such that the dose of cells is not tied to or based on the body surface area or weight of a subject.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total recombinant receptor (e.g., CAR)-expressing cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁸ total such cells, or the range between any two of the foregoing values.

In some embodiments, the dose of genetically engineered cells comprises from at or about 1×10⁵ to at or about 5×10⁸ total CAR-expressing (CAR-expressing) T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1 x 10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells. In some embodiments, the dose of genetically engineered cells comprises from or from about 2.5×10⁷ to at or about 1.5×10⁸ total CAR-expressing T cells, such as from or from about 5×10⁷ to or to about 1×10⁸ total CAR-expressing T cells.

In some embodiments, the dose of genetically engineered cells comprises at least at or about 1×10⁵ CAR-expressing cells, at least at or about 2.5×10⁵ CAR-expressing cells, at least at or about 5×10⁵ CAR-expressing cells, at least at or about 1×10⁶ CAR-expressing cells, at least at or about 2.5×10⁶ CAR-expressing cells, at least at or about 5×10⁶ CAR-expressing cells, at least at or about 1×10⁷ CAR-expressing cells, at least at or about 2.5×10⁷ CAR-expressing cells, at least at or about 5×10⁷ CAR-expressing cells, at least at or about 1×10⁸ CAR-expressing cells, at least at or about 1.5×10⁸ CAR-expressing cells, at least at or about 2.5×10⁸ CAR-expressing cells, or at least at or about 5×10⁸ CAR-expressing cells.

In some embodiments, the cell therapy comprises administration of a dose comprising a number of cell from or from about 1×10⁵ to or to about 5×10⁸ total recombinant receptor-expressing cells, total T cells, or total peripheral blood mononuclear cells (PBMCs), from or from about 5×10⁵ to or to about 1×10⁷ total recombinant receptor-expressing cells, total T cells, or total peripheral blood mononuclear cells (PBMCs) or from or from about 1×10⁶ to or to about 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3⁺ or CD8⁺, in some cases also recombinant receptor-expressing (e.g. CAR⁺) cells. In some embodiments, the cell therapy comprises administration of a dose comprising a number of cell from or from about 1×10⁵ to or to about 5×10⁸ CD3⁺ or CD8⁺ total T cells or CD3⁺ or CD8⁺ recombinant receptor-expressing cells, from or from about 5×10⁵ to or to about 1×10⁷ CD3⁺ or CD8⁺ total T cells or CD3⁺ or CD8⁺ recombinant receptor-expressing cells, or from or from about 1×10⁶ to or to about 1×10⁷ CD3⁺ or CD8⁺ total T cells or CD3⁺ or CD8⁺ recombinant receptor-expressing cells, each inclusive. In some embodiments, the cell therapy comprises administration of a dose comprising a number of cell from or from about 1×10⁵ to or to about 5×10⁸ total CD3⁺/CAR⁺ or CD8⁺/CAR+ cells, from or from about 5×10⁵ to or to about 1×10⁷ total CD3⁺/CAR⁺ or CD8⁺/CAR+ cells, or from or from about 1×10⁶ to or to about 1×10⁷ total CD3⁺/CAR⁺ or CD8⁺/CAR⁺ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 5×10⁸ total recombinant receptor (e.g., CAR)-expressing CD8+ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8+ T cells, from or from about 1×10⁷ to or to about 5×10⁷ total recombinant receptor-expressing CD8+ T cells, from or from about 1×10⁷ to or to about 0.25×10⁸ total recombinant receptor-expressing CD8+ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8⁺- and CD4⁺-enriched populations, respectively, e.g., CD4⁺ and/or CD8⁺ T cells each individually including cells genetically engineered to express the recombinant receptor. In some embodiments, the administration of the dose comprises administration of a first composition comprising a dose of CD8⁺ T cells or a dose of CD4⁺ T cells and administration of a second composition comprising the other of the dose of CD4⁺ T cells and the CD8⁺ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some embodiments, the first composition, e.g., first composition of the dose, comprises CD4⁺ T cells. In some embodiments, the first composition, e.g., first composition of the dose, comprises CD8⁺ T cells. In some embodiments, the first composition is administered prior to the second composition. In some embodiments, the second composition, e.g., second composition of the dose, comprises CD4+ T cells. In some embodiments, the second composition, e.g., second composition of the dose, comprises CD8+ T cells.

In some embodiments, the dose or composition of cells includes a defined or target ratio of CD4⁺ cells expressing a recombinant receptor to CD8⁺ cells expressing a recombinant receptor and/or of CD4⁺ cells to CD8⁺ cells, which ratio optionally is approximately 1:1 or is between approximately 1:3 and approximately 3:1, such as approximately 1:1. In some aspects, the administration of a composition or dose with the target or desired ratio of different cell populations (such as CD4⁺:CD8⁺ ratio or CAR⁺CD4⁺:CAR⁺CD8⁺ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios. For example, in some embodiments, the desired ratio (e.g., ratio of CD4⁺ to CD8⁺ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1), such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered. In some embodiments, the size of the dose is determined based upon predicted output cell composition attributes. In some of any of the above embodiments, the dose may be a predetermined dose and/or a predetermined regimen. In some embodiments, the size of the dose, concentration of the dose, and/or frequency of administering the dose may be modified to achieve positive clinical outcome (e.g., response). In some embodiments, altering the dose size, concentration, and/or frequency of administration results in altering a predetermined dose and/or treatment regime.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

4. Diagnostic Manufacturing

The successful manufacture of effective therapeutic cell compositions for cell therapy, and autologous cell therapy in particular, can be complicated by a variety of factors, including heterogeneity of starting materials derived from a subject, e.g., input compositions, from which the therapeutic cell composition is manufactured. See, e.g., Piscopo, 2018, Biotechnol J. For example, some subjects, e.g., patients, for whom a therapeutic cell composition is to be generated may have received one or more previous treatments for malignancies, which can reduce the total number and/or quality, e.g., health, differentiation status, of the T cells derived from the subject, making it difficult to successfully manufacture a therapeutic cell composition. In some cases, input compositions including CD4+ and/or CD8+ T cells with advanced states of differentiation require longer manufacturing durations and in some cases manufacturing termination (e.g., manufacturing failure). However, not all input compositions are at risk of manufacturing failure or prolonged manufacturing. The methods provided herein are contemplated as useful for predicting attributes of the manufactured therapeutic cell composition prior to manufacturing such that selection of a manufacturing method capable of increasing the probability of successful manufacturing can be used to generate the therapeutic cell composition. In some embodiments, the statistical learning methods described herein allow for diagnostic manufacturing, wherein manufacturing processes to generate a successful therapeutic cell composition can be selected prior to initiating manufacturing.

In some embodiments, understanding the relationship (e.g., correlation) between input composition attributes and therapeutic cell composition attributes, as well as an ability to predict therapeutic cell composition attributes, can indicate the success of manufacturing an effective therapeutic cell composition from an input composition prior to producing the therapeutic composition. In some embodiments, predicting the attributes of the therapeutic cell composition before it is manufactured can assist in selecting a manufacturing process that will yield a success and/or effective therapeutic cell composition. For example, if input composition attributes predict reduced or suboptimal therapeutic cell composition attributes, e.g., reduced or suboptimal attributes known to correlate with positive clinical outcome, e.g., lacking desired attributes, a manufacturing process may be selected to bolster or improve the likelihood of therapeutic cell composition having desired attributes, e.g., as described in Section I-A-2-a. For example, in some embodiments, a second manufacturing process including altered processing steps compared to a first manufacturing process, e.g., as described in Section II below, may be used to generate the therapeutic cell composition. Alternatively, in some embodiments, if the therapeutic cell composition is predicted, according to the statistical learning methods described herein, to have one or more desired attributes the therapeutic cell composition may be generated using a first manufacturing process, e.g., as described in Section II below.

In some embodiments, the second manufacturing process includes one or more steps that are altered compared to the first manufacturing procedure, e.g., as described in Section II below. In some embodiments, the one or more altered steps of the second manufacturing process include an altered T cell expansion step, a selection step to enrich for naive and/or naïve-like cells, a selection step to deplete terminally differentiated cells and/or cells with reduced proliferative capacity, and a threshold number of naïve or naïve-like cells. In some embodiments, the second manufacturing process includes one or more altered steps. In some embodiments, the second manufacturing process includes one altered step. In some embodiments, the steps of the second manufacturing process are identical to the steps of the first manufacturing process except for inclusion of the altered step(s).

In some embodiments, the first manufacturing process is selected from a process including a step of introducing T cells of the input composition with a nucleic acid encoding a recombinant receptor to generate an engineered T cell composition, and cultivating the engineered T cell compositions under conditions for expansion of T cells. In some embodiments, the first manufacturing process is an expanded process resulting in more than 2-fold increase in cells in the therapeutic cell composition compared to the input composition. In some embodiments, the increase in cells in therapeutic cell composition is more than 4-fold compared to the input composition. In some embodiments, obtaining the input composition for manufacturing by the first manufacturing process does not include enriching or selecting for naïve-like T cells or T cells having a central memory phenotype from a biological sample. In some embodiments, obtaining the input composition for manufacturing by the first manufacturing process does not comprise depleting T cells comprising a phenotype of a terminally differentiated T cell or cell with reduced proliferative capacity. In some embodiments, the phenotype of a terminally differentiated T cell or cell with reduced proliferative capacity is CD57+.

In some embodiments, the second manufacturing process is selected from a process including a step of introducing T cells of the input composition with a nucleic acid encoding a recombinant receptor to generate an engineered T cell composition, and incubating the engineered T cell composition that does not expand T cells in the composition or that minimally expands T cells in the composition. In some embodiments, the second manufacturing process includes obtaining the input composition by enriching or selecting for naïve-like T cells or T cells having a central memory phenotype from the biological sample. In some embodiments, the input composition includes a threshold number of naïve-like cells or central memory T cells which allows for initiation of the second manufacturing process. In some embodiments, the input composition includes depleting T cells comprising a phenotype of a terminally differentiated T cells or cells with reduced proliferative capacity. In some embodiments, the phenotype of a terminally differentiated T cell or cell with reduced proliferative capacity is CD57+. In some embodiments, the remaining steps of the second manufacturing process are the same or nearly identical to the steps of the first manufacturing process.

a. Expansion of Engineered Cells

In some embodiments, the first manufacturing process includes a step of expanding cells, e.g., T cells, of the therapeutic cell composition. In some embodiments, the first manufacturing process including the expansion step is referred to as an expanded process. In some embodiments, the expanded process includes a step of cultivating or incubating cells, which have been engineered (e.g. introduced or transduced) with a nucleic acid encoding a recombinant receptor, with one or more recombinant cytokines (e.g. IL-2, IL-7 and/or IL-15) under conditions to support expansion of T cells in the composition. In some embodiments, the expansion can be carried out under perfusion or under continuous perfusion. In some embodiments, the incubation under conditions for expansion results in a greater than 2-fold increase in the number of cells in the composition compared to the starting number of cells of the input composition or the starting number of cells just prior to the cultivation of the cells for expansion. In some embodiments, such incubation under conditions for expansion results in greater than 3-fold, greater than 4-fold, greater than 5-fold, greater than 6-fold, greater than 7-fold, greater than 8-fold, greater than 9-fold, greater than 10-fold or more of such expansion. In some embodiments, the first manufacturing process includes a step for cultivating the cells under conditions for expansion, such as described in Section II-D.

In some embodiments, the first manufacturing process is a process as described in published International Applications WO 2019/089855 and WO2019113557 which are incorporated herein by reference.

b. Non-Expansion or Minimal Expansion of Engineered Cells

In some embodiments, the second manufacturing process does not include a cell expansion step or contain steps where the cells, e.g., engineered T cells of a therapeutic cell composition, are expanded to a threshold amount or concentration. In some embodiments, the resulting therapeutic cell compositions are composed of T cells that are less differentiated, less exhausted, and more potent than T cell compositions generated by other means, such as by processes that involve expanding the cells, e.g., a first manufacturing process. In some embodiments, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24(5):563-571.

In some embodiments, the second manufacturing process is a process as described in published International Application WO 2020/033927 which is incorporated herein by reference.

In some embodiments, cells, e.g., T cells, undergoing a second manufacturing process lacking an expansion step are incubated or cultivated under conditions that may result in expansion, but the incubating or cultivating conditions are not carried out for purposes of expanding the cell population. In some embodiments, the cells, e.g., T cells, of the therapeutic cell composition may have undergone expansion despite having been manufactured by the second manufacturing process that does not include an expansion step. In some embodiments, a second manufacturing process that does not include an expansion step is referred to as a non-expanded or minimally expanded process. A “non-expanded” process may also be referred to as a “minimally expanded” process. In some embodiments, a non-expanded or minimally expanded process may result in cells having undergone expansion despite the process not including a step for expansion.

In some embodiments, the non-expanded or minimally expanded process does not include a step of cultivating or incubating cells, which have been engineered (e.g. introduced or transduced) with a nucleic acid encoding a recombinant receptor, with one or more recombinant cytokines (e.g. IL-2, IL-7 and/or IL-15) under conditions to support expansion of T cells in the composition. In some embodiments, a non-expanded or minimally expanded process may include subsequent incubation of the engineered cells under conditions to support their maintenance or healthy but without inducing proliferation of cells in the composition. In some embodiments, the incubation can be carried out in a basal medium or serum free media without recombinant cytokines. In some embodiments, any further incubation may be carried out for a sufficient time to allow integration of a viral vector into the genome of the cells. In some embodiments, the incubation results in less than 2-fold increase in the number of cells in the composition compared to the starting number of cells of the input composition or the starting number of cells just prior to such incubation of the cells. In some embodiments, such incubation results in less than 1.5-fold, expansion. In some embodiments, such incubation does not result in any increase in the number of cells in the composition compared to the starting number of cells of the input composition or the starting number of cells just prior to the such incubation of the cells.

In some embodiments, the first manufacturing process includes a step for cultivating the cells under conditions for expansion, such as described in Section II.D.

In some embodiments, the cells, e.g., T cells, that are harvested for the therapeutic cell composition may have undergone an incubation or cultivating step that includes a media composition designed to reduce, suppress, minimize, or eliminate expansion of a cell population as a whole.

In some embodiments, the cells, e.g., T cells, of the therapeutic cell composition manufactured by the second manufacturing process lacking an expansion step have a greater portion and/or frequency of naïve-like cells and central memory cells than T cells of a therapeutic cell composition generated from the first manufacturing process that involves cell expansion (e.g. that include an expansion unit operation and/or include steps intended to cause expansion of cells).

In certain embodiments, the cells, e.g., T cells, of the therapeutic cell composition manufactured by the second manufacturing process lacking an expansion step include a high portion and/or frequency of naïve-like T cells or T cells that are surface positive for a marker expressed on naïve-like T cells. In certain embodiments, the cells, e.g., T cells, of the therapeutic cell compositions manufactured by the second manufacturing process lacking an expansion step have a greater portion and/or frequency of naïve-like cells than therapeutic cell compositions generated from the first manufacturing process that involves expansion (e.g. that include an expansion unit operation and/or include steps intended to cause expansion of cells).

In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+CCR7+, where the cells are CD45RA+ or CD45RA−. In certain embodiments, naïve-like T cells or the T cells that are surface positive for a marker expressed on naïve-like T cells are CD62L-CCR7+.

c. Enrichment for Naïve or Naïve-Like Cells

In some embodiments, the second manufacturing process includes a step to enrich naïve and/or naïve-like cells in the input composition from a starting biological sample. In some embodiments, the first manufacturing process does not include a step to enrich naïve and/or naïve-like cells in the input composition from a starting biological sample. In some embodiments, enriching naïve and/or naïve-like cells in the input composition can increase the number, percentage, proportion, and/or ratio of naïve and/or naïve-like cells in the therapeutic cell composition.

In some embodiments, the second manufacturing process is a process as described in published International Applications WO 2020/033927, WO 2019/113557, WO 2019/113559, and WO2020089343 which are incorporated herein by reference.

In some embodiments, the selection step to enrich for naive and/or naïve-like cells occurs prior to or after selecting T cells from a sample from the subject to produce the input composition containing CD4, CD8, or CD4 and CD8 T cells. In some embodiments, the selection step to enrich for naive and/or naïve-like cells occurs prior to selecting T cells from the sample from the subject to produce the input composition containing CD4, CD8, or CD4 and CD8 T cells. In some embodiments, the selection step to enrich for naive and/or naïve-like cells occurs after selecting T cells from the sample from the subject to produce the input composition containing CD4, CD8, or CD4 and CD8 T cells.

In some embodiments, naïve and/or naïve-like cells are selected according to any of the methods or techniques for cell selection described herein, for example in Section II-A.

In some embodiments, naïve cells are enriched by selecting for cell surface markers, e.g., selection markers. In some embodiments, the cell surface marker is CD27, CCR7, CD45RA, CD28, and the naïve T cells express one or more cell surface markers. In some embodiments, the enriched naïve cells are CD27+/CCR7+, CD27+, CCR7+, CCR7+/CD45RA+, or CD28+/CD27+.

In some embodiments, naïve-like cells are enriched by selecting for cell surface markers, e.g., selection markers. In some embodiments, the cell surface marker is CCR7, CD45RA, CD28, CD27, and the naïve-like cells express one or more cell surface markers. In some embodiments, the enriched naïve-like cells express low levels or are negative for CD25, CD45RO, CD56, CD62L, and/or KLRG1 cell surface markers.

In some embodiments, the naïve and/or naïve-like cells are CCR7+/CD45RA+, CD27+/CCR7+, CD62L−/CCR7+, CD27+/CCR7+, CD27+, CCR7+, CD28+/CD27+, or CD28+.

d. Depletion of Terminally Differentiated T Cells or T Cells with Reduced Proliferative Capacity

In some embodiments, the second manufacturing process includes a selection step to deplete terminally differentiated T cells or T cells with reduced proliferative capacity. In some embodiments, the first manufacturing process does not include a selection step to deplete terminally differentiated T cells or T cells with a reduced proliferative capacity.

In some embodiments, the second manufacturing process is a process as described in published International Application WO2020097132, which is incorporated herein by reference.

In some embodiments, the selection step to deplete terminally differentiated T cells or T cells with reduced proliferative capacity occurs prior to or after selecting T cells from the sample from the subject to produce the input composition containing CD4, CD8, or CD4 and CD8 T cells. In some embodiments, the selection step to deplete terminally differentiated T cells or T cells with reduced proliferative capacity occurs prior to selecting T cells from the sample from the subject to produce the input composition containing CD4, CD8, or CD4 and CD8 T cells. In some embodiments, the selection step to deplete terminally differentiated T cells or T cells with reduced proliferative capacity occurs after selecting T cells from the sample from the subject to produce the input composition containing CD4, CD8, or CD4 and CD8 T cells.

In some embodiments, the selection step to deplete terminally differentiated T cells or T cells with reduced proliferative capacity includes removing cells having a cell surface marker indicative of terminal differentiation or reduced proliferative capacity. In some embodiments, the cell surface marker is CD57.

In some aspects, it is contemplated that depletion of CD57+ cells (e.g. CD57+ T cells) is advantageous, such as by improving the consistency of the cell populations in downstream manufacturing processes. For example, in some embodiments, depleting CD57+ cells may deplete cells with less or reduced proliferative capacity, such that depleted compositions exhibit improved consistency in cell proliferation rates. Relatedly, improving consistency in cell proliferation rates may improve consistency in the duration required for cell populations to reach a harvest criterion during manufacturing. It is additionally observed herein that depleting CD57+ cells prior to transducing the cell population with a vector encoding a chimeric antigen receptor (CAR) may improve consistency in the CAR expression of the transduced cells.

In some aspects, pre-selecting cells from input compositions with improved proliferative capacity, e.g., by removing CD57+ T cells or screening for low amounts of CD57+ T cells in the second manufacturing process, can offer improved manufacturing process control over the number of cells used in a process to generate a cell therapy. In certain embodiments, expression of CD57 may serve as a biomarker indicating cells that exhibit delayed or poor growth. Thus, in some embodiments, the second manufacturing process includes methods that utilize one or more selection reagents or techniques to selectively remove CD57+ cells.

In some aspects, it may be advantageous to deplete CD57+ cells by negative selection (as opposed to positive selection). In some aspects, depleting CD57+ cells by negative selection, reduces the likelihood of the CD57-depleted population being contaminated by one or more reagents or solutions used in the CD57 selection step. Any suitable method of positive or negative cell selection, for example as disclosed Section II-A, is contemplated for depleting terminally differentiated T cells or T cells with reduced proliferative capacity from the input composition.

e. Cell Type Specific Manufacturing Thresholds

In some embodiments, the second manufacturing process includes a threshold number of naïve and/or naïve-like T cells, e.g. central memory T cells, in the input composition in order to initiate the manufacturing process. In some embodiments, the threshold number is not achieved by selective enrichment of the input composition for naïve and/or naïve-like T cells. In some embodiments, the presence of at least a threshold number of naïve and/or naïve-like T cells in the input composition is useful for manufacturing a therapeutic cell composition including naïve and/or naïve-like cells, some advantages of which are described above. In some embodiments, the first manufacturing process is carried out with a fixed composition of total T cells, regardless of the specific number or percentage of naïve and/or naïve-like T cells in the composition.

In some embodiments, the second manufacturing process is a process as described in published International Application WO 2019/032929 which is incorporated herein by reference.

In some embodiments, the threshold number of naïve or naïve-like cells to initiate the manufacturing process is from or from about 0.1×10⁸ to 5×10⁸, from or from about 0.1×10⁸ to 4×10⁸, from or from about 0.1×10⁸ to 2×10⁸, from or from about 0.1×10⁸ to 1×10⁸, from or from about 1×10⁸ to 5×10⁸ from or from about 1×10⁸ to 4×10⁸, from or from about 1×10⁸ to 2×10⁸, from or from about 2×10⁸ to 5×10⁸, from or from about 2×10⁸ to 4×10⁸ of the naïve-like T cells or a CD8+ or CD4+ T cell subset thereof.

In some embodiments, the threshold number of naïve or naïve-like cells to initiate the manufacturing process is at least or at least about or is or is about 0.5×10⁸, 0.75×10⁸, 1×10⁸, 1.5×10⁸, 2×10⁸, or 4×10⁸ of the naïve-like T cells or a CD8+ or CD4+ T cell subset thereof. In some embodiments, the threshold number of naïve or naïve-like cells to initiate the manufacturing process is at least or at least about or is or is about 0.5×10⁸ of the naïve-like T cells or a CD8+ or CD4+ T cell subset thereof. In some embodiments, the threshold number of naïve or naïve-like cells to initiate the manufacturing process is at least or at least about or is or is about 0.75×10⁸ of the naïve-like T cells or a CD8+ or CD4+ T cell subset thereof. In some embodiments, the threshold number of naïve or naïve-like cells to initiate the manufacturing process is at least or at least about or is or is about 1×10⁸ of the naïve-like T cells or a CD8+ or CD4+ T cell subset thereof. In some embodiments, the threshold number of naïve or naïve-like cells to initiate the manufacturing process is at least or at least about or is or is about 1.5×10⁸ of the naïve-like T cells or a CD8+ or CD4+ T cell subset thereof. In some embodiments, the threshold number of naïve or naïve-like cells to initiate the manufacturing process is at least or at least about or is or is about 2×10⁸ of the naïve-like T cells or a CD8+ or CD4+ T cell subset thereof. In some embodiments, the threshold number of naïve or naïve-like cells to initiate the manufacturing process is at least or at least about or is or is about 4×10⁸ of the naïve-like T cells or a CD8+ or CD4+ T cell subset thereof.

In some embodiments, the threshold number of naïve or naïve-like cells to initiate the manufacturing process is at least or at least about or is or is about 2×10⁸ of the naïve-like T cells or a CD8+ or CD4+ T cell subset thereof.

In some embodiments, if a threshold number of naïve and/or naïve-like cells in the input composition is not reached, the manufacturing process is not initiated. In some embodiments, if a threshold number of naïve or naïve-like cells in the input composition is not reached further samples, e.g., biological samples, may be taken from the subject, and input compositions pooled to reach a threshold to initiate manufacturing.

### II. Methods for Generating Engineered T Cells

In some embodiments, the methods of correlating and/or predicting attributes of a therapeutic cell composition provided herein can be used in connection with generating a therapeutic composition of engineered cells (e.g., output composition), such as engineered CD4+ T cells and/or engineered CD8+ T cells, that express a recombinant protein, e.g., a recombinant receptor such as a T cell receptor (TCR) or a chimeric antigen receptor (CAR). In some embodiments, the methods provided herein are used in connection with manufacturing, generating, or producing a cell therapy, and may be used in connection with additional processing steps, such as steps for the isolation, separation, selection, activation or stimulation, transduction, washing, suspension, dilution, concentration, and/or formulation of the cells. In some embodiments, the methods of generating or producing engineered cells, e.g., engineered CD4+ T cells and/or engineered CD8+ T cells, include one or more of isolating cells from a subject, preparing, processing, incubating under stimulating conditions, and/or engineering (e.g. transducing) the cells. In some embodiments, the method includes processing steps carried out in an order in which: input cells, e.g. primary cells, are first isolated, such as selected or separated, from a biological sample; input cells are incubated under stimulating conditions, engineered with vector particles, e.g., viral vector particles, to introduce a recombinant polynucleotide into the cells, e.g., by transduction or transfection; cultivating the engineered cells, e.g., transduced cells, such as to expand the cells; and collecting, harvesting, and/or filling a container with all or a portion of the cells for formulating the cells in an output composition. In some embodiments, CD4+ and CD8+ T cells are manufactured independently from one another, e.g., in separate input compositions, but the process for manufacturing includes the same processing steps. In some embodiments, CD4+ and CD8+ T cells are manufactured together, e.g., in the same input composition. In some embodiments, the attributes of the selected cells (e.g., input composition) are determined and used as input to a statistical method (e.g., pCCA or lasso regression) to identify correlated input composition and therapeutic cell composition attributes. In some embodiments, the attributes of the selected cells (e.g., input composition) are determined and used as input to a process including statistical learning models (e.g., CCA or lasso regression) to predict therapeutic cell composition attributes. In some embodiments, the statistical methods and/or learning models are used regardless of how the input compositions are processed to create the therapeutic cell composition. For example, if the input compositions are processed separately, the attributes for each input composition may be used to correlate or predict the therapeutic cell composition attributes for either or both resulting therapeutic cell compositions. In some embodiments, the cells of the generated output composition (e.g., therapeutic cell composition) are re-introduced into the same subject, before or after cryopreservation. In some embodiments, the output compositions of engineered cells (e.g., therapeutic cell composition) are suitable for use in a therapy, e.g., an autologous cell therapy). Exemplary manufacturing methods are described in published international patent application, publication no. WO 2019/089855, the contents of which are incorporated herein by reference in their entirety.

A. Samples and Cell Preparations

In particular embodiments, the provided methods are used in connection with isolating, selecting, and/or enriching cells from a biological sample to generate one or more input compositions of enriched cells, e.g., T cells. In some embodiments, the provided methods include isolation of cells or compositions thereof from biological samples, such as those obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the preparation methods include steps for freezing, e.g., cryopreserving, the cells, either before or after isolation, selection and/or enrichment and/or incubation for transduction and engineering, and/or after cultivation and/or harvesting of the engineered cells. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to or to about −80° C. at a rate of or of about 1° per minute and stored in the vapor phase of a liquid nitrogen storage tank.

In some embodiments, isolation of the cells or populations includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g. bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from 5 minutes to 6 hours or from about 5 minutes to about 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g. at or about or at least 600 rpm, 1000 rpm, or 1500 rpm or 1700 rpm), such as at an RCF at the sample or wall of the chamber or other container of from 80 g to 100 g or from about 80 g to about 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

Such separation steps can be based on positive selection, in which the cells having bound the reagents, e.g. antibody or binding partner, are retained for further use, and/or negative selection, in which the cells having not bound to the reagent, e.g., antibody or binding partner, are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the process steps further include negative and/or positive selection of the incubated and cells, such as using a system or apparatus that can perform an affinity-based selection. In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively. Multiple rounds of the same selection step, e.g., positive or negative selection step, can be performed. In certain embodiments, the positively or negatively selected fraction subjected to the process for selection, such as by repeating a positive or negative selection step. In some embodiments, selection is repeated twice, three times, four times, five times, six times, seven times, eight times, nine times or more than nine times. In certain embodiments, the same selection is performed up to five times. In certain embodiments, the same selection step is performed three times.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. In certain embodiments, one or more separation steps are repeated and/or performed more than once. In some embodiments, the positively or negatively selected fraction resulting from a separation step is subjected to the same separation step, such as by repeating the positive or negative selection step. In some embodiments, a single separation step is repeated and/or performed more than once, for example, to increase the yield of positively selected cells, to increase the purity of negatively selected cells, and/or to further remove the positively selected cells from the negatively selected fraction. In certain embodiments, one or more separation steps are performed and/or repeated two times, three times, four times, five times, six times, seven times, eight times, nine times, ten times, or more than ten times. In certain embodiments, the one or more selection steps are performed and/or repeated between one and ten times, between one and five times, or between three and five times. In certain embodiments, one or more selection steps are repeated three times.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some embodiments, such cells are selected by incubation with one or more antibody or binding partner that specifically binds to such markers. In some embodiments, the antibody or binding partner can be conjugated, such as directly or indirectly, to a solid support or matrix to effect selection, such as a magnetic bead or paramagnetic bead. For example, CD3+, CD28+ T cells can be positively selected using CD3/CD28 conjugated magnetic beads (e.g., DYNABEADS® M-450 CD3/CD28 T Cell Expander, and/or ExpACT® beads).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naïve, memory, and/or effector T cell subpopulations.

In some embodiments, CD8+ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L.

Such selections in some aspects are carried out simultaneously and in other aspects are carried out sequentially, in either order. In some aspects, the same CD4 expression-based selection step used in preparing the CD8+ T cell population or subpopulation, also is used to generate the CD4+ T cell population or sub-population, such that both the positive and negative fractions from the CD4-based separation are retained and used in subsequent steps of the methods, optionally following one or more further positive or negative selection steps. In some embodiments, the selection for the CD4+ T cell population and the selection for the CD8+ T cell population are carried out simultaneously. In some embodiments, the CD4+ T cell population and the selection for the CD8+ T cell population are carried out sequentially, in either order. In some embodiments, methods for selecting cells can include those as described in published U.S. App. No. US20170037369. In some embodiments, the selected CD4+ T cell population and the selected CD8+ T cell population may be combined subsequent to the selecting. In some aspects, the selected CD4+ T cell population and the selected CD8+ T cell population may be combined in a bioreactor bag as described herein. In some embodiments, the selected CD4+ T cell population and the selected CD8+ T cell population are separately processed, whereby the selected CD4+ T cell population is enriched in CD4+ T cells and incubated with a stimulatory reagent (e.g. anti-CD3/anti-CD28 magnetic beads), transduced with a viral vector encoding a recombinant protein (e.g. CAR) and cultivated under conditions to expand T cells and the selected CD8+ T cell population is enriched in CD8+ T cell and incubated with a stimulatory reagent (e.g. anti-CD3/anti-CD28 magnetic beads), transduced with a viral vector encoding a recombinant protein (e.g. CAR), such as the same recombinant protein as for engineering of the CD4+ T cells from the same donor, and cultivated under conditions to expand T cells, such as in accord with the provided methods.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In a particular example, a sample of PBMCs or other white blood cell sample is subjected to selection of CD4+ T cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells may be sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, or CD4+ T cells. In some embodiments, central memory CD4+ T cells are CD62L+ and CD45RO+. In some embodiments, effector CD4+ T cells are CD62L− and CD45RO−.

In one example, to enrich for CD4+ T cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the incubated sample or composition of cells to be separated is incubated with a selection reagent containing small, magnetizable or magnetically responsive material, such as magnetically responsive particles or microparticles, such as paramagnetic beads (e.g., such as Dynalbeads or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. Many well-known magnetically responsive materials for use in magnetic separation methods are known, e.g., those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 also may be used.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some aspects, separation is achieved in a procedure in which the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotech, Auburn, Calif.). Magnetic Activated Cell Sorting (MACS), e.g., CliniMACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In some embodiments, attributes of the one or more input compositions are assessed, for example as described in Sections I-A and I-A-1. In some embodiments, the attributes are cell phenotypes. In some embodiments, the attributes are first attributes. In some embodiments, the attributes, e.g., cell phenotypes, are quantified to provide a number, percentage, proportion, and/or ratio of cells having an attribute in the input composition. In some embodiments, the attributes are used as input to a process including a statistical method, for example as described herein, to identify correlations between attributes of the input composition and the resulting therapeutic cell composition. In some embodiments, the attributes are used as input to a process including a statistical learning model, for example as described herein, to predict therapeutic cell composition attributes. In some embodiments, the predicted therapeutic cell attributes are used to select a manufacturing process for generating the therapeutic cell composition. In some embodiments, for example if desired attributes are predicted for the therapeutic cell composition, the manufacturing process may proceed according to the steps described in Sections II-B to II-E below. In some embodiments, for example if desired attributes are not predicted for the therapeutic cell composition, a manufacturing process including one or more step described in Section I-C-4 may be used to generate the therapeutic cell composition.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In particular embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

In some embodiments, the one or more input compositions of enriched T cells are frozen, e.g., cryopreserved and/or cryofrozen, after isolation, selection and/or enrichment. In some embodiments, the one or more input compositions of frozen e.g., cryopreserved and/or cryofrozen, prior to any steps of incubating, activating, stimulating, engineering, transducing, transfecting, cultivating, expanding, harvesting, and/or formulating the composition of cells. In particular embodiments, the one or more cryofrozen input compositions are stored, e.g., at or at about −80° C., for between 12 hours and 7 days, between 24 hours and 120 hours, or between 2 days and 5 days. In particular embodiments, the one or more cryofrozen input compositions are stored at or at about −80° C., for an amount of time of less than 10 days, 9 days, 8 days, 7 days, 6 days, or 5 days, 4 days, 3 days, 2 days, or 1 day. In some embodiments, the one or more cryofrozen input compositions are stored at or at about −80° C., for or for about 1 day, 2 days, 3 days, 4 days, 5 days, or 6 days.

B. Activation and Stimulation of Cells

In some embodiments, the provided methods are used in connection with incubating cells under stimulating conditions. In some embodiments, the stimulating conditions include conditions that activate or stimulate, and/or are capable of activating or stimulating a signal in the cell, e.g., a CD4+ T cell or CD8+ T cell, such as a signal generated from a TCR and/or a coreceptor. In some embodiments, the stimulating conditions include one or more steps of culturing, cultivating, incubating, activating, propagating the cells with and/or in the presence of a stimulatory reagent, e.g., a reagent that activates or stimulates, and/or is capable of activating or stimulating a signal in the cell. In some embodiments, the stimulatory reagent stimulates and/or activates a TCR and/or a coreceptor. In particular embodiments, the stimulatory reagent is a reagent described in Section II-B-1.

In certain embodiments, one or more compositions of enriched T cells are incubated under stimulating conditions prior to genetically engineering the cells, e.g., transfecting and/or transducing the cell such as by a technique provided in Section II-C. In particular embodiments, one or more compositions of enriched T cells are incubated under stimulating conditions after the one or more compositions have been isolated, selected, enriched, or obtained from a biological sample. In particular embodiments, the one or more compositions are input compositions. In particular embodiments, the one or more input compositions have been previously cryofrozen and stored, and are thawed prior to the incubation.

In certain embodiments, the one or more compositions of enriched T cells are or include two separate compositions, e.g., separate input compositions, of enriched T cells. In particular embodiments, two separate compositions of enriched T cells, e.g., two separate compositions of enriched T cells selected, isolated, and/or enriched from the same biological sample, are separately incubated under stimulating conditions. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells are separately incubated under stimulating conditions.

In some embodiments, a single composition of enriched T cells is incubated under stimulating conditions. In certain embodiments, the single composition is a composition of enriched CD4+ T cells. In some embodiments, the single composition is a composition of enriched CD4+ and CD8+ T cells that have been combined from separate compositions prior to the incubation.

In some embodiments, the composition of enriched CD4+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are incubated under stimulating conditions. In certain embodiments, separate stimulated compositions of enriched CD4+ and enriched CD8+ T cells are combined into a single composition after the incubation has been performed and/or completed. In some embodiments, separate stimulated compositions of stimulated CD4+ and stimulated CD8+ T cells are separately processed after the incubation has been performed and/or completed, whereby the stimulated CD4+ T cell population (e.g. incubated with stimulatory an anti-CD3/anti-CD28 magnetic bead stimulatory reagent) is transduced with a viral vector encoding a recombinant protein (e.g. CAR) and cultivated under conditions to expand T cells and the stimulated CD8+ T cell population (e.g. incubated with stimulatory an anti-CD3/anti-CD28 magnetic bead stimulatory reagent) is transduced with a viral vector encoding a recombinant protein (e.g. CAR), such as the same recombinant protein as for engineering of the CD4+ T cells from the same donor, and cultivated under conditions to expand T cells, such as in accord with the provided methods.

In some embodiments, the incubation under stimulating conditions can include culture, cultivation, stimulation, activation, propagation, including by incubation in the presence of stimulating conditions, for example, conditions designed to induce proliferation, expansion, activation, and/or survival of cells in the population, to mimic antigen exposure, and/or to prime the cells for genetic engineering, such as for the introduction of a recombinant antigen receptor. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, the stimulation and/or incubation under stimulating conditions is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the cells, e.g., T cells, compositions of cells, and/or cell populations, such as CD4⁺ and CD8⁺ T cells or compositions, populations, or subpopulations thereof, are expanded by adding to the culture-initiating composition feeder cells, such as non-dividing peripheral blood mononuclear cells (PBMCs) (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. In some embodiments, a temperature shift is effected during culture, such as from 37 degrees Celsius to 35 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, populations of CD4⁺ and CD8⁺ that are antigen specific can be obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen. Naive T cells may also be used.

In particular embodiments, the stimulating conditions include incubating, culturing, and/or cultivating the cells with a stimulatory reagent. In particular embodiments, the stimulatory reagent is a reagent described in Section I-B-1. In certain embodiments, the stimulatory reagent contains or includes a bead. An exemplary stimulatory reagent is or includes anti-CD3/anti-CD28 magnetic beads. In certain embodiments, the start and/or initiation of the incubation, culturing, and/or cultivating cells under stimulating conditions occurs when the cells come into contact with and/or are incubated with the stimulatory reagent. In particular embodiments, the cells are incubated prior to, during, and/or subsequent to genetically engineering the cells, e.g., introducing a recombinant polynucleotide into the cell such as by transduction or transfection.

In some embodiments, the composition of enriched T cells are incubated at a ratio of stimulatory reagent and/or beads, e.g. anti-CD3/anti-CD28 magnetic beads, to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of stimulatory reagent and/or beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

In particular embodiments, incubating the cells at a ratio of less than 3:1 or less than 3 stimulatory reagents, e.g. anti-CD3/anti-CD28 magnetic beads. per cell, such as a ratio of 1:1, reduces the amount of cell death that occurs during the incubation, e.g., such as by activation-induced cell death. In some embodiments, the cells are incubated with the stimulatory reagent, e.g. anti-CD3/anti-CD28 magnetic beads, at a ratio of beads to cells of less than 3 (or 3:1 or less than 3 beads per cell). In particular embodiments, incubating the cells at a ratio of less than 3:1 or less than 3 beads per cell, such as a ratio of 1:1, reduces the amount of cell death that occurs during the incubation, e.g., such as by activation-induced cell death.

In particular embodiments, the composition of enriched T cells is incubated with the stimulatory reagent, e.g. anti-CD3/anti-CD28 magnetic beads, at a ratio of less than 3:1 stimulatory reagents and/or beads per cell, such as a ratio of 1:1, and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programed cell death, or apoptosis, during or at least 1 day, 2 days, 3 days, 4 days, 5 days, 6 days, 7 days, or more than 7 days after the incubation is complete. In particular embodiments, the composition of enriched T cells is incubated with the stimulatory reagent at a ratio of less than 3:1 stimulatory reagents and/or beads per cell, e.g., a ratio of 1:1, and less than 50%, less than 40%, less than 30%, less than 25%, less than 20%, less than 15%, less than 10%, less than 5%, less than 1%, less than 0.1% or less than 0.01% of the cells undergo activation induced cell death during the incubation.

In certain embodiments, the composition of enriched T cells is incubated with the stimulatory reagent, e.g. anti-CD3/anti-CD28 magnetic beads, at a ratio of less than 3:1 beads per cell, e.g., a ratio of 1:1, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing an exemplary and/or alternative process where the composition of enriched T cells in incubated with the stimulatory reagent at a ratio of 3:1 or greater.

In some embodiments, the composition of enriched T cells incubated with the stimulatory reagent comprises from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the composition of enriched T cells incubated with the stimulatory reagent comprises about 0.5×10⁶ cells/mL, 1×10⁶ cells/mL, 1.5×10⁶ cells/mL, 2×10⁶ cells/mL, 2.5×10⁶ cells/mL, 3×10⁶ cells/mL, 3.5×10⁶ cells/mL, 4×10⁶ cells/mL, 4.5×10⁶ cells/mL, 5×10⁶ cells/mL, 5.5×10⁶ cells/mL, 6×10⁶ cells/mL, 6.5×10⁶ cells/mL, 7×10⁶ cells/mL, 7.5×10⁶ cells/mL, 8×10⁶ cells/mL, 8.5×10⁶ cells/mL, 9×10⁶ cells/mL, 9.5×10⁶ cells/mL, or 10×10⁶ cells/mL, such as about 2.4×10⁶ cells/mL.

In some embodiments, the composition of enriched T cells is incubated with the stimulatory reagent at a temperature from about 25 to about 38° C., such as from about 30 to about 37° C., for example at or about 37° C.±2° C. In some embodiments, the composition of enriched T cells is incubated with the stimulatory reagent at a CO₂ level from about 2.5% to about 7.5%, such as from about 4% to about 6%, for example at or about 5%±0.5%. In some embodiments, the composition of enriched T cells is incubated with the stimulatory reagent at a temperature of or about 37° C. and/or at a CO₂ level of or about 5%.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes IL-2. In some embodiments, the stimulating conditions include incubating composition of enriched T cells, such as enriched CD4+ T cells or enriched CD8+ T cells, in the presence of a stimulatory reagent, e.g. anti-CD3/anti-CD28 magnetic beads, as described and in the presence or one or more recombinant cytokines.

In particular embodiments, the composition of enriched CD4+ T cells are incubated with IL-2, e.g., recombinant IL-2. Without wishing to be bound by theory, particular embodiments contemplate that CD4+ T cells that are obtained from some subjects do not produce, or do not sufficiently produce, IL-2 in amounts that allow for growth, division, and expansion throughout the process for generating a composition of output cells, e.g., engineered cells suitable for use in cell therapy. In some embodiments, incubating a composition of enriched CD4+ T cells under stimulating conditions in the presence of recombinant IL-2 increases the probability or likelihood that the CD4+ T cells of the composition will continue to survive, grow, expand, and/or activate during the incubation step and throughout the process. In some embodiments, incubating the composition of enriched CD4+ T cells in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold as compared to an alternative and/or exemplary method that does not incubate the composition of enriched CD4+ T cells in the presence of recombinant IL-2.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength e.g., WHO 1st International Standard for Human IL-2, 86/504. International Units are the only recognized and standardized method to report biological activity units that are published and are derived from an international collaborative research effort. In particular embodiments, the IU for composition, sample, or source of a cytokine may be obtained through product comparison testing with an analogous WHO standard product. For example, in some embodiments, the IU/mg of a composition, sample, or source of human recombinant IL-2, IL-7, or IL-15 is compared to the WHO standard IL-2 product (NIBSC code: 86/500), the WHO standard IL-17 product (NIBSC code: 90/530) and the WHO standard IL-15 product (NIBSC code: 95/554), respectively.

In some embodiments, the biological activity in IU/mg is equivalent to (ED₅₀ in ng/ml)⁻¹×10⁶. In particular embodiments, the ED₅₀ of recombinant human IL-2 or IL-15 is equivalent to the concentration required for the half-maximal stimulation of cell proliferation (XTT cleavage) with CTLL-2 cells. In certain embodiments, the ED₅₀ of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94; hereby incorporated by reference in their entirety.

In particular embodiments, a composition of enriched CD8+ T cells is incubated under stimulating conditions in the presence of IL-2 and/or IL-15. In certain embodiments, a composition of enriched CD4+ T cells is incubated under stimulating conditions in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In some aspects, the incubation of the enriched T cell composition also includes the presence of a stimulatory reagent, e.g. anti-CD3/anti-CD28 magnetic beads.

In some embodiments, the cells are incubated with a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is incubated with IL-2, e.g., human recombinant IL-2, at a concentration between 1 IU/ml and 200 IU/ml, between 10 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is incubated with recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml. In some embodiments, the composition of enriched T cells is incubated in the presence of or of about 85 IU/ml recombinant IL-2. In some embodiments, the composition incubated with recombinant IL-2 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-2 may also be incubated with recombinant IL-7 and/or recombinant IL-15, such as in amounts described. In some embodiments, an enriched CD8+ T cell composition incubated with recombinant IL-2 may also be incubated with recombinant IL-15, such as in amounts described.

In some embodiments, a composition of enriched T cells is incubated with recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 IU/ml, or between 550 IU/ml and 650 IU/ml. In particular embodiments, the composition of enriched T cells is incubated with recombinant IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is incubated in the presence of or of about 600 IU/ml of recombinant IL-7. In some embodiments, the composition incubated with recombinant IL-7 is enriched for a population of T cells, e.g., CD4+ T cells. In some embodiments, an enriched CD4+ T cell composition incubated with recombinant IL-7 may also be incubated with recombinant IL-2 and/or recombinant IL-15, such as in amounts described. In particular embodiments, the composition of enriched T cells is enriched for CD4+ T cells, where CD8+ T cells are not enriched for and/or where CD8+ T cells are negatively selected for or depleted from the composition. In some embodiments, an enriched CD8+ T cell composition is not incubated with recombinant IL-7.

In some embodiments, a composition of enriched T cells is incubated with recombinant IL-15, e.g., human recombinant IL-15, at a concentration between 0.1 IU/ml and 100 IU/ml, between 1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is incubated with recombinant IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is incubated in or in about 10 IU/ml of recombinant IL-15. In some embodiments, the composition incubated with recombinant IL-15 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-15 may also be incubated with recombinant IL-7 and/or recombinant IL-2, such as in amounts described. In some embodiments, an enriched CD8+ T cell composition incubated with recombinant IL-15 may also be incubated with recombinant IL-2, such as in amounts described.

In particular embodiments, the cells, such as enriched CD4+ T cells and/or enriched CD8+ T cells, are incubated with the stimulatory reagent in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, lutein, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthathione, cystamine and cystathionine, and/or glycine-glycine-histidine. In some aspects, the incubation of the enriched T cell composition, such as enriched CD4+ T cells and/or enriched CD8+ T cells, with an antioxidant also includes the presence of a stimulatory reagent, e.g. anti-CD3/anti-CD28 magnetic beads, and one or more recombinant cytokines, such as described.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetylcysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, incubating the cells, such as enriched CD4+ T cells and/or enriched CD8+ T cells, under stimulating conditions includes incubating the cells in the presence of one or more antioxidants. In particular embodiments, the cells are stimulated with the stimulatory reagent in the presence of one or more antioxidants. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the one or more antioxidants is or includes N-acetyl cysteine (NAC). In some embodiments, incubating the cells, such as enriched CD4+ T cells and/or enriched CD8+ T cells, under stimulating conditions includes incubating the cells in the presence of NAC. In particular embodiments, the cells are stimulated with the stimulatory reagent in the presence of NAC. In some embodiments, the cells are incubated in the presence of between 1 ng/ml and 100 ng/ml, between 10 ng/ml and 1 pg/ml, between 100 ng/ml and 10 μg/ml, between 1 μg/ml and 100 μg/ml, between 10 μg/ml and 1 mg/ml, between 100 μg/ml and 1 mg/ml, between 1-500 μg/ml and 2 mg/ml, 500 μg/ml and 5 mg/ml, between 1 mg/ml and 10 mg/ml, or between 1 mg/ml and 100 mg/ml of NAC. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are incubated with or with about 0.8 mg/ml.

In particular embodiments, incubating the composition of enriched T cells, such as enriched CD4+ T cells and/or enriched CD8+ T cells, in the presence of one or more antioxidants, e.g., NAC, reduces the activation in the cells as compared to cells that are incubated in alternative and/or exemplary processes without the presence of antioxidants. In certain embodiments, the reduced activation is measured by the expression of one or more activation markers in the cell. In certain embodiments, markers of activation include, but are not limited to, increased intracellular complexity (e.g. as determined by measuring side scatter (SSC), increased cell size (e.g. as determined by measuring cell diameter and/or forward scatter (FSC), increased expression of CD27, and/or decreased expression of CD25. In some embodiments, the cells of the composition have negative, reduced, or low expression and/or degree of markers of activation when examined during or after the incubation, engineering, transduction, transfection, expansion, or formulation, or during or after any stage of the process occurring after the incubation. In some embodiments the cells of the composition have negative, reduced, or low expression and/or degree of markers of activation after the process is completed. In particular embodiments, the cells of the output composition have negative, reduced, or low expression and/or degree of markers of activation.

In some embodiments, flow cytometry is used to determine relative size of cells. In particular embodiments, the FSC and SSC parameters are used to analyze cells and distinguish the cells from one another based off of size and internal complexity. In particular embodiments, a particle or bead of a known size can be measured as a standard to determine the actual size of cells. In some embodiments, flow cytometry is used in combination with a stain, e.g., a labeled antibody, to measure or quantify the expression of a surface protein, such as a marker of activation, e.g., CD25 or CD27.

In some embodiments, the composition of enriched T cells, such as enriched CD4+ T cells and/or enriched CD8+ T cells, is incubated in the presence of one or more antioxidants e.g., NAC, and the cell diameter reduced by at least 0.25 μm, 0.5 μm, 0.75 μm, 1.0 μm, 1.5 μm, 2 μm, 2.5 μm, 3 μm, 3.5 μm, 4 μm, 4.5 μm, 5 μm, or more than 5 μm as compared to cells incubated in an alternative and/or exemplary process where the incubation is not performed in the presence of an antioxidant. In particular embodiments, the composition of enriched T cells is incubated in the presence of one or more antioxidants e.g., NAC, and the cell size, as measured by the FSC is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In some embodiments, the composition of enriched T cells, such as enriched CD4+ T cells and/or enriched CD8+ T cells, is incubated in the presence of one or more antioxidants e.g., NAC, and the intracellular complexity, as measured by the SSC, is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In particular embodiments, the composition of enriched T cells, such as enriched CD4+ T cells and/or enriched CD8+ T cells, is incubated in the presence of one or more antioxidants e.g., NAC, and the expression of CD27, e.g., as measured by the flow cytometry, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In certain embodiments, the composition of enriched T cells, such as enriched CD4+ T cells and/or enriched CD8+ T cells, is incubated in the presence of one or more antioxidants, e.g., NAC, and the expression of CD25, e.g., as measured by the flow cytometry, is increased by at least 1%, at least 5%, at least 10%, at least 20%, at least 30%, at least 40%, at least 50%, at least 60%, at least 70%, at least 75%, at least 80%, at least 85%, at least 90%, at least 100%, at least 150%, at least 1-fold, at least 2-fold, at least 3-fold, at least 4-fold, at least 5-fold, at least 10-fold, at least 25-Fold, at least 50-fold, or at least 100-fold as compared to cells incubated in an alternative and/or exemplary process where the incubation is not performed in the presence of an antioxidant.

In particular embodiments, incubating the composition of enriched T cells, such as enriched CD4+ T cells and/or enriched CD8+ T cells, in the presence of one or more antioxidants, e.g., NAC, increases the expansion, e.g., during the incubation or cultivation step or stage as described in Section I-D. In some embodiments, a composition of enriched cells achieves a 2-fold, a 2.5 fold, a 3 fold, a 3.5 fold, a 4 fold, a 4.5 fold a 5 fold, a 6 fold, a 7 fold, an 8 fold, a nine fold, a 10-fold, or greater than a 10 fold expansion within 14 days, 12 days, 10 days, 9 days, 8 days, 7 days, 6 days, 5 days, 4 days, or within 3 days of the start of the cultivation. In some embodiments, the composition of enriched T cells is incubated in the presence of one or more antioxidants and the cells of the compositions undergo at least 10%, at least a 20%, at least a 30%, at least a 40%, at least a 50%, at least a 60%, at least a 70%, at least a 75%, at least an 80%, at least an 85%, at least a 90%, at least a 100%, at least a 150%, at least a 1-fold, at least a 2-fold, at least a 3-fold, at least a 4-fold, at least a 5-fold, at least a 10-fold faster rate of expansion during the cultivation than cultivated cells that were incubated in an alternative and/or exemplary process where the incubation is not performed in the presence of an antioxidant.

In particular embodiments, incubating the composition of enriched cells, such as enriched CD4+ T cells and/or enriched CD8+ T cells, in the presence of one or more antioxidants, e.g., NAC, reduces the amount of cell death, e.g., by apoptosis. In some embodiments, the composition of enriched T cells is incubated in the presence of a one or more antioxidants, e.g., NAC, and at least 50%, at least 60%, at least 70%, at least 75%, at least 80%, at least 85%, at least 90%, at least 95%, at least 99%, or at least 99.9% of the cells survive, e.g., do not undergo apoptosis, during or at least 1 day, 2 days, 3 days, 4 days, 5 days, 6 days, 7 days, or more than 7 days after the incubation is complete. In some embodiments, the composition is incubated in the presence of one or more antioxidants, e.g., NAC, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing an exemplary and/or alternative process where cells are not incubated in the presence or one or more antioxidants.

In particular embodiments, the composition of enriched T cells, such as enriched CD4+ T cells and/or enriched CD8+ T cells, is incubated in the presence of one or more antioxidants e.g., NAC, and caspase expression, e.g., caspase 3 expression, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In some embodiments, the compositions or cells, such as enriched CD4+ T cells and/or enriched CD8+ T cells, are incubated in the presence of stimulating conditions or a stimulatory agent, such as described. Such conditions include those designed to induce proliferation, expansion, activation, and/or survival of cells in the population, to mimic antigen exposure, and/or to prime the cells for genetic engineering, such as for the introduction of a recombinant antigen receptor. Exemplary stimulatory reagents, such as anti-CD3/anti-CD28 magnetic beads, are described below. The incubation with the stimulatory reagent may also be carried out in the presence of one or more stimulatory cytokine, such as in the presence of one or more of recombinant IL-2, recombinant IL-7 and/or recombinant IL-15 and/or in the presence of at least one antioxidant such as NAC, such as described above. In some embodiments, a composition of enriched CD4+ T cells are incubated under stimulatory conditions with a stimulatory agent, recombinant IL-2, recombinant IL-7, recombinant IL-15 and NAC, such as in amounts as described. In some embodiments, a composition of enriched CD8+ T cells are incubated under stimulatory conditions with a stimulatory agent, recombinant IL-2, recombinant IL-15 and NAC, such as in amounts as described.

In some embodiments, the conditions for stimulation and/or activation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g. in a tube or bag with periodic shaking or rotation. In some embodiments, the incubation is performed with the addition of an incubation buffer to the cells and stimulating agent to achieve a target volume with incubation of the reagent of, for example, about 10 mL to about 200 mL, or about 20 mL to about 125 mL, such as at least or about at least or about 10 mL, 20 mL, 30 mL, 40 mL, 50 mL, 60 mL, 70 mL, 80 mL, 90 mL, 100 mL, 105 mL, 110 mL, 115 mL, 120 mL, 125 mL, 130 mL, 135 mL, 140 mL, 145 mL, 150 mL, 160 mL, 170 mL, 180 mL, 190 mL, or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g. at or about or at least 600 rpm, 1000 rpm, or 1500 rpm or 1700 rpm), such as at an RCF at the sample or wall of the chamber or other container of from 80 g to 100 g or from about 80 g to about 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours, 18 hours and 30 hours, or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the cells are cultured, cultivated, and/or incubated under stimulating conditions prior to and/or during a step for introducing a polynucleotide, e.g., a polynucleotide encoding a recombinant receptor, to the cells, e.g., by transduction and/or transfection, such as described by Section I-C. In certain embodiments the cells are cultured, cultivated, and/or incubated under stimulating conditions for an amount of time between 30 minutes and 2 hours, between 1 hour and 8 hours, between 1 hour and 6 hours, between 6 hours and 12 hours, between 12 hours and 18 hours, between 16 hours and 24 hours, between 12 hours and 36 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days prior to the genetic engineering. In some embodiments, the cells are incubated for or for about 2 days prior to the engineering.

In certain embodiments, the cells are incubated with and/or in the presence of the stimulatory reagent prior to and/or during genetically engineering the cells. In certain embodiments the cells are incubated with and/or in the presence of the stimulatory reagent for an amount of time between 12 hours and 36 hours, between 24 hours and 48 hours, between 24 hours and 72 hours, between 42 hours and 54 hours, between 60 hours and 120 hours between 96 hours and 120 hours, between 90 hours and between 2 days and 7 days, between 3 days and 8 days, between 1 day and 8 days, between 4 days and 6 days, or between 4 days and 5 days. In particular embodiments, the cells are cultured, cultivated, and/or incubated under stimulating conditions prior to and/or during genetically engineering the cells for an amount of time of less than 10 days, 9 days, 8 days, 7 days, 6 days, or 5 days, 4 days, or for an amount of time less than 168 hours, 162 hours, 156 hours, 144 hours, 138 hours, 132 hours, 120 hours, 114 hours, 108 hours, 102 hours, or 96 hours. In particular embodiments, the cells are incubated with and/or in the presence of the stimulatory reagent for or for about 4 days, 5 days, 6 days, or 7 days. In some embodiments, the cells are incubated with and/or in the presence of the stimulatory reagent for or for about 4 days. In particular embodiments, the cells are incubated with and/or in the presence of the stimulatory reagent for or for about 5 days. In certain embodiments, the cells are incubated with and/or in the presence of the stimulatory reagent for less than 7 days.

In some embodiments, incubating the cells under stimulating conditions includes incubating the cells with a stimulatory reagent that is described in Section I-B-1. In some embodiments, the stimulatory reagent contains or includes a bead, such as a paramagnetic bead, and the cells are incubated with the stimulatory reagent at a ratio of less than 3:1 (beads:cells), such as a ratio of 1:1. In particular embodiments, the cells are incubated with the stimulatory reagent in the presence of one or more cytokines and/or one or more antioxidants. In some embodiments, a composition of enriched CD4+ T cells is incubated with the stimulatory reagent at a ratio of 1:1 (beads:cells) in the presence of recombinant IL-2, IL-7, IL-15, and NAC. In certain embodiments, a composition of enriched CD8+ T cells is incubated with the stimulatory reagent at a ratio of 1:1 (beads:cells) in the presence of recombinant IL-2, IL-15, and NAC. In some embodiments, the stimulatory reagent is removed and/or separated from the cells at, within, or within about 6 days, 5 days, or 4 days from the start or initiation of the incubation, e.g., from the time the stimulatory reagent is added to or contacted with the cells.

1. Stimulatory Reagents

In some embodiments, incubating a composition of enriched cells under stimulating conditions is or includes incubating and/or contacting the composition of enriched cells with a stimulatory reagent that is capable of activating and/or expanding T cells. In some embodiments, the stimulatory reagent is capable of stimulating and/or activating one or more signals in the cells. In some embodiments, the one or more signals are mediated by a receptor. In particular embodiments, the one or more signals are or are associated with a change in signal transduction and/or a level or amount of secondary messengers, e.g., cAMP and/or intracellular calcium, a change in the amount, cellular localization, confirmation, phosphorylation, ubiquitination, and/or truncation of one or more cellular proteins, and/or a change in a cellular activity, e.g., transcription, translation, protein degradation, cellular morphology, activation state, and/or cell division. In particular embodiments, the stimulatory reagent activates and/or is capable of activating one or more intracellular signaling domains of one or more components of a TCR complex and/or one or more intracellular signaling domains of one or more costimulatory molecules.

In certain embodiments, the stimulatory reagent contains a particle, e.g., a bead, that is conjugated or linked to one or more agents, e.g., biomolecules, that are capable of activating and/or expanding cells, e.g., T cells. In some embodiments, the one or more agents are bound to a bead. In some embodiments, the bead is biocompatible, i.e., composed of a material that is suitable for biological use. In some embodiments, the beads are non-toxic to cultured cells, e.g., cultured T cells. In some embodiments, the beads may be any particles which are capable of attaching agents in a manner that permits an interaction between the agent and a cell.

In some embodiments, a stimulatory reagent contains one or more agents that are capable of activating and/or expanding cells, e.g., T cells, that are bound to or otherwise attached to a bead, for example to the surface of the bead. In certain embodiments, the bead is a non-cell particle. In particular embodiments, the bead may include a colloidal particle, a microsphere, nanoparticle, a magnetic bead, or the like. In some embodiments the beads are agarose beads. In certain embodiments, the beads are sepharose beads.

In particular embodiments, the stimulatory reagent contains beads that are monodisperse. In certain embodiments, beads that are monodisperse comprise size dispersions having a diameter standard deviation of less than 5% from each other.

In some embodiments, the bead contains one or more agents, such as an agent that is coupled, conjugated, or linked (directly or indirectly) to the surface of the bead. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of methods known and available in the art. The attachment may be covalent, noncovalent, electrostatic, or hydrophobic and may be accomplished by a variety of attachment means, including for example, a chemical means, a mechanical means, or an enzymatic means. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the stimulatory reagent contains a bead and one or more agents that directly interact with a macromolecule on the surface of a cell. In certain embodiments, the bead (e.g., a paramagnetic bead) interacts with a cell via one or more agents (e.g., an antibody) specific for one or more macromolecules on the cell (e.g., one or more cell surface proteins). In certain embodiments, the bead (e.g., a paramagnetic bead) is labeled with a first agent described herein, such as a primary antibody (e.g., an anti-biotin antibody) or other biomolecule, and then a second agent, such as a secondary antibody (e.g., a biotinylated anti-CD3 antibody) or other second biomolecule (e.g., streptavidin), is added, whereby the secondary antibody or other second biomolecule specifically binds to such primary antibodies or other biomolecule on the particle.

In some embodiments, the stimulatory reagent contains one or more agents (e.g. antibody) that is attached to a bead (e.g., a paramagnetic bead) and specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD1 1a (LFA-1), CD62L (L-selectin), CD29/CD49d (VLA-4), Notch ligand (e.g. Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, an agent (e.g. antibody) attached to the bead specifically binds to one or more of the following macromolecules on a cell (e.g. a T cell): CD28, CD62L, CCR7, CD27, CD127, CD3, CD4, CD8, CD45RA, and/or CD45RO.

In some embodiments, one or more of the agents attached to the bead is an antibody. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the stimulatory reagent is an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species). In some embodiments, the agent is an antibody that binds to and/or recognizes one or more components of a T cell receptor. In particular embodiments, the agent is an anti-CD3 antibody. In certain embodiments, the agent is an antibody that binds to and/or recognizes a co-receptor. In some embodiments, the stimulatory reagent comprises an anti-CD28 antibody. In some embodiments, the bead has a diameter of greater than about 0.001 μm, greater than about 0.01 μm, greater than about 0.1 μm, greater than about 1.0 μm, greater than about 10 μm, greater than about 50 μm, greater than about 100 μm or greater than about 1000 μm and no more than about 1500 μm. In some embodiments, the bead has a diameter of about 1.0 μm to about 500 μm, about 1.0 μm to about 150 μm, about 1.0 μm to about 30 μm, about 1.0 μm to about 10 μm, about 1.0 μm to about 5.0 μm, about 2.0 μm to about 5.0 μm, or about 3.0 μm to about 5.0 μm. In some embodiments, the bead has a diameter of about 3 μm to about 5 μm. In some embodiments, the bead has a diameter of at least or at least about or about 0.001 μm, 0.01 μm, 0.1 μm, 0.5 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 12 μm, 14 μm, 16 μm, 18 μm or 20 μm. In certain embodiments, the bead has a diameter of or about 4.5 μm. In certain embodiments, the bead has a diameter of or about 2.8 μm.

In some embodiments, the beads have a density of greater than 0.001 g/cm³, greater than 0.01 g/cm³, greater than 0.05 g/cm³, greater than 0.1 g/cm³, greater than 0.5 g/cm³, greater than 0.6 g/cm³, greater than 0.7 g/cm³, greater than 0.8 g/cm³, greater than 0.9 g/cm³, greater than 1 g/cm³, greater than 1.1 g/cm³, greater than 1.2 g/cm³, greater than 1.3 g/cm³, greater than 1.4 g/cm³, greater than 1.5 g/cm³, greater than 2 g/cm³, greater than 3 g/cm³, greater than 4 g/cm³, or greater than 5 g/cm³. In some embodiments, the beads have a density of between about 0.001 g/cm³ and about 100 g/cm³, about 0.01 g/cm³ and about 50 g/cm³, about 0.1 g/cm³ and about 10 g/cm³, about 0.1 g/cm³ and about 0.5 g/cm³, about 0.5 g/cm³ and about 1 g/cm³, about 0.5 g/cm³ and about 1.5 g/cm³, about 1 g/cm³ and about 1.5 g/cm³, about 1 g/cm³ and about 2 g/cm³, or about 1 g/cm³ and about 5 g/cm³. In some embodiments, the beads have a density of about 0.5 g/cm³, about 0.5 g/cm³, about 0.6 g/cm³, about 0.7 g/cm³, about 0.8 g/cm³, about 0.9 g/cm³, about 1.0 g/cm³, about 1.1 g/cm³, about 1.2 g/cm³, about 1.3 g/cm³, about 1.4 g/cm³, about 1.5 g/cm³, about 1.6 g/cm³, about 1.7 g/cm³, about 1.8 g/cm³, about 1.9 g/cm³, or about 2.0 g/cm³. In certain embodiments, the beads have a density of about 1.6 g/cm³. In particular embodiments, the beads or particles have a density of about 1.5 g/cm³. In certain embodiments, the particles have a density of about 1.3 g/cm³.

In certain embodiments, a plurality of the beads has a uniform density. In certain embodiments, a uniform density comprises a density standard deviation of less than 10%, less than 5%, or less than 1% of the mean bead density.

In some embodiments, the beads have a surface area of between about 0.001 m² per each gram of particles (m²/g) to about 1,000 m²/g, about 0.010 m²/g to about 100 m²/g, about 0.1 m²/g to about 10 m²/g, about 0.1 m²/g to about 1 m²/g, about 1 m²/g to about 10 m²/g, about 10 m²/g to about 100 m²/g, about 0.5 m²/g to about 20 m²/g, about 0.5 m²/g to about 5 m²/g, or about 1 m²/g to about 4 m²/g. In some embodiments, the particles or beads have a surface area of about 1 m²/g to about 4 m²/g.

In some embodiments, the bead contains at least one material at or near the bead surface that can be coupled, linked, or conjugated to an agent. In some embodiments, the bead is surface functionalized, i.e. comprises functional groups that are capable of forming a covalent bond with a binding molecule, e.g., a polynucleotide or a polypeptide. In particular embodiments, the bead comprises surface-exposed carboxyl, amino, hydroxyl, tosyl, epoxy, and/or chloromethyl groups. In particular embodiments, the beads comprise surface exposed agarose and/or sepharose. In certain embodiments, the bead surface comprises attached stimulatory reagents that can bind or attach binding molecules. In particular embodiments, the biomolecules are polypeptides. In some embodiments, the beads comprise surface exposed protein A, protein G, or biotin.

In some embodiments, the bead reacts in a magnetic field. In some embodiments, the bead is a magnetic bead. In some embodiments, the magnetic bead is paramagnetic. In particular embodiments, the magnetic bead is superparamagnetic. In certain embodiments, the beads do not display any magnetic properties unless they are exposed to a magnetic field.

In particular embodiments, the bead comprises a magnetic core, a paramagnetic core, or a superparamagnetic core. In some embodiments, the magnetic core contains a metal. In some embodiments, the metal can be, but is not limited to, iron, nickel, copper, cobalt, gadolinium, manganese, tantalum, zinc, zirconium or any combinations thereof. In certain embodiments, the magnetic core comprises metal oxides (e.g., iron oxides), ferrites (e.g., manganese ferrites, cobalt ferrites, nickel ferrites, etc.), hematite and metal alloys (e.g., CoTaZn). In some embodiments, the magnetic core comprises one or more of a ferrite, a metal, a metal alloy, an iron oxide, or chromium dioxide. In some embodiments, the magnetic core comprises elemental iron or a compound thereof. In some embodiments, the magnetic core comprises one or more of magnetite (Fe3O4), maghemite (γFe2O3), or greigite (Fe3S4). In some embodiments, the inner core comprises an iron oxide (e.g., Fe3O4).

In certain embodiments, the bead contains a magnetic, paramagnetic, and/or superparamagnetic core that is covered by a surface functionalized coat or coating. In some embodiments, the coat can contain a material that can include, but is not limited to, a polymer, a polysaccharide, a silica, a fatty acid, a protein, a carbon, agarose, sepharose, or a combination thereof. In some embodiments, the polymer can be a polyethylene glycol, poly (lactic-co-glycolic acid), polyglutaraldehyde, polyurethane, polystyrene, or a polyvinyl alcohol. In certain embodiments, the outer coat or coating comprises polystyrene. In particular embodiments, the outer coating is surface functionalized.

In some embodiments, the stimulatory reagent comprises a bead that contains a metal oxide core (e.g., an iron oxide core) and a coat, wherein the metal oxide core comprises at least one polysaccharide (e.g., dextran), and wherein the coat comprises at least one polysaccharide (e.g., amino dextran), at least one polymer (e.g., polyurethane) and silica. In some embodiments the metal oxide core is a colloidal iron oxide core. In certain embodiments, the one or more agents include an antibody or antigen-binding fragment thereof. In particular embodiments, the one or more agents include an anti-CD3 antibody and an anti-CD28 antibody or antigen-binding fragments thereof. In some embodiments, the stimulatory reagent comprises an anti-CD3 antibody, anti-CD28 antibody, and an anti-biotin antibody. In some embodiments, the stimulatory reagent comprises an anti-biotin antibody. In some embodiments, the bead has a diameter of about 3 μm to about 10 μm. In some embodiments, the bead has a diameter of about 3 μm to about 5 μm. In certain embodiments, the bead has a diameter of about 3.5 μm.

In some embodiments, the stimulatory reagent comprises one or more agents that are attached to a bead comprising a metal oxide core (e.g., an iron oxide inner core) and a coat (e.g., a protective coat), wherein the coat comprises polystyrene. In certain embodiments, the beads are monodisperse, paramagnetic (e.g., superparamagnetic) beads comprising a paramagnetic (e.g., superparamagnetic) iron core, e.g., a core comprising magnetite (Fe₃O₄) and/or maghemite (γFe₂O₃) c and a polystyrene coat or coating. In some embodiments, the bead is non-porous. In some embodiments, the beads contain a functionalized surface to which the one or more agents are attached. In certain embodiments, the one or more agents are covalently bound to the beads at the surface. In some embodiments, the one or more agents include an antibody or antigen-binding fragment thereof. In some embodiments, the one or more agents include an anti-CD3 antibody and an anti-CD28 antibody. In some embodiments, the stimulatory reagent is or comprises anti-CD3/anti-CD28 magnetic beads, In some embodiments, the one or more agents include an anti-CD3 antibody and/or an anti-CD28 antibody, and an antibody or antigen fragment thereof capable of binding to a labeled antibody (e.g., biotinylated antibody), such as a labeled anti-CD3 or anti-CD28 antibody. In certain embodiments, the beads have a density of about 1.5 g/cm³ and a surface area of about 1 m²/g to about 4 m²/g. In particular embodiments; the beads are monodisperse superparamagnetic beads that have a diameter of about 4.5 μm and a density of about 1.5 g/cm³. In some embodiments, the beads the beads are monodisperse superparamagnetic beads that have a mean diameter of about 2.8 μm and a density of about 1.3 g/cm³.

In some embodiments, the composition of enriched T cells is incubated with stimulatory reagent a ratio of beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

2. Removal of the Stimulatory Reagent from Cells

In certain embodiments, the stimulatory reagent, e.g. anti-CD3/anti-CD28 magnetic beads, is removed and/or separated from the cells. Without wishing to be bound by theory, particular embodiments contemplate that the binding and/or association between a stimulatory reagent and cells may, in some circumstances, be reduced over time during the incubation. In certain embodiments, one or more agents may be added to reduce the binding and/or association between the stimulatory reagent and the cells. In particular embodiments, a change in cell culture conditions, e.g., media temperature of pH, may reduce the binding and/or association between the stimulatory reagent and the cells. Thus, in some embodiments, the stimulatory reagent may be removed from an incubation, cell culture system, and/or a solution separately from the cells, e.g., without removing the cells from the incubation, cell culture system, and/or a solution as well.

Methods for removing stimulatory reagents (e.g. stimulatory reagents that are or contain particles such as bead particles or magnetizable particles) from cells are known. In some embodiments, the use of competing antibodies, such as non-labeled antibodies, can be used, which, for example, bind to a primary antibody of the stimulatory reagent and alter its affinity for its antigen on the cell, thereby permitting for gentle detachment. In some cases, after detachment, the competing antibodies may remain associated with the particle (e.g. bead particle) while the unreacted antibody is or may be washed away and the cell is free of isolating, selecting, enriching and/or activating antibody. Exemplary of such a reagent is DETACaBEAD (Friedl et al. 1995; Entschladen et al. 1997). In some embodiments, particles (e.g. bead particles) can be removed in the presence of a cleavable linker (e.g. DNA linker), whereby the particle-bound antibodies are conjugated to the linker (e.g. CELLection, Dynal). In some cases, the linker region provides a cleavable site to remove the particles (e.g. bead particles) from the cells after isolation, for example, by the addition of DNase or other releasing buffer. In some embodiments, other enzymatic methods can also be employed for release of a particle (e.g. bead particle) from cells. In some embodiments, the particles (e.g. bead particles or magnetizable particles) are biodegradable.

In some embodiments, the stimulatory reagent is magnetic, paramagnetic, and/or superparamagnetic, and/or contains a bead that is magnetic, paramagnetic, and/or superparamagnetic, and the stimulatory reagent may be removed from the cells by exposing the cells to a magnetic field. Examples of suitable equipment containing magnets for generating the magnetic field include DynaMag CTS (Thermo Fisher), Magnetic Separator (Takara) and EasySep Magnet (Stem Cell Technologies).

In particular embodiments, the stimulatory reagent is removed or separated from the cells prior to the completion of the provided methods, e.g., prior to harvesting, collecting, and/or formulating engineered cells produced by the methods provided herein. In some embodiments, the stimulatory reagent is removed and/or separated from the cells prior to engineering, e.g., transducing or transfecting, the cells. In particular embodiments, the stimulatory reagent is removed and/or separated from the cells after the step of engineering the cells. In certain embodiments, the stimulatory reagent is removed prior to the cultivation of the cells, e.g., prior to the cultivation of the engineered, e.g., transfected or transduced, cells under conditions to promote proliferation and/or expansion.

In certain embodiments, the stimulatory reagent is separated and/or removed from the cells after an amount of time. In particular embodiments, the amount of time is an amount of time from the start and/or initiation of the incubation under stimulating conditions. In particular embodiments the start of the incubation is considered at or at about the time the cells are contacted with the stimulatory reagent and/or a media or solution containing the stimulatory reagent. In particular embodiments, the stimulatory reagent is removed or separated from the cells within or within about 10 days, 9 days, 8 days, 7 days, 6 days, 5 days, 4 days, 3 days, or 2 days after the start or initiation of the incubation. In particular embodiments, the stimulatory reagent is removed and/or separated from the cells at or at about 9 days, 8 days, 7 days, 6 days, 5 days, 4 days, 3 days, or 2 days after the start or initiation of the incubation. In certain embodiments, the stimulatory reagent is removed and/or separated from the cells at or at about 168 hours, 162 hours, 156 hours, 144 hours, 138 hours, 132 hours, 120 hours, 114 hours, 108 hours, 102 hours, or 96 hours after the start or initiation of the incubation. In particular embodiments, the stimulatory reagent is removed and/or separated from the cells at or at about 5 days after the start and/or initiation of the incubation. In some embodiments, the stimulatory reagent is removed and/or separated from the cells at or at about 4 days after the start and/or initiation of the incubation.

C. Engineering Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the methods provided herein are used in association with engineering one or more compositions of enriched T cells. In certain embodiments, the engineering is or includes the introduction of a polynucleotide, e.g., a recombinant polynucleotide encoding a recombinant protein. In particular embodiments, the recombinant proteins are recombinant receptors, such as any described in Section II. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In certain embodiments, one or more compositions of enriched T cells are engineered, e.g., transduced or transfected, prior to cultivating the cells, e.g., under conditions that promote proliferation and/or expansion, such as by a method provided in Section II-D. In particular embodiments, one or more compositions of enriched T cells are engineered after the one or more compositions have been stimulated, activated, and/or incubated under stimulating conditions, such as described in methods provided in Section II-B. In particular embodiments, the one or more compositions are stimulated compositions. In particular embodiments, the one or more stimulated compositions have been previously cryofrozen and stored, and are thawed prior to engineering.

In certain embodiments, the one or more compositions of stimulated T cells are or include two separate stimulated compositions of enriched T cells. In particular embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells, such as following incubation under stimulating conditions as described above, are genetically engineered separately. In some embodiments, a single composition of enriched T cells is genetically engineered. In certain embodiments, the single composition is a composition of enriched CD4+ T cells. In some embodiments, the single composition is a composition of enriched CD4+ and CD8+ T cells that have been combined from separate compositions prior to the engineering.

In some embodiments, the composition of enriched CD4+ T cells, such as stimulated CD4+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated CD8+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that, such as stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are genetically engineered, e.g., transduced or transfected. In certain embodiments, separate engineered compositions of enriched CD4+ and enriched CD8+ T cells are combined into a single composition after the genetic engineering has been performed and/or completed. In particular embodiments, separate compositions of enriched CD4+ and CD8+ T cells, such as separate compositions of stimulated CD4+ and CD8+T cells are separately engineered and are separately processed for cultivation and/or expansion of T cells after the genetic engineering and been performed and/or completed.

In some embodiments, the introduction of a polynucleotide, e.g., a recombinant polynucleotide encoding a recombinant protein, is carried out by contacting enriched CD4+ or CD8+ T cells, such as stimulated CD4+ or CD8+ T cells, with a viral particles containing the polynucleotide. In some embodiments, contacting can be effected with centrifugation, such as spinoculation (e.g., centrifugal inoculation). In some embodiments, the composition containing cells, viral particles and reagent can be rotated, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g., at or about or at least at or about 100 g, 200 g, 300 g, 400 g, 500 g, 1000 g, 1500 g, 2000 g, 2500 g, 3000 g or 3200 g), such as at or about 693 g, as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured). In some embodiments, at least a portion of the contacting, incubating, and/or engineering of the cells, e.g., cells from an stimulated composition of enriched CD4+ T cell or enriched CD8+ T cells, with the virus is performed with a rotation of between about 100 g and 3200 g, 1000 g and 2000 g, 1000 g and 3200 g, 500 g and 1000 g, 400 g and 1200 g, 600 g and 800 g, 600 and 700 g, or 500 g and 700 g. In some embodiments, the rotation is between 600 g and 700 g, e.g., at or about 693 g.

In certain embodiments, at least a portion of the engineering, transduction, and/or transfection is performed with rotation, e.g., spinoculation and/or centrifugation. In some embodiments, the rotation is performed for, for about, or for at least or about 5 minutes, 10 minutes, 15 minutes, 30 minutes, 60 minutes, 90 minutes, 1 hour, 2 hours, 3 hours, 4 hours, 6 hours, 8 hours, 12 hours, 24 hours, 48 hours, 72 hours, 2 days, 3 days, 4 days, 5 days, 6 days, or for at least 7 days. In some embodiments, the rotation is performed for or for about 60 minutes. In certain embodiments, the rotation is performed for about 30 minutes. In some embodiments, the rotation performed for about 30 minutes at between 600 g and 700 g, e.g., at or about 693 g.

In certain embodiments, the number of viable cells to be engineered, transduced, and/or transfected ranges from about 5×10⁶ cells to about 100×10⁷ cells, such as from about 10×10⁶ cells to about 100×10⁶ cells, from about 100×10⁶ cells to about 200×10⁶ cells, from about 200×10⁶ cells to about 300×10⁶ cells, from about 300×10⁶ cells to about 400×10⁶ cells, from about 400×10⁶ cells to about 500×10⁶ cells, or from about 500×10⁶ cells to about 100×10⁷ cells. In particular examples, the number of viable cells to be engineered, transduced, and/or transfected is about or less than about 300×10⁶ cells.

In certain embodiments, at least a portion of the engineering, transduction, and/or transfection is conducted at a volume (e.g., the spinoculation volume) from about 5 mL to about 100 mL, such as from about 10 mL to about 50 mL, from about 15 mL to about 45 mL, from about 20 mL to about 40 mL, from about 25 mL to about 35 mL, or at or at about 30 mL. In certain embodiments, the cell pellet volume after spinoculation ranges from about 1 mL to about 25 mL, such as from about 5 mL to about 20 mL, from about 5 mL to about 15 mL, from about 5 mL to about 10 mL, or at or at about 10 mL.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under stimulating conditions, such as by any of the methods described in Section I-B.

In some embodiments, methods for genetic engineering are carried out by contacting one or more cells of a composition with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the system is included with and/or placed into association with other instrumentation, including instrumentation to operate, automate, control and/or monitor aspects of the transduction step and one or more various other processing steps performed in the system, e.g. one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, the composition containing cells and composition containing viral vector particles, and optionally air, can be combined or mixed prior to providing the compositions to the cavity. In some embodiments, the composition containing cells and composition containing viral vector particles, and optionally air, are provided separately and combined and mixed in the cavity. In some embodiments, a composition containing cells, a composition containing viral vector particles, and optionally air, can be provided to the internal cavity in any order. In any of such some embodiments, a composition containing cells and viral vector particles is the input composition once combined or mixed together, whether such is combined or mixed inside or outside the centrifugal chamber and/or whether cells and viral vector particles are provided to the centrifugal chamber together or separately, such as simultaneously or sequentially.

In some embodiments, intake of a volume of gas, such as air, occurs prior to the incubating the cells and viral vector particles, such as rotation, in the transduction method. In some embodiments, intake of the volume of gas, such as air, occurs during the incubation of the cells and viral vector particles, such as rotation, in the transduction method.

In some embodiments, the liquid volume of the cells or viral vector particles that make up the transduction composition, and optionally the volume of air, can be a predetermined volume. The volume can be a volume that is programmed into and/or controlled by circuitry associated with the system.

In some embodiments, intake of the transduction composition, and optionally gas, such as air, is controlled manually, semi-automatically and/or automatically until a desired or predetermined volume has been taken into the internal cavity of the chamber. In some embodiments, a sensor associated with the system can detect liquid and/or gas flowing to and from the centrifuge chamber, such as via its color, flow rate and/or density, and can communicate with associated circuitry to stop or continue the intake as necessary until intake of such desired or predetermined volume has been achieved. In some aspects, a sensor that is programmed or able only to detect liquid in the system, but not gas (e.g. air), can be made able to permit passage of gas, such as air, into the system without stopping intake. In some such embodiments, a non-clear piece of tubing can be placed in the line near the sensor while intake of gas, such as air, is desired. In some embodiments, intake of gas, such as air, can be controlled manually.

In aspects of the provided methods, the internal cavity of the centrifuge chamber is subjected to high speed rotation. In some embodiments, rotation is effected prior to, simultaneously, subsequently or intermittently with intake of the liquid input composition, and optionally air. In some embodiments, rotation is effected subsequent to intake of the liquid input composition, and optionally air. In some embodiments, rotation is by centrifugation of the centrifugal chamber at a relative centrifugal force at the inner surface of side wall of the internal cavity and/or at a surface layer of the cells of at or about or at least at or about 800 g, 1000 g, 1100 g, 1500, 1600 g, 1800 g, 2000 g, 2200 g, 2500 g, 3000 g, 3500 g or 4000 g. In some embodiments, rotation is by centrifugation at a force that is greater than or about 1100 g, such as by greater than or about 1200 g, greater than or about 1400 g, greater than or about 1600 g, greater than or about 1800 g, greater than or about 2000 g, greater than or about 2400 g, greater than or about 2800 g, greater than or about 3000 g or greater than or about 3200 g. In some embodiments, rotation is by centrifugation at a force that is or is about 1600 g.

In some embodiments, the method of transduction includes rotation or centrifugation of the transduction composition, and optionally air, in the centrifugal chamber for greater than or about 5 minutes, such as greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes. In some embodiments, the transduction composition, and optionally air, is rotated or centrifuged in the centrifugal chamber for greater than 5 minutes, but for no more than 60 minutes, no more than 45 minutes, no more than 30 minutes or no more than 15 minutes. In particular embodiments, the transduction includes rotation or centrifugation for or for about 60 minutes.

In some embodiments, the method of transduction includes rotation or centrifugation of the transduction composition, and optionally air, in the centrifugal chamber for between or between about 10 minutes and 60 minutes, 15 minutes and 60 minutes, 15 minutes and 45 minutes, 30 minutes and 60 minutes or 45 minutes and 60 minutes, each inclusive, and at a force at the internal surface of the side wall of the internal cavity and/or at a surface layer of the cells of at least or greater than or about 1000 g, 1100 g, 1200 g, 1400 g, 1500 g, 1600 g, 1800 g, 2000 g, 2200 g, 2400 g, 2800 g, 3200 g or 3600 g. In particular embodiments, the method of transduction includes rotation or centrifugation of the transduction composition, e.g., the cells and the viral vector particles, at or at about 1600 g for or for about 60 minutes.

In some embodiments, the gas, such as air, in the cavity of the chamber is expelled from the chamber. In some embodiments, the gas, such as air, is expelled to a container that is operably linked as part of the closed system with the centrifugal chamber. In some embodiments, the container is a free or empty container. In some embodiments, the air, such as gas, in the cavity of the chamber is expelled through a filter that is operably connected to the internal cavity of the chamber via a sterile tubing line. In some embodiments, the air is expelled using manual, semi-automatic or automatic processes. In some embodiments, air is expelled from the chamber prior to, simultaneously, intermittently or subsequently with expressing the output composition containing incubated cells and viral vector particles, such as cells in which transduction has been initiated or cells have been transduced with a viral vector, from the cavity of the chamber.

In some embodiments, the transduction and/or other incubation is performed as or as part of a continuous or semi-continuous process. In some embodiments, a continuous process involves the continuous intake of the cells and viral vector particles, e.g., the transduction composition (either as a single pre-existing composition or by continuously pulling into the same vessel, e.g., cavity, and thereby mixing, its parts), and/or the continuous expression or expulsion of liquid, and optionally expelling of gas (e.g. air), from the vessel, during at least a portion of the incubation, e.g., while centrifuging. In some embodiments, the continuous intake and continuous expression are carried out at least in part simultaneously. In some embodiments, the continuous intake occurs during part of the incubation, e.g., during part of the centrifugation, and the continuous expression occurs during a separate part of the incubation. The two may alternate. Thus, the continuous intake and expression, while carrying out the incubation, can allow for a greater overall volume of sample to be processed, e.g., transduced.

In some embodiments, the incubation is part of a continuous process, the method including, during at least a portion of the incubation, effecting continuous intake of said transduction composition into the cavity during rotation of the chamber and during a portion of the incubation, effecting continuous expression of liquid and, optionally expelling of gas (e.g. air), from the cavity through the at least one opening during rotation of the chamber.

In some embodiments, the semi-continuous incubation is carried out by alternating between effecting intake of the composition into the cavity, incubation, expression of liquid from the cavity and, optionally expelling of gas (e.g. air) from the cavity, such as to an output container, and then intake of a subsequent (e.g., second, third, etc.) composition containing more cells and other reagents for processing, e.g., viral vector particles, and repeating the process. For example, in some embodiments, the incubation is part of a semi-continuous process, the method including, prior to the incubation, effecting intake of the transduction composition into the cavity through said at least one opening, and subsequent to the incubation, effecting expression of fluid from the cavity; effecting intake of another transduction composition comprising cells and the viral vector particles into said internal cavity; and incubating the another transduction composition in said internal cavity under conditions whereby said cells in said another transduction composition are transduced with said vector. The process may be continued in an iterative fashion for a number of additional rounds. In this respect, the semi-continuous or continuous methods may permit production of even greater volume and/or number of cells.

In some embodiments, a portion of the transduction incubation is performed in the centrifugal chamber, which is performed under conditions that include rotation or centrifugation.

In some embodiments, the method includes an incubation in which a further portion of the incubation of the cells and viral vector particles is carried out without rotation or centrifugation, which generally is carried out subsequent to the at least portion of the incubation that includes rotation or centrifugation of the chamber. In certain embodiments, the incubation of the cells and viral vector particles is carried out without rotation or centrifugation for at least 1 hour, 6 hours, 12 hours, 24 hours, 32 hours, 48 hours, 60 hours, 72 hours, 90 hours, 96 hours, 3 days, 4 days, 5 days, or greater than 5 days. In certain embodiments, the incubation is carried out for or for about 72 hours.

In some such embodiments, the further incubation is effected under conditions to result in integration of the viral vector into a host genome of one or more of the cells. It is within the level of a skilled artisan to assess or determine if the incubation has resulted in integration of viral vector particles into a host genome, and hence to empirically determine the conditions for a further incubation. In some embodiments, integration of a viral vector into a host genome can be assessed by measuring the level of expression of a recombinant protein, such as a heterologous protein, encoded by a nucleic acid contained in the genome of the viral vector particle following incubation. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

In some embodiments, the composition containing cells, the vector, e.g., viral particles, and reagent can be rotated, generally at relatively low force or speed, such as speed lower than that used to pellet the cells, such as from 600 rpm to 1700 rpm or from about 600 rpm to about 1700 rpm (e.g. at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g. at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, during at least a part of the genetic engineering, e.g. transduction, and/or subsequent to the genetic engineering the cells are transferred to the bioreactor bag assembly for culture of the genetically engineered cells, such as for cultivation or expansion of the cells, as described above.

In certain embodiments, a composition of enriched T cells in engineered, e.g., transduced or transfected, in the presence of a transduction adjuvant. In some embodiments, a composition of enriched T cells is engineered in the presence of one or more polycations. In some embodiments, a composition of enriched T cells is transduced, e.g., incubated with a viral vector particle, in the presence of one or more transduction adjuvants. In particular embodiments, a composition of enriched T cells is transfected, e.g., incubated with a non-viral vector, in the presence of one or more transduction adjuvants. In certain embodiments, the presence of one or more transduction adjuvants increases the efficiency of gene delivery, such as by increasing the amount, portion, and/or percentage of cells of the composition that are engineered (e.g., transduced or transfected). In certain embodiments, the presence of one or more transduction adjuvants increases the efficiency of transfection. In certain embodiments, the presence of one or more transduction adjuvants increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold more cells of a composition are engineered to contain or express the recombinant transduction adjuvants in the presence of a polycation as compared to an alternative and/or exemplary method of engineering cells without the presence of a transduction adjuvant.

In some embodiments, the composition of enriched cells are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of a transduction adjuvant. In certain embodiments, transduction adjuvants suitable for use with the provided methods include, but are not limited to polycations, fibronectin or fibronectin-derived fragments or variants, RetroNectin, and combinations thereof.

In some embodiments, the cells are engineered in the presence of a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is engineered in the presence of IL-2, e.g., human recombinant IL-2, at a concentration between 1 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is engineered in the presence of recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml. In some embodiments, the composition of enriched T cells is engineered in the presence of or of about 85 IU/ml. In some embodiments, the population of T cells is a population of CD4+ T cells. In particular embodiments, the composition of enriched T cells is enriched for CD4+ T cells, where CD8+ T cells are not enriched for and/or where CD8+ T cells are negatively selected for or depleted from the composition. In particular embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition.

In some embodiments, a composition of enriched T cells is engineered in the presence of recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 IU/ml, or between 550 IU/ml and 650 IU/ml. In particular embodiments, the composition of enriched T cells is engineered in the presence of IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is engineered in the presence of or of about 600 IU/ml of IL-7. In some embodiments, the composition engineered in the presence of recombinant IL-7 is enriched for a population of T cells, e.g., CD4+ T cells. In particular embodiments, the composition of enriched T cells is enriched for CD4+ T cells, where CD8+ T cells are not enriched for and/or where CD8+ T cells are negatively selected for or depleted from the composition.

In some embodiments, a composition of enriched T cells is engineered in the presence of recombinant IL-15, e.g., human recombinant IL-15, at a concentration between 0.1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is engineered in the presence of IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is engineered in or in about 10 IU/ml of IL-15. In some embodiments, the composition of enriched T cells is incubated in or in about 10 IU/ml of recombinant IL-15. In some embodiments, the composition engineered in the presence of recombinant IL-15 is enriched for a population of T cells, e.g., CD4+ T cells and/or CD8+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition.

In particular embodiments, a composition of enriched CD8+ T cells is engineered in the presence of IL-2 and/or IL-15. In certain embodiments, a composition of enriched CD4+ T cells is engineered in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cells are engineered in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthathione, cystamine and cystathionine, and/or glycine-glycine-histidine.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetylcysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, the cells are engineered in the presence of one or more antioxidants. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the cells are engineered in the presence of NAC. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1,500 μg/ml and 2 mg/ml, 500 μg/ml and 5 mg/ml, between 1 mg/ml and 10 mg/ml, or between 1 mg/ml and 100 mg/ml of NAC. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are engineered with or with about 0.8 mg/ml.

In some embodiments, a composition of enriched T cells, such as stimulated T cells, e.g. stimulated CD4+ T cells or stimulated CD8+ T cells, is engineered in the presence of one or more polycations. In some embodiments, a composition of enriched T cells, such as stimulated T cells, e.g. stimulated CD4+ T cells or stimulated CD8+ T cells, is transduced, e.g., incubated with a viral vector particle, in the presence of one or more polycations. In particular embodiments, a composition of enriched T cells, such as stimulated T cells, e.g. stimulated CD4+ T cells or stimulated CD8+ T cells, is transfected, e.g., incubated with a non-viral vector, in the presence of one or more polycations. In certain embodiments, the presence of one or more polycations increases the efficiency of gene delivery, such as by increasing the amount, portion, and/or percentage of cells of the composition that are engineered (e.g., transduced or transfected). In certain embodiments, the presence of one or more polycations increases the efficiency of transfection. In certain embodiments, the presence of one or more polycations increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold more cells of a composition are engineered to contain or express the recombinant polynucleotide in the presence of a polycation as compared to an alternative and/or exemplary method of engineering cells without the presence of a polycation.

In certain embodiments, the composition of enriched cells, e.g., the composition of enriched CD4+ T cells or enriched CD8+ T cells, such as stimulated T cells thereof, is engineered in the presence of a low concentration or amount of a polycation, e.g., relative to an exemplary and/or alternative method of engineering cells in the presence of a polycation. In certain embodiments, the composition of enriched cells, such as stimulated T cells, e.g. stimulated CD4+ T cells or stimulated CD8+ T cells, is engineered in the presence of less than 90%, less than 80%, less than 75%, less than 70%, less than 60%, less than 50%, less than 40%, less than 30%, less than 25%, less than 20%, less than 10%, less than 5%, less than 1%, less than 0.1%, of less than 0.01% of the amount and/or concentration of the polycation of an exemplary and/or alternative process for engineering cells. In some embodiments, the composition of enriched cells, such as stimulated T cells, e.g. stimulated CD4+ T cells or stimulated CD8+ T cells, are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than pg/ml, less than 10 μg/ml of the polycation. In particular embodiments, the composition of enriched cells, such as stimulated T cells, e.g. stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, or 50 μg/ml, of the polycation.

In particular embodiments, engineering the composition of enriched cells, such as stimulated T cells, e.g. stimulated CD4+ T cells or stimulated CD8+ T cells, in the presence of a polycation reduces the amount of cell death, e.g., by necrosis, programed cell death, or apoptosis. In some embodiments, the composition of enriched T cells, such as stimulated T cells, e.g. stimulated CD4+ T cells or stimulated CD8+ T cells, is engineered in the presence of a low amount of a polycation, e.g., less than 100 μg/ml, 50 μg/ml, or 10 μg/ml, and at least 50%, at least 60%, at least 70%, at least 75%, at least 80%, at least 85%, at least 90%, at least 95%, at least 99%, or at least 99.9% of the cells survive, e.g., do not undergo necrosis, programed cell death, or apoptosis, during or at least 1 day, 2 days, 3 days, 4 days, 5 days, 6 days, 7 days, or more than 7 days after the engineering step is complete. In some embodiments, the composition is engineered in the presence of a low concentration or amount of polycation as compared to the alternative and/or exemplary method of engineering cells in the presence of higher amount or concentration of polycation, e.g., more than 50 μg/ml, 100 μg/ml, 500 μg/ml, or 1,000 μg/ml, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing the exemplary and/or alternative process.

In some embodiments, the polycation is positively-charged. In certain embodiments, the polycation reduces repulsion forces between cells and vectors, e.g., viral or non-viral vectors, and mediates contact and/or binding of the vector to the cell surface. In some embodiments, the polycation is polybrene, DEAE-dextran, protamine sulfate, poly-L-lysine, or cationic liposomes.

In particular embodiments, the polycation is protamine sulfate. In some embodiments, the composition of enriched T cells, such as stimulated T cells, e.g. stimulated CD4+ T cells or stimulated CD8+ T cells, are engineered in the presence of less than or about 500 μg/ml, less than or about 400 μg/ml, less than or about 300 μg/ml, less than or about 200 μg/ml, less than or about 150 μg/ml, less than or about 100 μg/ml, less than or about 90 μg/ml, less than or about 80 μg/ml, less than or about 75 μg/ml, less than or about 70 μg/ml, less than or about 60 μg/ml, less than or about 50 μg/ml, less than or about 40 μg/ml, less than or about 30 μg/ml, less than or about 25 μg/ml, less than or about 20 μg/ml, or less than or about 15 μg/ml, or less than or about 10 μg/ml of protamine sulfate. In particular embodiments, the composition of enriched cells, such as stimulated T cells, e.g. stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 75 μg/ml, 80 μg/ml, 85 μg/ml, 90 μg/ml, 95 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, or 150 μg/ml of protamine sulfate.

In some embodiments, the engineered composition of enriched CD4+ T cells, such as stimulated T cells, e.g. stimulated CD4+ T cells, includes at least 40, at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated T cells, e.g. stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated T cells, e.g. stimulated CD8+ T cells, that is engineered includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells, such as stimulated T cells, e.g. stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, engineering the cells includes a culturing, contacting, or incubation with the vector, e.g., the viral vector of the non-viral vector. In certain embodiments, the engineering includes culturing, contacting, and/or incubating the cells with the vector is performed for, for about, or for at least 4 hours, 6 hours, 8 hours, 12 hours, 16 hours, 18 hours, 24 hours, 30 hours, 36 hours, 40 hours, 48 hours, 54 hours, 60 hours, 72 hours, 84 hours, 1 day, 2 days, 3 days, 4 days, 5 days, 6 days, or 7 days, or more than 7 days. In particular embodiments, the engineering includes culturing, contacting, and/or incubating the cells with the vector for or for about 24 hours, 36 hours, 48 hours, 60 hours, 72 hours, or 84 hours, or for or for about 2 days, 3 days, 4 days, or 5 days. In some embodiments, the engineering step is performed for or for about 24 hours, 36 hours, 48 hours, 60 hours, 72 hours, or 84 hours. In certain embodiments, the engineering is performed for about 60 hours or about 84 hours, for or for about 72 hours, or for or for about 2 days.

In some embodiments, the engineering is performed at a temperature from about 25 to about 38° C., such as from about 30 to about 37° C., from about 36 to about 38° C., or at or about 37° C.±2° C. In some embodiments, the composition of enriched T cells is engineered at a CO₂ level from about 2.5% to about 7.5%, such as from about 4% to about 6%, for example at or about 5%±0.5%. In some embodiments, the composition of enriched T cells is engineered at a temperature of or about 37° C. and/or at a CO₂ level of or about 5%.

In some embodiments, the cells, e.g., the CD4+ and/or the CD8+ T cells, are cultivated, after one or more steps are performed for genetic engineering, e.g., transducing or transfection the cells to contain a polynucleotide encoding a recombinant receptor. In some embodiments, the cultivation may include culture, incubation, stimulation, activation, expansion, and/or propagation. In some such embodiments, the further cultivation is effected under conditions to result in integration of the viral vector into a host genome of one or more of the cells. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In some embodiments, the further incubation is carried out at temperatures greater than room temperature, such as greater than or greater than about 25° C., such as generally greater than or greater than about 32° C., 35° C. or 37° C. In some embodiments, the further incubation is effected at a temperature of at or about 37° C.±2° C., such as at a temperature of at or about 37° C.

In some embodiments, the further incubation is performed under conditions for stimulation and/or activation of cells, which conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent (e.g. stimulatory and/or accessory agents), e.g., ligand, which is capable of activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, and/or an agent that promotes a costimulatory signal, such as one specific for a T cell costimulatory receptor, e.g., anti-CD3, anti-CD28, or anti-41-BB, for example, optionally bound to solid support such as a bead, and/or one or more cytokines. Among the stimulating agents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti-CD28 antibody to the culture medium. In some embodiments, the stimulating agents include IL-2 and/or IL-15, for example, an IL-2 concentration of at least about 10 units/mL.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL, at least about 50 units/mL, at least about 100 units/mL or at least about 200 units/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the further incubation is carried out in the same container or apparatus in which the contacting occurred. In some embodiments, the further incubation is carried out without rotation or centrifugation, which generally is carried out subsequent to the at least portion of the incubation done under rotation, e.g. in connection with centrifugation or spinoculation. In some embodiments, the further incubation is carried out outside of a stationary phase, such as outside of a chromatography matrix, for example, in solution.

In some embodiments, the further incubation is carried out in a different container or apparatus from that in which the contacting occurred, such as by transfer, e.g. automatic transfer, of the cell composition into a different container or apparatus subsequent to contacting with the viral particles and reagent.

In some embodiments, the further culturing or incubation, e.g. to facilitate ex vivo expansion, is carried out of for greater than or greater than about 24 hours, 2 days, 3 days, 4 days, 5 days, 6 days, 7 days, 8 days, 9 days, 10 days, 11 days, 12 days, 13 days or 14 days. In some embodiments, the further culturing or incubation is carried out for no more than 6 days, no more than 5 days, no more than 4 days, no more than 3 days, no more than 2 days or no more than 24 hours.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the methods provided herein do not include further culturing or incubation, e.g. do not include ex vivo expansion step, or include a substantially shorter ex vivo expansion step.

In some embodiments, the stimulatory reagent is removed and/or separated from the cells prior to the engineering. In particular embodiments, the stimulatory reagent is removed and/or separated from the cells after the engineering. In certain embodiments, the stimulatory agent is removed and/or separated from the cells subsequent to the engineering and prior to cultivating the engineered cells, e.g., under conditions that promote proliferation and/or expansion. In certain embodiments, the stimulatory reagent is a stimulatory reagent that is described in Section I-B-1. In particular embodiments, the stimulatory reagent is removed and/or separated from the cells as described in Section I-B-2.

1. Vectors and Methods

Also provided are one or more polynucleotides (e.g., nucleic acid molecules) encoding recombinant receptors, vectors for genetically engineering cells to express such receptors in accord with provided methods for producing the engineered cells. In some embodiments, the vector contains the nucleic acid encoding the recombinant receptor. In particular embodiments, the vector is a viral vector a non-viral vector. In some cases, the vector is a viral vector, such as a retroviral vector, e.g., a lentiviral vector or a gammaretroviral vector.

In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 10 and encoded by the nucleotide sequence set forth in SEQ ID NO: 9, the CD8 alpha signal peptide set forth in SEQ ID NO: 11, or the CD33 signal peptide set forth in SEQ ID NO: 12.

In some embodiments, the vectors include viral vectors, e.g., retroviral or lentiviral, non-viral vectors or transposons, e.g. Sleeping Beauty transposon system, vectors derived from simian virus 40 (SV40), adenoviruses, adeno-associated virus (AAV), lentiviral vectors or retroviral vectors, such as gamma-retroviral vectors, retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV) or adeno-associated virus (AAV).

In some embodiments, the viral vector or the non-viral DNA contains a nucleic acid that encodes a heterologous recombinant protein. In some embodiments, the heterologous recombinant molecule is or includes a recombinant receptor, e.g., an antigen receptor, SB-transposons, e.g., for gene silencing, capsid-enclosed transposons, homologous double stranded nucleic acid, e.g., for genomic recombination or reporter genes (e.g., fluorescent proteins, such as GFP) or luciferase).

f. Viral Vector Particles

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

In some embodiments, the provided methods involve methods of transducing cells by contacting, e.g., incubating, a cell composition comprising a plurality of cells with a viral particle. In some embodiments, the cells to be transfected or transduced are or comprise primary cells obtained from a subject, such as cells enriched and/or selected from a subject.

In some embodiments, the concentration of cells to be transduced of the composition is from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or about at least or about 30 minutes, 1 hour, 2 hours, 6 hours, 12 hours, 24 hours, 36 hours or more.

In some embodiments, contacting is performed in solution. In some embodiments, the cells and viral particles are contacted in a volume of from 0.5 mL to 500 mL or from about 0.5 mL to about 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

g. Non-Viral Vectors

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, recombinant nucleic acids are transferred into T cells via transposons. Transposons (transposable elements), are mobile segments of DNA that can move from one locus to another within genomes. These elements move via a conservative, “cut-and-paste” mechanism: the transposase catalyzes the excision of the transposon from its original location and promotes its reintegration elsewhere in the genome. Transposase-deficient elements can be mobilized if the transposase is provided by another transposase gene. Thus, transposons can be utilized to incorporate a foreign DNA into a host genome without the use of a viral transduction system. Examples of transposons suitable for use with mammalian cells, e.g., human primary leukocytes, include but are not limited to Sleeping Beauty and PiggyBacs.

Transposon-based transfection is a two-component system consisting of a transposase and a transposon. In some embodiments, the system comprises a transposon is engineered to comprise a foreign DNA (also referred herein as cargo DNA), e.g., a gene encoding a recombinant receptor, that is flanked by inverted repeat/direct repeat (IR/DR) sequences that are recognized by an accompanying transposase. In some embodiments, a non-viral plasmid encodes a transposase under the control of a promoter. Transfection of the plasmid into a host cell results in a transitory expression of the transposase, thus for an initial period following transfection, the transposase is expressed at sufficiently levels to integrate the transposon into the genomic DNA. In some embodiments, the transposase itself is not integrated into the genomic DNA, and therefor expression of the transposase decreases over time. In some embodiments, the transposase expression is expressed by the host cell at levels sufficient to integrate a corresponding transposon for less than about 4 hours, less than about 8 hours, less than about 12 hours, less than about 24 hours, less than about 2 days, less than about 3 days, less than about 4 days, less than about 5 days, less than about 6 days, less than about 7 days, less than about 2 weeks, less than about 3 weeks, less than about 4 weeks, less than about weeks, or less than about 8 weeks. In some embodiments, the cargo DNA that is introduced into the host's genome is not subsequently removed from the host's genome, at least because the host dose not express an endogenous transposase capable of excising the cargo DNA.

Sleeping Beauty (SB) is a synthetic member of the Tc/1-mariner superfamily of transposons, reconstructed from dormant elements harbored in the salmonid fish genome. SB transposon-based transfection is a two-component system consisting of a transposase and a transposon containing inverted repeat/direct repeat (IR/DR) sequences that result in precise integration into a TA dinucleotide. The transposon is designed with an expression cassette of interest flanked by IR/DRs. The SB transposase binds specific binding sites that are located on the IR of the Sleeping beauty transposon. The SB transposase mediates integration of the transposon, a mobile element encoding a cargo sequence flanked on both sides by inverted terminal repeats that harbor binding sites for the catalytic enzyme (SB). Stable expression results when SB inserts gene sequences into vertebrate chromosomes at a TA target dinucleotide through a cut-and-paste mechanism. This system has been used to engineer a variety of vertebrate cell types, including primary human peripheral blood leukocytes. In some embodiments, the cells are contacted, incubated, and/or treated with an SB transposon comprising a cargo gene, e.g., a gene encoding a recombinant receptor or a CAR, flanked by SB IR sequences. In particular embodiments, the cells to be transfected are contacted, incubated, and/or treated with a plasmid comprising an SB transposon comprising a cargo gene, e.g., a gene encoding a CAR, flanked by SB IR sequences. In certain embodiments, the plasmid further comprises a gene encoding an SB transposase that is not flanked by SB IR sequences.

PiggyBac (PB) is another transposon system that can be used to integrate cargo DNA into a host's, e.g., a human's, genomic DNA. The PB transposase recognizes PB transposon-specific inverted terminal repeat sequences (ITRs) located on both ends of the transposon and efficiently moves the contents from the original sites and efficiently integrates them into TTAA chromosomal sites. The PB transposon system enables genes of interest between the two ITRs in the PB vector to be mobilized into target genomes. The PB system has been used to engineer a variety of vertebrate cell types, including primary human cells. In some embodiments, the cells to be transfected are contacted, incubated, and/or treated with an PB transposon comprising a cargo gene, e.g., a gene encoding a CAR, flanked by PB IR sequences. In particular embodiments, the cells to be transfected are contacted, incubated, and/or treated with a plasmid comprising a PB transposon comprising a cargo gene, e.g., a gene encoding a CAR, flanked by PB IR sequences. In certain embodiments, the plasmid further comprises a gene encoding an SB transposase that is not flanked by PB IR sequences.

In some embodiments, the various elements of the transposon/transposase the employed in the subject methods, e.g., SB or PB vector(s), may be produced by standard methods of restriction enzyme cleavage, ligation and molecular cloning. One protocol for constructing the subject vectors includes the following steps. First, purified nucleic acid fragments containing desired component nucleotide sequences as well as extraneous sequences are cleaved with restriction endonucleases from initial sources, e.g., a vector comprising the transposase gene. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g., sequences corresponding to the various elements of the subject vectors, as described above is produced. Where desired, the circular molecules so constructed are then amplified in a prokaryotic host, e.g., E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc. An example of how to construct the vectors employed in the subject methods is provided in the Experimental section, infra. The preparation of a representative Sleeping Beauty transposon system is also disclosed in WO 98/40510 and WO 99/25817).

In some embodiments, transduction with transposons is performed with a plasmid that comprises a transposase gene and a plasmid that comprises a transposon that contains a cargo DNA sequence that is flanked by inverted repeat/direct repeat (IR/DR) sequences that are recognized by the transposase. In certain embodiments, the cargo DNA sequence encodes a heterologous protein, e.g., a recombinant T cell receptor or a CAR. In some embodiments, the plasmid comprises transposase and the transposon. In some embodiments, the transposase is under control of a ubiquitous promoter, or any promoter suitable to drive expression of the transposase in the target cell. Ubiquitous promoters include, but are not limited to, EF1a, CMB, SV40, PGK1, Ubc, human β-actin, CAG, TRE, UAS, Ac5, CaMKIIa, and U6. In some embodiments, the cargo DNA comprises a selection cassette allowing for the selection of cells with stable integration of the cargo DNA into the genomic DNA. Suitable selection cassettes include, but are not limited to, selection cassettes encoding a kanamycin resistance gene, spectinomycin resistance gene, streptomycin resistance gene, ampicillin resistance gene, carbenicillin resistance gene, hygromycin resistance gene, bleomycin resistance gene, erythromycin resistance gene, and polymyxin B resistance gene.

In some embodiments, the components for transduction with a transposon, e.g., plasmids comprising an SB transposase and SB transposon, are introduced into the target cell. Any convenient protocol may be employed, where the protocol may provide for in vitro or in vivo introduction of the system components into the target cell, depending on the location of the target cell. For example, where the target cell is an isolated cell, the system may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

In some embodiments, the SB transposon and the SB transposase source are introduced into a target cell of a multicellular organism, e.g., a mammal or a human, under conditions sufficient for excision of the inverted repeat flanked nucleic acid from the vector carrying the transposon and subsequent integration of the excised nucleic acid into the genome of the target cell. Some embodiments further comprise a step of ensuring that the requisite transposase activity is present in the target cell along with the introduced transposon. Depending on the structure of the transposon vector itself, i.e. whether or not the vector includes a region encoding a product having transposase activity, the method may further include introducing a second vector into the target cell which encodes the requisite transposase activity.

In some embodiments, the amount of vector nucleic acid comprising the transposon and the amount of vector nucleic acid encoding the transposase that is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed, e.g., the particular ex vivo administration protocol that is employed.

Once the vector DNA has entered the target cell in combination with the requisite transposase, the nucleic acid region of the vector that is flanked by inverted repeats, i.e. the vector nucleic acid positioned between the Sleeping Beauty transposase recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell. In particular embodiments, the vector is integrated into the genomes of at least 1%, at least 2%, at least 3%, at least 4%, at least 5%, at least 6% at least 7% at least 8%, at least 9%, at least 10%, at least 15%, or at least 20% of the cells that are transfected with the SB transposon and/or SB transposase. In some embodiments, integration of the nucleic acid into the target cell genome is stable, i.e., the vector nucleic acid remains present in the target cell genome for more than a transient period of time and is passed on a part of the chromosomal genetic material to the progeny of the target cell.

In certain embodiments, the transposons are used to integrate nucleic acids, i.e. polynucleotides, of various sizes into the target cell genome. In some embodiments, the size of DNA that is inserted into a target cell genome using the subject methods ranges from about 0.1 kb to 200 kb, from about 0.5 kb to 100 kb, from about 1.0 kb to about 8.0 kb, from about 1.0 to about 200 kb, from about 1.0 to about 10 kb, from about 10 kb to about 50 kb, from about 50 kb to about 100 kb, or from about 100 kb to about 200 kb. In some embodiments, the size of DNA that is inserted into a target cell genome using the subject methods ranges from about from about 1.0 kb to about 8.0 kb. In some embodiments, the size of DNA that is inserted into a target cell genome using the subject methods ranges from about 1.0 to about 200 kb. In particular embodiments, the size of DNA that is inserted into a target cell genome using the subject methods ranges from about 1.0 kb to about 8.0 kb.

D. Cultivation and/or Expansion of Cells

In some embodiments, the provided methods include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. In some embodiments, the cultivation produces one or more cultivated compositions of enriched T cells.

In certain embodiments, one or more compositions of enriched T cells, including stimulated and transduced T cells, such as separate compositions of such CD4+ and CD8+ T cells, are cultivated, e.g., under conditions that promote proliferation and/or expansion, prior to formulating the cells. In some aspects, the methods of cultivation, such as for promoting proliferation and/or expansion include methods provided herein, such as in Section I-F. In particular embodiments, one or more compositions of enriched T cells are cultivated after the one or more compositions have been engineered, e.g., transduced or transfected. In particular embodiments, the one or more compositions are engineered compositions. In particular embodiments, the one or more engineered compositions have been previously cryofrozen and stored, and are thawed prior to cultivating.

In certain embodiments, the one or more compositions of engineered T cells are or include two separate compositions of enriched T cells. In particular embodiments, two separate compositions of enriched T cells, e.g., two separate compositions of enriched T cells selected, isolated, and/or enriched from the same biological sample, that are introduced with a recombinant receptor (e.g. CAR), are separately cultivated under conditions that promote proliferation and/or expansion of the cells. In some embodiments, the conditions are stimulating conditions. In certain embodiments, the two separate compositions include a composition of enriched CD4+ T cells, such as engineered CD4+ T cells that were introduced with the nucleic acid encoding the recombinant receptor and/or that express the recombinant receptor. In particular embodiments, the two separate compositions include a composition of enriched CD8+ T cells, such as engineered CD8+ T cells that were introduced with the nucleic acid encoding the recombinant receptor and/or that express the recombinant receptor. In some embodiments, two separate compositions of enriched CD4+ T cells and enriched CD8+ T cells, such as engineered CD4+ T cells and engineered CD8+ T cells, are separately cultivated, e.g., under conditions that promote proliferation and/or expansion. In some embodiments, a single composition of enriched T cells is cultivated. In certain embodiments, the single composition is a composition of enriched CD4+ T cells. In some embodiments, the single composition is a composition of enriched CD4+ and CD8+ T cells that have been combined from separate compositions prior to the cultivation.

In some embodiments, the composition of enriched CD4+ T cells, such as engineered CD4+ T cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express the recombinant receptor and/or have been transduced or transfected with the recombinant polynucleotide encoding the recombinant receptor. In certain embodiments, the composition of enriched CD4+ T cells that is cultivated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as engineered CD8+ t cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In particular embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide encoding the recombinant receptor. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells, such as separate compositions of engineered CD4+ and engineered CD8+ T cells, are combined into a single composition and are cultivated, e.g., under conditions that promote proliferation and/or expansion. In certain embodiments, separate cultivated compositions of enriched CD4+ and enriched CD8+ T cells are combined into a single composition after the cultivation has been performed and/or completed. In particular embodiments, separate compositions of enriched CD4+ and CD8+ T cells, such as separate compositions of engineered CD4+ and engineered CD8+ T cells, are separately cultivated, e.g., under conditions that promote proliferation and/or expansion.

In some embodiments, the cells, e.g., the engineered cells are cultivated in a volume of media that is, is about, or is at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL, or 2,400 mL. In some embodiments, the cells are cultivated at an initial volume that is later adjusted to a different volume. In particular embodiments, the volume is later adjusted during the cultivation. In particular embodiments, the volume is increased from the initial volume during the cultivation. In certain embodiments, the volume is increased when the cells achieve a density during the cultivation. In certain embodiment, the initial volume is or is about 500 mL.

In particular embodiments, the volume is increased from the initial volume when the cells achieve a density or concentration during the cultivation. In particular embodiments, the volume is increased when the cells achieve a density and/or concentration of, of about, or of at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the volume is increased from the initial volume when the cells achieve a density and/or concentration of, of at least, or of about 0.6×10⁶ cells/ml. In some embodiments, the density and/or concentration is of viable cells in the culture. In particular embodiments, the volume is increased when the cells achieve a density and/or concentration of, of about, or of at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, the volume is increased from the initial volume when the viable cells achieve a density and/or concentration of, of at least, or of about 0.6×10⁶ viable cells/ml. In some embodiments, density and/or concentration of the cells or viable cells can be determined or monitored during the cultivation, such as by using methods as described, including optical methods, including digital holography microscopy (DHM) or differential digital holography microscopy (DDHM).

In some embodiments, the cells achieve a density and/or concentration, and the volume is increased by, by about, or by at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL. In some embodiments, the volume is increased by 500 mL. In particular embodiments, the volume is increased to a volume of, of about, or of at least 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL. In certain embodiments, the volume is increased to a volume of 1,000 mL. In certain embodiments, the volume is increase at a rate of, of at least, or of about 5 mL, 10 mL, 20 mL, 25 mL, 30 mL, 40 mL, 50 mL, 60 mL, 70 mL, 75 mL, 80 mL, 90 mL, or 100 mL, every 1, 2, 3, 4, 5, 6, 7, 8, 9, or 10 minutes. In certain embodiments, the rate is or is about 50 mL every 8 minutes.

In some embodiments, a composition of enriched T cells, such as engineered T cells, is cultivated under conditions that promote proliferation and/or expansion. In some embodiments, such conditions may be designed to induce proliferation, expansion, activation, and/or survival of cells in the population. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In some embodiments, the cultivation is performed under conditions that generally include a temperature suitable for the growth of primary immune cells, such as human T lymphocytes, for example, at least about 25 degrees Celsius, generally at least about 30 degrees, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g. cultivation or expansion, results in a desired or threshold density, concentration, number or dose of cells. In some embodiments, the incubation is carried out for a time period until the culture, e.g. cultivation or expansion, results in a desired or threshold density, concentration, number or dose of viable cells. In some embodiments, the incubation is greater than or greater than about or is for about or 24 hours, 48 hours, 72 hours, 96 hours, 5 days, 6 days, 7 days, 8 days, 9 days or more. In some embodiments, density, concentration and/or number or dose of the cells can be determined or monitored during the cultivation, such as by using methods as described, including optical methods, including digital holography microscopy (DHM) or differential digital holography microscopy (DDHM).

In some embodiments, the stimulatory reagent is removed and/or separated from the cells prior to the cultivation. In certain embodiments, the stimulatory agent is removed and/or separated from the cells subsequent to the engineering and prior to cultivating the engineered cells, e.g., under conditions that promote proliferation and/or expansion. In some embodiments, the stimulatory reagent is a stimulatory reagent that is described herein, e.g., in Section I-B-1. In particular embodiments, the stimulatory reagent is removed and/or separated from the cells as described herein, e.g., in Section I-B-2.

In particular embodiments, a composition of enriched T cells, such as engineered T cells, for example separate compositions of engineered CD4+ T cells and engineered CD8+ T cells, is cultivated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In particular embodiments, the composition of enriched CD4+ T cells, such as engineered CD4+ T cells, is cultivated with recombinant IL-2. In some embodiments, cultivating a composition of enriched CD4+ T cells, such as engineered CD4+ T cells, in the presence of recombinant IL-2 increases the probability or likelihood that the CD4+ T cells of the composition will continue to survive, grow, expand, and/or activate during the cultivation step and throughout the process. In some embodiments, cultivating the composition of enriched CD4+ T cells, such as engineered CD4+ T cells, in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, or at least 200% CD4+ as compared to an alternative and/or exemplary method that does not cultivate the composition of enriched CD4+ T cells in the presence of recombinant IL-2.

In some embodiments, the cells, such as separate compositions of engineered CD4+ T cells and engineered CD8+ T cells, are cultivated with a cytokine, e.g., a recombinant human cytokine, at a concentration of between 1 IU/ml and 2,000 IU/ml, between 10 IU/ml and 100 IU/ml, between 50 IU/ml and 500 IU/ml, between 100 IU/ml and 200 IU/ml, between 500 IU/ml and 1400 IU/ml, between 250 IU/ml and 500 IU/ml, or between 500 IU/ml and 2,500 IU/ml.

In some embodiments, a composition of enriched of T cells, such as separate compositions of engineered CD4+ T cells and CD8+ T cells, is cultivated with recombinant IL-2, e.g., human recombinant IL-2, at a concentration between 2 IU/ml and 500 IU/ml, between 10 IU/ml and 250 IU/ml, between 100 IU/ml and 500 IU/ml, or between 100 IU/ml and 400 IU/ml. In particular embodiments, the composition of enriched T cells is cultivated with IL-2 at a concentration at or at about 50 IU/ml, 75 IU/ml, 100 IU/ml, 125 IU/ml, 150 IU/ml, 175 IU/ml, 200 IU/ml, 225 IU/ml, 250 IU/ml, 300 IU/ml, or 400 IU/ml. In some embodiments, the composition of enriched T cells is cultivated with recombinant IL-2 at a concentration of 200 IU/ml. In some embodiments, the composition of enriched T cells is a composition of enriched CD4+ T cells, such as a composition of engineered CD4+ T cells. In particular embodiments, the composition of enriched T cells is a composition of enriched CD8+ T cells, such as a composition of engineered CD8+ T cells.

In some embodiments, a composition of enriched T cells, such as separate compositions of engineered CD4+ T cells and CD8+ T cells, is cultivated with IL-7, e.g., human recombinant IL-7, at a concentration between 10 IU/ml and 5,000 IU/ml, between 500 IU/ml and 2,000 IU/ml, between 600 IU/ml and 1,500 IU/ml, between 500 IU/ml and 2,500 IU/ml, between 750 IU/ml and 1,500 IU/ml, or between 1,000 IU/ml and 2,000 IU/ml. In particular embodiments, the composition of enriched T cells is cultivated with IL-7 at a concentration at or at about 100 IU/ml, 200 IU/ml, 300 IU/ml, 400 IU/ml, 500 IU/ml, 600 IU/ml, 700 IU/ml, 800 IU/ml, 900 IU/ml, 1,000 IU/ml, 1,200 IU/ml, 1,400 IU/ml, or 1,600 IU/ml. In some embodiments, the cells are cultivated in the presence of recombinant IL-7 at a concertation of or of about 1,200 IU/ml. In some embodiments, the composition of enriched T cells is a composition of enriched CD4+ T cells, such as engineered CD4+ T cells.

In some embodiments, a composition of enriched T cells, such as separate compositions of engineered CD4+ T cells and CD8+ T cells, is cultivated with IL-15, e.g., human recombinant IL-15, at a concentration between 0.1 IU/ml and 200 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 00 IU/ml. In particular embodiments, the composition of enriched T cells is cultivated with IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, 50 IU/ml, 100 IU/ml, or 200 IU/ml. In particular embodiments, a composition of enriched T cells is cultivated with recombinant IL-15 at a concentration of 20 IU/ml. In some embodiments, the composition of enriched T cells is a composition of enriched CD4+ T cells, such as engineered CD4+ T cells. In particular embodiments, the composition of enriched T cells is a composition of enriched CD8+ T cells, such as engineered CD8+ T cells.

In particular embodiments, a composition of enriched CD8+ T cells, such as engineered CD8+ T cells, is cultivated in the presence of IL-2 and/or IL-15, such as in amounts as described. In certain embodiments, a composition of enriched CD4+ T cells, such as engineered CD4+ T cells, is cultivated in the presence of IL-2, IL-7, and/or IL-15, such as in amounts as described. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cultivation is performed in a closed system. In certain embodiments, the cultivation is performed in a closed system under sterile conditions. In particular embodiments, the cultivation is performed in the same closed system as one or more steps of the provided systems. In some embodiments the composition of enriched T cells is removed from a closed system and placed in and/or connected to a bioreactor for the cultivation. Examples of suitable bioreactors for the cultivation include, but are not limited to, GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the cells during at least a portion of the cultivation step.

In some embodiments, cells cultivated while enclosed, connected, and/or under control of a bioreactor undergo expansion during the cultivation more rapidly than cells that are cultivated without a bioreactor, e.g., cells that are cultivated under static conditions such as without mixing, rocking, motion, and/or perfusion. In some embodiments, cells cultivated while enclosed, connected, and/or under control of a bioreactor reach or achieve a threshold expansion, cell count, and/or density within 14 days, 10 days, 9 days, 8 days, 7 days, 6 days, 5 days, 4 days, 3 days, 2 days, 60 hours, 48 hours, 36 hours, 24 hours, or 12 hours. In some embodiments, cells cultivated while enclosed, connected, and/or under control of a bioreactor reach or achieve a threshold expansion, cell count, and/or density at least 50%, at least 60%, at least 70%, at least 80%, at least 90%, at least 95%, at least 100%, at least 150%, at least 1-fold, at least 2-fold, at least 3-fold, at least 4-fold, at least 5-fold than cells cultivated in an exemplary and/or alternative process where cells are not cultivated while enclosed, connected, and/or under control of a bioreactor.

In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 17°, 16°, 15°, 14°, 13°, 12°, 11°, 10°, 9°, 8°, 7° 6°, 5° 4° 3° 2° or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 112, 13, 14 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, the bioreactor maintains the temperature at or near 37° C. and CO₂ levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the cultivation is performed with perfusion, such as with a rate of 290 ml/day, 580 ml/day, and/or 1160 ml/day, e.g., depending on the timing in relation to the start of the cultivation and/or density of the cultivated cells. In some embodiments, at least a portion of the cell culture expansion is performed with a rocking motion, such as at an angle of between 5° and 10°, such as 6°, at a constant rocking speed, such as a speed of between 5 and 15 RPM, such as 6 RPM or 10 RPM.

In some embodiments, the at least a portion of the cultivation step is performed under constant perfusion, e.g., a perfusion at a slow steady rate. In some embodiments, the perfusion is or include an outflow of liquid e.g., used media, and an inflow of fresh media. In certain embodiments, the perfusion replaces used media with fresh media. In some embodiments, at least a portion of the cultivation is performed under perfusion at a steady rate of or of about or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day.

In particular embodiments, cultivation is started under conditions with no perfusion, and perfusion started after a set and/or predetermined amount of time, such as or as about or at least 12 hours, 24 hours, 36 hours, 48 hours, 60 hours, 72 hours, or more than 72 hours after the start or initiation of the cultivation. In particular embodiments, perfusion is started when the density or concentration of the cells reaches a set or predetermined density or concentration. In some embodiments, the perfusion is started when the cultivated cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In particular embodiments, perfusion is started when the density or concentration of viable cells reaches a set or predetermined density or concentration. In some embodiments, the perfusion is started when the cultivated viable cells reach a density or concentration of, of about, or at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml.

In particular embodiments, the perfusion is performed at different speeds during the cultivation. For example, in some embodiments, the rate of the perfusion depends on the density and/or concentration of the cultivated cells. In certain embodiments, the rate of perfusion is increased when the cells reach a set or predetermined density or concentration. The perfusion rate may change, e.g., change from one steady perfusion rate to an increased steady perfusion rate, once, twice, three times, four times, five times, more than five times, more than ten times, more than 15 times, more than 20 times, more than 25 times, more than 50 times, or more than 100 times during the cultivation. In some embodiments, the steady perfusion rate increases when the cells reach a set or predetermined cell density or concentration of, of about, or at least 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the steady perfusion rate increases when the cells reach a set or predetermined viable cell density or concentration of, of about, or at least 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, density and/or concentration of the cells or of the viable cells during the cultivation, such as under perfusion, can be determined or monitored, such as by using methods as described, including optical methods, including digital holography microscopy (DHM) or differential digital holography microscopy (DDHM).

In some embodiments, cultivation is started under conditions with no perfusion, and, perfusion is started when the density or concentration of the cells reaches a set or predetermined density or concentration. In some embodiments, the perfusion is started at a rate of, of about, or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml.

In certain embodiments, at least part of the cultivation is performed with perfusion at a certain rate, and the perfusion rate is increased to, to about, or to at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the perfusion is performed when the cells are cultivated in a volume of, of about, or at least 300 mL, 400 mL, 500 mL, 600 mL, 700 mL, 800 mL, 900 mL, or 1000 mL. In some embodiments, the volume is 1000 mL.

In certain embodiments, cultivation is started under conditions with either no perfusion or perfusion at a certain rate, and the perfusion rate is increased to, to about, or to at 290 ml/day when the density or concentration of the cells reaches a concentration of, of about, or of at least 0.61×10⁶ cells/ml. In certain embodiments, the cells are perfused at a rate of, of about, or at least 290 ml/day when the density or concentration of the cells reaches a concentration of, of about, or of at least 0.61×10⁶ cells/ml when the cells are cultivated at a volume of, of about, or at least 1000 mL. In some embodiments, the perfusion rate is increased to, to about, or to at 580 ml/day when the density or concentration of the cells reaches a concentration of, of about, or of at least 0.81×10⁶ cells/ml. In certain embodiments, the perfusion rate is increased to, to about, or to at 1160 ml/day when the density or concentration of the cells reaches a concentration of, of about, or of at least 1.01×10⁶ cells/ml. In some embodiments, the perfusion rate is increased to, to about, or to at 1160 ml/day when the density or concentration of the cells reaches a concentration of, of about, or of at least 1.2×10⁶ cells/ml.

In aspects of the provided embodiments, the rate of perfusion, including the timing of when it is started or increased as described herein and above, is determined from assessing density and/or concentration of the cells or assessing the density and/or concentration of viable cells during the cultivation. In some embodiments, density and/or concentration of the cells can be determined using methods as described, including optical methods, including digital holography microscopy (DHM) or differential digital holography microscopy (DDHM).

In some embodiments, a composition of enriched cells, such as engineered T cells, e.g. engineered CD4+ T cells or engineered CD8+ T cells, is cultivated in the presence of a surfactant. In particular embodiments, cultivating the cells of the composition reduces the amount of shear stress that may occur during the cultivation, e.g., due to mixing, rocking, motion, and/or perfusion. In particular embodiments, the composition of enriched T cells, such as engineered T cells, e.g. engineered CD4+ T cells or engineered CD8+ T cells, is cultivated with the surfactant and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programed cell death, or apoptosis, during or at least 1 day, 2 days, 3 days, 4 days, 5 days, 6 days, 7 days, or more than 7 days after the cultivation is complete. In particular embodiments, the composition of enriched T cells, such as engineered T cells, e.g. engineered CD4+ T cells or engineered CD8+ T cells, is cultivated in the presence of a surfactant and less than 50%, less than 40%, less than 30%, less than 25%, less than 20%, less than 15%, less than 10%, less than 5%, less than 1%, less than 0.1% or less than 0.01% of the cells undergo cell death, e.g., programmed cell death, apoptosis, and/or necrosis, such as due to shearing or shearing-induced stress.

In particular embodiments, a composition of enriched T cells, such as engineered T cells, e.g. engineered CD4+ T cells or engineered CD8+ T cells, is cultivated in the presence of between 0.1 μl/ml and 10.0 μl/ml, between 0.2 μl/ml and 2.5 μl/ml, between 0.5 μl/ml and 5 μl/ml, between 1 μl/ml and 3 μl/ml, or between 2 μl/ml and 4 μl/ml of the surfactant. In some embodiments, the composition of enriched T cells, such as engineered T cells, e.g. engineered CD4+ T cells or engineered CD8+ T cells, is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2.0 μl/ml, 2.5 μl/ml, 5.0 μl/ml, 10 μl/ml, 25 μl/ml, or 50 μl/ml of the surfactant. In certain embodiments, the composition of enriched T cells is cultivated in the presence of or of about 2 μl/ml of the surfactant.

In some embodiments, a surfactant is or includes an agent that reduces the surface tension of liquids and/or solids. For example, a surfactant includes a fatty alcohol (e.g., steryl alcohol), a polyoxyethylene glycol octylphenol ether (e.g., Triton X-100), or a polyoxyethylene glycol sorbitan alkyl ester (e.g., polysorbate 20, 40, 60). In certain embodiments the surfactant is selected from the group consisting of Polysorbate 80 (PS80), polysorbate 20 (PS20), poloxamer 188 (P188). In an exemplary embodiment, the concentration of the surfactant in chemically defined feed media is about 0.0025% to about 0.25% (v/v) of PS80; about 0.0025% to about 0.25% (v/v) of PS20; or about 0.1% to about 5.0% (w/v) of P188.

In some embodiments, the surfactant is or includes an anionic surfactant, a cationic surfactant, a zwitterionic surfactant, or a nonionic surfactant added thereto. Suitable anionic surfactants include but are not limited to alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl glycerol, phosphatidyl inosine, phosphatidylinositol, diphosphatidylglycerol, phosphatidylserine, phosphatidic acid and their salts, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate).

In some embodiments, suitable nonionic surfactants include: glyceryl esters, polyoxyethylene fatty alcohol ethers, polyoxyethylene sorbitan fatty acid esters (polysorbates), polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethyl cellulose, hydroxypropyl cellulose, hydroxypropylmethyl cellulose, noncrystalline cellulose, polysaccharides including starch and starch derivatives such as hydroxyethylstarch (HES), polyvinyl alcohol, and polyvinylpyrrolidone. In certain embodiments, the nonionic surfactant is a polyoxyethylene and polyoxypropylene copolymer and preferably a block copolymer of propylene glycol and ethylene glycol. Such polymers are sold under the tradename POLOXAMER, also sometimes referred to as PLURONIC® F68 or Kolliphor® P188. Among polyoxyethylene fatty acid esters is included those having short alkyl chains. One example of such a surfactant is SOLUTOL® HS 15, polyethylene-660-hydroxystearate.

In some embodiments, suitable cationic surfactants may include, but are not limited to, natural phospholipids, synthetic phospholipids, quaternary ammonium compounds, benzalkonium chloride, cetyltrimethyl ammonium bromide, chitosans, lauryl dimethyl benzyl ammonium chloride, acyl carnitine hydrochlorides, dimethyl dioctadecyl ammonium bromide (DDAB), dioleyoltrimethyl ammonium propane (DOTAP), dimyristoyl trimethyl ammonium propane (DMTAP), dimethyl amino ethane carbamoyl cholesterol (DC-Chol), 1,2-diacylglycero-3-(O-alkyl) phosphocholine, O-alkylphosphatidylcholine, alkyl pyridinium halides, or long-chain alkyl amines such as, for example, n-octylamine and oleylamine.

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof. The phospholipid, whether anionic, zwitterionic or a mixture of phospholipids, may be salted or desalted, hydrogenated or partially hydrogenated or natural semi-synthetic or synthetic.

In certain embodiments, the surfactant is poloxamer, e.g., poloxamer 188. In some embodiments, a composition of enriched T cells is cultivated in the presence of between 0.1 μl/ml and 10.0 μl/ml, between 0.2 μl/ml and 2.5 μl/ml, between 0.5 μl/ml and 5 μl/ml, between 1 μl/ml and 3 μl/ml, or between 2 μl/ml and 4 μl/ml of poloxamer. In some embodiments, the composition of enriched T cells is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2.0 μl/ml, 2.5 μl/ml, 5.0 μl/ml, 10 μl/ml, 25 μl/ml, or 50 μl/ml of the surfactant. In certain embodiments, the composition of enriched T cells is cultivated in the presence of or of about 2 μl/ml of poloxamer.

In particular embodiments, the cultivation ends, such as by harvesting cells, when cells achieve a threshold amount, concentration, and/or expansion. In particular embodiments, the cultivation ends when the cell achieve or achieve about or at least a 1.5-fold expansion, a 2-fold expansion, a 2.5-fold expansion, a 3-fold expansion, a 3.5-fold expansion, a 4-fold expansion, a 4.5-fold expansion, a 5-fold expansion, a 6-fold expansion, a 7-fold expansion, a 8-fold expansion, a 9-fold expansion, a 10-fold expansion, or greater than a 10-fold expansion, e.g., with respect and/or in relation to the amount of density of the cells at the start or initiation of the cultivation. In some embodiments, the threshold expansion is a 4-fold expansion, e.g., with respect and/or in relation to the amount of density of the cells at the start or initiation of the cultivation.

In some embodiments, the cultivation ends, such as by harvesting cells, when the cells achieve a threshold total amount of cells, e.g., threshold cell count. In some embodiments, the cultivation ends when the cells achieve a threshold total nucleated cell (TNC) count. In some embodiments, the cultivation ends when the cells achieve a threshold viable amount of cells, e.g., threshold viable cell count. In some embodiments, the threshold cell count is or is about or is at least of 50×10⁶ cells, 100×10⁶ cells, 200×10⁶ cells, 300×10⁶ cells, 400×10⁶ cells, 600×10⁶ cells, 800×10⁶ cells, 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, 5000×10⁶ cells, 10,000×10⁶ cells, 12,000×10⁶ cells, 15,000×10⁶ cells or 20,000×10⁶ cells, or any of the foregoing threshold of viable cells. In particular embodiments, the cultivation ends when the cells achieve a threshold cell count. In some embodiments, the cultivation ends at, at about, or within 6 hours, 12 hours, 24 hours, 36 hours, 1 day, 2 days, 3 days, 4 days, 5 days, 6 days, or 7 or more days, after the threshold cell count is achieved. In particular embodiments, the cultivation is ended at or about 1 day after the threshold cell count is achieved. In certain embodiments, the threshold density is, is about, or is at least 0.1×10⁶ cells/ml, 0.5×10⁶ cells/ml, 1×10⁶ cells/ml, 1.2×10⁶ cells/ml, 1.5×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml, or any of the foregoing threshold of viable cells. In particular embodiments, the cultivation ends when the cells achieve a threshold density. In some embodiments, the cultivation ends at, at about, or within 6 hours, 12 hours, 24 hours, 36 hours, 1 day, 2 days, 3 days, 4 days, 5 days, 6 days, or 7 or more days, after the threshold density is achieved. In particular embodiments, the cultivation is ended at or about 1 day after the threshold density is achieved.

In some embodiments, the cultivation step is performed for the amount of time required for the cells to achieve a threshold amount, density, and/or expansion. In some embodiments, the cultivation is performed for or for about, or for less than, 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In particular embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In certain embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks.

In certain embodiments, the cultivation step is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the cultivation step is performed until 1 day after the cells achieve a threshold cell count of or of about 1200×10⁶ cells and are cultured for a minimum of 10 days, and/or until 1 day after the cells achieve a threshold cell count of or of about 5000×10⁶ cells. In some embodiments, the cultivation step is performed until 1 day after the cells achieve a threshold cell count of or of about 1200×10⁶ cells and are cultured for a minimum of 9 days, and/or until 1 day after the cells achieve a threshold cell count of or of about 5000×10⁶ cells. In some embodiments, the cultivation step is performed until 1 day after the cells achieve a threshold cell count of or of about 1000×10⁶ cells and are cultured for a minimum of 8 days, and/or until 1 day after the cells achieve a threshold cell count of or of about 4000×10⁶ cells. In certain embodiments, the cultivation is an expansion step and is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the expansion step is performed until 1 day after the cells achieve a threshold cell count of or of about 1200×10⁶ cells and are expanded for a minimum of 10 days, and/or until 1 day after the cells achieve a threshold cell count of or of about 5000×10⁶ cells. In some embodiments, the expansion step is performed until 1 day after the cells achieve a threshold cell count of or of about 1200×10⁶ cells and are expanded for a minimum of 9 days, and/or until 1 day after the cells achieve a threshold cell count of or of about 5000×10⁶ cells. In some embodiments, the expansion step is performed until 1 day after the cells achieve a threshold cell count of or of about 1000×10⁶ cells and are expanded for a minimum of 8 days, and/or until 1 day after the cells achieve a threshold cell count of or of about 4000×10⁶ cells. In some embodiments, the expansion step is performed until 1 day after the cells achieve a threshold cell count of or of about 1400×10⁶ cells and are expanded for a minimum of 5 days, and/or until 1 day after the cells achieve a threshold cell count of or of about 4000×10⁶ cells.

In some embodiments, the cultivation is performed for at least a minimum amount of time. In some embodiments, the cultivation is performed for at least 14 days, at least 12 days, at least 10 days, at least 7 days, at least 6 days, at least 5 days, at least 4 days, at least 3 days, at least 2 days, at least 36 hours, at least 24 hours, at least 12 hours, or at least 6 hours, even if the threshold is achieved prior to the minimum amount of time. In some embodiments, increasing the minimum amount of time that the cultivation is performed, may, in some cases, reduce the activation and/or reduce the level or one or more activation markers, in the cultivated cells, formulated cells, and/or cells of the output composition. In some embodiments, the minimum cultivation time counts from a determined point an exemplary process (e.g. a selection step; a thaw step; and/or an activation step) to the day the cells are harvested.

In aspects of the provided embodiments, the density and/or concentration of the cells or of the viable cells during the cultivation is monitored or carried out during the cultivation, such as until a threshold amount, density, and/or expansion is achieved as described. In some embodiments such methods include those as described, including optical methods, including digital holography microscopy (DHM) or differential digital holography microscopy (DDHM).

In certain embodiments, the cultivated cells are output cells. In some embodiments, a composition of enriched T cells, such as engineered T cells, that has been cultivated is an output composition of enriched T cells. In particular embodiments, CD4+ T cells and/or CD8+ T cells that have been cultivated are output CD4+ and/or CD8+ T cells. In particular embodiments, a composition of enriched CD4+ T cells, such as engineered CD4+ T cells, that has been cultivated is an output composition of enriched CD4+ T cells. In some embodiments, a composition of enriched CD8+ T cells, such as engineered CD8+ T cells, that has been cultivated is an output composition of enriched CD8+ T cells.

In some embodiments, the cells are cultivated under conditions that promote proliferation and/or expansion in presence of one or more cytokines. In particular embodiments, at least a portion of the cultivation is performed with constant mixing and/or perfusion, such as mixing or perfusion controlled by a bioreactor. In some embodiments, the cells are cultivated in the presence or one or more cytokines and with a surfactant, e.g., poloxamer, such as poloxamer 188, to reduce shearing and/or shear stress from constant mixing and/or perfusion. In some embodiments, a composition of enriched CD4+ T cells, such as engineered CD4+ T cells, is cultivated in the presence of recombinant IL-2, IL-7, IL-15, and poloxamer, wherein at least a portion of the cultivating is performed with constant mixing and/or perfusion. In certain embodiments, a composition of enriched CD8+ T cells, such as engineered CD8+ T cells, is cultivated in the presence of recombinant IL-2, IL-15, and poloxamer, wherein at least a portion of the cultivating is performed with constant mixing and/or perfusion. In some embodiments, the cultivation is performed until the cells reach as threshold expansion of at least 4-fold e.g., as compared to the start of the cultivation.

Monitoring Cells During Cultivation

In some embodiments, the cells are monitored during the cultivation step. Monitoring may be performed, for example, to ascertain (e.g., measure, quantify) cell morphology, cell viability, cell death, and/or cell concentration (e.g., viable cell concentration). In some embodiments, the monitoring is performed manually, such as by a human operator. In some embodiments, the monitoring is performed by an automated system. The automated system may require minimal or no manual input to monitor the cultivated cells. In some embodiments, the monitoring is performed both manually and by an automated system.

In certain embodiments, the cells are monitored by an automated system requiring no manual input. In some embodiments, the automated system is compatible with a bioreactor, for example a bioreactor as described herein, such that cells undergoing cultivation can be removed from the bioreactor, monitored, and subsequently returned to the bioreactor. In some embodiments, the monitoring and cultivation occur in a closed loop configuration. In some aspects, in a closed loop configuration, the automated system and bioreactor remain sterile. In embodiments, the automated system is sterile. In some embodiments, the automated system is an in-line system.

In some embodiments, the automated system includes the use of optical techniques (e.g., microscopy) for detecting cell morphology, cell viability, cell death, and/or cell concentration (e.g., viable cell concentration). Any optical technique suitable for determining, for example, cell features, viability, and concentration are contemplated herein. Non-limiting examples of useful optical techniques include bright field microscopy, fluorescence microscopy, differential interference contrast (DIC) microscopy, phase contrast microscopy, digital holography microscopy (DHM), differential digital holography microscopy (DDHM), or a combination thereof. Differential digital holography microscopy, DDHM, and differential DHM may be used herein interchangeably. In certain embodiments, the automated system includes a differential digital holography microscope. In certain embodiments, the automated system includes a differential digital holography microscope including illumination means (e.g., laser, led). Descriptions of DDHM methodology and use may be found, for example, in U.S. Pat. No. 7,362,449; EP 1,631,788; U.S. Pat. Nos. 9,904,248; and 9,684,281, which are incorporated herein by reference in their entirety.

DDHM permits label-free, non-destructive imaging of cells, resulting in high-contrast holographic images. The images may undergo object segmentation and further analysis to obtain a plurality of morphological features that quantitatively describe the imaged objects (e.g., cultivated cells, cellular debris). As such, various features (e.g., cell morphology, cell viability, cell concentration) may be directly assessed or calculated from DDHM using, for example, the steps of image acquisition, image processing, image segmentation, and feature extraction. In some embodiments, the automated system includes a digital recording device to record holographic images. In some embodiments, the automated system includes a computer including algorithms for analyzing holographic images. In some embodiments, the automated system includes a monitor and/or computer for displaying the results of the holographic image analysis. In some embodiments, the analysis is automated (i.e., capable of being performed in the absence of user input). An example of a suitable automated system for monitoring cells during the cultivating step includes, but is not limited to, Ovizio iLine F (Ovizio Imaging Systems NV/SA, Brussels, Belgium).

In certain embodiments, the monitoring is performed continuously during the cultivation step. In some embodiments, the monitoring is performed in real-time during the cultivation step. In some embodiments, the monitoring is performed at discrete time points during the cultivation step. In some embodiments, the monitoring is performed at least every 15 minutes for the duration of the cultivating step. In some embodiments, the monitoring is performed at least every 30 minutes for the duration of the cultivating step. In some embodiments, the monitoring is performed at least every 45 minutes for the duration of the cultivating step. In some embodiments, the monitoring is performed at least every hour for the duration of the cultivating step. In some embodiments, the monitoring is performed at least every 2 hours for the duration of the cultivating step. In some embodiments, the monitoring is performed at least every 4 hours for the duration of the cultivating step. In some embodiments, the monitoring is performed at least every 6 hours for the duration of the cultivating step. In some embodiments, the monitoring is performed at least every 8 hours for the duration of the cultivating step. In some embodiments, the monitoring is performed at least every 10 hours for the duration of the cultivating step. In some embodiments, the monitoring is performed at least every 12 hours for the duration of the cultivating step. In some embodiments, the monitoring is performed at least every 14 hours for the duration of the cultivating step. In some embodiments, the monitoring is performed at least every 16 hours for the duration of the cultivating step. In some embodiments, the monitoring is performed at least every 18 hours for the duration of the cultivating step. In some embodiments, the monitoring is performed at least every 20 hours for the duration of the cultivating step. In some embodiments, the monitoring is performed at least every 22 hours for the duration of the cultivating step. In some embodiments, the monitoring is performed at least once a day for the duration of the cultivating step. In some embodiments, the monitoring is performed at least once every second day for the duration of the cultivating step. In some embodiments, the monitoring is performed at least once every third day for the duration of the cultivating step. In some embodiments, the monitoring is performed at least once every fourth day for the duration of the cultivating step. In some embodiments, the monitoring is performed at least once every fifth day for the duration of the cultivating step. In some embodiments, the monitoring is performed at least once every sixth day for the duration of the cultivating step. In some embodiments, the monitoring is performed at least once every seventh day for the duration of the cultivating step. In some embodiments, the monitoring is performed at least once every eighth day for the duration of the cultivating step. In some embodiments, the monitoring is performed at least once every ninth day for the duration of the cultivating step. In some embodiments, the monitoring is performed at least once every tenth day for the duration of the cultivating step. In some embodiments, the monitoring is performed at least once during the cultivating step.

In some embodiments, features of the cells that can be determined by the monitoring, including using optical techniques such as DHM or DDHM, include cell viability, cell concentration, cell number and/or cell density. In some embodiments, cell viability is characterized or determined. In some embodiments, cell concentration, density and/or number is characterized or determined. In some embodiments, viable cell concentration, viable cell number and/or viable cell density is characterized or determined. In some embodiments, the cultivated cells are monitored by the automated system until a threshold of expansion is reached, such as described above. In some embodiments, once a threshold of expansion is reached, the cultivated cells are harvested, such as by automatic or manual methods, for example, by a human operator. The threshold of expansion may depend on the total concentration, density and/or number of cultured cells determined by the automated system. Alternatively, the threshold of expansion may depend on the viable cell concentration, density and/or number.

In some embodiments, the harvested cells are formulated as described, such as in the presence of a pharmaceutically acceptable carrier. In some embodiments, the harvested cells are formulated in the presence of a cryoprotectant.

E. Formulating the Cells

In some embodiments, the provided methods for manufacturing, generating or producing a cell therapy and/or engineered cells may include formulation of cells, such as formulation of genetically engineered cells resulting from the provided processing steps prior to or after the incubating, engineering, and cultivating, and/or one or more other processing steps as described. In some embodiments, the provided methods associated with formulation of cells include processing transduced cells, such as cells transduced and/or expanded using the processing steps described above, in a closed system. In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods.

In some cases, the cells are processed in one or more steps (e.g. carried out in the centrifugal chamber and/or closed system) for manufacturing, generating or producing a cell therapy and/or engineered cells may include formulation of cells, such as formulation of genetically engineered cells resulting from the provided transduction processing steps prior to or after the culturing, e.g. cultivation and expansion, and/or one or more other processing steps as described. In some cases, the cells can be formulated in an amount for dosage administration, such as for a single unit dosage administration or multiple dosage administration. In some embodiments, the provided methods associated with formulation of cells include processing transduced cells, such as cells transduced and/or expanded using the processing steps described above, in a closed system.

In certain embodiments, one or more compositions of enriched T cells, such as engineered and cultivated T cells, e.g. output T cells, therapeutic cell composition, are formulated. In particular embodiments, one or more compositions of enriched T cells, such as engineered and cultivated T cells, e.g. output T cells, therapeutic cell composition, are formulated after the one or more compositions have been engineered and/or cultivated. In particular embodiments, the one or more compositions are input compositions. In some embodiments, the one or more input compositions have been previously cryofrozen and stored, and are thawed prior to the incubation.

In certain embodiments, the one or more therapeutic compositions of enriched T cells, such as engineered and cultivated T cells, e.g. output T cells, are or include two separate compositions, e.g., separate engineered and/or cultivated compositions, of enriched T cells. In particular embodiments, two separate therapeutic compositions of enriched T cells, e.g., two separate compositions of enriched CD4+ T cells and CD8+ T cells selected, isolated, and/or enriched from the same biological sample, separately engineered and separately cultivated, are separately formulated. In certain embodiments, the two separate therapeutic cell compositions include a composition of enriched CD4+ T cells, such as a composition of engineered and/or cultivated CD4+ T cells. In particular embodiments, the two separate therapeutic cell compositions include a composition of enriched CD8+ T cells, such as a composition of engineered and/or cultivated CD8+ T cells. In some embodiments, two separate therapeutic compositions of enriched CD4+ T cells and enriched CD8+ T cells, such as separate compositions of engineered and cultivated CD4+ T cells and engineered and cultivated CD8+ T cells, are separately formulated. In some embodiments, a single therapeutic composition of enriched T cells is formulated. In certain embodiments, the single therapeutic composition is a composition of enriched CD4+ T cells, such as a composition of engineered and/or cultivated CD4+ T cells. In some embodiments, the single therapeutic composition is a composition of enriched CD4+ and CD8+ T cells that have been combined from separate compositions prior to the formulation.

In some embodiments, separate therapeutic compositions of enriched CD4+ and CD8+ T cells, such as separate compositions of engineered and cultivated CD4+ and CD8+ T cells, are combined into a single therapeutic composition and are formulated. In certain embodiments, separate formulated therapeutic compositions of enriched CD4+ and enriched CD8+ T cells are combined into a single therapeutic composition after the formulation has been performed and/or completed. In particular embodiments, separate therapeutic compositions of enriched CD4+ and CD8+ T cells, such as separate compositions of engineered and cultivated CD4+ and CD8+ T cells, are separately formulated as separate compositions.

In some embodiments, the therapeutic composition of enriched CD4+ T cells, such as an engineered and cultivated CD4+ T cells, e.g. output CD4+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express a recombinant receptor and/or have been transduced or transfected with the recombinant polynucleotide. In certain embodiments, the therapeutic composition of enriched CD4+ T cells, such as an engineered and cultivated CD4+ T cells, e.g. output CD4+ T cells, that is formulated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the therapeutic composition of enriched CD8+ T cells, such as an engineered and cultivated CD8+ T cells, e.g. output CD8+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the therapeutic composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide. In certain embodiments, the therapeutic composition of enriched CD8+ T cells, such as an engineered and cultivated CD8+ T cells, e.g. output CD8+ T cells, that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, attributes of the one or more therapeutic compositions are assessed, for example as described in Sections I-A and I-A-2, prior to formulation. In some embodiments, the attributes are cell phenotypes and recombinant receptor-dependent activity. In some embodiments, the attributes are second attributes. In some embodiments, the attributes, e.g., cell phenotypes and recombinant receptor-dependent activity, are quantified to provide a number, percentage, proportion, and/or ratio of cells having an attribute in the therapeutic cell composition. In some embodiments, the attributes are used as input to a process including a statistical method, for example as described herein, to identify correlations between attributes of the input composition and the resulting therapeutic cell composition. In some embodiments, the attributes are used as training data, for example with input attributes, to train a process including a statistical learning model, for example as described herein, to predict therapeutic cell composition attributes.

In certain embodiments, the formulated cells are output cells. In some embodiments, a formulated therapeutic composition of enriched T cells, such as a formulated composition of engineered and cultivated T cells, is an output composition of enriched T cells. In particular embodiments, the formulated CD4+ T cells and/or formulated CD8+ T cells are the output CD4+ and/or CD8+ T cells. In particular embodiments, a formulated composition of enriched CD4+ T cells is an output composition of enriched CD4+ T cells. In some embodiments, a formulated composition of enriched CD8+ T cells is an output composition of enriched CD8+ T cells.

In some embodiments, cells can be formulated into a container, such as a bag or vial. In some embodiments, the cells are formulated between 0 days and 10 days, between 0 and 5 days, between 2 days and 7 days, between 0.5 days, and 4 days, or between 1 day and 3 days after the cells after the threshold cell count, density, and/or expansion has been achieved during the cultivation. In certain embodiments, the cells are formulated at or at or about or within 12 hours, 18 hours, 24 hours, 1 day, 2 days, or 3 days after the threshold cell count, density, and/or expansion has been achieved during the cultivation. In some embodiments, the cells are formulated within or within about 1 day after the threshold cell count, density, and/or expansion has been achieved during the cultivation.

Particular embodiments contemplate that cells are in a more activated state at early stages during the cultivation than at later stages during the cultivation. Further, in some embodiments, it may be desirable to formulate cells that are in a less activated state than the peak activation that occurs or may occur during the cultivation. In certain embodiments, the cells are cultivated for a minimum duration or amount of time, for example, so that cells are harvested in a less activated state than if they were formulated at an earlier time point during the cultivation, regardless of when the threshold is achieved. In some embodiments, the cells are cultivated between 1 day and 3 days after the threshold cell count, density, and/or expansion has been achieved during the cultivation. In certain embodiments, the cells achieve the threshold cell count, density, and/or expansion and remain cultivated for a minimum time or duration prior to the formulation. In some embodiments, cells that have achieved the threshold are not formulated until they have been cultivated for a minimum duration and/or amount of time, such as a minimum time or duration of between 1 day and 14 days, 2 days and 7 days, or 3 days and 6 days, or a minimum time or duration of the cultivation of or of about 2 days, 3 days, 4 days, 5 days, 6 days, 7 days, or more than 7 days. In some embodiments, the minimum time or duration of the cultivation is between 3 days and 6 days.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cell are formulated with a cryopreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the therapeutic composition of enriched T cells, e.g., T cells that have been stimulated, engineered, and/or cultivated, are formulated, cryofrozen, and then stored for an amount of time. In certain embodiments, the formulated, cryofrozen cells are stored until the cells are released for infusion. In particular embodiments, the formulated cryofrozen cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryofrozen and stored for, for about, or for less than 1 days, 2 days, 3 days, 4 days, 5 days, 6 days, or 7 days. In certain embodiments, the cells are thawed and administered to a subject after the storage. In certain embodiments, the cells are stored for or for about 5 days.

In some embodiments, the formulation is carried out using one or more processing step including washing, diluting or concentrating the cells, such as the cultured or expanded cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or expanded cells. In some embodiments, the volume of formulation buffer is from 10 mL to 1000 mL or from about 10 mL to about 1000 mL, such as at least or about at least or about 50 mL, 100 mL, 200 mL, 300 mL, 400 mL, 500 mL, 600 mL, 700 mL, 800 mL, 900 mL or 1000 mL.

In some embodiments, such processing steps for formulating a cell composition is carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as the vials of the biomedical material vessels described herein, that is operably linked as part of a closed system with the centrifugal chamber. In some embodiments, the biomedical material vessels are configured for integration and/or operable connection and/or is integrated or operably connected, to a closed system or device that carries out one or more processing steps. In some embodiments, the biomedical material vessel is connected to a system at an output line or output position. In some cases, the closed system is connected to the vial of the biomedical material vessel at the inlet tube. Exemplary close systems for use with the biomedical material vessels described herein include the Sepax® and Sepax® 2 system.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of vials, can be sterilely connected to one or more, generally two or more, such as at least 3, 4, 5, 6, 7, 8 or more of the ports of the multi-port output. For example, in some embodiments, one or more containers, e.g., biomedical material vessels, can be attached to the ports, or to fewer than all of the ports. Thus, in some embodiments, the system can effect expression of the output composition into a plurality of vials of the biomedical material vessels.

In some aspects, cells can be expressed to the one or more of the plurality of output containers, e.g., vials of the biomedical material vessels, in an amount for dosage administration, such as for a single unit dosage administration or multiple dosage administration. For example, in some embodiments, the vials of the biomedical material vessels, may each contain the number of cells for administration in a given dose or fraction thereof. Thus, each vial, in some aspects, may contain a single unit dose for administration or may contain a fraction of a desired dose such that more than one of the plurality of vials, such as two of the vials, or 3 of the vials, together constitutes a dose for administration.

Thus, the vials described herein, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, each of the containers, e.g., bags of vials individually comprises a unit dose of the cells. Thus in some embodiments, each of the containers comprises the same or approximately or substantially the same number of cells. In some embodiments, each unit dose contains at least or about at least 1×10⁶, 2×10⁶, 5×10⁶, 1×10⁷, 5×10⁷, or 1×10⁸ engineered cells, total cells, T cells, or PBMCs. In some embodiments, the volume of the formulated cell composition in each container, e.g. bag or vial, is 10 mL to 100 mL, such as at least or about at least or about 20 mL, 30 mL, 40 mL, 50 mL, 60 mL, 70 mL, 80 mL, 90 mL or 100 mL. In some embodiments, the cells in the container, e.g. bag or vials, can be cryopreserved. In some embodiments, the container, e.g. vials, can be stored in liquid nitrogen until further use.

In some embodiments, such cells produced by the method, or a composition comprising such cells, are administered to a subject for treating a disease or condition.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software.

Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### V. Exemplary Embodiments

Among the provided embodiments are:

1. A method of predicting attributes of a cell composition, the method comprising:

(a) determining a percentage, number, ratio, and/or proportion of cells in an input cell composition that have first attributes, wherein the first attributes comprise cell phenotypes, and wherein the input composition comprises T cells selected from a biological sample from a subject; and

(b) applying the first attributes as input to a process configured to predict, based on the first attributes, a percentage, number, ratio and/or proportion of cells in a therapeutic cell composition that have second attributes, wherein the second attributes comprise cell phenotypes and recombinant receptor-dependent activity, and wherein:

the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells.

2. The method of embodiment 1, further comprising (c) determining, based on the predicted second attributes, whether the therapeutic cell composition is predicted to have a desired attribute.

3. The method of embodiment 2, wherein:

if the therapeutic cell composition is predicted to have a desired attribute, a predetermined treatment regimen comprising the therapeutic cell composition is administered to a subject; or

if the therapeutic cell composition is predicted to not have a desired attribute, the predetermined treatment regimen comprising the therapeutic cell composition is altered and the altered treatment regimen comprising the therapeutic cell composition is administered to the subject.

4. A method of predicting attributes of a cell composition, the method comprising:

(a) determining a percentage, number, ratio, and/or proportion of cells in an input cell composition that have first attributes, wherein the first attributes comprise cell phenotypes, and wherein the input composition comprises T cells selected from a sample from a subject;

(b) applying the first attributes as input to a process configured to predict, based on the first attributes, a percentage, number, ratio and/or proportion of cells in an therapeutic cell composition that have one second attribute, wherein the one second attribute comprises a cell phenotype or recombinant receptor-dependent activity, and wherein:

the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells.

5. The method of embodiment 4, further comprising (c) determining, based on the one predicted second attribute, whether the therapeutic cell composition is predicted to have a desired attribute.

6. The method of embodiment 5, wherein:

(i) if the therapeutic cell composition is predicted to have a desired attribute, a predetermined treatment regimen comprising the therapeutic cell composition is administered to a subject; or

(ii) if the therapeutic cell composition is predicted to not have a desired attribute, the predetermined treatment regimen comprising the therapeutic cell composition is altered and the altered treatment regimen comprising the therapeutic cell composition is administered to the subject.

7. A method of treating a subject, the method comprising:

(a) selecting T cells from a sample from a subject to produce an input composition comprising T cells;

(b) determining a percentage, number, ratio, and/or proportion of T cells in the input composition having first attributes, wherein the first attributes comprise cell phenotypes;

(c) applying the first attributes as input to a process configured to predict, based on the first attributes, a percentage, number, ratio, and/or proportion of cells in a therapeutic cell composition that have second attributes, wherein the second attributes comprises a cell phenotype and recombinant receptor-dependent activity, and wherein the therapeutic cell composition comprises the recombinant receptor, and wherein:

the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells;

(d) determining, based on the predicted second attributes, whether the therapeutic cell composition is predicted to have a desired attribute; and

(e) administering a treatment to the subject wherein:
(i) if the therapeutic cell composition is predicted to have the desired attribute, a predetermined treatment regimen comprising the therapeutic cell composition is administered; or
(ii) if the therapeutic cell composition is predicted to not have the desired attribute, administering to the subject a treatment regimen comprising the therapeutic cell composition that is altered compared to the predetermined treatment regimen comprising the therapeutic cell composition.

8. A method of treating a subject, the method comprising:

(a) selecting T cells from a sample from a subject to produce an input composition comprising T cells;

(b) determining a percentage, number, ratio, and/or proportion of T cells in the input composition having first attributes, wherein the first attributes comprise cell phenotypes;

(c) applying the first attributes as input to a process configured to predict, based on the first attributes, a percentage, number, ratio and/or proportion of cells in an therapeutic cell composition that have one second attribute, wherein the one second attribute comprises a cell phenotype or recombinant receptor-dependent activity, and wherein the therapeutic cell composition comprises the recombinant receptor, and wherein

the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells;

(d) determining, based on the one predicted second attribute, whether the therapeutic cell composition is predicted to have a desired attribute; and

(e) administering a treatment to the subject wherein:
(i) if the therapeutic cell composition is predicted to have the desired attribute, a predetermined treatment regimen comprising the therapeutic cell composition is administered; or
(ii) if the therapeutic cell composition is predicted to not have the desired attribute, administering to the subject a treatment regimen comprising the therapeutic cell composition that is altered compared to the predetermined treatment regimen comprising the therapeutic cell composition.

9. A method comprising:

(a) determining a percentage, number, ratio, and/or proportion of cells in an input cell composition that have first attributes, wherein the first attributes comprise cell phenotypes, and wherein the input composition comprises T cells selected from a sample from a subject;

(b) determining a percentage, number, ratio, and/or proportion of cells in a therapeutic cell composition that have second attributes, wherein the second attributes comprise cell phenotypes and recombinant receptor-dependent activity, wherein the therapeutic cell composition comprises the recombinant receptor, and wherein the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells;

(c) training a canonical correlation analysis statistical learning model on the first and second attributes.

10. The method of any of embodiments 1-3 and 7, wherein:

the process comprises a canonical correlation analysis statistical learning model trained according to the method of embodiment 9; and

applying the first attributes as input to the process comprises applying the first attributes to the canonical correlation analysis statistical learning model.

11. A method comprising:

(a) determining a percentage, number, ratio, and/or proportion of cells in an input cell composition that have first attributes, wherein the first attributes comprise cell phenotypes, and wherein the input composition comprises T cells selected from a sample from a subject;

(b) determining a percentage, number, ratio, and/or proportion of cells in an therapeutic cell composition that have one second attribute, wherein the one second attribute comprises cell phenotypes and recombinant receptor-dependent activity, wherein the therapeutic cell composition comprises the recombinant receptor, and wherein:

the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells;

(c) training a lasso regression statistical learning model on the first attributes and the one second attribute.

12. The method of any of embodiments 4-6 and 8, wherein:

the process comprises a lasso regression statistical learning model trained according to the method of embodiment 11; and

applying the first attributes as input to the process comprises applying the first attributes to the lasso regression statistical learning model.

13. A method of determining attributes of an input cell composition correlated with attributes of an therapeutic cell composition, the method comprising:

(a) determining a percentage, number, ratio and/or proportion of cells in an input cell composition that have first attributes, wherein the first attributes comprise cell phenotypes, and wherein the input composition comprises T cells selected from a sample from a subject;

(b) determining a percentage, number, ratio, and/or proportion of cells in a therapeutic cell composition that have second attributes, wherein the second attributes comprise cell phenotypes and recombinant receptor-dependent activity, wherein the therapeutic cell composition comprises the recombinant receptor, and wherein:

the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells;

(c) performing canonical correlation analysis (CCA) between the first attributes and the second attributes; and

(d) identifying, based on the canonical correlation analysis, the first attributes correlated with the second attributes.

14. The method of embodiment 13, wherein the CCA comprises a penalty function capable of regularizing the first and second attributes.

15. The method of embodiment 14, wherein the penalty function comprises a constant, said constant determined by performing permutations on the first and second attributes, independently, and performing canonical correlation analysis.

16. The method of embodiment 14 or embodiment 15, wherein the penalty function is lasso regularization.

17. The method of any of embodiments 13-16, wherein the method further comprises constraining the square of the L2 norm of canonical vectors to be less than or equal to 1.

18. A method of determining attributes of an input composition correlated with attributes of an therapeutic cell composition, the method comprising:

(a) determining a percentage, number, ratio, and/or proportion of cells in an input cell composition that have first attributes, wherein the first attributes comprises cell phenotypes, and wherein the input composition comprises T cells selected from a sample from a subject;

(b) determining a percentage, number, ratio, and/or proportion of cells in a therapeutic cell composition that have one second attribute, wherein the one second attribute comprises a cell phenotype or a recombinant receptor-dependent activity, wherein the therapeutic cell composition comprises the recombinant receptor, and wherein

the input composition comprises CD4+, CD8+, or CD4+ and CD8+ T cells and the therapeutic cell composition comprises the recombinant receptor and is produced from the input composition; or

the input composition is a first input composition comprising CD4+ or CD8 T cells and the output cell composition comprises the recombinant receptor and is produced from another input composition comprising the other of the CD4+ or CD8+ T cells;

(c) performing lasso regression between the first attributes and the second attributes; and

(d) identifying, based on the lasso regression, the first attributes correlated with the one second attribute.

19. The method of any of embodiments 1-6, and 9-18, wherein the method further comprises prior to (a) selecting T cells from the sample from the subject to produce the input composition comprising CD4, CD8, or CD4 and CD8 T cells.

20. The method of any of embodiments 1-19, wherein the sample comprises a whole blood sample, a buffy coat sample, a peripheral blood mononuclear cell (PBMC) sample, an unfractionated T cell sample, a lymphocyte sample, a white blood cell sample, an apheresis product, or a leukapheresis product.

21. The method of embodiment 20, wherein the sample is an apheresis product or leukapheresis product.

22. The method of embodiment 21, wherein the apheresis product or leukapheresis product has been previously cryopreserved.

23. The method of any of embodiments 1-22, wherein the T cells comprise primary cells obtained from the subject.

25. The method of any of embodiments 1-24, wherein the recombinant receptor is a chimeric antigen receptor (CAR).

26. The method of any of embodiments 1-25, wherein the first attributes comprise one or more cell phenotypes comprising 3CAS−/CCR7−/CD27−, 3CAS−/CCR7−/CD27+, 3CAS−/CCR7+, 3CAS−/CCR7+/CD27−, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and CAS+/CD3+.

27. The method of any of embodiments 1-26, wherein the first attributes comprise one or more cell phenotypes comprising 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and CAS+/CD3+ of an input composition that is CD8+ cells.

28. The method of any of embodiments 1 to 27, wherein the second attributes comprise one or more cell phenotypes and/or recombinant receptor-dependent activity comprising 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD19+, CD3+, CYTO−/CAR+, EGFRt+, IFNG+, viable cell concentration (VCC), vector copy number (VCN), viability, GMCSF+/CD19+, CD3+/CAR+, CD3+/CD56+, CAR+, IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+/CD19+, IFNG+/CD19+, IL10+/CD19+, IL13+/CD19+, IL2+/CD19+, IL5+/CD19+, MIP1A+/CD19+, MIP1B+/CD19+, sCD137+/CD19+, and TNFa+/CD19+.

29. The method of any of embodiments 1 to 27, wherein the second attributes comprise one or more cell phenotypes and/or recombinant receptor-dependent activity comprising 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD19+, CD3+, CD3+/CD4+, CD4+/EGFRt+, CYTO−/CD4+/CAR+, EGFRt+, IFNG+, VCC, VCN, viability, GMCSF+/CD19+, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD19+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+/CAR+, IL-2+ of CD8+/CAR+, IL-2+/TNFA+/CD8+/CAR+, cytolytic CD8+, TNFA+ of CD8+CAR+, IFNG+/CD19+, IL-10+/CD19+, IL-13+/CD19+, IL-2+/CD19+, IL-5+/CD19+, MIP1A+/CD19+, MIP1B+/CD19+, sCD137+/CD19+, and/or TNFa+/CD19+.

30. The method of any of embodiments 1 to 29, wherein the first attributes comprise or comprise about 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 cell phenotypes.

31. The method of any of embodiments 1 to 30, wherein the first attributes comprise or comprise about or at least 2, 4, 6, 8, 10, 12, or more cell phenotypes.

32. The method of any of embodiments 1 to 31, wherein the first attributes comprise greater than or greater than about 5, 10, 15, or 20 cell attributes.

33. The method of any of embodiments 1 to 32, wherein the second attributes comprise or comprise about 101, 90, 80, 70, 60, 50, 40, 30, 20, 15, 10, 5, 4, 3, 2, or 1 cell phenotypes and recombinant receptor-dependent activity.

34. The method of any of embodiments 1 to 33, wherein the second attributes comprise about or at least 1, 2, 4, 6, 8, 10, 12, or more cell phenotypes and recombinant receptor-dependent activity.

35. The method of any of embodiments 1 to 34, wherein the second attributes comprise 1 cell phenotype or recombinant receptor-dependent activity.

36. The method of any of embodiments 2, 3, 7, 10, and 19-35, wherein the desired attribute is at least one attribute that is correlated to clinical response of the therapeutic cell composition.

37. The method of any of embodiments 5, 6, 8, 12, and 19-35, wherein the desired attribute is an attribute that is correlated to clinical response of the therapeutic cell composition.

38. The method of embodiment 36 or embodiment 37, wherein the clinical response is a durable response and/or progression free survival.

39. The method of any of embodiments 2, 3, 5-12, and 19-38, wherein the desired attribute is a threshold percentage of CD27+/CCR7+ T cells in the therapeutic cell composition.

40. The method of embodiment 39, wherein the threshold percentage is at least or at least about 60% of the cells in the therapeutic cell composition are CD27+/CCR7+.

41. The method of embodiment 39 or embodiment 40, wherein the CD27+/CCR7+ cells are CD4+/CAR+ T cells and/or CD8+/CAR+ T cells.

42. The method of any of embodiments 2, 3, 5-12, and 19-38, wherein the desired attribute is a threshold percentage of IL-2+ of CD4+/CAR+ and IL-2+/TNFA+/CD4+/CAR+ T cells in the therapeutic cell composition.

43. The method of embodiment 42, wherein the threshold percentage is at least at or about 70%, 80%, 85%, 90%, 91%, 92%, 93%, 94%, 95%, 96%, 97%, 98%, 99% or 100% of the total number of CD4+ T cells in the therapeutic cell composition.

44. The method of any of embodiments 2, 3, 5-12, and 19-38, wherein the desired attribute is a threshold percentage of IFNG+/IL-2+/CD4+/CAR+, IFNG+/IL-2+/IL-17+/TNFA+/CD4+/CAR+, IFNG+/IL-2+/TNFA+/CD4+/CAR+, IFNG+/TNFA+/CD4+/CAR+, IL-17+ of CD4+CAR+, IL-2+ of CD4+CAR+, and/or IL-2+/TNFA+/CD4+/CAR+ T cells in the therapeutic cell composition.

45. The method of embodiment 44, wherein the threshold percentage is at least at or about 10%, 15%, 20%, 25%, 30%, 40%, 50%, 60% or more of the total number of CAR+/CD4+ T cells in the therapeutic cell composition.

46. The method of any of embodiments 3, 6-8, 10, 12, and 19-45, wherein altering the predetermined treatment regimen comprises increasing a dosing frequency or the volume of a unit dose.

47. The method of embodiment 46, wherein increasing the dosing frequency or the volume of the unit dose improves clinical response.

48. The method of any of embodiments 3, 6-8, 10, 12, and 19-45, wherein altering the predetermined treatment regimen comprises administering the therapeutic cell composition in combination with a second therapeutic agent.

49. The method of embodiment 48, wherein the second therapeutic agent is a cytokine.

50. The method of embodiment 49, wherein the cytokine is IL-2.

51. The method of embodiment 48, wherein the second therapeutic agent is a chemotherapeutic agent.

52. A method of determining attributes of a therapeutic cell composition, the method comprising assessing an input composition comprising T cells for a phenotype, or a percentage, number, ratio and/or proportion of cells of the phenotype, thereby determining, from the phenotype, the likelihood or presence of an attribute in a therapeutic cell composition, or a percentage, number, ratio and/or proportion of cells having the attribute in the therapeutic cell composition, wherein:

the therapeutic cell composition comprises a recombinant receptor, and wherein

the input composition comprises CD4+, CD8+, or CD4+ and CD8+ T cells and the therapeutic cell composition comprises a recombinant receptor and is produced from the input composition; or

the input composition is a first input composition comprising CD4+ or CD8 T cells and the output cell composition comprises a recombinant receptor and is produced from another input composition comprising the other of the CD4+ or CD8+ T cells, and the phenotype and attribute is selected from:

(a) a phenotype that is CD27+/CCR7+, CD27+, CCR7+, or CCR7+/CD45RA+ of CD4+ T cells in the input composition and an attribute that is CD27+/CCR7+, CD27+, CCR7+, CCR7+/CD45RA+ of CD4+ T cells and CD8+ T cells in the therapeutic cell composition;

(b) a phenotype that is CD27+/CCR7+, CD27+, CCR7+, CD28+/CD27+, or CD28+ of CD4+ T cells in the input composition and an attribute that is CD27+/CCR7+, CD27+, CCR7+, or CCR7+/CD45RA+ of CD8+ T cells in the therapeutic cell composition;

(c) a phenotype that is CD28−/CD27−, CCR7−/CD27−, or CCR7+/CD45RA+ of CD4+ T cells in the input composition and an attribute that is CD28−/CD27−, CCR7−/CD27−, or CCR7+/CD45RA+ of CD8+ T cells in the therapeutic cell composition;

(d) a phenotype that is CD27+/CCR7+, CD27+, CCR7+, CD28+/CD27+, or CD28+ of CD8+ cells in the input composition and an attribute that is CD28−/CD27−, CCR7−/CD27−, or CCR7+/CD45RA+ of CD8+ T cells in the therapeutic cell composition;

(e) a phenotype that is CD28−/CD27−, CCR7−/CD27−, or CCR7+/CD45RA+ of CD8+ T cells in the input composition and an attribute that is CD28−/CD27−, CCR7−/CD27−, or CCR7+/CD45RA+ of CD8+ T cells of the therapeutic T cell composition;

(f) a phenotype that is CCR7−/CD45RA−, CCR7−/CD27−, or CD28+/CD27− of CD4+ T cells in the input composition and an attribute that is IFNg+, IL-5+, or GMCSF+ of CD4+ T cells of the therapeutic cell composition;

(g) a phenotype that is CCR7−/CD45RA−, CCR7−/CD27−, or CD28+/CD27− of CD4+ T cells in the input composition and an attribute that is IL-2+ or TNFa+ of CD8+ T cells of the output composition;

(h) a phenotype that is CCR7+/CD27−, CD28+/CD27−, or CCR7+/CD45RA− of CD8 T cells in the input composition and an attribute that is IL-5+, IL-13+, TNF-a+, or IL-2+ of CD8+ T cells in the output composition

(i) a phenotype that is CCR7+/CD27+ or CCR7+CD45RA+ of CD8+ and CD4+ cells in the input composition and an attribute that is CCR7+/CD27+ or CCR7+CD45RA+ of CD8+ T cells in the therapeutic cell composition;

(j) a phenotype that is CCR7−/CD27− of CD4+ and CD8+ T cells in the input composition and an attribute that is IFNg+, TNF-a+, IL-13+, IL-2+, or IL-5+ of CD8+ T cells in the therapeutic cell composition.

53. The method of embodiment 52, wherein the method further comprises selecting T cells from the sample from the subject to produce the input composition comprising CD4, CD8, or CD4 and CD8 T cells.

54. The method of any of embodiments 1-53, wherein the therapeutic cell composition is generated by manufacturing the input composition.

55. The method of any of embodiments 1-54, wherein the manufacturing comprises stimulating the input cell composition.

56. The method of any of embodiments 1-55, wherein the manufacturing comprises transducing the input composition with a vector comprising a recombinant receptor.

57. The method of embodiment 56, wherein the recombinant receptor is a chimeric antigen receptor (CAR).

58. The method of ay of embodiments 1-57 wherein the phenotypes of the input composition are assessed or determined prior to stimulation.

### VI. EXAMPLES

The following examples are included for illustrative purposes only and are not intended to limit the scope of the invention.

### Example 1: Statistical Methods for Predicting Attributes of Therapeutic Compositions of CD4+ and CD8+ Cells Expressing an Anti-CD19 CAR

To investigate the influence of patient-derived starting material on manufactured therapeutic composition characteristics, various attributes of patient materials and the resulting manufactured therapeutic compositions from 119 patients with Diffuse Large B-Cell Lymphoma (DLBCL) were assessed using two statistical methods: canonical correlation analysis and lasso regression.

Therapeutic compositions of engineered CD4+ T cells and engineered CD8+ T cells each expressing the same anti-CD19 chimeric antigen receptor (CAR) were produced by a process involving subjecting enriched CD4+ and enriched CD8+ cell populations, separately, to processing steps. CD4+ and CD8+ cells were separately selected from human peripheral blood mononuclear cells (PBMCs) that had been obtained by leukapheresis, generating separate enriched CD4+ and enriched CD8+ cell compositions (e.g., input compositions), which then were cryopreserved. The CD4+ and CD8+ compositions were subsequently thawed and separately underwent steps for stimulation, transduction, and expansion.

The thawed CD4+ and CD8+ cells were separately stimulated in the presence of paramagnetic polystyrene-coated beads coupled to anti-CD3 and anti-CD28 antibodies at a 1:1 bead to cell ratio. The stimulation was carried out in media containing human recombinant IL-2, human recombinant IL-15, and N-Acetyl Cysteine (NAC). The CD4+ cell media also included human recombinant IL-7.

Following the introduction of the beads, CD4+ and CD8+ cells were separately transduced with a lentiviral vector encoding the same anti-CD19 CAR. The CAR contained an anti-CD19 scFv derived from a murine antibody, an immunoglobulin spacer, a transmembrane domain derived from CD28, a costimulatory region derived from 4-1BB, and a CD3-zeta intracellular signaling domain. The vector also encoded a truncated EGFR (EGFRt) that served as a surrogate marker for CAR expression that was connected to the CAR construct by a T2A sequence. The cells were transduced in the presence of 10 μg/ml protamine sulfate.

After transduction, the beads were removed from the cell compositions by exposure to a magnetic field. The CD4+ and CD8+ cell compositions were then separately cultivated for expansion with continual mixing and oxygen transfer by a bioreactor (Xuri W25 Bioreactor). Poloxamer was added to the media. Both cell compositions were cultivated in the presence of IL-2 and IL-15. The CD4+ cell media also included IL-7. The CD4+ and CD8+ cells were each cultivated, prior to harvest, to 4-fold expansion. One day after reaching the threshold, cells from each composition were separately harvested, formulated, and cryopreserved. The exemplary process is summarized in Table E1.

TABLE E1
Summary of the exemplary process for generating
CD4+ and CD8+ CAR-T cells

| Stage | CD4+ cells | CD8+ cells |
|---|---|---|
| Stimulation | anti-CD3/CD28 antibody | anti-CD3/CD28 antibody |
| (day 1-2) | conjugated beads | conjugated beads |
| | 1:1 bead to cell ratio | 1:1 bead to cell ratio |
| | media: IL-2, IL-7, IL-15, | media: IL-2, IL-15, and |
| | and NAC | NAC |
| Transduction | transduction adjuvant (e.g. | transduction adjuvant |
| (day 2-5) | 10 μg/ml protamine sulfate) | (e.g. 10 μg/ml protamine |
| | | sulfate) |
| Bead removal | magnetic bead removal | magnetic bead removal |
| (day 5*) | | |
| Expansion | rocking motion bioreactor | rocking motion bioreactor |
| (day 5*-Harvest) | and/or continuous mixing | and/or continuous mixing |
| | media: IL-2, IL-7, IL15, | media: IL-2, IL15, and |
| | and poloxamer | poloxamer |

*Approximate

Thirty-four cell phenotypes of the enriched CD4+ and enriched CD8+ cell populations (e.g., input compositions), and 101 cell phenotype and functional attributes (e.g., recombinant receptor-dependent activity) from the therapeutic compositions (e.g., output compositions) were assessed (Table E2). The results were used as input to the statistical learning methods to assess the relationship between input composition attributes and therapeutic composition attributes.

TABLE E2
Summary of phenotype, health, and functional attributes assessed.

| Attribute | Composition | Description |
|---|---|---|
| 3CAS−/CCR7−/CD27−/CD4+ | CD4 INPUT | T cell phenotype |
| 3CAS−/CCR7−/CD27+/CD4+ | CD4 INPUT | T cell phenotype |
| *3CAS−/CCR7+/CD4+ | CD4 INPUT | T cell phenotype |
| 3CAS−/CCR7+/CD27−/CD4+ | CD4 INPUT | T cell phenotype |
| 3CAS−/CCR7+/CD27+/CD4+ | CD4 INPUT | T cell phenotype |
| *3CAS−/CD27+/CD4+ | CD4 INPUT | T cell phenotype |
| *3CAS−/CD28−/CD27−/CD4+ | CD4 INPUT | T cell phenotype |
| *3CAS−/CD28−/CD27+/CD4+ | CD4 INPUT | T cell phenotype |
| *3CAS−/CD28+/CD4+ | CD4 INPUT | T cell phenotype |
| *3CAS−/CD28+/CD27− CD4+ | CD4 INPUT | T cell phenotype |
| *3CAS−/CD28+/CD27+/CD4+ | CD4 INPUT | T cell phenotype |
| *3CAS−/CCR7−/CD45RA−/CD4+ | CD4 INPUT | T cell phenotype |
| *3CAS−/CCR7−/CD45RA+/CD4+ | CD4 INPUT | T cell phenotype |
| *3CAS−/CCR7+/CD45RA−/CD4+ | CD4 INPUT | T cell phenotype |
| *3CAS−/CCR7+/CD45RA+/CD4+ | CD4 INPUT | T cell phenotype |
| CAS+ of CD3+ | CD4 INPUT | T cell phenotype |
| CAS+ of CD4+ | CD4 INPUT | T cell phenotype |
| 3CAS−/CCR7−/CD27−/CD8+ | CD8 INPUT | T cell phenotype |
| 3CAS−/CCR7−/CD27+/CD8+ | CD8 INPUT | T cell phenotype |
| *3CAS−/CCR7+/CD8+ | CD8 INPUT | T cell phenotype |
| 3CAS−/CCR7+/CD27−/CD8+ | CD8 INPUT | T cell phenotype |
| 3CAS−/CCR7+/CD27+/CD8+ | CD8 INPUT | T cell phenotype |
| *3CAS−/CD27+/CD8+ | CD8 INPUT | T cell phenotype |
| *3CAS−/CD28−/CD27−/CD8+ | CD8 INPUT | T cell phenotype |
| *3CAS−/CD28−/CD27+/CD8+ | CD8 INPUT | T cell phenotype |
| *3CAS−/CD28+/CD8+ | CD8 INPUT | T cell phenotype |
| *3CAS−/CD28+/CD27−/CD8+ | CD8 INPUT | T cell phenotype |
| *3CAS−/CD28+/CD27+/CD8+ | CD8 INPUT | T cell phenotype |
| *3CAS−/CCR7−/CD45RA−/CD8+ | CD8 INPUT | T cell phenotype |
| *3CAS−/CCR7−/CD45RA+/CD8+ | CD8 INPUT | T cell phenotype |
| *3CAS−/CCR7+/CD45RA−/CD8+ | CD8 INPUT | T cell phenotype |
| *3CAS−/CCR7+/CD45RA+/CD8+ | CD8 INPUT | T cell phenotype |
| CAS+ of CD3 | CD8 INPUT | T cell phenotype |
| CAS+ of CD8+ | CD8 INPUT | T cell phenotype |
| *3CAS−/CCR7−/CD27−/CD4+/ | CD4 | T cell phenotype |
| CAR+ | THERAPEUTIC | |
| *3CAS−/CCR7−/CD27+/CD4+/ | CD4 | T cell phenotype |
| CAR+ | THERAPEUTIC | |
| *3CAS−/CCR7+/CD4+/CAR+ | CD4 | T cell phenotype |
| | THERAPEUTIC | |
| *3CAS−/CCR7+/CD27−/CD4+/ | CD4 | T cell phenotype |
| CAR+ | THERAPEUTIC | |
| *3CAS−/CCR7+/CD27+/CD4+/ | CD4 | T cell phenotype |
| CAR+ | THERAPEUTIC | |
| *3CAS−/CD27+/CD4+/CAR+ | CD4 | T cell phenotype |
| | THERAPEUTIC | |
| *3CAS−/CD28−/CD27−/CD4+/ | CD4 | T cell phenotype |
| CAR+ | THERAPEUTIC | |
| *3CAS−/CD28−/CD27+/CD4+/ | CD4 | T cell phenotype |
| CAR+ | THERAPEUTIC | |
| *3CAS−/CD28+/CD4+/CAR+ | CD4 | T cell phenotype |
| | THERAPEUTIC | |
| *3CAS−/CD28+/CD27−/CD4+/ | CD4 | T cell phenotype |
| CAR+ | THERAPEUTIC | |
| *3CAS−/CD28+/CD27+/CD4+/ | CD4 | T cell phenotype |
| CAR+ | THERAPEUTIC | |
| *3CAS−/CCR7−/CD45RA−/CD4+/ | CD4 | T cell phenotype |
| CAR+ | THERAPEUTIC | |
| *3CAS−/CCR7−/CD45RA+/CD4+/ | CD4 | T cell phenotype |
| CAR+ | THERAPEUTIC | |
| *3CAS−/CCR7+/CD45RA−/CD4+/ | CD4 | T cell phenotype |
| CAR+ | THERAPEUTIC | |
| *3CAS−/CCR7+/CD45RA+/CD4+/ | CD4 | T cell phenotype |
| CAR+ | THERAPEUTIC | |
| *CAS+ of CD3+/CAR+ | CD4 | T cell phenotype |
| | THERAPEUTIC | |
| CD19 | CD4 | T cell phenotype |
| | THERAPEUTIC | |
| CD3 | CD4 | T cell phenotype |
| | THERAPEUTIC | |
| CD3+/CD4+ | CD4 | T cell phenotype |
| | THERAPEUTIC | |
| CD4+/EGFRt+ | CD4 | T cell phenotype |
| | THERAPEUTIC | |
| CYTO−/CD4+/CAR+ | CD4 | T cell phenotype |
| | THERAPEUTIC | |
| EGFRt+ | CD4 | T cell phenotype |
| | THERAPEUTIC | |
| *IFNG+ (intracellular) | CD4 | Recombinant |
| | THERAPEUTIC | receptor-dependent |
| | | activity |
| IFNG+/IL2+/CD4+/CAR+ | CD4 | Recombinant |
| (intracellular) | THERAPEUTIC | receptor-dependent |
| | | activity |
| IFNG+/IL2+/IL17+/TNFA+/CD4+/ | CD4 | Recombinant |
| CAR+ (intracellular) | THERAPEUTIC | receptor-dependent |
| | | activity |
| IFNG+/IL2+/TNFA+/CD4+/CAR+ | CD4 | Recombinant |
| (intracellular) | THERAPEUTIC | receptor-dependent |
| | | activity |
| IFNG+ OF CD4+/CAR+ | CD4 | Recombinant |
| (intracellular) | THERAPEUTIC | receptor-dependent |
| | | activity |
| IFNG+/TNFA+/CD4+/CAR+ | CD4 | Recombinant |
| (intracellular) | THERAPEUTIC | receptor-dependent |
| | | activity |
| IL13+ of CD4+/CAR+ | CD4 | Recombinant |
| (intracellular) | THERAPEUTIC | receptor-dependent |
| | | activity |
| IL17+ of CD4+ CAR+ | CD4 | Recombinant |
| (intracellular) | THERAPEUTIC | receptor-dependent |
| | | activity |
| IL2+ of CD4+ CAR+ | CD4 | Recombinant |
| (intracellular) | THERAPEUTIC | receptor-dependent |
| | | activity |
| IL2+/TNFA+/CD4+/CAR+ | CD4 | Recombinant |
| (intracellular) | THERAPEUTIC | receptor-dependent |
| | | activity |
| TNFA+ of CD4+/CAR+ | CD4 | Recombinant |
| (intracellular) | THERAPEUTIC | receptor-dependent |
| | | activity |
| *Viable cell concentration | CD4 | T cell phenotype |
| | THERAPEUTIC | |
| *Vector copy number | CD4 | T cell phenotype |
| | THERAPEUTIC | |
| *Viability | CD4 | T cell phenotype |
| | THERAPEUTIC | |
| *GMCSF+/CD19+ (secreted) | CD4 | Recombinant |
| | THERAPEUTIC | receptor-dependent |
| | | activity |
| *IFNG+/CD19+ (secreted) | CD4 | Recombinant |
| | THERAPEUTIC | receptor-dependent |
| | | activity |
| *IL10+/CD19+ (secreted) | CD4 | Recombinant |
| | THERAPEUTIC | receptor-dependent |
| | | activity |
| *IL13+/CD19+ (secreted) | CD4 | Recombinant |
| | THERAPEUTIC | receptor-dependent |
| | | activity |
| *IL2+/CD19+ (secreted) | CD4 | Recombinant |
| | THERAPEUTIC | receptor-dependent |
| | | activity |
| *IL5+/CD19+ (secreted) | CD4 | Recombinant |
| | THERAPEUTIC | receptor-dependent |
| | | activity |
| *MIP1A+/CD19+ (secreted) | CD4 | Recombinant |
| | THERAPEUTIC | receptor-dependent |
| | | activity |
| *MIP1B+/CD19+ (secreted) | CD4 | Recombinant |
| | THERAPEUTIC | receptor-dependent |
| | | activity |
| *sCD137+/CD19+ (secreted) | CD4 | Recombinant |
| | THERAPEUTIC | receptor-dependent |
| | | activity |
| *TNFa+/CD19+ (secreted) | CD4 | Recombinant |
| | THERAPEUTIC | receptor-dependent |
| | | activity |
| *CD3+/CAR+ | CD4 | T cell phenotype |
| | THERAPEUTIC | |
| CD3+/CD56+ | CD4 | T cell phenotype |
| | THERAPEUTIC | |
| *CD4+/CAR+ | CD4 | T cell phenotype |
| | THERAPEUTIC | |
| *CD3+/CD8+/CAR+ | CD4 | T cell phenotype |
| | THERAPEUTIC | |
| *CAR+ (anti-idiotype antibody | CD4 | T cell phenotype |
| CAR detection) | THERAPEUTIC | |
| *CD4+/CAR+ (anti-idiotype | CD4 | T cell phenotype |
| antibody CAR detection) | THERAPEUTIC | |
| *3CAS−/CCR7−/CD27−/CD8+/ | CD8 | T cell phenotype |
| CAR+ | THERAPEUTIC | |
| *3CAS−/CCR7−/CD27+/CD8+/ | CD8 | T cell phenotype |
| CAR+ | THERAPEUTIC | |
| *3CAS−/CCR7+/CD8+/CAR+ | CD8 | T cell phenotype |
| | THERAPEUTIC | |
| *3CAS−/CCR7+/CD27−/CD8+/ | CD8 | T cell phenotype |
| CAR+ | THERAPEUTIC | |
| *3CAS−/CCR7+/CD27+/CD8+/ | CD8 | T cell phenotype |
| CAR+ | THERAPEUTIC | |
| *3CAS−/CD27+/CD8+/CAR+ | CD8 | T cell phenotype |
| | THERAPEUTIC | |
| *3CAS−/CD28−/CD27−/CD8+/ | CD8 | T cell phenotype |
| CAR+ | THERAPEUTIC | |
| *3CAS−/CD28−/CD27+/CD8+/ | CD8 | T cell phenotype |
| CAR+ | THERAPEUTIC | |
| *3CAS−/CD28+/CD8+/CAR+ | CD8 | T cell phenotype |
| | THERAPEUTIC | |
| *3CAS−/CD28+/CD27−/CD8+/ | CD8 | T cell phenotype |
| CAR+ | THERAPEUTIC | |
| *3CAS−/CD28+/CD27+/CD8+/ | CD8 | T cell phenotype |
| CAR+ | THERAPEUTIC | |
| *3CAS−/CCR7−/CD45RA−/ | CD8 | T cell phenotype |
| CD8+/CAR+ | THERAPEUTIC | |
| *3CAS−/CCR7−/CD45RA+/ | CD8 | T cell phenotype |
| CD8+/CAR+ | THERAPEUTIC | |
| *3CAS−/CCR7+/CD45RA−/ | CD8 | T cell phenotype |
| CD8+/CAR+ | THERAPEUTIC | |
| *3CAS−/CCR7+/CD45RA+/ | CD8 | T cell phenotype |
| CD8+/CAR+ | THERAPEUTIC | |
| CAS+ of CD3+/CAR+ | CD8 | T cell phenotype |
| | THERAPEUTIC | |
| CD19+ | CD8 | T cell phenotype |
| | THERAPEUTIC | |
| CD3+ | CD8 | T cell phenotype |
| | THERAPEUTIC | |
| *CD3+/CD8+ | CD8 | T cell phenotype |
| | THERAPEUTIC | |
| CD8+/EGFRt+ | CD8 | T cell phenotype |
| | THERAPEUTIC | |
| CYTO−/CD8+/CAR+ | CD8 | T cell phenotype |
| | THERAPEUTIC | |
| EGFRt+ | CD8 | T cell phenotype |
| | THERAPEUTIC | |
| *CD3+/CD4+/CAR+ | CD8 | T cell phenotype |
| | THERAPEUTIC | |
| *CAR+ (anti-idiotype antibody | CD8 | T cell phenotype |
| CAR detection) | THERAPEUTIC | |
| *CD8+/CAR+ (anti-idiotype | CD8 | T cell phenotype |
| antibody CAR detection) | THERAPEUTIC | |
| IFNG+ (intracellular) | CD8 | Recombinant |
| | THERAPEUTIC | receptor-dependent |
| | | activity |
| IFNG+/IL2+/CD8+/CAR+ | CD8 | Recombinant |
| (intracellular) | THERAPEUTIC | receptor-dependent |
| | | activity |
| IFNG+/IL2+/IL17+/TNFA+/ | CD8 | Recombinant |
| CD8+/CAR+ (intracellular) | THERAPEUTIC | receptor-dependent |
| | | activity |
| IFNG+/IL2+/TNFA+/CD8+/CAR+ | CD8 | Recombinant |
| (intracellular) | THERAPEUTIC | receptor-dependent |
| | | activity |
| *IFNG+ of CD8+/CAR+ | CD8 | Recombinant |
| (intracellular) | THERAPEUTIC | receptor-dependent |
| | | activity |
| IFNG+/TNFA+/CD8+/CAR+ | CD8 | Recombinant |
| (intracellular) | THERAPEUTIC | receptor-dependent |
| | | activity |
| IL13+ of CD8+/CAR+ | CD8 | Recombinant |
| (intracellular) | THERAPEUTIC | receptor-dependent |
| | | activity |
| IL17+ of CD8+ CAR+ | CD8 | Recombinant |
| (intracellular) | THERAPEUTIC | receptor-dependent |
| | | activity |
| IL2+ of CD8+ CAR+ | CD8 | Recombinant |
| (intracellular) | THERAPEUTIC | receptor-dependent |
| | | activity |
| IL2+/TNFA+/CD8+/CAR+ | CD8 | Recombinant |
| (intracellular) | THERAPEUTIC | receptor-dependent |
| | | activity |
| *Cytolytic activity | CD8 | Recombinant |
| | THERAPEUTIC | receptor-dependent |
| | | activity |
| TNFA+ of CD8+ CAR+ | CD8 | Recombinant |
| | THERAPEUTIC | receptor-dependent |
| | | activity |
| Viable cell concentration | CD8 | T cell phenotype |
| | THERAPEUTIC | |
| *Vector copy number | CD8 | T cell phenotype |
| | THERAPEUTIC | |
| Viability | CD8 | T cell phenotype |
| | THERAPEUTIC | |
| *GMCSF+/CD19+ (secreted) | CD8 | Recombinant |
| | THERAPEUTIC | receptor-dependent |
| | | activity |
| *IFNG+/CD19+ (secreted) | CD8 | Recombinant |
| | THERAPEUTIC | receptor-dependent |
| | | activity |
| *IL10+/CD19+ (secreted) | CD8 | Recombinant |
| | THERAPEUTIC | receptor-dependent |
| | | activity |
| *IL13+/CD19+ (secreted) | CD8 | Recombinant |
| | THERAPEUTIC | receptor-dependent |
| | | activity |
| *IL2+/CD19+ (secreted) | CD8 | Recombinant |
| | THERAPEUTIC | receptor-dependent |
| | | activity |
| *IL5+/CD19+ (secreted) | CD8 | Recombinant |
| | THERAPEUTIC | receptor-dependent |
| | | activity |
| *MIP1A+/CD19+ (secreted) | CD8 | Recombinant |
| | THERAPEUTIC | receptor-dependent |
| | | activity |
| *MIP1B+/CD19+ (secreted) | CD8 | Recombinant |
| | THERAPEUTIC | receptor-dependent |
| | | activity |
| *sCD137+/CD19+ (secreted) | CD8 | Recombinant |
| | THERAPEUTIC | receptor-dependent |
| | | activity |
| *TNFa+/CD19+ (secreted) | CD8 | Recombinant |
| | THERAPEUTIC | receptor-dependent |
| | | activity |
| *CD3+/CAR+ | CD8 | T cell phenotype |
| | THERAPEUTIC | |
| CD3+/CD56+ | CD8 | T cell phenotype |
| | THERAPEUTIC | |
| CD8+/CAR+ | CD8 | T cell phenotype |
| | THERAPEUTIC | |

*Denotes attributes used for pCCA in Example 3

A. Penalized Canonical Correlation Analysis

Penalized canonical correlation analysis (pCCA) was used to identify input composition attributes that correlate with attributes of the resultant therapeutic compositions. Linear combinations of input composition attributes and linear combinations of therapeutic composition attributes were found to maximize the correlation between the input composition attributes and therapeutic composition attributes. Equation 1 iteratively solves for the maximum correlation between sets of variables:

argmax_{u,v}u^{T}X^{T}Yv subject to ∥u∥₂²≤1,∥v∥₂²≤1 (Eq. 1)

where X and Y represent sets of high dimensional variables (e.g., input attributes and therapeutic composition attributes) and u and v are canonical vectors, each constrained by the requirement that the square of the L2 norm is less than or equal to 1. In some cases, a closed form solution may be used.

Model complexity was reduced by optionally evoking a sparsity penalty to down-weight (regularize) attributes with small, independent effects. Equation 2 includes implementation of the penalty constraint:

argmax_{u,v}u^{T}X^{T}Yv subject to ∥u∥₂²≤1,∥v∥₂²≤1,P₁(u)≤C₁,P₂(v)≤C₂ (Eq. 2)

where variables X, Y, u, and v are as described above; P₁ and P₂ are convex penalty functions (lasso, L1 regularization); and C₁ and C₂ are constants determined using a permutation scheme (e.g., cca.permute R v3.5). Small fractions of missing values for either set of attributes were imputed. Analysis was performed using the PMA package in R v3.5 or 3.6.

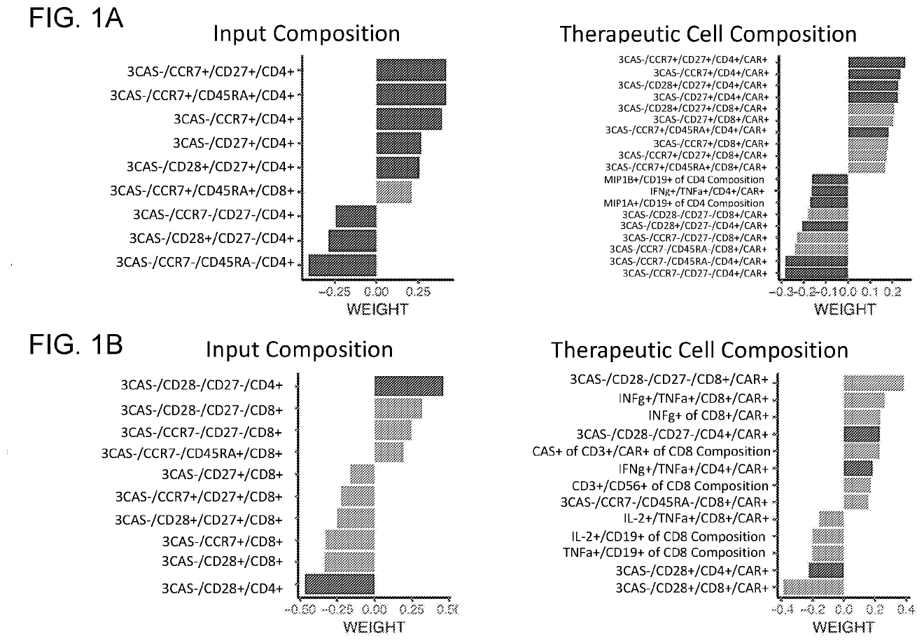

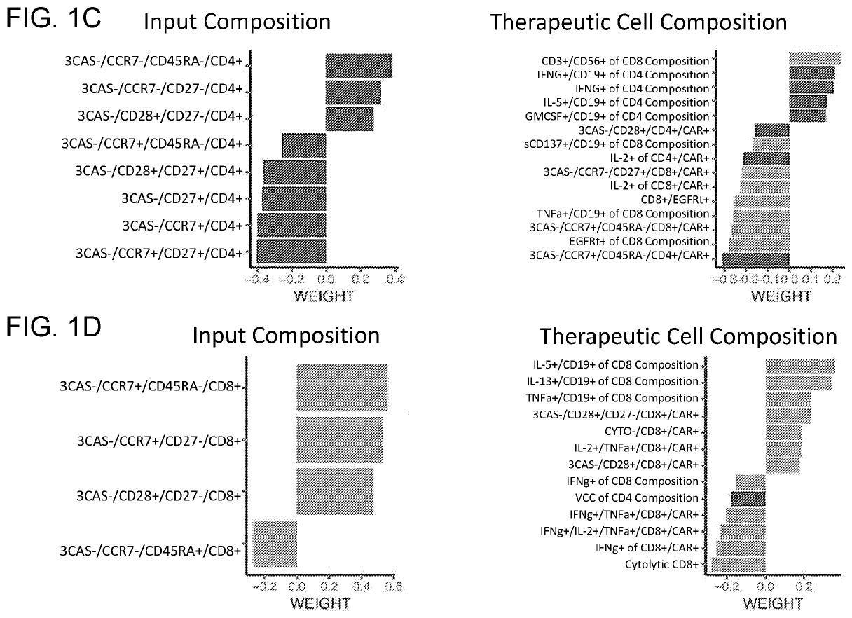

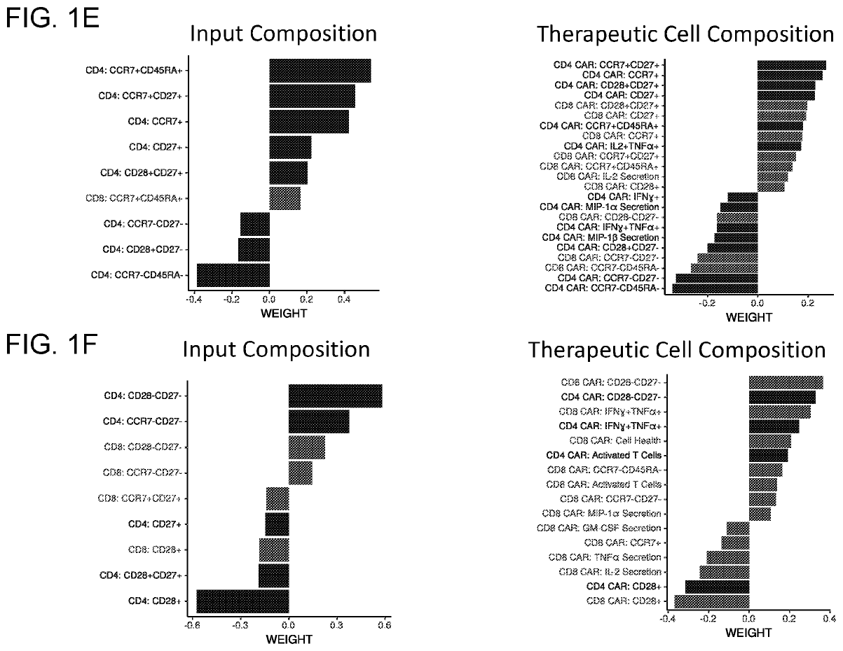

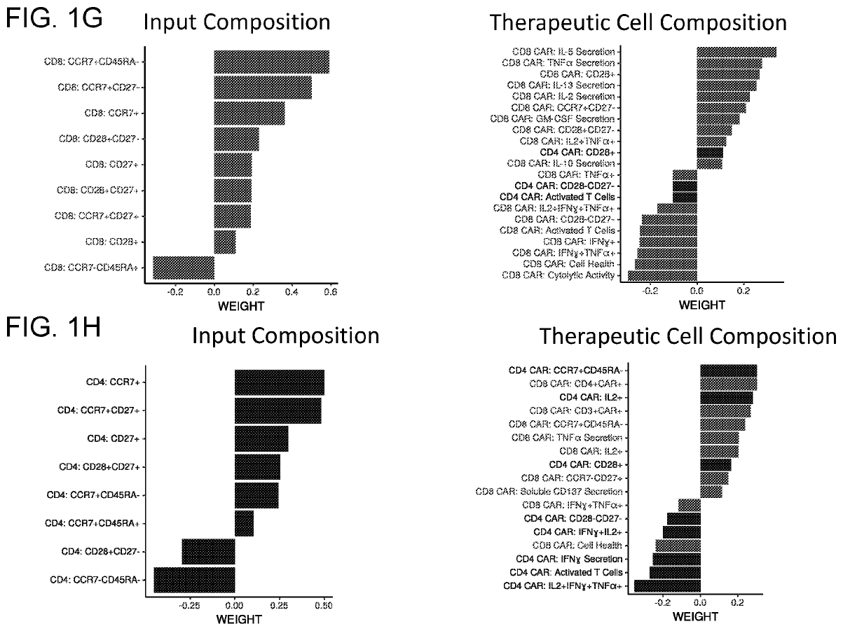

pCCA identified subsets of input composition attributes that were highly correlated with subsets of therapeutic composition attributes. FIGS. 1A-1D are examples showing attribute contributions (weights) to the top four exemplary input and therapeutic composition attribute pairs. Attributes with absolute weights less than or equal to 0.15 were excluded.

The first pair, which has the highest canonical correlation and captures the highest explained shared variance, can be seen in FIG. 1A. This result is supportive of proportions of naïve (e.g., CD27+/CCR7+, CD27+, CCR7+, CCR7+/CD45RA+, CD28+/CD27+) CD4 T cells in the input composition being highly correlated with proportions of naïve and naïve-like CD4 and naïve and naïve-like CD8 CAR+ T cells in the therapeutic compositions. The second pair, shown in FIG. 1B, is supportive of naïve (e.g., CD27+/CCR7+, CD27+, CCR7+, CD28+/CD27+, CD28+) and stem cell memory (e.g., CD28−/CD27−, CCR7−CD27−, CCR7+/CD45RA+) CD4 and CD8 T cell proportions in the input composition being correlated with naïve and stem cell memory CD8 CAR T cell proportions in the therapeutic composition. The third pair, shown in FIG. 1C, is supportive of effector memory CD4 T cell (e.g., CCR7−/CD45RA−, CCR7−/CD27−, CD28+/CD27−) proportions in the input composition being correlated with CD4 and CD8 CAR T cell effector functions, including antigen-specific cytokine production (e.g., IFNg, IL-5, GMCSF) in the therapeutic compositions. The fourth pair, shown in FIG. 1D, is supportive of effector memory CD8 T cell (CCR7+/CD27−, CD28+/CD27−, CCR7+/CD45RA−) proportions in the input composition being correlated with CD8 CAR T cell effector functions, for example, cytokine expression (e.g., IL-5, IL-13, TNF-a, IL-2), in the therapeutic composition.

A second pCCA run was performed with modified parameters on the same data. The first attribute pair identified can be seen in FIG. 1E. This result is supportive of proportions of naïve (e.g., CCR7+/CD45RA+, CD27+/CCR7+, CCR7+, CD27+, CD28+/CD27+) CD4 T cells in the input composition being highly correlated with proportions of naïve and naïve-like CD4 and naïve and naïve-like CD8 CAR+ T cells in the therapeutic compositions. The second pair, shown in FIG. 1F, is supportive of differentiated effector memory CD4 and CD8 T cell proportions in the input composition being correlated with differentiated effector memory CD4 and CD8 CAR T cell proportions in the therapeutic composition. The third pair, shown in FIG. 1G, is supportive of central memory CD8 T cell (e.g., CCR7+/CD45RA−) proportions in the input composition being correlated with CD8 CAR T cell effector functions, including antigen-specific cytokine production (e.g., IL-2 and TNFα) in the therapeutic compositions. The fourth pair, shown in FIG. 1H, is supportive of central memory CD4 T cell (CCR7+, CCR7+/CD27+, CD27+) proportions in the input composition being correlated with CD4 and CD8 CAR T cell central memory proportions in the therapeutic composition. Table E3 provides a summary of the relationships identified between input composition attributes and therapeutic cell composition attributes.

Each pCCA run identified similar attribute pairings. In particular, both runs identified as the first canonical correlation a relationship between the proportion of naïve CD4 cells in the input composition and the proportion of naïve and naive-like CD4+ and CD8+ T cells in the therapeutic cell composition.

TABLE E3
Summary of relationships between input composition attributes
and therapeutic cell composition attributes.

| | Therapeutic Cell Composition |
|---|---|
| Input Composition Attributes | Attributes |
| ↑ Naïve CD4 | ↑ CD4 and CD8 Naïve |
| ↓ Tem CD4 | ↓ CD4 and CD8 Tem |
| ↑ Tem CD4 and CD8 | ↑ CD4 and CD8 Tem and IFNγ |
| | ↓ CD4 and CD8 IL-2 |
| ↑ Tcm CD8 | ↑ CD8 IL-2 and TNFα |
| ↓ Temra CD8 | ↓ CD8 IFNγ |
| ↑ Tcm CD4 | ↑ CD4 and CD8 Tcm and IL-2 |
| ↓ Tem CD4 | ↓ CD4 IFNγ |

B. Lasso Regression Model

pCCA, as described above, identified correlated sets of input and therapeutic composition attributes simultaneously, providing insight on potential association between the attributes. In some instances, pCCA may have a reduced capacity to predict single attributes. To complement learning about the data structure, and in some cases enhance predictive accuracy of single attributes one at a time, lasso regression, which performs both variable selection and regularization, was performed to identify groups of input composition attributes that can collectively predict a single therapeutic composition attribute. Analysis was performed using the glmnet package in R v3.5.

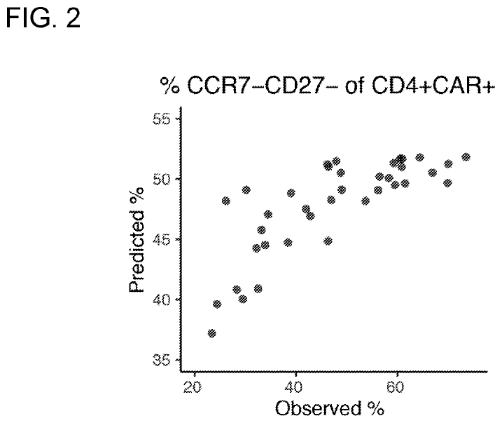

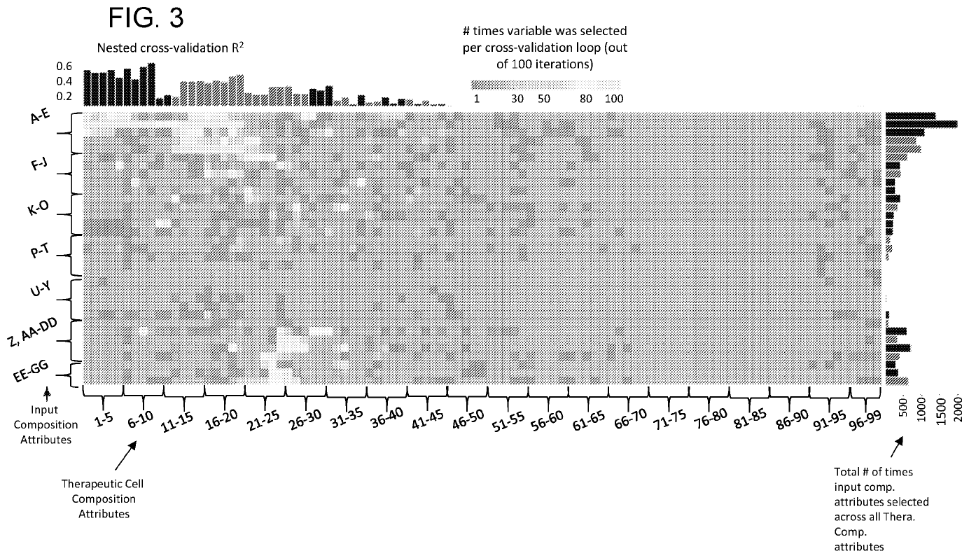

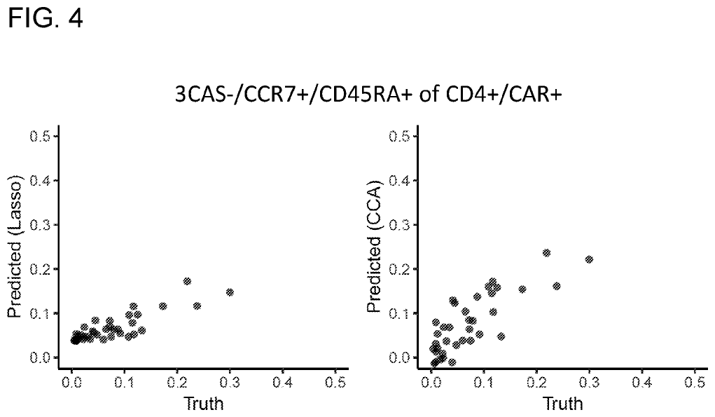

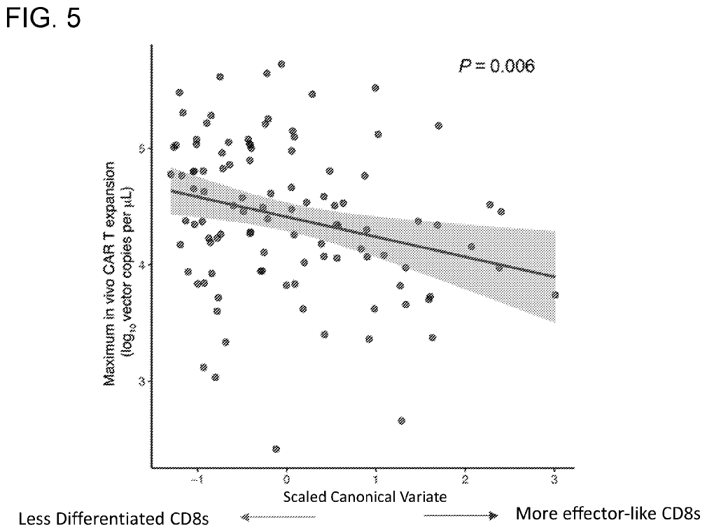

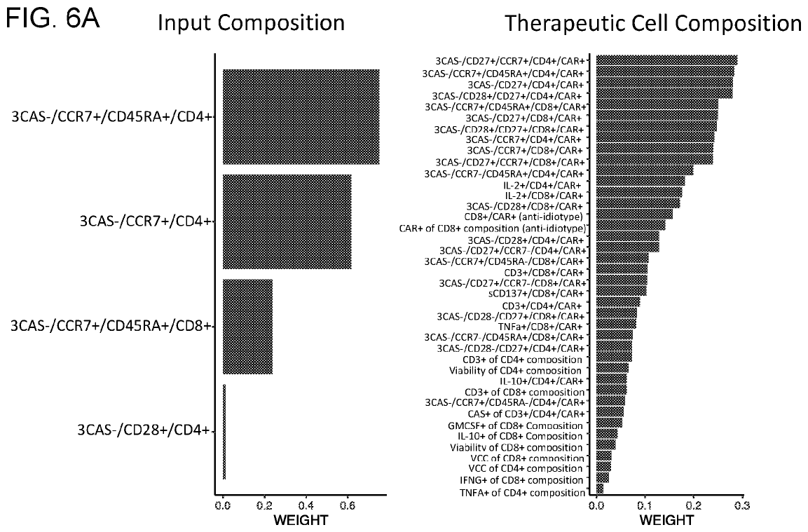

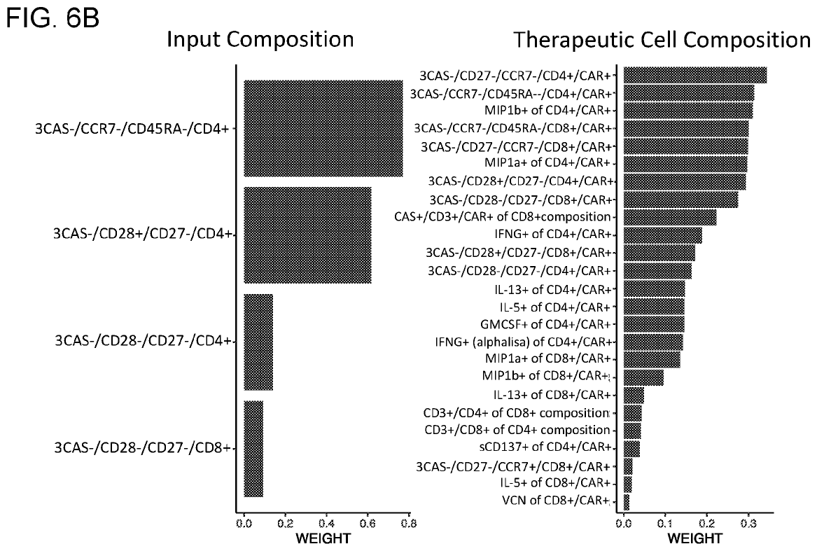

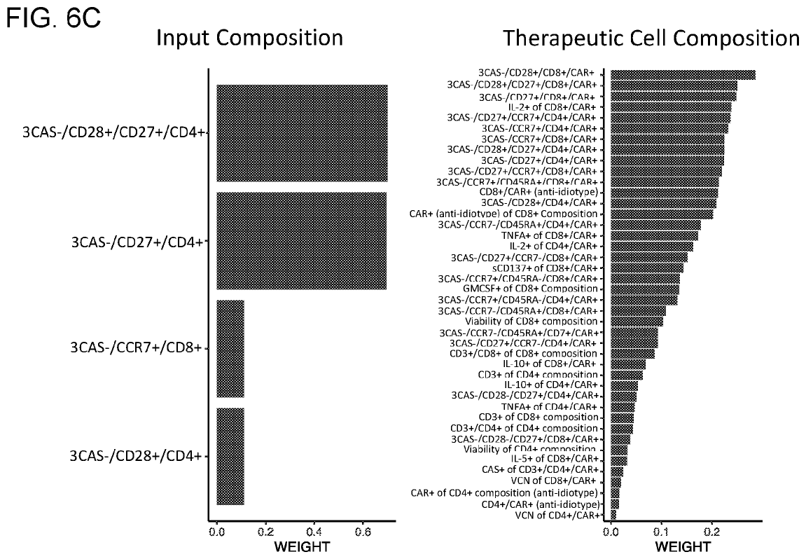

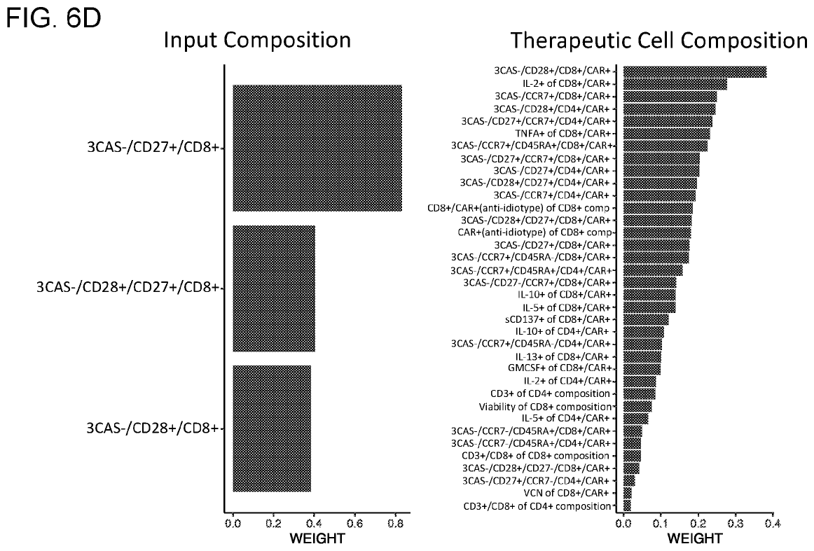

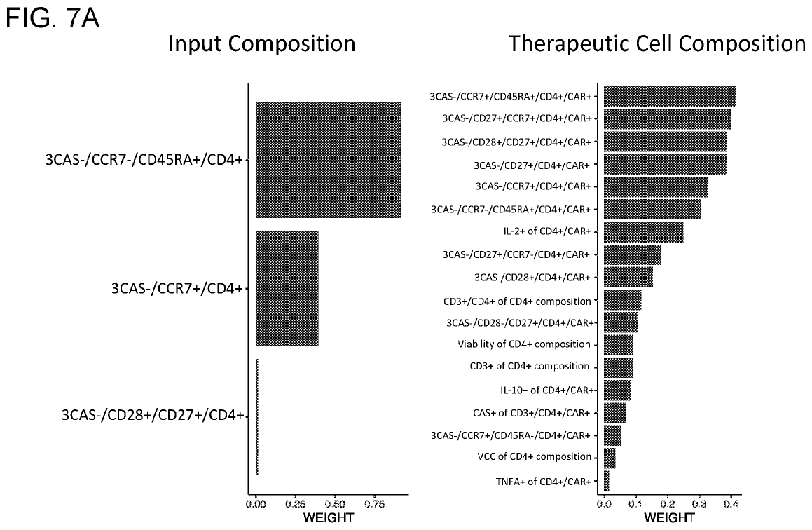

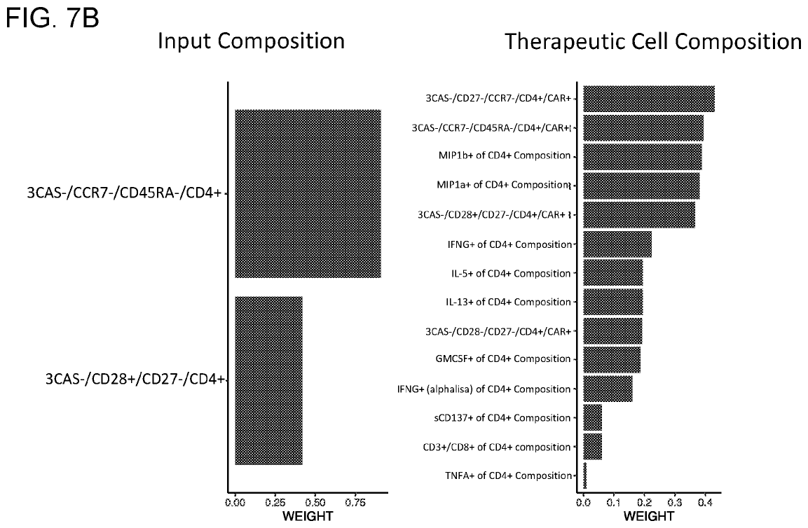

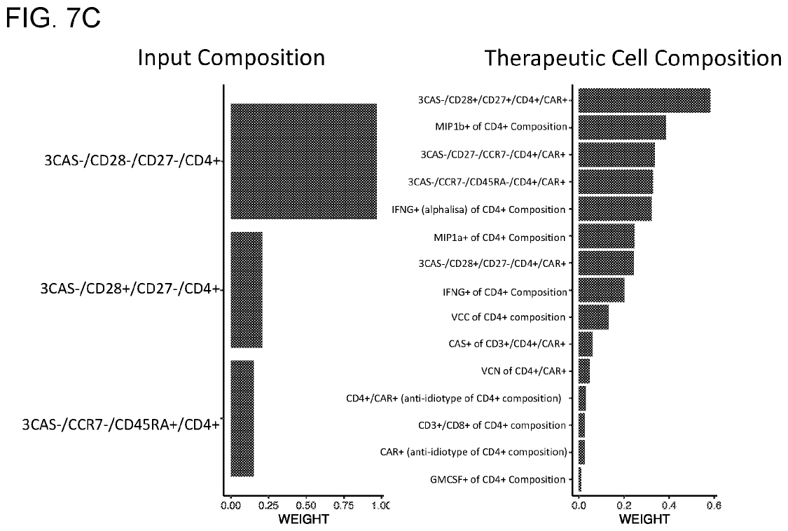

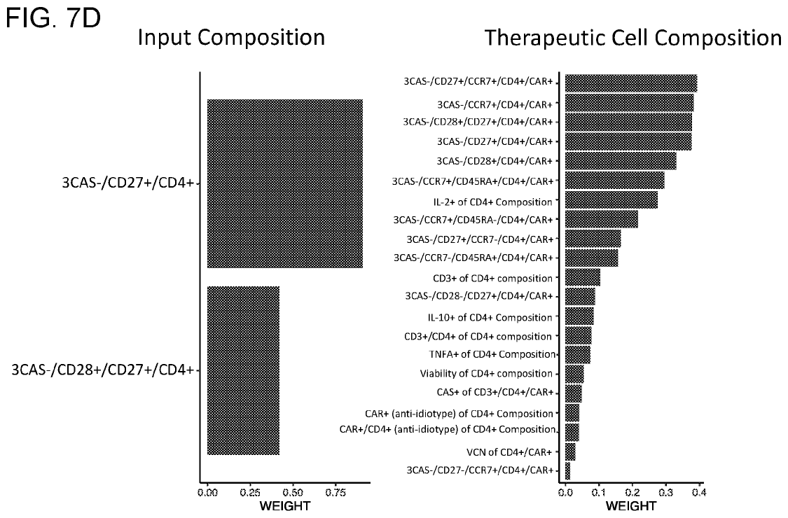

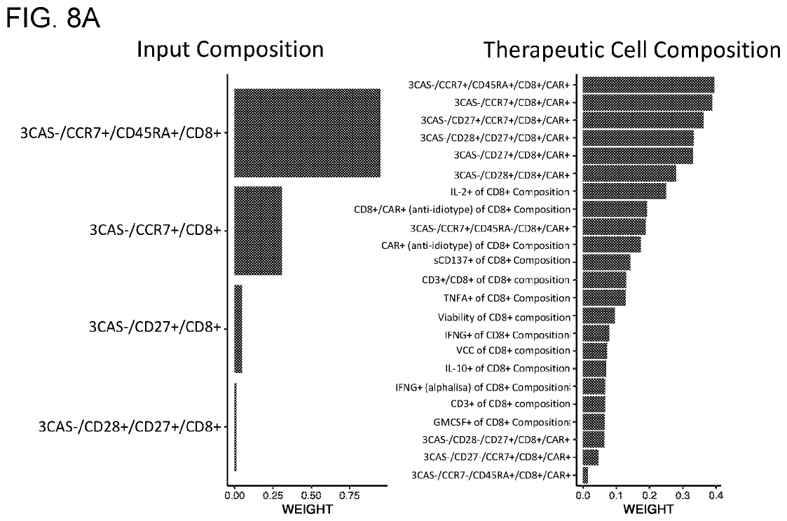

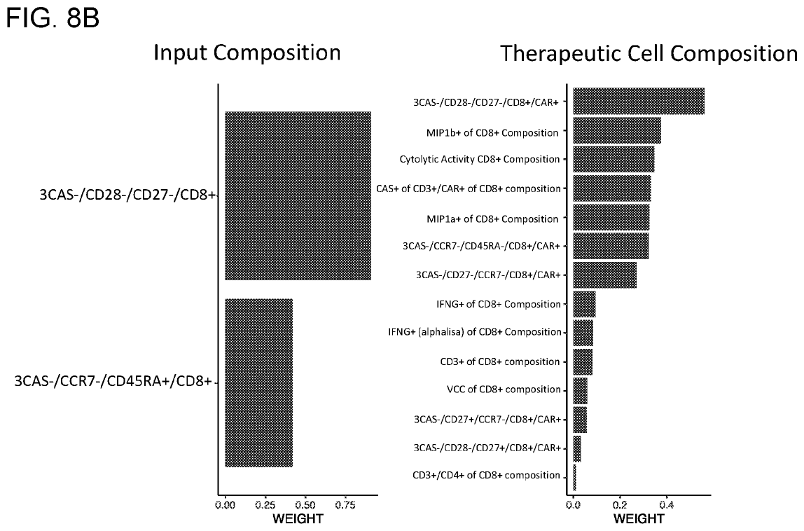

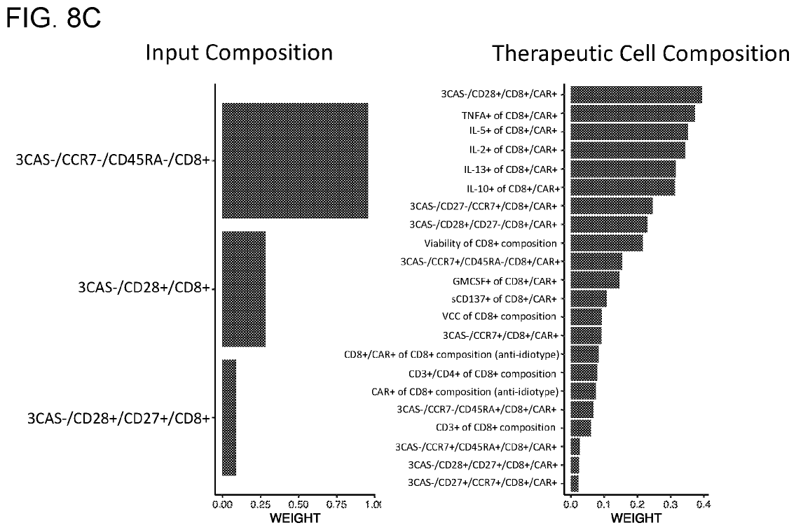

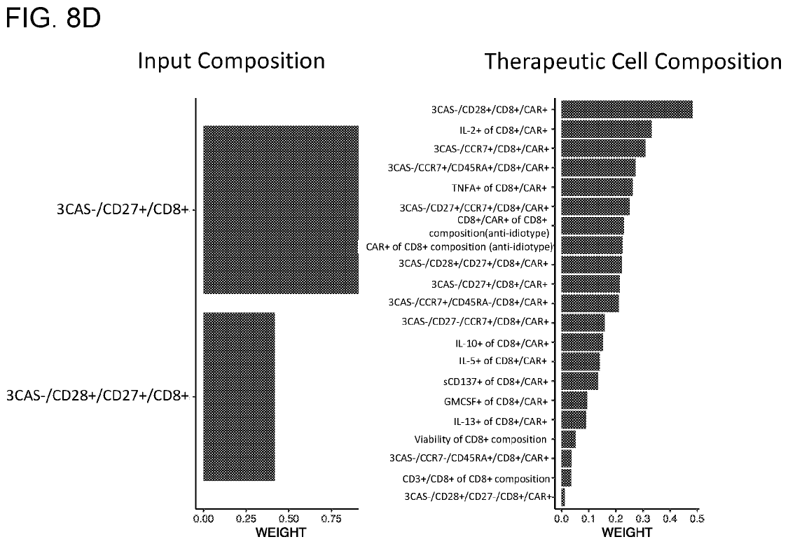

The lasso regression model was constructed by selecting a single therapeutic composition attribute for prediction, and using all attributes from the input compositions (see Table E2) as input to the model. The model was trained on 90% of the data to tune the penalization parameter, lambda, using ten-fold cross-validation. The tuning procedure trained the model to identify a subset of input composition attributes relevant for predicting the selected therapeutic composition attribute. The predictive accuracy of the trained model was then tested using the remaining 10% of the data (e.g., test data). FIG. 2 shows a scatter plot of one example prediction plotted against the observed values. Model construction and training was repeated 100 times for each therapeutic composition attribute. FIG. 3 shows a heatmap depicting the number of times an input composition attribute was identified as relevant for predicting a given therapeutic cell composition attribute. The total number of times an input composition attribute was selected across all therapeutic composition attributes is shown to the right of the heatmap. The average nested cross-validation R-squared value across all 100 iterations is shown at the top of the heatmap for each selected therapeutic composition attribute.

Lasso regression analysis was supportive of (1) naïve CD4 T cell (e.g., CCR7+/CD27+, CCR7+/CD45RA+) proportions in the input composition being predictive of naïve CD4 CAR T cell proportions in the therapeutic composition; (2) naïve CD4 and CD8 T cell proportions in input compositions being predictive of naïve CD8 CAR T cell proportions in the therapeutic composition; and (3) effector proportions of CD4 and CD8 (e.g., CCR7−/CD27−) cells in the input compositions being predictive of cytokine production (e.g., IFNg, TNF-a, IL-13, IL-2, IL-5) by CD8 CAR T cells of the therapeutic composition when stimulated with antigen. The analysis also was supportive of the CCR7+CD45RA+naïve CD4 T cell population being the most informative input composition attribute for predicting the most therapeutic cell composition attributes.

C. Predictive Accuracy: CCA and Lasso Regression

CCA, without the use of a penalty, was used as a statistical learning model and trained to predict therapeutic composition attributes from input composition attributes. Predictive accuracy was assessed for CCA using the telefit package for R v3.5 with 10-fold cross-validation. Small fractions of missing values for either set of attributes were imputed. The resulting CCA predictions were compared to the predictive accuracy of the lasso regression model described above.

CCA achieved prediction accuracies up to an R² of 42% for predicting proportions of CD4+/CAR+ naïve-like (CCR7+/CD45RA+) T cells in therapeutic compositions (P=0.008) from input composition attributes. Lasso regression achieved up to an R² of 67% for the same therapeutic composition CAR T cell attribute (P=6×10⁻²⁷⁵) (FIG. 4).

Both CCA and lasso regression performed best at predicting classically naïve (e.g. CCR7+/CD45RA+, CCR7+/CD27+, CD27+/CD28+) and T_{EMRA} T cells (e.g., CD27−/CD28−, CCR7−/CD45RA+) in the therapeutic compositions, as well as subsets of cytokines (e.g., MIP1A+ of CD4, MIP1B+ of CD4, IL-2+/TNFa+ of CD4, IL-2+ of CD8, IFNg+ of CD8) produced by therapeutic compositions. CCA and lasso regression achieved nominally significant predictions for 53 of the 101 therapeutic compositions CAR T cell attributes using only 34 attributes from input compositions as input.

### Example 2: Assessment of the Relationship Between Input and Therapeutic Cell Composition Attributes and CAR T Cell Pharmacokinetics

To determine whether attributes identified by the statistical learning methods correlated with pharmacokinetics of CAR+ T cells in the blood, attribute pairs identified by pCCA, as described in Example 1A, were compared to measured peak (maximum) CAR T cells expansion in the blood of subjects that had been separately administered equal doses of the CD4+CAR+ and CD8+CAR+ therapeutic T cell compositions (1:1). Blood samples were analyzed at various time points following administration of dose of the cell compositions by flow cytometry for the presence and number of CAR-expressing cells in the blood. The number of CAR+ T cells per μL of blood was determined.

For a patient lot, the dot product of the weights of attributes and the measured values of attributes was calculated to obtain the canonical variate on an attribute pair. The scaled canonical variates among patients were then plotted against the maximum CAR+ T cell concentration in the blood for the same patient who had received treatment with the therapeutic cell composition.

FIG. 5 shows an exemplary relationship between scaled canonical variates and maximum in vivo CAR T cell expansion. The exemplary attribute pair selected for comparison against the clinical response, and from which the canonical variate was derived, is supportive of more differentiated effector memory CD4 and CD8 T cell (e.g., CD28−/CD27−, CCR7−/CD27−) proportions in the input composition being correlated with the proportion of CD8 CAR T cells capable of antigen-specific cytokine production of IFNγ and TNFα in the therapeutic composition and the proportion of CD4 and CD8 CAR effector memory T cells (e.g., CD28−/CD27−) in the therapeutic composition. As shown in FIG. 5, input compositions and therapeutic cell compositions containing less differentiated CD8+ T cells, as indicated by the scaled canonical variates determined for the patient lot, were associated with increased maximum in vivo CAR T cell expansion.

These data indicate that methods for relating input composition attributes to therapeutic composition attributes are useful for predicting maximum CAR T cell expansion.

### Example 3: Methods for Assessing Relationships Between Attributes of Patient-Derived Material for Manufacturing a Cell Therapy and Attributes of the Resulting Therapeutic Cell Composition

To investigate the relationship between attributes of patient-derived material for manufacturing a cell therapy, also referred to as input composition attributes, and attributes of therapeutic cell compositions manufactured therefrom, penalized canonical correlation analysis (pCCA) was used to identify correlations between groups of input composition attributes and therapeutic cell composition attributes. Attributes of CD4+ T cell lots (n=129) and CD8+ T cell lots (n=129) derived from 129 patients with Diffuse Large B-Cell Lymphoma (DLBCL) were determined before and after manufacturing according to the methods described in Example 1 to produce therapeutic cell compositions containing CD4+ and CD8+ T cells expressing an exemplary anti-CD19 CAR. The assessed input composition attributes, e.g., cell phenotype, and therapeutic cell composition attributes, e.g., cell phenotype and functional attributes, are shown in Table E2 and denoted by asterisks.

pCCA was performed as described in Example 1A to identify correlations between groups of input composition and therapeutic cell composition attributes. CD4+ and CD8+ attributes of the input compositions and therapeutic cell compositions were analyzed together by pCCA such that correlations between attributes were not limited to a specific cell types. For example, correlations between CD4+ attributes and CD8+ attributes could be identified by the analysis. Cell type specific pCCA was also performed to determine how cell type specific attributes, e.g., CD4+-specific attributes or CD8+-specific attributes, correlated between input and therapeutic cell compositions.

A. Correlation of CD4+ and CD8+ Attributes Across Input and Therapeutic Cell Compositions

pCCA was performed using attributes from CD4+ and CD8+ T cells in input compositions and therapeutic cell compositions to identify groups of correlated attributes. The first four input composition and therapeutic cell composition attribute pairs were assessed because they explained the majority of the covariance between the input composition attributes and therapeutic cell composition attributes. FIGS. 6A-6D show the first four attribute pairs for CD4+ and CD8+ T cells in the input composition and therapeutic cell composition. Canonical correlation and explained covariance decreased from the first to the fourth attribute pair.

FIG. 6A shows the first attribute pair which had the highest canonical correlation and explained covariance. The results shown in FIG. 6A are supportive of naïve CD4+ and CD8+ T cells (e.g., CCR7+/CD45RA+, CCR7+, CD28+/CD27+) in the input composition being positively correlated with the proportion of naïve CD4+ and CD8+ T cells (e.g., CCR7+/CD45RA+, CCR7+, CD28+/CD27+) in the therapeutic cell composition.

FIG. 6B shows the second attribute pair, which is supportive of effector-like CD4+ T cells (e.g., CCR7−/CD45RA−, CCR7−/CD27−) in the input composition being positively correlated with the proportion of effector CD4+ and CD8+ T cells in the therapeutic cell composition. FIG. 6B further demonstrates that the proportion of effector-like CD4+ cells correlates positively with the proportion of CD4+ cells that express MIP1a or MIP1b following antigen-specific stimulation.

FIG. 6C shows the third attribute pair, which is supportive of naïve to intermediate CD4+ T cells (e.g., CD28+/CD27+, CD27+, CD28+) in the input composition being positively correlated with the proportion of naïve to intermediate CD4+ and CD8+ T cells (e.g., CD28+/CD27+, CD27+, CD28+), the proportion of CD8+ T cells that express IL-2 following antigen-specific stimulation, and the proportion of CD8+/CAR+ T cells in the therapeutic cell composition.

The fourth attribute pair, shown in FIG. 6D, is supportive of naïve to intermediate CD8+ T cells (e.g., CD28+/CD27+, CD27+, CD28+) in the input composition being positively correlated with the proportion of naïve to intermediate CD4+ and CD8+ T cells (e.g., CD28+/CD27+, CD27+, CD28+) and proportion of CD8+ T cells that express IL-2 or TNF-α following antigen-specific stimulation in the therapeutic cell composition.

These data are supportive of the use of pCCA to identify groups of correlated attributes between input compositions and therapeutic cell compositions. Further, these results demonstrate that pCCA can be used to identify correlated attributes within and between specific cell types of the input and therapeutic cell compositions.

B. Correlation of Cell Type Specific Attributes Across Input Compositions and Therapeutic Cell Compositions

As described in Example 1 above, CD4+ and CD8+ T cells of the input composition may be manufactured separately to generate CD4+ and CD8+ CAR-T cells of the therapeutic cell composition. Since manufacturing of each cell type occurs independently, pCCA was performed as described in Example 1A to identify cell type specific attribute correlations between the input composition and therapeutic cell composition.

1. CD4+ Attribute Correlations

pCCA was performed using attributes from CD4+ T cells in the input composition and therapeutic cell composition to identify groups of correlated attributes. The first four input composition and therapeutic cell composition attribute pairs were assessed because they explained the majority of the covariance between the input composition attributes and therapeutic cell composition attributes. FIG. 7A-7D show the first four attribute pairs for CD4+ T cells in the input composition and therapeutic cell composition. The canonical correlation and explained covariance decreased from the first to the fourth attribute pair.

FIG. 7A shows the first attribute pair which had the highest canonical correlation and explained covariance. The results shown in FIG. 7A are supportive of naïve CD4+ T cells (e.g., CCR7+/CD45RA+, CCR7+, CD28+/CD27+) in the input composition being positively correlated with the proportion of naïve CD4+ T cells (e.g., CCR7+/CD45RA+, CCR7+, CD28+/CD27+) in the therapeutic cell composition.

FIG. 7B shows the second attribute pair, which is supportive of effector-like CD4+ T cells (e.g., CCR7−/CD45RA−, CCR7−/CD27−) in the input composition being positively correlated with the proportion of effector CD4+ T cells and the proportion of CD4+ T cells that express MIP1a or MIP1b following antigen-specific stimulation.

FIG. 7C shows the third attribute pair, which is supportive of effector CD4+ T cells (e.g., CD28−/CD27−) in the input composition being positively correlated with the proportion of effector CD4+ T cells (e.g., CD28−/CD27−, CD28+/CD27−, CCR7−/CD45RA+) and CD4+ T cells that express MIP1a, MIP1b, or IFNg following antigen-specific stimulation in the therapeutic cell composition.

The fourth attribute pair, shown in FIG. 7D, is supportive of naïve to intermediate CD4+ T cells (e.g., CD28+/CD27+, CD27+) in the input composition being positively correlated with the proportion of naïve to intermediate CD4+ T cells (e.g., CD27+/CCR7+, CCR7+, CD28+/CD27+, CD27+, CD28+, CCR7+/CD45RA+) and proportion of CD4+ T cells that express IL-2 following antigen-specific stimulation in the therapeutic cell composition.

These data are consistent with the ability of pCCA to identify cell type specific attribute correlations between the input composition and therapeutic cell composition.

2. CD8+ Attribute Correlations

pCCA was performed using attributes from C84+ T cells in the input composition and therapeutic cell composition to identify correlated attributes. The first four input composition and therapeutic cell composition attribute pairs were assessed because they explained the majority of the covariance between the input composition attributes and therapeutic cell composition attributes. FIG. 8A-8D show the first four attribute pairs for CD8+ T cells in the input composition and therapeutic cell composition. The canonical correlation and explained covariance decrease from the first to the fourth attribute pair.

FIG. 8A shows the first attribute pair which had the highest canonical correlation and explained covariance. The results shown in FIG. 8A are supportive of naïve CD8+ T cells (e.g., CCR7+/CD45RA+, CCR7+, CD27+, CD28+/CD27+) in the input composition being positively correlated with the proportion of naïve CD8+ T cells (e.g., CCR7+/CD45RA+, CCR7+, CD27+/CCR7+, CD28+/CD27+) in the therapeutic cell composition.

FIG. 8B shows the second attribute pair, which is supportive of effector-like CD8+ T cells (e.g., CCR7−/CD45RA−, CD28−/CD27−) in the input composition being positively correlated with the proportion of effector CD8+ T cells and the proportion of effector-like CD8+ T cells, CD8+ T cells that express MIP1a or MIP1b following antigen-specific stimulation, and Cas+/CAR+ CD8 T cells.

FIG. 8C shows the third attribute pair, which is supportive of intermediate CD8+ T cells (e.g., CCR7+/CD45RA−, CD28+) in the input composition being positively correlated with the proportion of intermediate CD8+ T cells (e.g., CD28+, CD27−/CCR7+, CD28+/CD27−) and CD8+ T cells that express TNFa, IL-5, IL-2, IL-13, or IL-10 following antigen-specific stimulation in the therapeutic cell composition.

The fourth attribute pair, shown in FIG. 8D, is supportive of naïve to intermediate CD8+ T cells (e.g., CD28+/CD27+, CD27+) in the input composition being positively correlated with the proportion of naïve to intermediate CD8+ T cells (e.g., CD27+/CCR7+, CCR7+, CD28+/CD27+, CD27+, CD28+, CCR7+/CD45RA+) and proportion of CD8+ T cells that express IL-2 and/or TNFa following antigen-specific stimulation in the therapeutic cell composition.

These data are also consistent with the ability of pCCA to identify cell type specific attribute correlations between the input composition and therapeutic cell composition.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | LEGGGEGRGSLLTCGDVEENPGPR | T2A |
| 2 | MLLLVTSLLLCELPHPAFLLIPRKV | tEGFR |
| | CNGIGIGEFKDSLSINATNIKHFKN | |
| | CTSISGDLHILPVAFRGDSFTHTPP | |
| | LDPQELDILKTVKEITGFLLIQAWP | |
| | ENRTDLHAFENLEIIRGRTKQHGQF | |
| | SLAVVSLNITSLGLRSLKEISDGDV | |
| | IISGNKNLCYANTINWKKLFGTSGQ | |
| | KTKIISNRGENSCKATGQVCHALCS | |
| | PEGCWGPEPRDCVSCRNVSRGRECV | |
| | DKCNLLEGEPREFVENSECIQCHPE | |
| | CLPQAMNITCTGRGPDNCIQCAHYI | |
| | DGPHCVKTCPAGVMGENNTLVWKYA | |
| | DAGHVCHLCHPNCTYGCTGPGLEGC | |
| | PTNGPKIPSIATGMVGALLLLLVVA | |
| | LGIGLFM | |
| 3 | RKVCNGIGIGEFKDSLSINATNIKH | tEGFR |
| | FKNCTSISGDLHILPVAFRGDSFTH | |
| | TPPLDPQELDILKTVKEITGFLLIQ | |
| | AWPENRTDLHAFENLEIIRGRTKQH | |
| | GQFSLAWSLNITSLGLRSLKEISDG | |
| | DVIISGNKNLCYANTINWKKLFGTS | |
| | GQKTKIISNRGENSCKATGQVCHAL | |
| | CSPEGCWGPEPRDCVSCRNVSRGRE | |
| | CVDKCNLLEGEPREFVENSECIQCH | |
| | PECLPQAMNITCTGRGPDNCIQCAH | |
| | YIDGPHCVKTCPAGVMGENNTLVWK | |
| | YADAGHVCHLCHPNCTYGCTGPGLE | |
| | GCPTNGPKIPSIATGMVGALLLLLV | |
| | VALGIGLFM | |
| 4 | EGRGSLLTCGDVEENPGP | T2A |
| 5 | GSGATNFSLLKQAGDVEENPGP | P2A |
| 6 | ATNFSLLKQAGDVEENPGP | P2A |
| 7 | QCTNYALLKLAGDVESNPGP | E2A |
| 8 | VKQTLNFDLLKLAGDVESNPGP | F2A |
| 9 | atgcttctcctggtgacaagccttc | GMCSFR alpha chain |
| | tgctctgtgagttaccacacccagc | signal sequence |
| | attcctcctgatccca | |
| 10 | MLLLVTSLLLCELPHPAFLLIP | GMCSFR alpha chain |
| | | signal sequence |
| 11 | MALPVTALLLPLALLLHA | CD8 alpha signal |
| | | peptide |
| 12 | MPLLLLLPLLWAGALA | CD33 signal peptide |

1. A method of predicting attributes of a therapeutic cell composition, the method comprising:
(a) determining a percentage, number, ratio, and/or proportion of T cells in an input composition that have first attributes, wherein the first attributes comprise T cell phenotypes, and wherein the input composition comprises T cells selected from a biological sample from a subject; and
(b) applying the first attributes as input to a process configured to predict, based on the first attributes, a percentage, number, ratio and/or proportion of T cells in a therapeutic cell composition that have second attributes, wherein:
the therapeutic cell composition comprises T cells expressing the recombinant receptor and is to be produced from cells of the input composition;
the second attributes comprise T cell phenotypes and recombinant receptor-dependent activity; and
the process comprises a canonical correlation analysis statistical learning model trained on training data comprising (i) the percentage, number, ratio and/or proportion of T cells that have the first attributes from each of a plurality of input compositions comprising T cells and (ii) the percentage, number, ratio and/or proportion of T cells that have the second attributes from each of a plurality of therapeutic cell compositions, wherein each of the therapeutic cells compositions comprises T cells expressing the recombinant receptor and has been produced from one of the input compositions.

2. The method of claim 1, further comprising (c) determining, based on the predicted second attributes, whether the therapeutic cell composition is predicted to have a desired attribute.

3. A method of predicting attributes of a therapeutic cell composition, the method comprising:
(a) determining a percentage, number, ratio, and/or proportion of T cells in an input composition that have first attributes, wherein the first attributes comprise T cell phenotypes, and wherein the input composition comprises T cells selected from a biological sample from a subject;
(b) applying the first attributes as input to a process configured to predict, based on the first attributes, a percentage, number, ratio and/or proportion of T cells in a therapeutic cell composition that have one second attribute, wherein:
the therapeutic cell composition comprises T cells expressing the recombinant receptor and is to be produced from cells of the input composition;
the one second attribute comprises a cell phenotype or recombinant receptor-dependent activity; and
the process comprises a lasso regression statistical learning model trained on training data comprising (i) the percentage, number, ratio and/or proportion of T cells that have the first attributes from each of a plurality of input compositions comprising T cells and (ii) the percentage, number, ratio and/or proportion of T cells that have the one second attribute from each of a plurality of therapeutic cell compositions, wherein each of the therapeutic cells compositions comprises T cells expressing the recombinant receptor and has been produced from one of the input compositions.

4. The method of claim 3, further comprising (c) determining, based on the one predicted second attribute, whether the therapeutic cell composition is predicted to have a desired attribute.

5. The method of claim 2 or claim 4, wherein:
if the therapeutic cell composition is predicted to have the desired attribute, selecting a first manufacturing process to manufacture the therapeutic cell composition from the input composition; or
if the therapeutic cell composition is predicted to not have the desired attribute, selecting a second manufacturing process to manufacture the therapeutic cell composition from the input composition, optionally wherein the second manufacturing process is associated with producing a therapeutic cell composition that has the desired attribute.

6. The method of claim 5, wherein the second manufacturing process comprises one or more steps that are altered compared to steps of the first manufacturing process.

7. The method of claim 2 or claim 4, wherein:
if the therapeutic cell composition is predicted to have the desired attribute, a predetermined treatment regimen comprising the therapeutic cell composition is selected to be administered to the subject; or
if the therapeutic cell composition is predicted to not have the desired attribute, an altered treatment regimen comprising the therapeutic cell composition is selected to be administered to the subject, wherein the altered treatment regimen is altered compared to the predetermined treatment regimen.

8. The method of any of claims 1-7, wherein the first attributes comprises T cell phenotypes that are phenotypes positive or negative for CCR7, CD27, CD28, CD45RA, or an apoptotic marker.

9. The method of any of claims 1-8, wherein:
the T cell phenotype(s) of the second attributes are phenotypes positive or negative for CCR7, CD27, CD28, CD45RA, an apoptotic marker, positive recombinant receptor expression (recombinant receptor+), optionally CAR+, viability, viable cell concentration, vector copy number (VCN); and/or
the recombinant receptor-dependent activity is recombinant receptor-dependent production of a cytokine or a cytotoxic activity.

10. The method of claim 8 or claim 9, wherein the apoptotic marker is activated caspase 3 (3CAS) or annexin V.

11. A method of manufacturing a therapeutic cell composition, the method comprising:
(a) selecting T cells from a biological sample from a subject to produce an input composition comprising T cells;
(b) determining a percentage, number, ratio, or proportion of T cells in the input composition having first attributes, wherein the first attributes comprise T cell phenotypes;
(c) applying the first attributes as input to a process configured to predict, based on the first attributes, a percentage, number, ratio, or proportion of T cells in a therapeutic cell composition that have second attributes, wherein:
the therapeutic cell composition comprises T cells expressing the recombinant receptor and is to be produced from cells of the input composition;
the second attributes comprise T cell phenotypes and recombinant receptor-dependent activity; and
the process comprises a canonical correlation analysis statistical learning model trained on training data comprising (i) the percentage, number, ratio and/or proportion of T cells that have the first attributes from each of a plurality of input compositions comprising T cells and (ii) the percentage, number, ratio and/or proportion of T cells that have the second attributes from each of a plurality of therapeutic cell compositions, wherein each of the therapeutic cells compositions comprises T cells expressing the recombinant receptor and has been produced from one of the input compositions;
(d) determining, based on the predicted second attributes, whether the T cells of the therapeutic cell composition will have a desired attribute; and
(e) manufacturing the therapeutic cell composition, wherein:
(i) if the therapeutic cell composition is predicted to have the desired attribute, the therapeutic cell composition is manufactured from the input composition using a first manufacturing process; or
(ii) if the therapeutic cell composition is predicted to not have the desired attribute, selecting a second manufacturing process to manufacture the therapeutic cell composition from the input composition, optionally wherein the second manufacturing process is associated with producing a therapeutic cell composition that has the desired attribute.

12. A method of manufacturing a therapeutic cell composition, the method comprising:
(a) selecting T cells from a biological sample from a subject to produce an input composition comprising T cells;
(b) determining a percentage, number, ratio, or proportion of T cells in the input composition having first attributes, wherein the first attributes comprise T cell phenotypes;
(c) applying the first attributes as input to a process configured to predict, based on the first attributes, a percentage, number, ratio, or proportion of T cells in a therapeutic cell composition that have one second attribute, wherein:
the therapeutic cell composition comprises T cells expressing the recombinant receptor and is to be produced from cells of the input composition;
the one second attributes comprise T cell phenotypes and recombinant receptor-dependent activity; and
the process comprises a lasso regression statistical learning model trained on training data comprising (i) the percentage, number, ratio and/or proportion of T cells that have the first attributes from each of a plurality of input compositions comprising T cells and (ii) the percentage, number, ratio and/or proportion of T cells that have the one second attribute from each of a plurality of therapeutic cell compositions, wherein each of the therapeutic cells compositions comprises T cells expressing the recombinant receptor and has been produced from one of the input compositions;
(d) determining, based on the predicted one second attribute, whether the T cells of the therapeutic cell composition will have a desired attribute; and
(e) manufacturing the therapeutic cell composition, wherein:
(i) if the therapeutic cell composition is predicted to have the desired attribute, the therapeutic cell composition is manufactured from the input composition using a first manufacturing process; or
(ii) if the therapeutic cell composition is predicted to not have the desired attribute, selecting a second manufacturing process to manufacture the therapeutic cell composition from the input composition, optionally wherein the second manufacturing process is associated with producing a therapeutic cell composition that has the desired attribute.

13. The method of claim 11 or claim 12, wherein the second manufacturing process comprises one or more steps that are altered compared to steps of the first manufacturing process.

14. The method of any of claims 1, 2, 5-11, and 13, wherein the CCA comprises a penalty function capable of regularizing the first and second attributes.

15. The method of claim 14, wherein the penalty function comprises a constant, said constant determined by performing permutations on the first and second attributes, independently, and performing CCA.

16. The method of claim 14 or claim 15, wherein the penalty function is lasso regularization.

17. The method of any of claims 1, 2, 5-11, and 13-16, wherein the CCA further comprises constraining the square of the L2 norm of canonical vectors to be less than or equal to one.

18. The method of any of claims 1, 2, 5-11, and 13-17, wherein:
the input composition comprises CD4+, CD8+, or CD4+ and CD8+ T cells, and the therapeutic cell composition comprises CD4+ and/or CD8+ T cells expressing the recombinant receptor and is to be produced from the input composition; and
the first attributes comprise first attributes from the input composition, and the second attributes are predicted for the therapeutic cell composition from the first attributes.

19. The method of any of claims 1, 2, 5-11, and 13-17, wherein:
the input composition comprises separate compositions of CD4+ and CD8+ T cells, and the therapeutic cell composition comprises separate compositions of CD4+ and CD8+ T cells expressing the recombinant receptor and is to be produced from the respective CD4+ or CD8+ T cell composition of the input composition; and
the first attributes comprise first attributes from the CD4+ and CD8+ T cell compositions of the input composition, and the second attributes are predicted for CD4+ and CD8+ T cells of each of the separate CD4+ and CD8+ T cell compositions of the therapeutic cell composition from the first attributes.

20. The method of any of claims 1, 2, 5-11, and 13-17, wherein:
the input composition comprises separate compositions of CD4+ and CD8+ T cells, and the therapeutic cell composition comprises a mixed composition of CD4+ and CD8+ T cells expressing the recombinant receptor and is to be produced from the CD4+ and CD8+ T cell compositions of the input composition; and
the first attributes comprise first attributes from the CD4+ and CD8+ T cell compositions of the input composition, and the second attributes are predicted for CD4+ and CD8+ cells of each of the separate CD4+ and CD8+ T cell compositions of the therapeutic cell composition from the first attributes.

21. The method of any of claims 3-10, 12, and 13, wherein:
the input composition comprises CD4+, CD8+, or CD4+ and CD8+ T cells, and the therapeutic cell composition comprises CD4+ and/or CD8+ T cells expressing the recombinant receptor and is to be produced from the input composition; and
the first attributes comprise first attributes from the input composition, and the one second attribute is predicted for the therapeutic cell composition from the first attributes.

22. The method of any of claims 3-10, 12, and 13, wherein:
the input composition comprises separate compositions of CD4+ and CD8+ T cells, and the therapeutic cell composition comprises separate compositions of CD4+ and CD8+ T cells expressing the recombinant receptor and is to be produced from the respective CD4+ or CD8+ T cell composition of the input composition; and
the first attributes comprise first attributes from the CD4+ and CD8+ T cell compositions of the input composition, and the one second attribute is predicted for CD4+ or CD8+ T cells of the separate CD4+ and CD8+ T cell compositions of the therapeutic cell composition from the first attributes.

23. The method of any of claims 3-10, 12, and 13, wherein:
the input composition comprises separate compositions of CD4+ and CD8+ T cells and the therapeutic cell composition comprises a mixed composition of CD4+ and CD8+ T cells expressing the recombinant receptor, and is to be produced from the respective CD4+ and CD8+ T cell compositions of the input composition; and
the first attributes comprise first attributes from the CD4+ and CD8+ T cell compositions of the input composition, and the one second attribute is predicted for CD4+ or CD8+ T cells of the separate CD4+ or CD8+ composition of the therapeutic cell composition from the first attributes.

24. The method of any of claims 1, 2, 5-11, and 13-17, wherein:
each of the plurality of input compositions comprised in the training data comprises CD4+, CD8+, or CD4+ and CD8+ T cells and each of the plurality of therapeutic cell compositions comprised in the training data comprises CD4+ and/or CD8+ T cells expressing the recombinant receptor and has been produced from one of the plurality of input compositions; and
the first attributes comprise first attributes from each of the plurality of input compositions comprised in the training data, and the second attributes comprise second attributes of each of the plurality of therapeutic cell compositions comprised in the training data.

25. The method of any of claims 1, 2, 5-11, and 13-17, wherein:
each of the plurality of input compositions comprised in the training data comprises separate compositions of CD4+ and CD8+ T cells, and each of the plurality of therapeutic cell compositions comprised in the training data comprises separate compositions of CD4+ and CD8+ T cells expressing the recombinant receptor, and has been produced from the respective CD4+ or CD8+ T cell composition of one of the plurality of input compositions; and
the first attributes comprise first attributes from the CD4+ and CD8+ T cell compositions of each of the plurality of input compositions comprised in the training data, and the second attributes comprise second attributes from CD4+ and CD8+ T cells of each of the separate CD4+ and CD8+ T cell compositions of each of the plurality of therapeutic cell compositions comprised in the training data.

26. The method of any of claims 1, 2, 5-11, and 13-17, wherein:
each of the plurality of input compositions comprised in the training data comprises separate compositions of CD4+ and CD8+ T cells, and each of the plurality of therapeutic cell compositions comprised in the training data comprises a mixed composition of CD4+ and CD8+ T cells expressing the recombinant receptor, and has been produced from the respective CD4+ and CD8+ T cell compositions of one of the plurality of input compositions; and
the first attributes comprise first attributes from the CD4+ and CD8+ T cell compositions of each of the plurality of input compositions comprises in the training data, and the second attributes comprise second attributes from CD4+ and CD8+ T cells of each of the separate CD4+ and CD8+ T cell compositions of each of the plurality of therapeutic cell compositions comprised in the training data.

27. The method of any of claims 3-10, 12, and 13, wherein:
each of the plurality of input compositions comprised in the training data comprises CD4+, CD8+, or CD4+ and CD8+ T cells, and each of the plurality of therapeutic cell compositions comprised in the training data comprises CD4+ and/or CD8+ T cells expressing the recombinant receptor and has produced from one of the plurality of input compositions; and
the first attributes comprise first attributes from each of the plurality of input compositions comprised in the training data, and the one second attribute comprises one second attribute of each of the plurality of therapeutic cell compositions comprised in the training data.

28. The method any of claims 3-10, 12, and 13, wherein:
each of the plurality of input compositions of the training data comprises separate compositions of CD4+ and CD8+ T cells, and each of the plurality of therapeutic cell compositions comprised in the training data comprises separate compositions of CD4+ and CD8+ T cells expressing the recombinant receptor and has been produced from the respective CD4+ or CD8+ T cell composition of one of the plurality of input compositions; and
the first attributes comprise first attributes from the CD4+ and CD8+ T cell compositions of each of the plurality of input compositions comprised in the training data, and the one second attribute comprises one second attribute of the CD4+ or CD8+ T cells of the separate CD4+ and CD8+ T cell compositions of each of the plurality of therapeutic cell compositions comprised in the training data.

29. The method any of claims 3-10, 12, and 13, wherein:
each of the plurality of input compositions comprised in the training data comprises separate compositions of CD4+ and CD8+ T cells, and each of the plurality of therapeutic cell compositions comprised in the training data comprises a mixed composition of CD4+ and CD8+ T cells expressing the recombinant receptor, and has been produced from the respective CD4+ and CD8+ T cell compositions of one of the plurality of input compositions; and
the first attributes comprise first attributes from the CD4+ and CD8+ T cell compositions of each of the plurality of input compositions comprised in the training data, and the one second attribute comprises one second attribute from CD4+ or CD8+ T cells of the separate CD4+ or CD8+ T cell compositions of each of the plurality of therapeutic cell compositions comprised in the training data.

30. The method of any of claims 1-10 and 14-29, wherein the method further comprises prior to (a) selecting T cells from the biological sample from the subject to produce the input composition, the input composition comprising CD4, CD8, or CD4 and CD8 T cells.

31. The method of any of claims 1-30, wherein the first attributes comprise one or more T cell phenotypes comprising 3CAS−/CCR7−/CD27−, 3CAS−/CCR7−/CD27+, 3CAS−/CCR7+, 3CAS−/CCR7+/CD27−, 3CAS−/CCR7+/CD27+, 3CAS−/CD27+, 3CAS−/CD28−/CD27−, 3CAS−/CD28−/CD27+, 3CAS−/CD28+, 3CAS−/CD28+/CD27−, 3CAS−/CD28+/CD27+, 3CAS−/CCR7−/CD45RA−, 3CAS−/CCR7−/CD45RA+, 3CAS−/CCR7+/CD45RA−, 3CAS−/CCR7+/CD45RA+, CAS+, and CAS+/CD3+.

32. The method of any of claims 1-31, wherein the first attributes comprise one or more T cell phenotypes comprising 3CAS−/CCR7−/CD27−/CD4+, 3CAS−/CCR7−/CD27+/CD4+, 3CAS−/CCR7+/CD4+, 3CAS−/CCR7+/CD27−/CD4+, 3CAS−/CCR7+/CD27+/CD4+, 3CAS−/CD27+/CD4+, 3CAS−/CD28−/CD27−/CD4+, 3CAS−/CD28−/CD27+/CD4+, 3CAS−/CD28+/CD4+, 3CAS−/CD28+/CD27−/CD4+, 3CAS−/CD28+/CD27+/CD4+, 3CAS−/CCR7−/CD45RA−/CD4+, 3CAS−/CCR7−/CD45RA+/CD4+, 3CAS−/CCR7+/CD45RA−/CD4+, 3CAS−/CCR7+/CD45RA+/CD4+, 3CAS−/CCR7−/CD27−/CD8+, 3CAS−/CCR7−/CD27+/CD8+, 3CAS−/CCR7+/CD8+, 3CAS−/CCR7+/CD27−/CD8+, 3CAS−/CCR7+/CD27+/CD8+, 3CAS−/CD27+/CD8+, 3CAS−/CD28−/CD27−/CD8+, 3CAS−/CD28−/CD27+/CD8+, 3CAS−/CD28+/CD8+, 3CAS−/CD28+/CD27−/CD8+, 3CAS−/CD28+/CD27+/CD8+, 3CAS−/CCR7−/CD45RA−/CD8+, 3CAS−/CCR7−/CD45RA+/CD8+, 3CAS−/CCR7+/CD45RA−/CD8+, 3CAS−/CCR7+/CD45RA+/CD8+, CAS+/CD4+, CAS+/CD8+, CAS+/CD3+ of an input composition that is CD4+ cells, and CAS+/CD3+ of an input composition that is CD8+ cells.

33. The method of any of claims 1-32, wherein the first attributes comprise one or more T cell phenotypes comprising CD4+/CCR7+/CD27+, CD4+/CCR7+/CD45RA+, CD4+/CD28+/CD27−, CD8+/CCR7+/CD45RA−, CD8+/CCR7+/CD45RA+, CD8+/CCR7+, CD4+/CCR7−/CD27−, CD8+/CCR7−/CD45RA+, CD4+/CD28+/CD27−, CD4+/CD28+, and CD28+/CD27−.

34. The method of any of claims 1-33, wherein the first attributes comprise one or more T cell phenotypes comprising CD4+/CCR7+/CD27+, CD4+/CCR7+/CD45RA+, CD4+/CD28+/CD27−, CD8+/CCR7+CD45RA−, and CD8+/CCR7+CD45RA+.

35. The method of any of claims 1-34, wherein the first attributes comprise one or more T cell phenotypes comprising CD8+/CCR7+, CD4+/CCR7−/CD27−, CD8+/CCR7−/CD45RA+, and CD4+/CD28+.

36. The method of any of claims 1-35, wherein the first attributes comprise or are CD4+/CCR7+/CD45RA+.

37. The method of any of claims 1-36, wherein the second attributes comprise one or more T cell phenotypes and/or recombinant receptor-dependent activity, wherein the T cell phenotypes and recombinant receptor-dependent activity comprise 3CAS−/CCR7−/CD27−/CAR+, 3CAS−/CCR7−/CD27+/CAR+, 3CAS−/CCR7+/CAR+, 3CAS−/CCR7+/CD27−/CAR+, 3CAS−/CCR7+/CD27+/CAR+, 3CAS−/CD27+/CAR+, 3CAS−/CD28−/CD27−/CAR+, 3CAS−/CD28−/CD27+/CAR+, 3CAS−/CD28+/CAR+, 3CAS−/CD28+/CD27−/CAR+, 3CAS−/CD28+/CD27+/CAR+, 3CAS−/CCR7−/CD45RA−/CAR+, 3CAS−/CCR7−/CD45RA+/CAR+, 3CAS−/CCR7+/CD45RA−/CAR+, 3CAS−/CCR7+/CD45RA+/CAR+, CAS+ of CD3+/CAR+, CD3+, CYTO−/CAR+, EGFRt+, IFNG+, viable cell concentration (VCC), vector copy number (VCN), viability, CD3+/CAR+, CD3+/CD56+, CAR+, IFNG+/IL-2+/CAR+, IFNG+/IL-2+/IL17+/TNFA+/CAR+, IFNG+/IL-2+/TNFA/+CAR+, IFNG+ of CAR+, IFNG+/TNFA/+CAR+, IL13+ of CAR+, IL17+ of CAR+, IL2+ of CAR+, IL-2+/TNFA+/CAR+, TNFA+ of CAR+, cytolytic CD8+, GMCSF+, IFNG+, IL10+, IL13+, IL2+, IL5+, MIP1A+, MIP1B+, sCD137+, and TNFa+.

38. The method of any of claims 1-37, wherein the second attributes comprise one or more cell phenotypes and/or recombinant receptor-dependent activity, wherein the T cell phenotypes and recombinant receptor-dependent activity comprise 3CAS−/CCR7−/CD27−/CD4+/CAR+, 3CAS−/CCR7−/CD27+/CD4+/CAR+, 3CAS−/CCR7+/CD4+/CAR+, 3CAS−/CCR7+/CD27−/CD4+/CAR+, 3CAS−/CCR7+/CD27+/CD4+/CAR+, 3CAS−/CD27+/CD4+/CAR+, 3CAS−/CD28−/CD27−/CD4+/CAR+, 3CAS−/CD28−/CD27+/CD4+/CAR+, 3CAS−/CD28+/CD4+/CAR+, 3CAS−/CD28+/CD27−/CD4+/CAR+, 3CAS−/CD28+/CD27+/CD4+/CAR+, 3CAS−/CCR7−/CD45RA−/CD4+/CAR+, 3CAS−/CCR7−/CD45RA+/CD4+/CAR+, 3CAS−/CCR7+/CD45RA−/CD4+/CAR+, 3CAS−/CCR7+/CD45RA+/CD4+/CAR+, CAS+ of CD3+/CAR+, CD3+, CD3+/CD4+, CD4+/EGFRt+, CYTO−/CD4+/CAR+, EGFRt+, IFNG+, VCC, VCN, viability, CD3+/CAR+, CD3+/CD56+, CD4+/CAR+, 3CAS−/CCR7−/CD27−/CD8+/CAR+, 3CAS−/CCR7−/CD27+/CD8+/CAR+, 3CAS−/CCR7+/CD8+/CAR+, 3CAS−/CCR7+/CD27−/CD8+/CAR+, 3CAS−/CCR7+/CD27+/CD8+/CAR+, 3CAS−/CD27+/CD8+/CAR+, 3CAS−/CD28−/CD27−/CD8+/CAR+, 3CAS−/CD28−/CD27+/CD8+/CAR+, 3CAS−/CD28+/CD8+/CAR+, 3CAS−/CD28+/CD27−/CD8+/CAR+, 3CAS−/CD28+/CD27+/CD8+/CAR+, 3CAS−/CCR7−/CD45RA−/CD8+/CAR+, 3CAS−/CCR7−/CD45RA+/CD8+/CAR+, 3CAS−/CCR7+/CD45RA−/CD8+/CAR+, 3CAS−/CCR7+/CD45RA+/CD8+/CAR+, CAS+ of CD3+/CAR+, CD3+, CD3+/CD8+, CD8+/EGFRt+, CYTO−/CD8+/CAR+, EGFRt+, IFNG+, VCC, VCN, viability, GMCSF+/CD19+, CD3+/CAR+, CD3+/CD56+, CD8+/CAR+, IFNG+/IL-2+/CD4+/CAR+, IFNG+/IL-2+/IL-17+/TNFA+/CD4+/CAR+, IFNG+/IL-2+/TNFA+/CD4+/CAR+, IFNG+ OF CD4+/CAR+, IFNG+/TNFA+/CD4+/CAR+, IL-13+ of CD4+/CAR+, IL-17+ of CD4+CAR+, IL-2+ of CD4+CAR+, IL-2+/TNFA+/CD4+/CAR+, TNFA+ of CD4+/CAR+, IFNG+/IL-2+/CD8+/CAR+, IFNG+/IL-2+/IL-17+/TNFA+/CD8+/CAR+, IFNG+/IL-2+/TNFA+/CD8+/CAR+, IFNG+ of CD8+/CAR+, IFNG+/TNFA+/CD8+/CAR+, IL-13+ of CD8+/CAR+, IL-17+ of CD8+/CAR+, IL-2+ of CD8+/CAR+, IL-2+/TNFA+/CD8+/CAR+, cytolytic CD8+, TNFA+ of CD8+CAR+, GMCSF+, IFNG+, IL10+, IL13+, IL2+, IL5+, MIP1A+, MIP1B+, sCD137+, and TNFa+.

39. The method of any of claims 1-38, wherein the second attributes comprise one or more T cell phenotypes and/or recombinant receptor-dependent activity, wherein the T cell phenotypes and recombinant receptor-dependent activity comprise CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, CCR7+/CD45RA+/CD4+/CAR+, CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, and CCR7+/CD45RA+/CD8+/CAR+.

40. The method of any of claims 1-39, wherein the second attributes comprise one or more T cell phenotypes and/or recombinant receptor-dependent activity, wherein the T cell phenotypes and recombinant receptor-dependent activity comprise CCR7−/CD27−/CD4+/CAR+, CD28+/CD27−/CD4+/CAR+, CD27+/CD4+/CAR+, CD28+/CD27+/CD4+/CAR+, CCR7+/CD4+/CAR+, CCR7+/CD27+CD4+/CAR+, CCR7−/CD45RA+/CD4+/CAR+, and CCR7+/CD45RA+/CD4+/CAR+.

41. The method of any of claims 1-40, wherein the second attributes comprise one or more T cell phenotypes and/or recombinant receptor-dependent activity, wherein the T cell phenotypes and recombinant receptor-dependent activity comprise CD28+/CD27−/CD8+/CAR+, CD27+/CD8+/CAR+, CD28+/CD27+/CD8+/CAR+, CCR7+/CD8+/CAR+, CCR7−/CD27−/CD8+/CAR+, CCR7−/CD45RA−/CD8+/CAR+, and CCR7+/CD45RA+/CD8+/CAR+.

42. The method of any of claims 1-41 wherein the first attributes comprise or comprise about or at least 2, 4, 6, 8, 10, 12, or more T cell phenotypes.

43. The method of any of claims 1-42, wherein the first attributes comprise greater than or greater than about 5, 10, 15, or 20 T cell phenotypes.

44. The method of any of claims 1-43, wherein the first attributes comprise or comprise about 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 T cell phenotypes.

45. The method of any of claims 1, 2, 5-11, 13-20, 24-26, and 30-44, wherein the second attributes comprise about or at least 1, 2, 4, 6, 8, 10, 12, or more T cell phenotypes and recombinant receptor-dependent activity.

46. The method of any of claims 1, 2, 5-11, 13-20, 24-26, and 30-45, wherein the second attributes comprise about or at least 15, 20, 30, 40, 50, 60, 70, 80, 90, or more T cell phenotypes and recombinant receptor-dependent activity.

47. The method of any of claims 1, 2, 5-11, 13-20, 24-26, and 30-46, wherein the second attributes comprise or comprise about 101, 90, 80, 70, 60, 50, 40, 30, 20, 15, 10, 5, 4, 3, 2, or 1 T cell phenotypes and recombinant receptor-dependent activity, or any value between any of the foregoing.

48. The method of any of claims 1-47, wherein the second attributes comprise one (1) T cell phenotype or recombinant receptor-dependent activity.

49. The method of any of claims 2 and 5-48, wherein the desired attribute is at least one attribute that is correlated with a positive clinical response to treatment with the therapeutic cell composition.

50. The method of any of claims 4-48, wherein the desired attribute is an attribute that is correlated with a positive clinical response to treatment with the therapeutic cell composition.

51. The method of claim 49 or claim 50, wherein the positive clinical response is a durable response and/or progression free survival.

52. The method of any of claims 2 and 4-51, wherein the desired attribute is or comprises a threshold percentage of naïve-like T cells or central memory T cells.

53. The method of claim 52, wherein the threshold percentage is at least or at least about 40% of the cells in the therapeutic cell composition that are naïve-like T cells or central memory T cells.

54. The method of claim 52, wherein the threshold percentage is at least or at least about 50% of the cells in the therapeutic cell composition that are naïve-like T cells or central memory T cells.

55. The method of claim 52, wherein the threshold percentage is at least or at least about 60% of the cells in the therapeutic cell composition that are naïve-like T cells or central memory T cells.

56. The method of claim 52, wherein the threshold percentage is at least or at least about 65% of the cells in the therapeutic cell composition that are naïve-like T cells or central memory T cells.

57. The method of claim 52, wherein the threshold percentage is at least or at least about 70% of the cells in the therapeutic cell composition that are naïve-like T cells or central memory T cells.

58. The method of any of claims 52-57, wherein the naïve-like T cells or central memory T cells have a phenotype comprising T cells surface positive for CD27+, CD28+, CD62L+, and/or CCR7+.

59. The method of any of claims 52-58, wherein the naïve-like T cells or central memory T cells have the phenotype CD62L+/CCR7+, CD27+/CCR7+, CD62L+/CD45RA−, CCR7+/CD45RA−, CD62L+/CCR7+/CD45RA−, CD27+/CD28+/CD62L+/CD45RA−, CD27+/CD28+/CCR7+/CD45RA−, CD27+/CD28+/CD62L+/CCR7+, or CD27+/CD28+/CD62L+/CCR7+/CD45RA−.

60. The method of any of claims 2 and 4-59, wherein the desired attribute is a threshold percentage of the phenotype CD27+/CCR7+ T cells in the therapeutic cell composition.

61. The method of claim 60, wherein the threshold percentage is at least or at least about 60% of the cells in the therapeutic cell composition are CD27+/CCR7+.

62. The method of claim 60 or claim 61, wherein the CD27+/CCR7+ cells are CD4+/CAR+ T cells and CD8+/CAR+ T cells.

63. The method of claim 60 or claim 61, wherein the CD27+/CCR7+ cells are CD4+/CAR+ T cells.

64. The method of claim 60 or claim 61, wherein the CD27+/CCR7+ cells are CD8+/CAR+ T cells.

65. The method of any of claims 2 and 4-51, wherein the desired attribute is or comprises a threshold percentage of IL-2+ of CD4+/CAR+ and IL-2+/TNFA+/CD4+/CAR+ T cells in the therapeutic cell composition.

66. The method of claim 65, wherein the threshold percentage is at least at or about 70%, 80%, 85%, 90%, 91%, 92%, 93%, 94%, 95%, 96%, 97%, 98%, 99% or 100% of the total number of CD4+ T cells in the therapeutic cell composition.

67. The method of any of claims 2 and 4-51, wherein the desired attribute is a threshold percentage of IL-2+ of CD8+/CAR+ and IL-2+/TNFA+/CD8+/CAR+ T cells in the therapeutic cell composition.

68. The method of claim 67, wherein the threshold percentage is at least at or about 70%, 80%, 85%, 90%, 91%, 92%, 93%, 94%, 95%, 96%, 97%, 98%, 99% or 100% of the total number of CD8+ T cells in the therapeutic cell composition.

69. The method of any of claims 2 and 4-51, wherein the desired attribute is or comprises a threshold percentage of IFNG+/IL-2+/CD4+/CAR+, IFNG+/IL-2+/IL-17+/TNFA+/CD4+/CAR+, IFNG+/IL-2+/TNFA+/CD4+/CAR+, IFNG+/TNFA+/CD4+/CAR+, IL-17+ of CD4+CAR+, IL-2+ of CD4+CAR+, and/or IL-2+/TNFA+/CD4+/CAR+ T cells in the therapeutic cell composition.

70. The method of claim 69, wherein the threshold percentage is at least at or about 10%, 15%, 20%, 25%, 30%, 40%, 50%, 60% or more of the total number of CAR+/CD4+ T cells in the therapeutic cell composition.

71. The method of any of claims 2 and 4-51, wherein the desired attribute is or comprises a threshold percentage of IFNG+/IL-2+/CD8+/CAR+, IFNG+/IL-2+/IL-17+/TNFA+/CD8+/CAR+, IFNG+/IL-2+/TNFA+/CD8+/CAR+, IFNG+/TNFA+/CD8+/CAR+, IL-17+ of CD8+CAR+, IL-2+ of CD8+CAR+, and/or IL-2+/TNFA+/CD8+/CAR+ T cells in the therapeutic cell composition.

72. The method of claim 71, wherein the threshold percentage is at least at or about 10%, 15%, 20%, 25%, 30%, 40%, 50%, 60% or more of the total number of CAR+/CD8+ T cells in the therapeutic cell composition.

73. The methods of any of claims 5, 6, and 8-72, wherein the first manufacturing process is selected from a process:
comprising a step of introducing T cells of the input composition with a nucleic acid encoding a recombinant receptor to generate an engineered T cell composition, and cultivating the engineered T cell compositions under conditions for expansion of T cells;
wherein obtaining the input composition does not comprise enriching or selecting for naïve-like T cells or T cells having a central memory phenotype from the biological sample; or
wherein obtaining the input composition does not comprise depleting T cells comprising a phenotype of a terminally differentiated T cell or cell with reduced proliferative capacity, optionally wherein the phenotype of a terminally differentiated T cell or T cell with reduced proliferative capacity is CD57+.

74. The methods of any of claims 5, 6, and 8-73, wherein the first manufacturing process is an expanded process resulting in more than 2-fold increase in cells in the therapeutic cell composition compared to the input composition, optionally more than 4-fold increase.

75. The methods of any of claims 5, 6, and 8-74, wherein the second manufacturing process is selected from a process:
comprising a step of introducing T cells of the input composition with a nucleic acid encoding a recombinant receptor to generate an engineered T cell composition, and incubating the engineered T cell composition that does not expand T cells in the composition or that minimally expands T cells in the composition
comprising obtaining the input composition by enriching or selecting for naïve-like T cells or T cells having a central memory phenotype from the biological sample;
wherein the input composition comprises a threshold number of naïve-like cells or central memory T cells; or
wherein the input composition comprises depleting T cells comprising a phenotype of a terminally differentiated T cell, optionally wherein the phenotype of a terminally differentiated T cell or T cell with reduced proliferative capacity is CD57+.

76. The methods of any of claims 5, 6, and 8-75, wherein the second manufacturing process is a non-expanded or minimally expanded process resulting in less than 2-fold more cells in the output composition compared to the input composition.

77. The method of any of claims 5, 6, and 8-75, wherein the first manufacturing process and second manufacturing process, independently, comprise:
stimulating the input cell composition with a T cell stimulatory agent(s), optionally wherein the T cell stimulatory agent(s) is or comprises an anti-CD3 antibody, an anti-CD28 antibody and one or more recombinant cytokines selected from IL-2, IL-15, IL-7 and IL-21 to produce a stimulated composition; and
introducing into cells of the stimulated composition a polynucleotide encoding the recombinant receptor, optionally wherein the introducing comprises transducing cells with a viral vector encoding the recombinant receptor.

78. The method of claim 77, wherein the first manufacturing process further comprises cultivating cells introduced with the polynucleotide under conditions for expansion of T cells in the composition.

79. The method of claim 77, wherein the second manufacturing process further comprises cultivating cells introduced with the polynucleotide under conditions for expansion of T cells in the composition.

80. The method of claim 77, wherein the second manufacturing process, does not comprise cultivating cells introduced with the polynucleotide under conditions for expansion of T cells in the composition.

81. The method of any of claims 1-80, wherein the biological sample comprises a whole blood sample, a buffy coat sample, a peripheral blood mononuclear cell (PBMC) sample, an unfractionated T cell sample, a lymphocyte sample, a white blood cell sample, an apheresis product, or a leukapheresis product.

82. The method of any of claims 1-81, wherein the biological sample is an apheresis product or leukapheresis product.

83. The method of claim 82, wherein the apheresis product or leukapheresis product has been previously cryopreserved.

84. The method of any of claims 1-83, wherein the T cells comprise primary cells obtained from the subject.

85. The method of any of claims 1-84, wherein the recombinant receptor is a chimeric antigen receptor (CAR).

86. The method of any of claims 1-85, wherein cells of the input composition are selected or enriched from a biological sample from a subject, optionally a human subject.

87. The method of any of claims 18-86, wherein the CD4+, CD8+, or CD4+ and CD8+ T cells in the input composition, or in each separate composition of the input composition, are enriched from a biological sample, optionally wherein the enriched composition comprises at or great than about 85%, 86%, 87%, 88%, 89%, 90%, 91%, 92%, 93%, 94%, 95%, 96%, 97%, 98%, or 99% or more of the respective CD4+, CD8+, or CD4+ and CD8+ T cells.
